(19) United States
(12) Patent Application Publication
(10) Pub. No.: US 2024/0352131 A1
(43) Pub. Date: Oct. 24, 2024

(54) METHODS OF TREATING EYE DISEASE

(71) Applicant: TrueBinding, Inc., Foster City, CA (US)

(72) Inventors: Dongxu Sun, Palo Alto, CA (US); Suhail Rasool, Foster City, CA (US); Barbara Rangel da Silva, Belmont, CA (US)

(21) Appl. No.: 18/637,068

(22) Filed: Apr. 16, 2024

Related U.S. Application Data
(60) Provisional application No. 63/496,917, filed on Apr. 18, 2023.

(51) Int. Cl. C07K 16/28; A61K 9/00; A61K 39/00; A61P 27/02; A61P 27/12

(52) U.S. Cl. CPC C07K 16/2851; A61K 9/0048; A61P 27/02; A61P 27/12; A61K 2039/505; C07K 2317/565

(57) ABSTRACT

Provided herein are anti-Gal3 antibodies and methods of use thereof. Such methods include therapies for treating one or more eye diseases including, but not limited to, dry eye disease, glaucoma, age-related macular degeneration, microvascular dysfunction, cataracts, retinal drusen, tear dysfunction, corneal sensitivity, diabetic retinopathy, ocular surface diseases, and other eye diseases.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### REFERENCE TO SEQUENCE LISTING

The present application is being filed along with a Sequence Listing in electronic format. The Sequence Listing is provided as a file entitled IMMUT043ASEQLIST.XML, which was created and last modified on Apr. 9, 2024, which is 2,982,948 bytes in size. The information in the electronic Sequence Listing is hereby incorporated by reference in its entirety.

### FIELD

Aspects of the present disclosure relate generally to therapies related to eye disease involving the use of antibodies that bind to Gal-3.

### BACKGROUND

Galectin-3 (Gal3, GAL3) is a lectin, or a carbohydrate-binding protein, with specificity towards beta-galactosides. In human cells, Gal3 is expressed and can be found in the nucleus, cytoplasm, cell surface, and in the extracellular space. Gal3 recognizes and interacts with beta-galactose conjugates on various proteins.

### SUMMARY

Some embodiments provided herein are described by way of the following provided embodiments and also provided as possible combinations or overlapping embodiments:

In some embodiments, a method of treating microglia mediated inflammation in a subject in need thereof is disclosed. In some embodiments, the method comprises: identifying a subject as having or at risk of having microglia mediated inflammation, and administering a therapeutically effective amount of an anti-Gal3 antibody to the subject, wherein administration of the antibody to the subject alleviates one or more symptoms of microglia mediated inflammation.

In some embodiments, a method of treating one or more diseases associated with microglia mediated inflammation in a subject in need thereof id disclosed. In some embodiments, the method comprises: identifying a subject as having or at risk of having one or more diseases associated with microglia mediated inflammation, and administering a therapeutically effective amount of an anti-Gal3 antibody to the subject, wherein administration of the antibody to the subject's eye alleviates one or more symptoms of a disease microglia mediated inflammation, is provided.

In some embodiments, a method of treating an ocular surface disease in a subject in need thereof is disclosed. In some embodiments, the method comprises: identifying a subject as having or at risk of having an ocular surface disease, and administering a therapeutically effective amount of an anti-Gal3 antibody to the subject, wherein administration of the antibody to the subject alleviates one or more symptoms of ocular surface disease.

In some embodiments, a method of treating macular degeneration in a subject in need thereof is disclosed. In some embodiments, the method comprises: identifying a subject as having or at risk of having macular degeneration, and administering a therapeutically effective amount of an anti-Gal3 antibody to the subject, wherein administration of the antibody to the subject alleviates one or more symptoms of macular degeneration, is provided.

In some embodiments, a method of treating dry eye disease in a subject in need thereof is disclosed. In some embodiments, the method comprises: identifying a subject as having or at risk of having dry eye disease, and administering a therapeutically effective amount of an anti-Gal3 antibody to the subject, wherein administration of the antibody to the subject alleviates one or more symptoms of dry eye disease, is provided.

In some embodiments, a method of age-related vascular dysfunction in a subject in need thereof is disclosed. In some embodiments, the method comprises: identifying a subject as having or at risk of having age-related vascular dysfunction, and administering a therapeutically effective amount of an anti-Gal3 antibody to the subject, wherein administration of the antibody to the subject alleviates one or more symptoms of age-related vascular dysfunction, is provided.

In some embodiments, a method of increasing tear formation in a subject in need thereof is disclosed. In some embodiments, the method comprises: identifying a subject as likely to benefit from increased tear production, administering a therapeutically effective amount of an anti-Gal3 antibody to the subject, wherein administration of the anti-Gal3 antibody to the subject increases tear production in the subject as compared to tear production in the subject prior to administration of the antibody, is provided.

In some embodiments, a method of decreasing corneal sensitivity in a subject is disclosed. In some embodiments, the method comprises: administering a therapeutically effective amount of an anti-Gal3 antibody to the subject, wherein administration of the anti-Gal3 antibody to the subject's eye decreases corneal sensitivity in the subject as corneal sensitivity in the subject prior to administration of the antibody, is provided.

In some embodiments, a method of treating cataracts in a subject in need thereof is disclosed. In some embodiments, the method comprises: identifying a subject as having or at risk of having cataracts, and administering a therapeutically effective amount of an anti-Gal3 antibody to the subject, wherein administration of the antibody to the subject reduces the severity of the subject's cataracts as compared to the severity of cataracts in the subjects prior to administration of the anti-Gal3 antibody, is provided.

In some embodiments, a method of preventing or reducing cataract formation in a subject in need thereof is disclosed. In some embodiments, the method comprises: identifying a subject as having or at risk of having cataracts, and administering a therapeutically effective amount of an anti-Gal3 antibody to the subject, wherein administration of the antibody to the subject reduces or prevents cataract formation in the subject acts as compared to formation of cataracts in the subjects prior to administration of the anti-Gal3 antibody, is provided.

In some embodiments, a method of treating diabetic retinopathy in a subject in need thereof is disclosed. In some embodiments, the method comprises: identifying a subject as having or at risk of having diabetic retinopathy, and administering a therapeutically effective amount of an anti-Gal3 antibody to the subject, wherein administration of the antibody to the subject alleviates one or more symptoms of diabetic retinopathy, is provided.

In some embodiments, a method of treating retinal drusen in a subject in need thereof is disclosed. In some embodiments, the method comprises: identifying a subject as having or at risk of having retinal drusen, and administering a therapeutically effective amount of an anti-Gal3 antibody to the subject, wherein administration of the antibody to the subject reduces retinal drusen in the subject as compared to retinal drusen in the subject prior to administration of the anti-Gal3 antibody, is provided.

### BRIEF DESCRIPTION OF THE DRAWINGS

FIG. 1 is a flowchart depicting some embodiments of a method of treating microglia mediated inflammation.

FIG. 2 is a flowchart depicting some embodiments of a method of treating one or more diseases associated with microglia mediated inflammation.

FIG. 3 is a flowchart depicting some embodiments of a method of treating an ocular surface disease.

FIG. 4 is a flowchart depicting some embodiments of a method of treating macular degeneration.

FIG. 5 is a flowchart depicting some embodiments of a method of treating dry eye disease (DED).

FIG. 6 is a flowchart depicting some embodiments of a method of treating age-related vascular dysfunction.

FIG. 7 is a flowchart depicting some embodiments of a method of increasing tear formation.

FIG. 8 is a flowchart depicting some embodiments of a method of decreasing corneal sensitivity.

FIG. 9 is a flowchart depicting some embodiments of a method of treating cataracts.

FIG. 10 is a flowchart depicting some embodiments of a method of preventing or reducing cataract formation.

FIG. 11 is a flowchart depicting some embodiments of a method of treating diabetic retinopathy.

FIG. 12 is a flowchart depicting some embodiments of a method of treating retinal drusen.

FIG. 13 is a representation of some embodiments of an anti-Gal3 dosing procedure for treatment of dry eye disease (DED).

FIG. 14 shows images of some embodiments of palpebral opening in mice with normal eyes and mice with dry eye disease (DED).

FIG. 15 is a graph quantifying palpebral opening in some embodiments of mice with normal and mice with dry eye disease (DED).

FIG. 16 is a graph depicting the results of a Von Frey filament test on some embodiments of mice with normal eyes and mice with dry eye disease treated with or without anti-Gal3 antibody.

FIG. 17 is a graph depicting the results of a Von Frey filament test on some embodiments of mice with normal eyes and mice with dry eye disease treated with or without anti-Gal3 antibody.

FIG. 18 is an illustration depicting some embodiments of a Schirmer's test for measuring tear production.

FIG. 19 is a pair of graphs quantifying tear production in female (top) and male (bottom) mice with dry eye disease following treatment with or without anti-Gal3 antibody.

FIG. 20 is a pair of graphs depicting quantification of Gal-3 concentration in the tears of mice with normal eyes and mice with dry eye disease with or without anti-Gal3 antibody administration.

FIG. 21 is a pair of graphs depicting the body weight of female (top) and male (bottom) mice treated with or without anti-Gal3 antibodies.

FIG. 22 shows images of some embodiments of cataract staging in mice with normal eyes and mice with dry eye disease (DED).

FIG. 23 is a pair of graphs depicting cataract staging in some embodiments of mice treated with or without anti-Gal3 antibody.

FIG. 24 depicts protein sequences of Gal3 and exemplary proteins that exhibit pathogenic aggregation.

FIG. 25 depicts peptide sequences of Gal3 used to generate and analyze antibodies.

FIG. 26A depicts exemplary variable heavy chain complementarity-determining region (CDR) 1 for anti-Gal3 antibodies disclosed herein. In some embodiments, any of the compositions or methods provided herein can include one or more of the variable heavy chain CDR1 provided herein.

FIG. 26B depicts exemplary variable heavy chain CDR2 for anti-Gal3 antibodies disclosed herein. In some embodiments, any of the compositions or methods provided herein can include one or more of the variable heavy chain CDR2 provided herein.

FIG. 26C depicts exemplary variable heavy chain CDR3 for anti-Gal3 antibodies disclosed herein. In some embodiments, any of the compositions or methods provided herein can include one or more of the variable heavy chain CDR3 provided herein.

FIG. 27A depicts exemplary variable light chain CDR1 for anti-Gal3 antibodies disclosed herein. In some embodiments, any of the compositions or methods provided herein can include one or more of the variable light chain CDR1 provided herein.

FIG. 27B depicts exemplary variable light chain CDR2 for anti-Gal3 antibodies disclosed herein. In some embodiments, any of the compositions or methods provided herein can include one or more of the variable light chain CDR2 provided herein.

FIG. 27C depicts exemplary variable light chain CDR3 for anti-Gal3 antibodies disclosed herein. In some embodiments, any of the compositions or methods provided herein can include one or more of the variable light chain CDR3 provided herein.

FIG. 28 depicts exemplary heavy chain variable region (VH) sequences for anti-Gal3 antibodies disclosed herein. In some embodiments, any of the compositions or methods provided herein can include one or more of the VH sequences provided herein.

FIG. 29 depicts exemplary light chain variable region (VL) sequences for anti-Gal3 antibodies disclosed herein. In some embodiments, any of the compositions or methods provided herein can include one or more of the VL sequences provided herein.

FIG. 30 depicts exemplary combinations of heavy and light chain CDRs (CDR1, CDR2, and CDR3) of exemplary anti-Gal3 antibodies disclosed herein. In some embodiments, any of the compositions or methods provided herein can include one or more of the heavy and light chain CDR combinations provided herein.

FIG. 31 depicts exemplary combinations of heavy and light chain variable regions of exemplary anti-Gal3 antibodies disclosed herein. In some embodiments, any of the compositions or methods provided herein can include one or more of the heavy and light chain variable region combinations provided herein.

FIG. 32 depicts exemplary heavy chain (HC) sequences and light chain (LC) sequences, and possible pairings for exemplary anti-Gal3 antibodies disclosed herein. In some embodiments, any of the compositions or methods provided herein can include one or more of the HC or LC, or pairs of HC and LC sequences provided herein.

FIG. 33 depicts antibody names used throughout the present disclosure refer to the same antibody (with exemplary peptide and nucleic acid sequences provided elsewhere in the disclosure and appropriately attributed to at least one of the depicted names) and may be used interchangeably. The names shown in a column correspond to the same antibody.

FIG. 34 depicts an alignment of hinge and constant heavy chain domain 2 (CH2) domain amino acid sequences of wild-type human immunoglobulin G1 (IgG1), IgG2 and IgG4 as well as their sigma variants. The alignment above uses EU numbering. Residues identical to wild-type IgG1 are indicated as dots; gaps are indicated with hyphens. Sequence is given explicitly if it differs from wild-type IgG1 or from the parental subtype for σ variants. Open boxes beneath the alignment correspond to International Immunogenetics Information System (IMGT) strand definitions. Boxes beneath the alignment correspond to the strand and helix secondary structure assignment for wild-type IgG1. Residues 267-273 form the BC loop and 322-332 form the FG loop. Also provided are exemplary constant regions for human IgG4 heavy (S228P mutant) and light (kappa) chains (SEQ ID NOs: 931-932) and murine IgG2A (LALAPG and LALA mutants) (SEQ ID NOs: 933-934). In some embodiments, any one or more of the VH/VL and/or CDRs provided in the other figures or otherwise disclosed herein can be paired with any one or more of the exemplary constant regions provided herein.

FIG. 35 depicts nucleic acid sequences that encode for exemplary heavy chain variable regions of anti-Gal3 antibodies disclosed herein. In some embodiments, any of the compositions or methods provided herein can include one or more of the heavy chain variable regions encoded by the nucleic acids provided herein.

FIG. 36 depicts nucleic acid sequences that encode for exemplary light chain variable regions of anti-Gal3 antibodies disclosed herein. In some embodiments, any of the compositions or methods provided herein can include one or more of the light chain variable regions encoded by the nucleic acids provided herein.

FIG. 37 depicts nucleic acid sequences that encode for exemplary heavy chains of anti-Gal3 antibodies disclosed herein. In some embodiments, any of the compositions or methods provided herein can include one or more of the heavy chains encoded by the nucleic acids provided herein.

FIG. 38 depicts nucleic acid sequences that encode for exemplary light chains of anti-Gal3 antibodies disclosed herein. In some embodiments, any of the compositions or methods provided herein can include one or more of the light chains encoded by the nucleic acids provided herein.

FIG. 39A depicts an exemplary alignment for the heavy chain CDRs (FIG. 39A) for some exemplary anti-Gal3 antibodies disclosed herein.

FIG. 39B depicts an exemplary alignment for the light chain CDRs for some exemplary anti-Gal3 antibodies disclosed herein.

FIG. 40 is a series of representative images and blots depicting some embodiments of time course aggregation of crystallin AA and crystalin AB incubated with or without Gal3.

FIG. 41 is a flowchart depicting some embodiments of a method of treating glaucoma.

### DETAILED DESCRIPTION

Ocular surface diseases including but not limited to dry eye disease (DED), age-related macular degeneration (AMD), glaucoma, and cataracts, result in symptoms of ocular discomfort, visual disturbance, and tear dysfunction, and potentially result in damage to the ocular surface. Common symptoms of ocular surface diseases include dryness of eye, foreign body sensation, burning sensation, itching, photophobia, eye redness, cloudy eyes, drusen, blurred vision, fluctuating vision, visual fatigue, loss of vision, blurry or fuzzy vision, difficulty recognizing familiar faces, straight lines appear wavy, a dark, empty area or blind spot appears in the center of vision, or loss of central vision, which is necessary for driving, reading, recognizing faces, and performing close-up work, among others. Although the pathogeneses of the various ocular pathologies are usually complex and still under intense and continuous investigation, many feature one or a combination of vascular dysfunction, angiogenesis, inflammation, and oxidative stress.

Galectin-3 levels found in tears has been associated with epithelial dysfunction in patients with DED and its concentration has significant relationship with the severity of ocular surface barrier disruption. Therefore Galectin-3 is a novel target whose antagonist antibodies can be useful therapeutic drugs for DED. Accordingly, some aspects of the present disclosure are directed to methods of treating DED and other eye diseases or conditions using anti-Gal3 antibodies or fragments thereof. In some embodiments, any of the antibodies or constructs comprising any of the antibody components having the sequences defined in any of the figures provided herein can be used in any of the methods provided herein.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The section headings used herein are for organizational purposes only and are not to be construed as limiting the subject matter described.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g., constant, or intermittent) of a health care worker (e.g., a doctor, a registered nurse, a nurse practitioner, a physician's assistant, an orderly or a hospice worker).

As used herein, the term “microglia” refers to a specialized population of macrophage-like cells in the central nervous system (CNS). Microglia are considered immune sentinels that are the primary innate immune effector cells of the CNS. Microglia are also involved in synaptic organization, trophic neuronal support during development, phagocytosis of apoptotic cells in the developing brain, myelin turnover, control of neuronal excitability, phagocytic debris removal as well as brain protection and repair.

As used herein, the term “macrophage” refers to a type of white blood cell that surrounds and kills microorganisms, removes dead cells, and stimulates the action of other immune system cells.

As used herein, the term “central nervous system” (CNS) refers to part of the nervous system that consists of the brain and spinal cord.

As used herein, the term “cataract” refers to a clouding of the normally clear lens of the eye. Cataracts may cause changes in a subject's vision. These changes may include cloudy or blurry vision, colors appearing faded, poor night vision, sensitivity to lamps, sunlight, headlights, or other light, the appearance of a halo around lights, double vision, or the need to alter eye glass or contact lens prescription often. Cataracts severity may be assessed by observing the opacity of the eye. Stage 0 cataracts refer to a normal transplant lens. Stage 1 cataracts show initial signs of nuclear opacity and little light scattering is observed with a white-light lamp. Stage 2 cataracts demonstrate little light scattering with the naked eye, and slight nuclear opacity, including swollen fibers or posterior sub-capsular scattering with a white-light lamp. Stage 3 cataracts diffuse nuclear opacity with the naked eye. Stage 4 cataracts demonstrate partial nuclear opacity with the naked eye. Stage 5 cataracts demonstrate dense opacity that does not involve the lens cortex. Stage 6 cataracts demonstrate mature dense opacity.

As used herein, the term “retinal drusen” or “drusen” refers to yellow deposits under the retina. Drusen are comprised of lipids and proteins. The presence of drusen is a biomarker for age-related macular degeneration (AMD). Drusen comprised of protein and calcium salts can also occur in the optic nerve and generally appear in both eyes. Unlike the drusen associated with AMD, optic nerve drusen or optic disc drusen are not related to aging, may be inherited, and typically appear in children. Optic nerve drusen usually do not affect vision, but some patients with these drusen may lose peripheral (side) vision.

As used herein, the term “age-related macular degeneration” (AMD) refers to a disease affecting a subject's central vision and with it, the ability to see fine details. In AMD, a part of the retina called the macula is damaged. In advanced stages, people lose their ability to drive, to see faces, and to read smaller print. In its early stages, AMD may have no signs or symptoms, so people may not suspect they have it. The two primary forms of AMD have different causes. Dry AMD is the most common form of AMD. About 80% of those with AMD have the dry form. This happens as the light-sensitive cells in the macula slowly break down, generally one eye at a time. The loss of vision in Dry AMD is usually slow and gradual. It is believed that the age-related damage of an important support membrane under the retina contributes to dry age-related macular degeneration. Wet AMD is less common; however, it usually leads to more severe vision loss in patients than dry AMD. It is the most common cause of severe loss of vision. Wet AMD happens when abnormal blood vessels start to grow beneath the retina. They leak fluid and blood and can create a large blind spot in the center of the visual field. AMD can result in severe loss of central vision but does not generally result in blindness. Risk factors for AMD include being 50 and older, smoking, having high blood pressure and eating a diet high in saturated fat. Symptoms of AMD may include blurry or fuzzy vision, difficulty recognizing familiar faces, straight lines appear wavy, a dark, empty area or blind spot appears in the center of vision, or loss of central vision, which is necessary for driving, reading, recognizing faces, and performing close-up work. The presence of drusen, which are tiny yellow deposits in the retina, is one of the most common early signs of age-related macular degeneration. It may mean the eye is at risk for developing more severe age-related macular degeneration. Diagnosis of age-related macular degeneration may comprise administration of a visual acuity test, pupil dilation, fluorescein angiography, or an Amsler Grid.

As used herein, the term “glaucoma” refers to a group of eye conditions that damage the optic nerve. The optic nerve sends visual information from the eye to the brain and is vital for good vision. Damage to the optic nerve is often related to high intra ocular pressure (IOP) although a subject may have glaucoma without increased IOP. Symptoms of glaucoma depend on the type and stage of the glaucoma. Open-angle glaucoma is the most common form of glaucoma. The drainage angle formed by the iris and cornea remains open. But other parts of the drainage system don't drain properly. This may lead to a slow, gradual increase in eye pressure. The symptoms of open-angle glaucoma include no symptoms in early stages, gradual development of patchy blind spots in side vision or peripheral vision, and, in later stages, difficulty seeing things in the central vision. Angle-closure glaucoma occurs when the iris bulges. The bulging iris partially or completely blocks the drainage angle. As a result, fluid can't circulate through the eye and pressure increases. Angle-closure glaucoma may occur suddenly or gradually. Symptoms of acute angle-closure glaucoma include severe headache, severe eye pain, nausea or vomiting, blurred vision, halos or colored rings around lights, and eye redness. Normal-tension glaucoma occurs when the optic nerve becomes damaged when eye pressure is normal. The optic nerve may be sensitive or experience less blood flow. This limited blood flow may be caused by the buildup of fatty deposits in the arteries or other conditions that damage circulation. The buildup of fatty deposits in the arteries also is known as atherosclerosis. Symptoms of normal-tension glaucoma include no symptoms in early stages, gradual development of blurred vision, and in later stages, loss of side or peripheral vision. Glaucoma in children may be present from birth or developed over the first few years of life. Blocked drainage, injury or an underlying medical condition may cause optic nerve damage. Symptoms of glaucoma in children include a dull or cloudy eye (infants), increased blinking (infants), tears without crying (infants), blurred vision, nearsightedness that gets worse, and headache. Pigmentary glaucoma occurs when small pigment granules flake off from the iris and block or slow fluid drainage from the eye. Activities such as jogging sometimes stir up the pigment granules. That leads to a deposit of pigment granules on tissue located at the angle where the iris and cornea meet. The granule deposits cause an increase in pressure. Symptoms of pigmentary glaucoma include halos around lights, blurred vision with exercise, and gradual loss of side vision. Risk factors for glaucoma include increased intraocular pressure, age over 55, black, ethnic and genetic factors, family history, certain medical conditions including diabetes, migraines, high blood pressure and sickle cell anemia, corneas that are thin in the center, extreme nearsightedness or farsightedness, eye injury or certain types of eye surgery, taking corticosteroid medicines, and especially eye drops, for a long time.

As used herein, the term “visual acuity test refers to a common eye chart test measures vision ability at various distances.

As used herein, the term “pupil dilation” refers to the widening of a subject's pupils to allow a close-up examination of the eye's retina.

As used herein, the term “palpebral opening” or “palpebral fissure” refers to the opening for the eyes between the eyelids.

As used herein, the term “fluorescein angiography” refers to an eye test that uses a dye and camera to look at blood flow in the retina and choroid. The dye is injected into a vein in the arm. Pictures are then taken as the dye passes through the blood vessels in the retina, helping a doctor evaluate if the blood vessels are leaking and whether or not the leaking can be treated. Fluorescein angiography may be used to detect wet age-related macular degeneration.

As used herein, the term “Amsler grid” refers to a simple square containing a grid pattern and a dot in the middle. This design, when used correctly, can show problem spots in a subject's field of vision. The Amsler grid may be used to detect wet age-related macular degeneration. If the straight lines in the checkerboard like grid appear wavy or missing to the patient the subject may have or be at risk of having AMD.

As used herein, the term “diabetic retinopathy” refers to a diabetes complication affecting a subject's eyes. Diabetic retinopathy is caused by damage to the blood vessels of the light-sensitive tissue at the back of the eye (retina) in subjects with type 1 or type 2 diabetes. As a result, the eye attempts to grow new blood vessels. There are two types of diabetic retinopathy: “early diabetic retinopathy” or “non-proliferative diabetic retinopathy” (NPDR) and “advanced diabetic retinopathy.” Subject's having NPDR experience a weakening of the walls of the blood vessels in their retina. Tiny bulges protrude from the walls of the smaller vessels, sometimes leaking fluid and blood into the retina. Larger retinal vessels can begin to dilate and become irregular in diameter as well. NPDR can progress from mild to severe as more blood vessels become blocked. Sometimes retinal blood vessel damage leads to a buildup of fluid (edema) in the center portion (macula) of the retina. If macular edema decreases vision, treatment is required to prevent permanent vision loss. Diabetic retinopathy left untreated can progress to “advanced diabetic retinopathy” or “proliferative diabetic retinopathy.” In “advanced diabetic retinopathy” or “proliferative diabetic retinopathy,” damaged blood vessels close off, causing the growth of new, abnormal blood vessels in the retina. These new blood vessels are fragile and can leak into the clear, jellylike substance (vitreous) that fills the center of the subject's eye. Scar tissue from the growth of new blood vessels may eventually cause the retina to detach from the back of the subject's eye. If the new blood vessels interfere with the normal flow of fluid out of the eye, pressure can build in the eyeball. This buildup can damage the nerve that carries images from the subject's eye to their brain (optic nerve), resulting in glaucoma. Early symptoms of diabetic retinopathy may be non-existent or include or only mild vision problems. Left untreated, diabetic retinopathy may eventually lead to spots or dark strings floating in a subject's vision (“floaters”), blurred vision, fluctuating vision, dark or empty areas in a subject's vision, vision loss, or blindness. The risk of developing diabetic retinopathy can increase as a result of having diabetes for a long time, poor control of blood sugar level, high blood pressure, high cholesterol, pregnancy, tobacco use, ethnic predisposition, or genetic predisposition.

As used herein, the term “ocular surface disease” refers to a multifactorial disease of the ocular surface that results in symptomatic discomfort, visual disturbances, and tear film instability with progressive histopathologic and clinical changes to the ocular surface. Ocular surface disease is characterized by an increased osmolarity of the tear film and upregulation of inflammatory mediators. Ocular surface diseases affect and damage the surface layers of the eyes including the cornea, the conjunctiva, and the glandular network. Ocular surface diseases include dry eye disease (DED), blepharitis, neurotrophic keratitis, ocular rosacea, meibomian gland dysfunction, decreased tear quality and/or production with age, chemical or thermal burns, side effects of medical treatments or prescription medications, conjunctivitis, Sjogren's disease, allergies, and other immunological conditions.

As used herein, the term “dry eye disease” (DED) is a condition in which the eyes either don't form enough tears or don't form functional tears. Tears can be inadequate and unstable for many reasons. For example, dry eyes may occur if a subject does not produce enough tears or produces poor-quality tears. This tear instability leads to inflammation and damage of the eye's surface. This can lead to eye infections and damage to the surface of the eyes, which can harm a subject's quality of life. Symptoms of DED may include: a stinging or burning feeling in the eyes, eye redness, watery eyes, blurred vision, stringy mucus in or around the eyes, difficulty wearing contact lenses, or difficulty driving. Risk factors for DED include being older than 50, being a woman, eating a diet that is low in vitamin A, and wearing contact lenses or having a history of refractive surgery.

As used herein, the term “blepharitis” refers to the inflammation of the eyelids. Blepharitis usually occurs as a result of clogged oil ducts near the base of the eyelashes. Although blepharitis itself usually doesn't cause any issues with vision, it's uncomfortable and it can lead to issues like eyelash or eyelid skin problems, dry eyes, styes, chronic pinkeye, corneal injury, or a chalazion. Symptoms of blepharitis include watery or red eyes, crusted or sticky eyes, itchy, red, or swollen eyelids, frequent blinking, sensitivity to light, and blurred vision.

As used herein, the term “chalazion” refers to a blockage that can cause swelling and a hard lump in the eyelid.

As used herein, the term “stye” refers to a red, painful lump near the edge of the eyelid that may look like a boil or a pimple. Styes are often filled with pus. A stye usually forms on the outside of the eyelid, but sometimes it can form on the inner part of the eyelid. Signs and symptoms of a stye include: a red lump on the eyelid that is similar to a boil or a pimple, eyelid pain, eyelid swelling, or tearing.

As used herein, the term “neurotrophic keratitis” refers to a degenerative condition caused by nerve impairment. This impairment leads to reduced sensitivity of the cornea. As a result, a subject's eyes don't instinctively close when they need to, which can result in dry eyes or damage to the cornea. Symptoms of neurotrophic keratitis may include red or dry eyes, blurred vision, or sensitivity to light.

As used herein, the term “ocular rosacea” refers to inflammation that causes redness, burning and itching of the eyes. ocular rosacea often develops in people who have rosacea, a chronic skin condition that affects the face. Sometimes ocular (eye) rosacea is the first sign that a subject may later develop the facial type. Ocular rosacea primarily affects adults between the ages of 30 and 50. There's no cure for ocular rosacea, but medications and a good eye care routine can help control the signs and symptoms. Signs and symptoms of ocular rosacea can precede the skin symptoms of rosacea, develop at the same time, develop later, or occur on their own. Signs and symptoms of ocular rosacea may include: red, burning, itchy or watering eyes, dry eyes, grittiness or the feeling of having a foreign body in the eye or eyes, blurred vision, sensitivity to light (photophobia), dilated small blood vessels on the white part of the eye, red and swollen eyelids, recurrent eye or eyelid infections, such as pink eye (conjunctivitis), blepharitis, sties, or chalazion. The severity of ocular rosacea symptoms doesn't always match the severity of skin symptoms. Risk factors for developing ocular include heredity and environmental factors, bacterial involvement, blocked glands in the eyelids, or eyelash mites. A subject may risk aggravating ocular rosacea by ingesting or consuming hot or spicy foods or beverages, consuming or ingesting alcohol, exposure to sunlight, exposure to wind, exposure to temperature extremes, experience certain emotions, such as stress, anger, or embarrassment, engage in strenuous exercise, or take hot baths or saunas.

As used herein, the term “meibomian gland” refers to oil glands which line the margin of the eyelids (the edges which touch when the eyelids are closed). Meibomian glands secrete oil which coats the surface of the eyes and keeps the water component of tears from evaporating (drying out). Together, the water and the oil layer make up the tear film. The tear film lubricates and keeps the surface of the eyes healthy; it also affects how a subject sees. If either the water or oil layer is decreased, or is of poor quality, a subject may have symptoms of irritation and/or blurred vision.

As used herein, the term “meibomian gland dysfunction” refers to the condition where the meibomian glands are not secreting enough oil or when the oil they secrete is of poor quality. Often, the oil gland openings get plugged up so that less oil comes out of the glands. The oil that does make it out of the glands can be granular (crusty) or otherwise unhealthy and can cause irritation. Meibomian gland dysfunction is very common. In the early stages, subjects are often asymptomatic, but if left untreated, meibomian gland dysfunction can cause or exacerbate dry eye symptoms and eyelid inflammation. The oil glands become blocked with thickened secretions. Chronically clogged glands eventually become unable to secrete oil which results in permanent changes in the tear film and dry eyes. Symptoms of meibomian gland dysfunction include dryness, burning, itching, stickiness/crustiness, watering, light sensitivity, red eyes, foreign body sensation, chalazion/styes, or intermittent blurry vision.

As used herein, the term “cornea” refers to the clear, dome-shaped lens that covers the front of the eye. The cornea focuses the light coming into the eye.

As used herein, the term “corneal sensitivity” refers to the capability of the cornea to respond to stimulation. Sensitivity varies across the cornea, with the center being the most sensitive. Diseases and rigid contact lens wear greatly reduce the sensitivity of the cornea. Other corneal sensitivity threshold includes thermal thresholds and chemical thresholds. Corneal sensitivity may be assessed quantitatively or qualitatively. Corneal sensitivity to touch is assessed by an aesthesiometer that measures the corneal touch threshold (CTT) or mechanical threshold, which is the reciprocal of corneal sensitivity. The handheld esthesiometer (Cochet-Bonnet) is a device that contains a thin, retractable, nylon monofilament that extends up to 6 cm in length. Variable pressure can be applied by the device by adjusting the length. The monofilament ranges from 60 mm to 5 mm and as the length is decreased the pressure increases from 11 mm/gm to 200 mm/gm. Qualitative assessment typically involves use of cotton-tipped applicator or small piece of dental floss. In qualitative assessments, a wisp of the cotton-tipped applicator or floss is used to compare sensation in each eye. All four quadrants of the eye are tested and the sensation in each location is recorded as normal, reduced, or absent. A von Frey test may also be used. The von Frey test involves applying a punctate stimulus to a given region of the rodent's body and recording the stimulus intensity that evokes a withdrawal reflex. Stimuli are typically applied using calibrated fibers with a specific bending force.

As used herein, the term “conjunctiva” refers to a clear membrane that lines the inside of the eyelids and covers the whites of the eyes. The conjunctiva produces a layer of mucus that covers the eyes and becomes a part of the tears. It also creates a barrier that helps keep microorganisms and foreign objects out.

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence. Peptide sequences having at least 80%, 85%, 90%, 95%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods. The % homology or % identity of two sequences is well understood in the art and can be calculated by the number of conserved amino acids or nucleotides relative to the length of the sequences.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)2, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance, “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g., pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s) and include magnetic separation using antibody-coated magnetic beads, “panning” with antibody attached to a solid matrix (i.e., plate), and flow cytometry (e.g., U.S. Pat. No. 5,985,660, hereby expressly incorporated by reference in its entirety).

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, either alone or in combination.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196 (4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, which can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003) Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28:214-8. The Chothia definition is similar to the Kabat definition, but the Chothia definition takes into account positions of certain structural loop regions. See, e.g., Chothia et al., 1986, J. Mol. Biol., 196:901-17; Chothia et al., 1989, Nature, 342:877-83. The AbM definition uses an integrated suite of computer programs produced by Oxford Molecular Group that model antibody structure. Sec, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. Sec, e.g., Makabe et al., 2008, Journal of Biological Chemistry, 283:1156-1166. Still other CDR boundary definitions may not strictly follow one of the above approaches but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, IMGT, Paratome, AbM, and/or conformational definitions, or a combination of any of the foregoing.

The term “compete,” as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody “specifically binds” or “preferentially binds” to a target if it binds with greater affinity, and/or avidity, and/or more readily, and/or with greater duration than it binds to other substances. For example, an antibody that specifically or preferentially binds to a CFD epitope is an antibody that binds this epitope with greater affinity, and/or avidity, and/or more readily, and/or with greater duration than it binds to other CFD epitopes or non-CFD epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the term “antigen binding molecule” refers to a molecule that comprises an antigen binding portion that binds to an antigen and, optionally, a scaffold or framework portion that allows the antigen binding portion to adopt a conformation that promotes binding of the antigen binding portion or provides some additional properties to the antigen binding molecule. In some embodiments, the antigen is Gal3. In some embodiments, the antigen binding portion comprises at least one CDR from an antibody that binds to the antigen. In some embodiments, the antigen binding portion comprises all three CDRs from a heavy chain of an antibody that binds to the antigen or from a light chain of an antibody that binds to the antigen. In some embodiments, the antigen binding portion comprises all six CDRs from an antibody that binds to the antigen (three from the heavy chain and three from the light chain). In some embodiments, the antigen binding portion is an antibody fragment.

Non-limiting examples of antigen binding molecules include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, pig, dog, cat, horse, donkey, guinea pig, goat, or camelid. Antibody fragments may compete for binding of a target antigen with an intact antibody and the fragments may be produced by the modification of intact antibodies (e.g., enzymatic, or chemical cleavage) or synthesized de novo using recombinant DNA technologies or peptide synthesis. The antigen binding molecule can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold.

An antigen binding molecule can also include a protein comprising one or more antibody fragments incorporated into a single polypeptide chain or into multiple polypeptide chains. For instance, antigen binding molecule can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (VH—CH1-VH—CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc).

In certain embodiments, an antigen binding molecule can have, for example, the structure of an immunoglobulin. An “immunoglobulin” is a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Unless otherwise specified, the complementarity defining regions disclosed herein follow the IMGT definition. In some embodiments, the CDRs can instead by Kabat, Chothia, or other definitions accepted by those of skill in the art.

As used herein, the terms “treating” or “treatment” (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the recurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some embodiments, chronic administration may be required.

The terms “effective amount” or “effective dose” as used herein have their plain and ordinary meaning as understood in light of the specification and refer to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein. In some non-limiting examples, an effective amount or effective dose of a composition or compound may relate to the amount or dose that provides a significant, measurable, or sufficient therapeutic effect towards the treatment of a neurological disease or proteopathy, such as Alzheimer's disease, or a symptom thereof. In some embodiments, the effective amount or effective dose of a composition or compound may treat, ameliorate, or prevent the progression of memory loss, dementia, disorientation, or any other symptom of Alzheimer's disease.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy (e.g., PD1/PDL1 or CTLA4 blockade therapy).

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable, or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol, and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), tris(hydroxymethyl)aminomethane (Tris), citric acid, ascorbic acid, acetic acid, salts, phosphates, citrates, acetates, succinates, chlorides, bicarbonates, borates, sulfates, sodium chloride, sodium bicarbonate, sodium phosphate, sodium borate, sodium citrate, potassium chloride, potassium phosphate, magnesium sulfate sugars, dextrose, dextran 40, fructose, mannose, lactose, trehalose, galactose, sucrose, sorbitol, mannitol, cellulose, serum, amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, polysorbate 20, polysorbate 40, polysorbate, 60, polysorbate 80, poloxamer, poloxamer 188, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the formulation at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

The term “% w/w” or “% wt/wt” means a percentage expressed in terms of the weight of the ingredient or agent over the total weight of the composition multiplied by 100.

### EXEMPLARY EMBODIMENTS

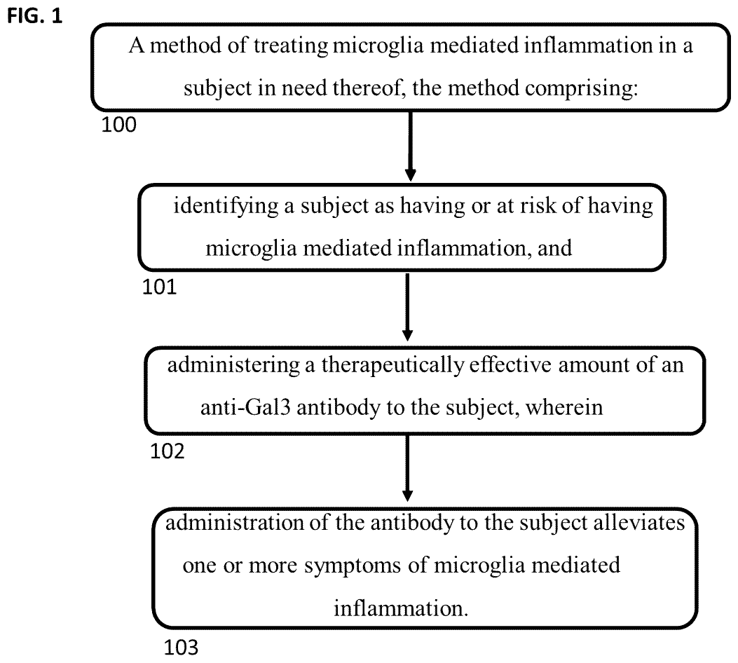

FIG. 1 is a flowchart depicting some embodiments of a method of treating microglia mediated inflammation.

In some embodiments, a method of treating microglia mediated inflammation 100 in a subject in need thereof is disclosed. In some embodiments, the method comprises: identifying a subject as having or at risk of having microglia mediated inflammation 101 and administering a therapeutically effective amount of an anti-Gal3 antibody to the subject, wherein administration of the antibody to the subject alleviates one or more symptoms of microglia mediated inflammation 103. The anti-Gal3 antibody may be administered through any effective means, for example, intravenously, intraperitoneally, or topically. In some embodiments, the microglia mediated inflammation is related to AMD and/or glaucoma. In some embodiments, identifying a subject at risk of having microglia mediated inflammation may comprise identifying the subject as at risk of having AMD and/or glaucoma. In some embodiments, administration of the anti-Gal3 antibody to the subject may relieve one or more symptoms associated with AMD or glaucoma. The anti-Gal3 antibody may be administered through any effective means, for example, intraperitoneally or topically. In some embodiments the anti-Gal3 antibody comprises TB001, TB006, or a fragment thereof.

Some embodiments herein are directed to a method of treating one or more diseases associated with microglia mediated inflammation in a subject in need thereof, the method comprising: identifying a subject as having or at risk of having one or more diseases associated with microglia mediated inflammation, and administering a therapeutically effective amount of an anti-Gal3 antibody to the subject, wherein administration of the antibody to the subject alleviates one or more symptoms of a disease microglia mediated inflammation.

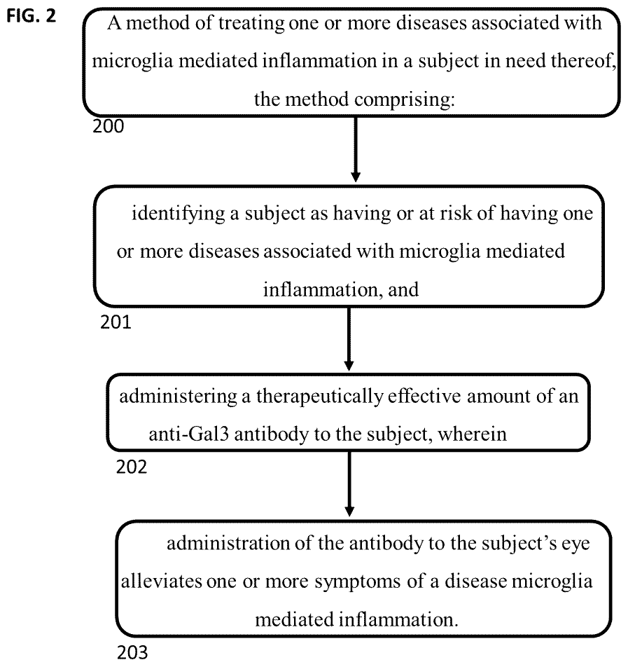

FIG. 2 is a flowchart depicting some embodiments of a method of treating one or more diseases associated with microglia mediated inflammation.

In some embodiments, a method of treating one or more diseases associated with microglia mediated inflammation 200 in a subject in need thereof is disclosed. In some embodiments, the method comprises identifying a subject as having or at risk of having one or more diseases associated with microglia mediated inflammation 201 and administering a therapeutically effective amount of an anti-Gal3 antibody to the subject 202, wherein administration of the antibody to the subject alleviates one or more symptoms of a disease microglia mediated inflammation 203. The anti-Gal3 antibody may be administered through any effective means, for example, intraperitoneally or topically. In some embodiments the anti-Gal3 antibody comprises TB001, TB006, or a fragment thereof. In some embodiments, the microglia mediated inflammation is related to AMD and/or glaucoma. In some embodiments, identifying a subject at risk of having microglia mediated inflammation may comprise identifying the subject as at risk of having AMD and/or glaucoma. In some embodiments, administration of the anti-Gal3 antibody to the subject may relieve one or more symptoms associated with AMD or glaucoma.

Some embodiments herein are directed to a method of treating an ocular surface disease in a subject in need thereof, the method comprising: identifying a subject as having or at risk of having an ocular surface disease, and administering a therapeutically effective amount of an anti-Gal3 antibody to the subject, wherein administration of the antibody to the subject alleviates one or more symptoms of ocular surface disease.

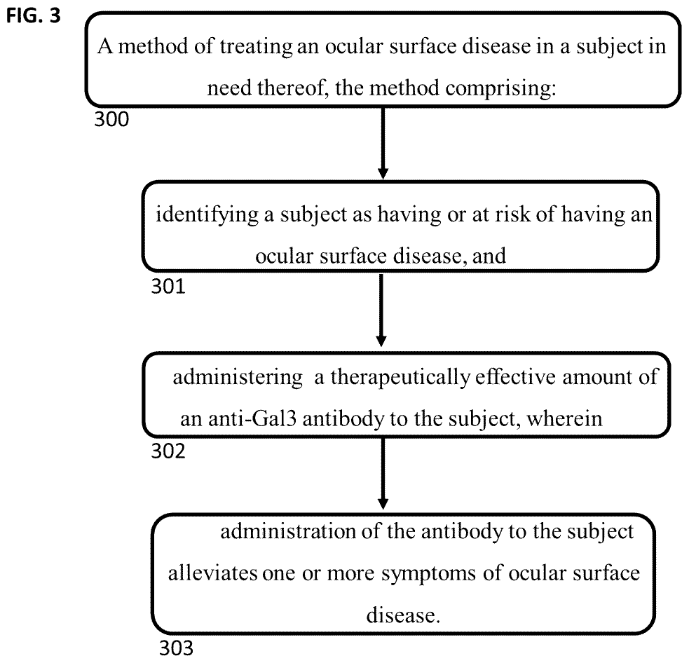

FIG. 3 is a flowchart depicting some embodiments of a method of treating an ocular surface disease.

In some embodiments, a method of treating an ocular surface disease in a subject in need thereof 300 is disclosed. In some embodiments, the method comprises identifying a subject as having or at risk of having an ocular surface disease 301 and administering a therapeutically effective amount of an anti-Gal3 antibody to the subject 302, wherein administration of the antibody to the subject alleviates one or more symptoms of the one or more ocular surface diseases 303. The anti-Gal3 antibody may be administered through any effective means, for example, intraperitonecally or topically. In some embodiments the anti-Gal3 antibody comprises TB001, TB006, or a fragment thereof. In some embodiments, the ocular surface disease comprises DED, blepharitis, neurotrophic keratitis, ocular rosacea, meibomian gland dysfunction, decreased tear quality and/or production with age, chemical or thermal burns, side effects of medical treatments or prescription medications, conjunctivitis, Sjogren's disease, allergies and other immunological conditions, or any combination thereof. Each type of ocular surface disease may have its own symptoms, although many of them include dry eyes. In some embodiments, administration of the anti-Gal3 antibody alleviates one or more symptoms of DED, blepharitis, neurotrophic keratitis, ocular rosacea, meibomian gland dysfunction, decreased tear quality and/or production with age, chemical or thermal burns, side effects of medical treatments or prescription medications, conjunctivitis, Sjogren's disease, allergies and other immunological conditions, or any combination thereof.

Some embodiments herein are directed to a method of treating age-related macular degeneration (AMD) in a subject in need thereof, the method comprising: identifying a subject as having or at risk of having AMD, and administering a therapeutically effective amount of an anti-Gal3 antibody to the subject, wherein administration of the antibody to the subject alleviates one or more symptoms of AMD.

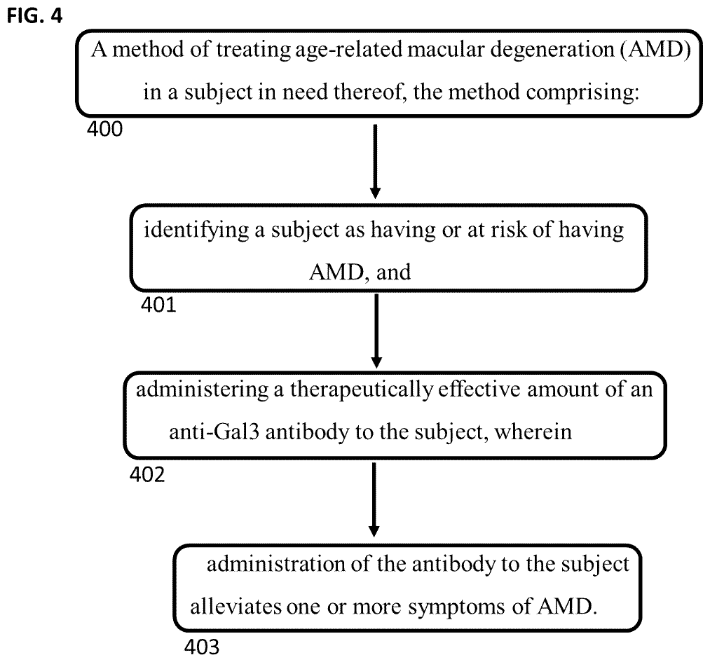

FIG. 4 is a flowchart depicting some embodiments of a method of treating age-related macular degeneration (AMD).

In some embodiments, a method of treating age-related macular degeneration (AMD) in a subject in need thereof 400, is disclosed. In some embodiments, the method comprises identifying a subject as having or at risk of having AMD 401 and administering a therapeutically effective amount of an anti-Gal3 antibody to the subject 402, wherein administration of the antibody to the subject alleviates one or more symptoms of AMD 403. The anti-Gal3 antibody may be administered through any effective means, for example, intraperitoneally or topically. In some embodiments the anti-Gal3 antibody comprises TB001, TB006, or a fragment thereof. In some embodiments, the one or more symptoms of AMD comprises Syblurry or fuzzy vision, difficulty recognizing familiar faces, straight lines appear wavy, a dark, empty area or blind spot appears in the center of vision, or loss of central vision, difficulty driving, difficulty reading, difficulty recognizing faces, difficulty performing close-up work, drusen, or any combination thereof. In some embodiments, identification of a subject or at risk of having AMD may comprise administration of a visual acuity test, pupil dilation, fluorescein angiography, or an Amsler Grid.

Some embodiments herein are directed to a method of treating dry eye disease in a subject in need thereof, the method comprising: identifying a subject as having or at risk of having dry eye disease, and administering a therapeutically effective amount of an anti-Gal3 antibody to the subject, wherein administration of the antibody to the subject alleviates one or more symptoms of dry eye disease.

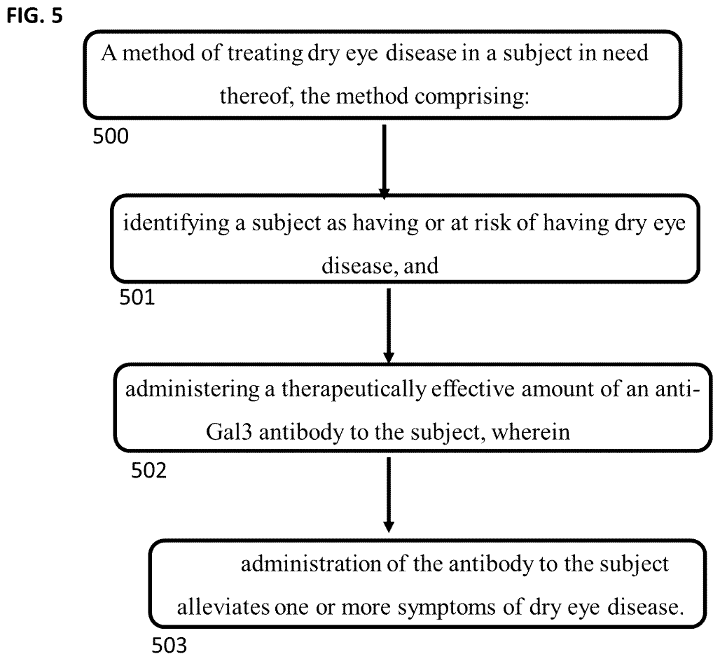

FIG. 5 is a flowchart depicting some embodiments of a method of treating dry eye disease (DED).

In some embodiments, a method of treating dry eye disease in a subject in need thereof 500, is disclosed. In some embodiments, the method comprises identifying a subject as having or at risk of having dry eye disease 501 and administering a therapeutically effective amount of an anti-Gal3 antibody to the subject 502, wherein administration of the antibody to the subject alleviates one or more symptoms of dry eye disease 503. The anti-Gal3 antibody may be administered through any effective means, for example, intraperitoneally or topically. In some embodiments the anti-Gal3 antibody comprises TB001, TB006, or a fragment thereof. In some embodiments, symptoms of DED may comprise a stinging or burning feeling in the eyes, eye redness, watery eyes, blurred vision, stringy mucus in or around the eyes, difficulty wearing contact lenses, or difficulty driving. In some embodiments, identification of a subject as having or at risk of having DED comprises identifying a subject as being older than 50, being a woman, eating a diet that is low in vitamin A, wearing contact lenses or having a history of refractive surgery, or any combination thereof.

Some embodiments herein are directed to a method of treating microvascular dysfunction in a subject in need thereof, the method comprising: identifying a subject as having or at risk of having microvascular dysfunction, and administering a therapeutically effective amount of an anti-Gal3 antibody to the subject, wherein administration of the antibody to the subject alleviates one or more symptoms of microvascular dysfunction. In some embodiments, symptoms of microvascular disfunction may comprise a stinging or burning feeling in the eyes, eye redness, watery eyes, blurred vision, stringy mucus in or around the eyes, difficulty wearing contact lenses, or difficulty driving. In some embodiments, identification of a subject as having or at risk of having microvascular disfunction comprises identifying a subject as being older than 50, being a woman, eating a diet that is low in vitamin A, wearing contact lenses or having a history of refractive surgery, or any combination thereof.

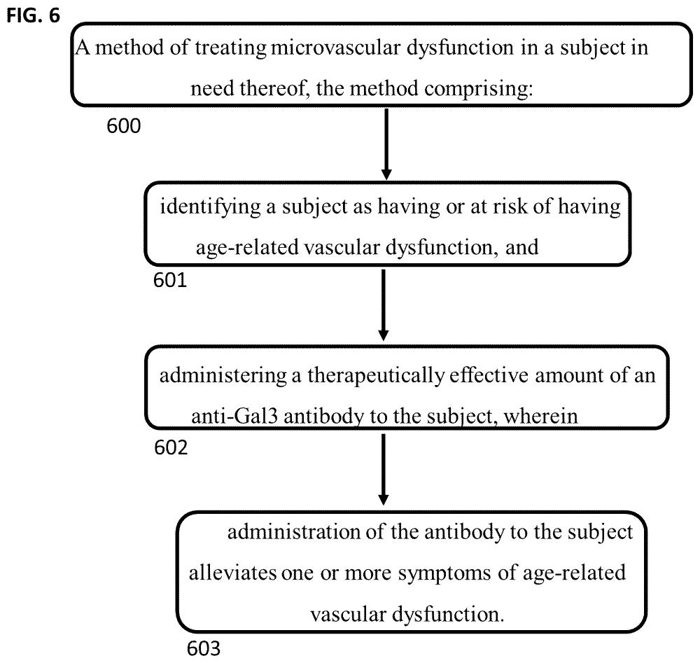

FIG. 6 is a flowchart depicting some embodiments of a method of treating microvascular dysfunction.

In some embodiments, a method of treating microvascular dysfunction in a subject in need thereof 600, is disclosed. In some embodiments, the method comprises identifying a subject as having or at risk of having microvascular dysfunction 601 and administering a therapeutically effective amount of an anti-Gal3 antibody to the subject 602, wherein administration of the antibody to the subject alleviates one or more symptoms of microvascular dysfunction 603. The anti-Gal3 antibody may be administered through any effective means, for example, intraperitoneally or topically. In some embodiments the anti-Gal3 antibody comprises TB001, TB006, or a fragment thereof.

Some embodiments herein are directed to a method of treating tear dysfunction in a subject in need thereof, the method comprising: identifying a subject as having or at risk of having tear dysfunction, and administering a therapeutically effective amount of an anti-Gal3 antibody to the subject, wherein administration of the anti-Gal3 antibody to the subject alleviates on or more symptoms of tear dysfunction.

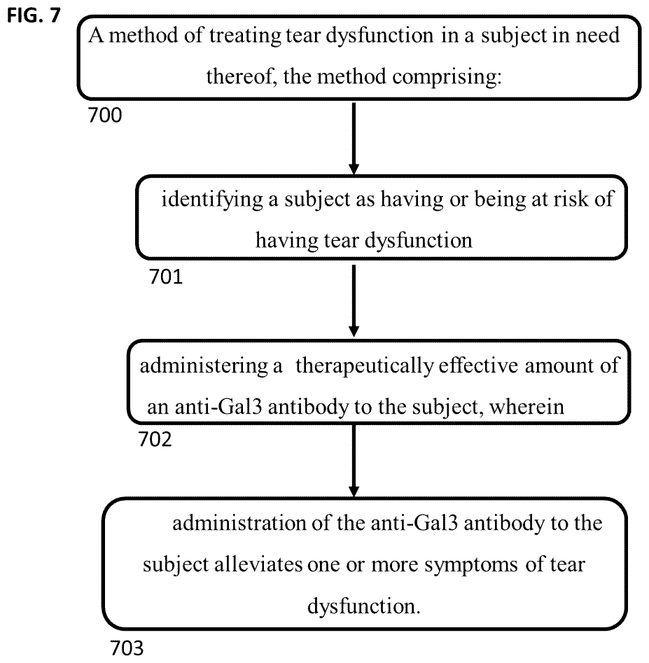

FIG. 7 is a flowchart depicting some embodiments of a method of treating tear dysfunction.

In some embodiments, a method of treating tear dysfunction in a subject in need thereof 700, is disclosed. In some embodiments, the method comprises identifying a subject as having or at risk of having tear dysfunction 701, administering a therapeutically effective amount of an anti-Gal3 antibody to the subject 702, wherein administration of the anti-Gal3 antibody to the alleviates one or more symptoms of tear dysfunction 703. The anti-Gal3 antibody may be administered through any effective means, for example, intraperitoneally or topically. In some embodiments the anti-Gal3 antibody comprises TB001, TB006, or a fragment thereof. In some embodiments, the tear dysfunction comprises decreased tear formation and/or decreased tear stability. In some embodiments, the method further comprises measuring tear formation. In some embodiments, the method further comprises measuring tear stability. In some embodiments, the method further comprises measuring tear evaporation. In some embodiments, formation and/or stability is measured by tear clearance/fluorophotometry, Schirmer strips, phenol red threads, or any combination thereof. In some embodiments, symptoms of tear dysfunction may comprise a stinging or burning feeling in the eyes, eye redness, watery eyes, blurred vision, stringy mucus in or around the eyes, difficulty wearing contact lenses, difficulty driving, corneal nerve de-sensitivity caused by contact lens use, nerve damage or laser eye surgery, or any combination thereof. In some embodiments, identifying a subject as having or at risk of having tear disfunction comprises detecting one or more risk factors of tear dysfunction. In some embodiments, the one or more risk factors comprises Sjogren's syndrome, allergic eye disease, rheumatoid arthritis, lupus, scleroderma, graft vs. host disease, sarcoidosis, thyroid disorders or vitamin A deficiency, antihistamines, decongestants, hormone replacement therapy, antidepressants, and medicines for high blood pressure, acne, birth control and Parkinson's disease, being older than 50, being a woman, eating a diet that is low in vitamin A, wearing contact lenses or having a history of refractive surgery, or any combination thereof.

Some embodiments herein are directed to a method of decreasing corneal sensitivity in a subject, the method comprising: administering a therapeutically effective amount of an anti-Gal3 antibody to the subject, wherein administration of the anti-Gal3 antibody to the subject's eye decreases corneal sensitivity in the subject as corneal sensitivity in the subject prior to administration of the antibody.

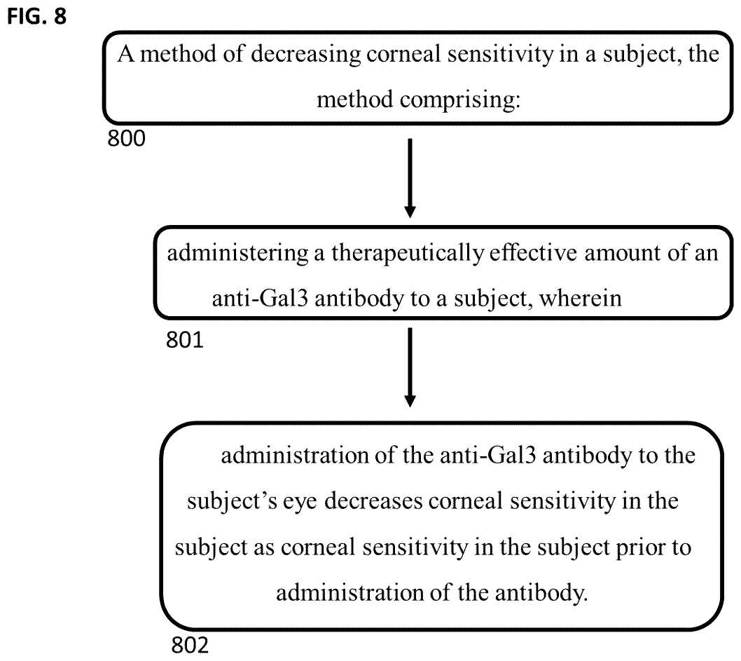

FIG. 8 is a flowchart depicting some embodiments of a method of decreasing corneal sensitivity.

In some embodiments, a method of decreasing corneal sensitivity in a subject 800, is disclosed. In some embodiments, the method comprises identifying a subject as likely to benefit from reduced corneal sensitivity 801, administering a therapeutically effective amount of an anti-Gal3 antibody to the subject 802, wherein administration of the anti-Gal3 antibody to the subject decreases corneal sensitivity in the subject as corneal sensitivity in the subject prior to administration of the antibody 803. The anti-Gal3 antibody may be administered through any effective means, for example, intraperitoneally or topically. In some embodiments the anti-Gal3 antibody comprises TB001, TB006, or a fragment thereof. In some embodiments, the method further comprises measuring corneal sensitivity before and or after administration of the anti-Gal3 antibody. In some embodiments, measuring corneal sensitivity comprises measuring a subject's mechanical, thermal, and/or chemical corneal sensitivity. In some embodiments, measuring corneal sensitivity comprises an esthesiometer, or a Cochet-Bonnet aesthesiometer. In some embodiments, measurement of corneal sensitivity comprises a Von Frey test.

Some embodiments herein are directed to a method of treating cataracts in a subject in need thereof, the method comprising: identifying a subject as having or at risk of having cataracts, and administering a therapeutically effective amount of an anti-Gal3 antibody to the subject, wherein administration of the antibody to the subject reduces the severity of the subject's cataracts as compared to the severity of cataracts in the subjects prior to administration of the anti-Gal3 antibody.

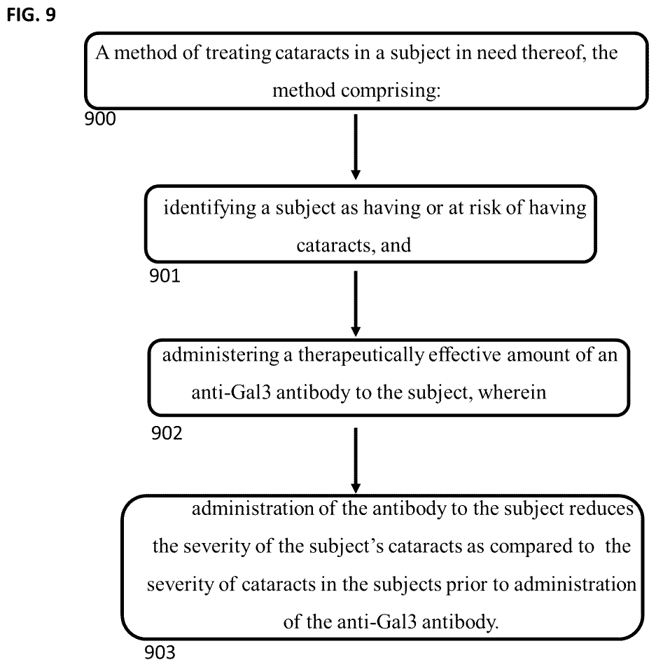

FIG. 9 is a flowchart depicting some embodiments of a method of treating cataracts.

In some embodiments, a method of treating cataracts in a subject in need thereof 1000, is disclosed. In some embodiments, the method comprises identifying a subject as having or at risk of having cataracts 901, and administering a therapeutically effective amount of an anti-Gal3 antibody to the subject 902, wherein administration of the antibody to the subject reduces the severity of the subject's cataracts as compared to the severity of cataracts in the subjects prior to administration of the anti-Gal3 antibody 903. The anti-Gal3 antibody may be administered through any effective means, for example, intraperitoneally or topically. In some embodiments the anti-Gal3 antibody comprises TB001, TB006, or a fragment thereof. In some embodiments, administration of the anti-Gal3 antibody may alleviate one or more symptoms of cataracts, including, for example, cloudy or blurry vision, colors appearing faded, poor night vision, sensitivity to lamps, sunlight, headlights, or other light, the appearance of a halo around lights, double vision, or the need to alter eye glass or contact lens prescription often. In some embodiments, the method further comprises assessing the severity of the subject's cataracts prior to and or after administration of the anti-Gal3 antibody. In some embodiments, the subject has stage 1, 2, 3, 4, 5, or 6 cataracts.

Some embodiments herein are directed to a method of preventing or reducing cataract formation in a subject in need thereof, the method comprising: identifying a subject as having or at risk of having cataracts, and administering a therapeutically effective amount of an anti-Gal3 antibody to the subject, wherein administration of the antibody to the subject reduces or prevents cataract formation in the subject acts as compared to formation of cataracts in the subjects prior to administration of the anti-Gal3 antibody.

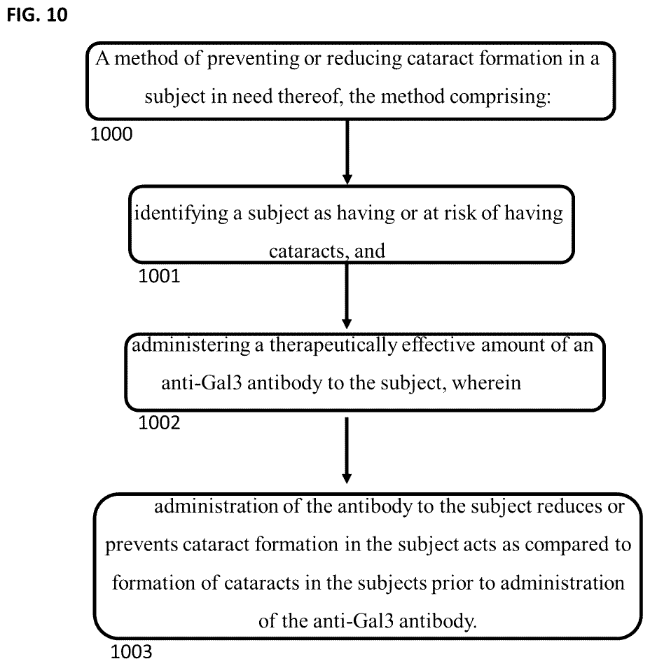

FIG. 10 is a flowchart depicting some embodiments of a method of preventing or reducing cataract formation.

In some embodiments, a method of preventing or reducing cataract formation in a subject in need thereof 1000, is disclosed. In some embodiments, the method comprises identifying a subject as having or at risk of having cataracts 1001, and administering a therapeutically effective amount of an anti-Gal3 antibody to the subject 1002, wherein administration of the antibody to the subject reduces or prevents cataract formation in the subject acts as compared to formation of cataracts in the subjects prior to administration of the anti-Gal3 antibody 1003. In some embodiments, the method further comprises evaluating cataract severity. The anti-Gal3 antibody may be administered through any effective means, for example, intraperitoneally or topically. In some embodiments the anti-Gal3 antibody comprises TB001, TB006, or a fragment thereof.

Some embodiments herein are directed to a method of treating diabetic retinopathy in a subject in need thereof, the method comprising: identifying a subject as having or at risk of having diabetic retinopathy, and administering a therapeutically effective amount of an anti-Gal3 antibody to the subject, wherein administration of the antibody to the subject alleviates one or more symptoms of diabetic retinopathy.

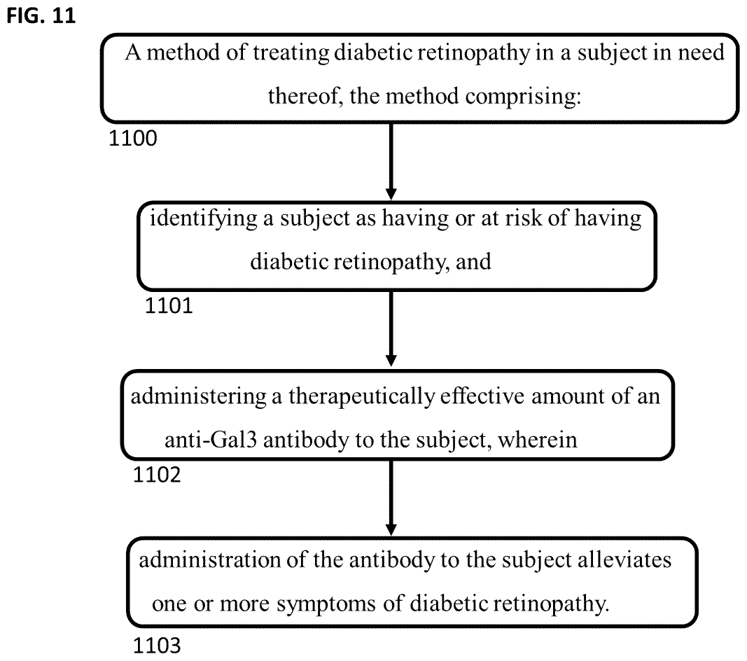

FIG. 11 is a flowchart depicting some embodiments of a method of treating diabetic retinopathy.

In some embodiments, a method of treating diabetic retinopathy in a subject in need thereof 1100, is disclosed. In some embodiments, the method comprises identifying a subject as having or at risk of having diabetic retinopathy 1101 and administering a therapeutically effective amount of an anti-Gal3 antibody to the subject 1102, wherein administration of the antibody to the subject alleviates one or more symptoms of diabetic retinopathy 1103. The anti-Gal3 antibody may be administered through any effective means, for example, intraperitoneally or topically. In some embodiments the anti-Gal3 antibody comprises TB001, TB006, or a fragment thereof. In some embodiments, the subject has or is at risk of having advanced diabetic retinopathy or proliferative diabetic retinopathy. In some embodiments, the subject has or is at risk of having early diabetic retinopathy or non-proliferative diabetic retinopathy. In some embodiment the one or more symptoms of diabetic retinopathy comprise spots or dark strings floating in a subject's vision (“floaters”), blurred vision, fluctuating vision, dark or empty areas in a subject's vision, vision loss, blindness, or any combination thereof. In some embodiments, the identification of a subject as having or at risk of having diabetic retinopathy comprises identifying the subject as having one or more risk factors for diabetic retinopathy. In some embodiments, the one or more risk factors comprise diabetes, poor control of blood sugar level, high blood pressure, high cholesterol, pregnancy, tobacco use, ethnic predisposition, genetic predisposition, or any combination thereof.

Some embodiments herein are directed to a method of treating retinal drusen in a subject in need thereof, the method comprising: identifying a subject as having or at risk of having retinal drusen, and administering a therapeutically effective amount of an anti-Gal3 antibody to the subject, wherein administration of the antibody to the subject reduces retinal drusen in the subject as compared to retinal drusen in the subject prior to administration of the anti-Gal3 antibody.

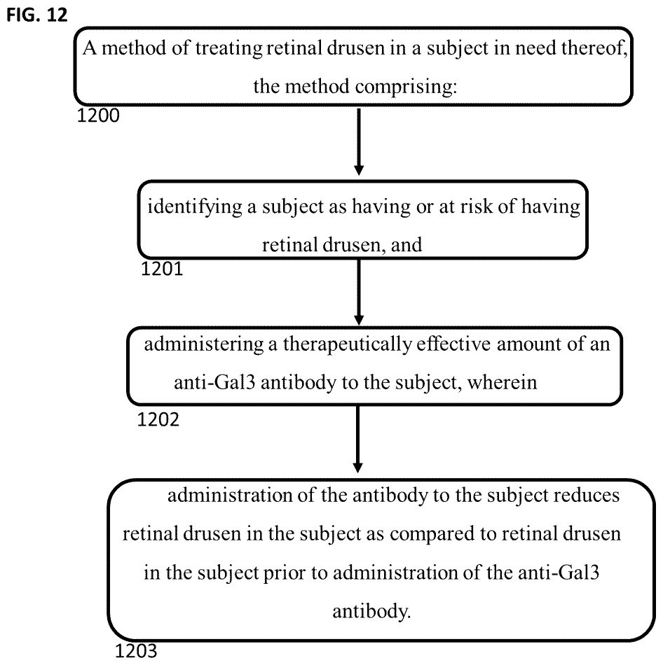

FIG. 12 is a flowchart depicting some embodiments of a method of treating retinal drusen.

In some embodiments, a method of treating retinal drusen in a subject in need thereof 1200, is disclosed. In some embodiments, the method comprises identifying a subject as having or at risk of having retinal drusen 1201 and administering a therapeutically effective amount of an anti-Gal3 antibody to the subject 1202, wherein administration of the antibody to the subject reduces retinal drusen in the subject as compared to retinal drusen in the subject prior to administration of the anti-Gal3 antibody 1203. The anti-Gal3 antibody may be administered through any effective means, for example, intraperitoneally or topically. In some embodiments the anti-Gal3 antibody comprises TB001, TB006, or a fragment thereof.

In some embodiments, the subject has a decreased palpebral opening as compared to a subject with normal eyes. In some embodiments, the subject's palpebral opening is decreased by about 1, 2, 3, 4, 5, 6, 7, 8, 9, 10, 15, 20, 25, 30, 40, 50, 60, 70, 75, 80, 90, or 100%, decreased as compared to a subject with normal eyes, or is decreased by a range that is defined by any two of the preceding values. For example, in some embodiments, the subject's palpebral opening is decreased by between about 1-100, 1-90, 1-75, 1-50, 1-25, 1-10, 1-5, 5-100, 5-90, 5-75, 5-50, 5-25 5-10, 10-100, 10-90, 10-75, 10-50, 10-25, 25-100, 25-90, 25-75, 25-50, 50-100, 50-90, 50-75, 75-100, 75-90, or 90-100%, as compared to a subject with normal eyes.

In some embodiments, the subject's palpebral opening increases following administration of anti-Gal3 antibody. In some embodiments, the subject's palpebral opening increases by about 1, 2, 3, 4, 5, 6, 7, 8, 9, 10, 15, 20, 25, 30, 40, 50, 60, 70, 75, 80, 90, or 100%, following administration of an anti-Gal3 antibody, or is increased by a range that is defined by any two of the preceding values. For example, in some embodiments, the subject's palpebral opening is increased by between about 1-100, 1-90, 1-75, 1-50, 1-25, 1-10, 1-5, 5-100, 5-90, 5-75, 5-50, 5-25 5-10, 10-100, 10-90, 10-75, 10-50, 10-25, 25-100, 25-90, 25-75, 25-50, 50-100, 50-90, 50-75, 75-100, 75-90, or 90-100%, following administration of the anti-Gal3 antibody.

In some embodiments, corneal sensitivity is measured by a von Frey test. In some embodiments, the subject has a corneal sensitivity that is about 1, 2, 3, 4, 5, 7, 8, 9, 10, 15, 20, 25, 30, 40, 50, 60, 70, 75, 80, 90, or 100%, or a range that is defined by any two of the preceding values, higher than corneal sensitivity of normal eyes. For example, in some embodiments, the subject's corneal sensitivity is about 1-100, 1-90, 1-75, 1-50, 1-25, 1-10, 1-5, 5-100, 5-90, 5-75, 5-50, 5-25 5-10, 10-100, 10-90, 10-75, 10-50, 10-25, 25-100, 25-90, 25-75, 25-50, 50-100, 50-90, 50-75, 75-100, 75-90, or 90-100%, more sensitive as compared to a subject with normal eyes.

In some embodiments, the subject's corneal sensitivity is decreased following administration of the anti-Gal3 antibody. In some embodiments, the subject's corneal sensitivity is decreased by about 1, 2, 3, 4, 5, 6, 7, 8, 9, 10, 15, 20, 25, 30, 40, 50, 60, 70, 75, 80, 90, or 100%, or by a range that is defined by any two of the preceding values, as compare to the subject's corneal sensitivity corneal sensitivity prior to administration of the anti-Gal3 antibody or fragment thereof. For example, in some embodiments, the subject's corneal sensitivity is decreased by between about 1-100, 1-90, 1-75, 1-50, 1-25, 1-10, 1-5, 5-100, 5-90, 5-75, 5-50, 5-25 5-10, 10-100, 10-90, 10-75, 10-50, 10-25, 25-100, 25-90, 25-75, 25-50, 50-100, 50-90, 50-75, 75-100, 75-90, or 90-100%, as compared to the subject's corneal sensitivity prior to administration of the anti-Gal3 antibody. In some embodiments, the subject's corneal sensitivity is decreased by about 0.001, 0.005, 0.01, 0.02, 0.03, 0.04, 0.05, 0.06, 0.07, 0.08, 0.09, 0.1, 0.11. 0.12, 0.13, 0.14, 0.15, 0.16, 0.17, 0.18, 0.19, or 0.2 g, or by a range that is defined by any two of the preceding values, as compared to the subject's corneal sensitivity prior to administration of the anti-Gal3 antibody. For example, in some embodiments, the subject's corneal sensitivity is decreased by between about 0.001-0.2, 0.001-0.15, 0.001-0.1, 0.001-0.05, 0.001-0.01, 0.001-0.005, 0.005-0.2, 0.005-0.15, 0.005-0.1, 0.005-0.05, 0.005-0.01, 0.01-0.2, 0.01-0.15, 0.01-0.1, 0.01-0.05, 0.05-0.2, 0.05-0.15, 0.05-0.1, 0.1-0.2, 0.1-0.15, or 0.15-0.2 g, as compared to the subject's corneal sensitivity prior to administration of the anti-Gal3 antibody.

In some embodiments, the methods disclosed herein comprise measuring a subject's tear formation. In some embodiments, the subject's tear formation is measured using a Schirmer's test. In some embodiments, the subject's tear formation is increased by about 1, 2, 3, 4, 5, 7, 8, 9, 10, 15, 20, 25, 30, 40, 50, 60, 70, 75, 80, 90, 100, 250, 500, 750, or 1000%, or by a range that is defined by any two of the preceding values, following administration of the anti-GAl3 antibody. For example, in some embodiments, the subject's tear formation is increased by between about 1-1000, 1-750, 1-500, 1-250, 1-100, 1-75, 1-50, 1-25, 1-20, 1-5, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-0750, 250-500, 500-1000, 500-750, or 750-1000%, following administration of the anti-Gal3 antibody. In some embodiments, the subject's tear formation is increased by about 1, 2, 3, 4, 5, 6, 7, 8, 9, 10, 15, 20, 25, 30, 35, 40, 45, or 50 mm/60 s, or by a range that is defined by any two of the preceding values, following administration of the anti-Gal3 antibody or binding fragment thereof. For example, in some embodiments, the subject's tear formation is increased by between about 1-50, 1-40, 1-30, 1-25, 1-20, 1-10, 1-5, 5-50, 5-40, 5-30, 5-25, 5-10, 10-50, 10-40, 10-30, 10-25, 25-50, 25-40, 25-30, 30-50, 30-40, or 40-50 mm/60 s, following administration of the anti-Gal3 antibody.

In some embodiments, the subject is identified as having stage 1, 2, 3, 4, 5, or 6 cataracts prior to administration of the anti-Gal3 antibody. In some embodiments, the severity of the subject's cataracts is decreased by about 1, 2, 3, 4, 5 or 6 stages, or by a range that is defined by any two of the preceding values, as compared to the severity of the subject's cataracts prior to administration of the anti-Gal3 antibodies. For example, in some embodiments, the severity of the subject's cataracts is decreased by 1-6, 1-5, 1-4, 1-3, 1-2, 2-6, 2-5, 2-4, 2-3, 3-6, 3-5, 3-4, 4-6, 4-5, or 5-6 stages, following administration of the anti-Gal3 antibody as compared to the severity of the subject's cataracts prior to administration of the anti-Gal3 antibody. In some embodiments, administration of the anti-Gal3 antibody prevents the subject's cataracts from advancing to a more sever stage following administration of the anti-Gal3 antibody.

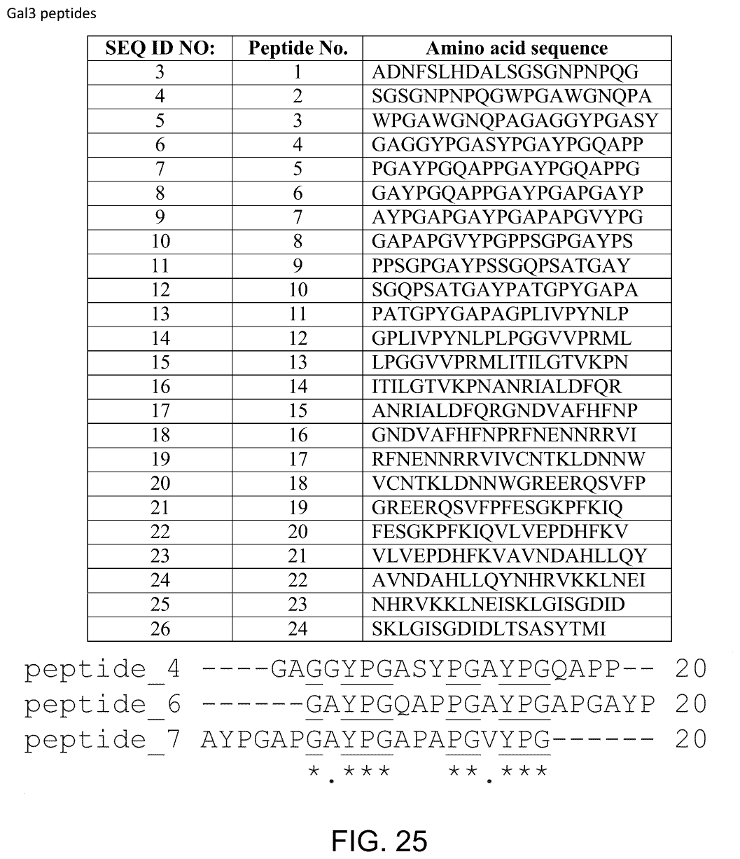

FIG. 25 shows images of some embodiments of cataract staging in mice with normal eyes and mice with dry eye disease (DED).

In some embodiments, the anti-Gal3 antibody is administered intraperitoneally.

In some embodiments, the anti-Gal3 antibody is administered topically.

In some embodiments, the anti-Gal3 antibody is administered intravenously.

In some embodiments, the anti-Gal3 antibody is administered daily, twice daily, weekly, bi-weekly, monthly, bimonthly, quarterly, and/or yearly.

In some embodiments, the anti-Gal3 antibody is administered for up to about 1, 2, 3, 4, 5, 6, 7, 8, 9, 10, 11, or 12 weeks, or for an amount of time that is in a range defined by any two of the preceding values. For example, in some embodiments, the anti-Gal3 antibody is administered for between bout 1 and 12, 1 and 10, 1 and 8, 1 and 6, 1 and 4, 1 and 2, 2 and 12, 2 and 10, 2 and 8, 2 and 6, 2 and 4, 4 and 12, 4 and 10, 4 and 8, 4 and 6, 6 and 12, 6 and 10, 6 and 8, 8 and 12, 8 and 10, or 10 and 12 weeks. In some embodiments, the anti-Gal3 antibody is administered for longer than 12 weeks. In some embodiments, the anti-Gal3 antibody is administered on an ongoing basis.

In some embodiments, the anti-Gal3 antibody is TB001 or a binding fragment thereof.

In some embodiments, the anti-Gal3 antibody is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the antibody comprises at least 1 VL-CDR having at least 90% identity to any one of the amino acid sequences of SEQ ID NOs: 170-296, 1814-1864.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the antibody comprises at least 3 VL-CDR having at least 90% identity to any one of the amino acid sequences of SEQ ID Nos: 170-296, 1814-1864.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the antibody comprises at least 1 VH-CDR having at least 90% identity to any one of the amino acid sequences of SEQ ID NOs: 27-169, 801, 802, 953, 954, 1763-1813.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the antibody comprises at least 3 VH-CDRs having at least 90% identity to any one of the amino acid sequences of SEQ ID NOs: 27-169, 801, 802, 953, 954, 1763-1813.

In some embodiments, the antibody comprises at least 1 CDR having at least 90% identity to any one of the amino acid sequences of SEQ ID NOs: 27-296, 801, 802, 953, 954, 1763-1813.

In some embodiments, the antibody comprises at least 6 CDRs having at least 90% identity to any one of the amino acid sequences of SEQ ID NOs: 27-296, 801, 802, 953, 954, 1763-1864.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the VH-CDR1 comprises an amino acid sequence having at least 90% identity to any one of the amino acid sequences of SEQ ID NOs: 27-70, 1763-1779; the VH-CDR2 comprises an amino acid sequence having at least 90% identity to any one of the amino acid sequences of SEQ ID NOS: 71-111, 801, 951, 952, 1780-1796; the VH-CDR3 comprises an amino acid sequence having at least 90% identity to any one of the amino acid sequences of SEQ ID NO: 112-169, 802, 953, 954, 1797-1813; the VL-CDR1 comprises an amino acid sequence having at least 90% identity to any one of the amino acid sequences of SEQ ID NOs: 170-220, 1814-1830; the VL-CDR2 comprises an amino acid sequence having at least 90% identity to any one of the amino acid sequences of SEQ ID NOs: 221-247, 1831-1847; and the VL-CDR3 comprises an amino acid sequence having at least 90% identity to any one of the amino acid sequences of SEQ ID NOs: 248-296. 1848-1864.

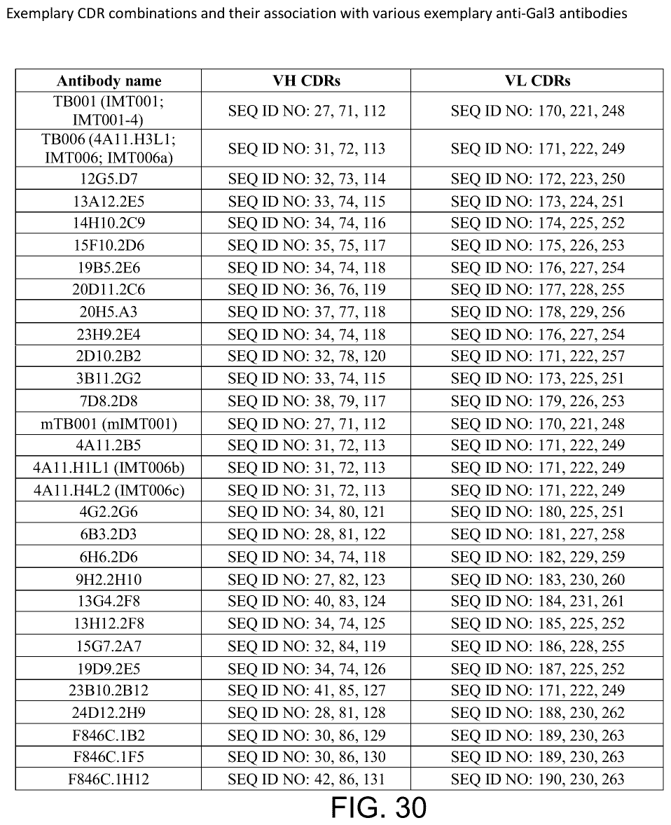

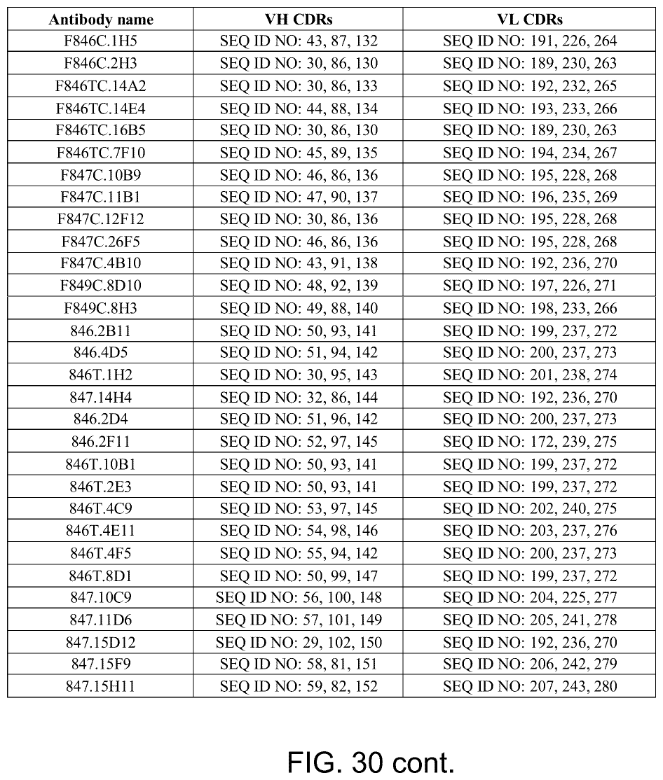

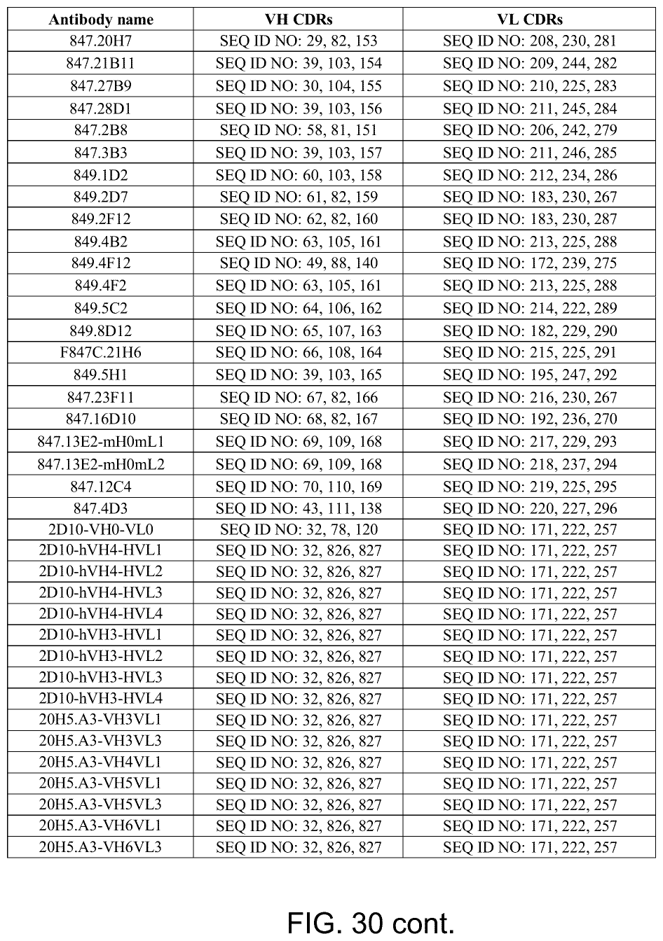

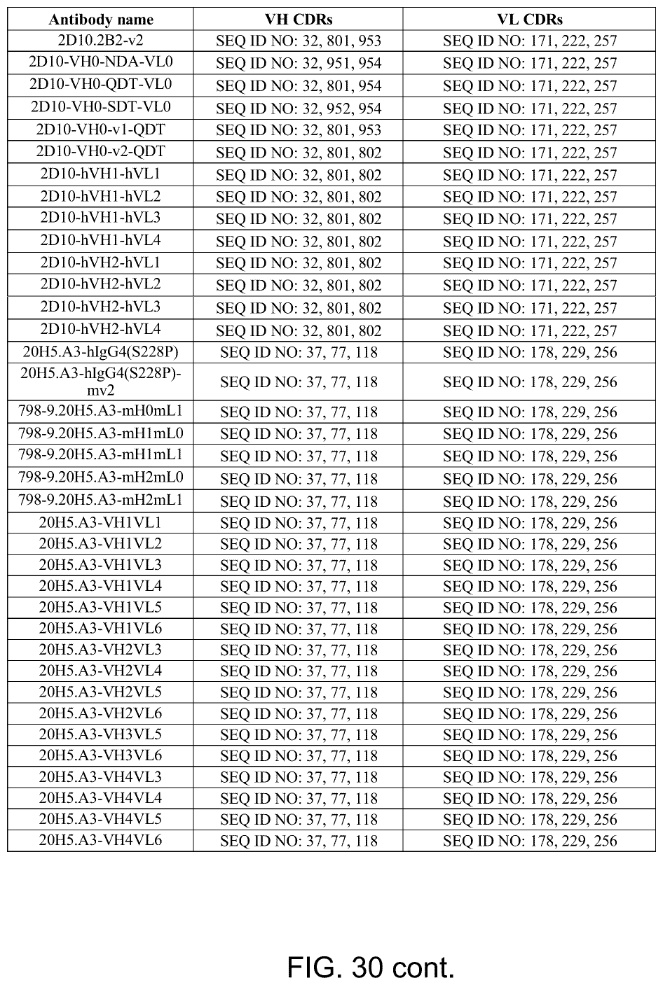

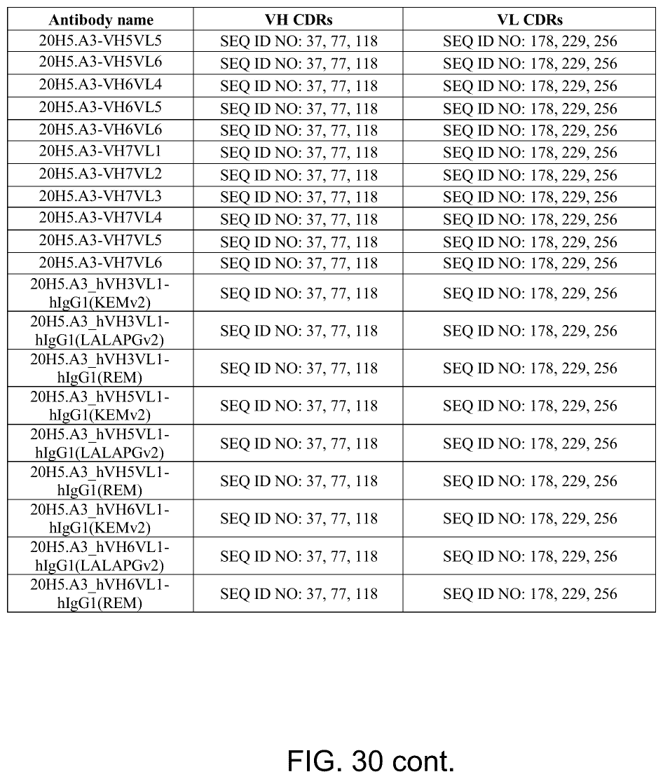

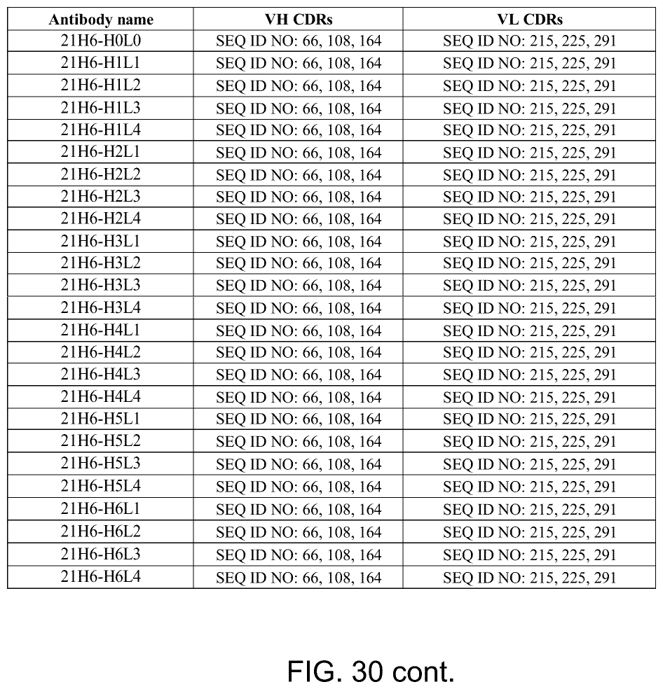

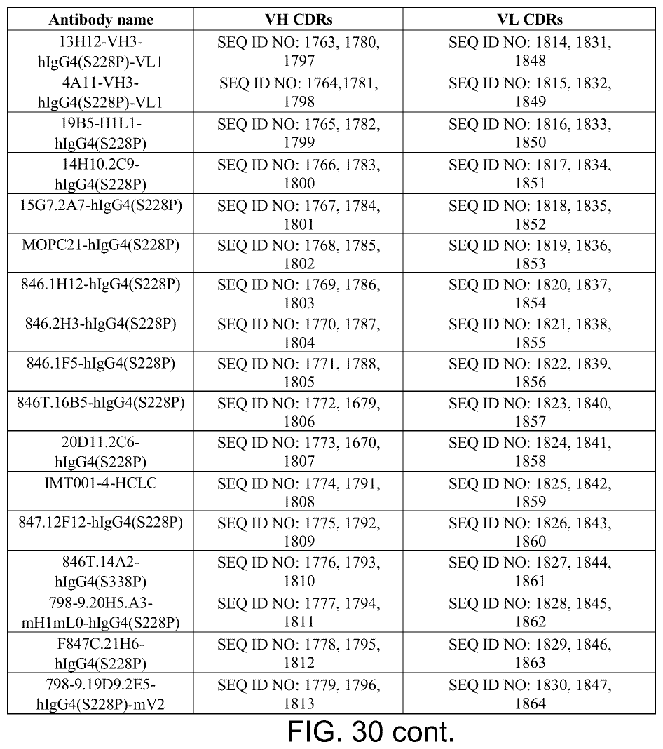

In some embodiments, the antibody comprises a combination of the VH-CDR1, VH-CDR2, VH-CDR3, VL-CDR1, VL-CDR2, and VL-CDR3 as illustrated in FIG. 30.

In some embodiments, the heavy chain variable region comprises a sequence having at least 90% identity to the sequence selected from SEQ ID NOs: 297-373, 803, 806-820, 940, 955-968, 1067-1109, 1415-1439, 1882-1898.

In some embodiments, the light chain variable region comprises a sequence having at least 90% identity to the sequence selected from SEQ ID NOs: 374-447, 821-835, 941-943, 969-982, 1110-1152, 1440-1464, 1899-1914, 1921.

In some embodiments, the antibody comprises a heavy chain, wherein the heavy chain comprises a sequence having at least 90% identity to the sequence selected from SEQ ID NOs: 448-494, 804, 836-850, 983-996, 1153-1195, 1411, 1465-1489, 1679-1694.

In some embodiments, the antibody comprises a light chain, wherein the light chain comprises a sequence having at least 90% identity to the sequence selected from SEQ ID NOs: 495-538, 805, 851-865, 997-1010, 1196-1238, 1412, 1490-1514, 1713-1728.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the VH-CDR1 comprises an amino acid sequence having at least 90% identity to SEQ ID NO: 27; the VH-CDR2 comprises an amino acid sequence having at least 90% identity to SEQ ID NO: 71; the VH-CDR3 comprises an amino acid sequence having at least 90% identity to SEQ ID NO: 112; the VH-CDR1 comprises an amino acid sequence having at least 90% identity to SEQ ID NO: 170; the VH-CDR1 comprises an amino acid sequence having at least 90% identity to SEQ ID NO: 221; and the VH-CDR1 comprises an amino acid sequence having at least 90%, to SEQ ID NO: 248.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the antibody comprises at least 3 VH-CDRs having at least 90% identity to any one of the amino acid sequences of SEQ ID NOs: 27, 71, 112.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the antibody comprises at least 3 VH-CDRs having at least 90% identity to any one of the amino acid sequences of SEQ ID NOs: 170, 221, 248.

In some embodiments, the heavy chain variable region comprises a sequence having at least 90% identity to SEQ ID NO: 297.

In some embodiments, the heavy chain variable region comprises a sequence having at least 90% identity to SEQ ID NO: 374.

In some embodiments, the antibody comprises a heavy chain, wherein the heavy chain comprises a sequence having at least 90% identity to SEQ ID NO: 448. In some embodiments, the heavy chain further comprises a signal peptide. In some embodiments, the heavy chain further comprises a signal peptide having at least 90% identity to SEQ ID NO: 1922. In some embodiments, the heavy chain further comprises a signal peptide that is SEQ ID NO: 1922.

In some embodiments, the antibody comprises a light chain, wherein the light chain comprises a sequence having at least 90% identity to SEQ ID NO: 495. In some embodiments, the light chain further comprises a signal peptide. In some embodiments, the light chain further comprises a signal peptide having at least 90% identity to SEQ ID NO: 1923 or SEQ ID NO: 1924. In some embodiments, the light chain further comprises a signal peptide that is SEQ ID NO: 1923. In some embodiments, the light chain further comprises a signal peptide that is SEQ ID NO: 1924.

In some embodiments, the antibody comprises (1) a heavy chain variable region comprising a VH-CDR1, a VH-CDR2, and a VH-CDR3; and (2) a light chain variable region comprising a VL-CDR1, a VL-CDR2, and a VL-CDR3, wherein the VH-CDR1 comprises an amino acid sequence having at least 90% identity to SEQ ID NO: 31; the VH-CDR2 comprises an amino acid sequence having at least 90% identity to SEQ ID NO: 72; the VH-CDR3 comprises an amino acid sequence having at least 90% identity to SEQ ID NO: 113; the VH-CDR1 comprises an amino acid sequence having at least 90% identity to SEQ ID NO: 170; the VH-CDR1 comprises an amino acid sequence having at least 90% identity to SEQ ID NO: 221; and the VH-CDR1 comprises an amino acid sequence having at least 90%, to SEQ ID NO: 248.

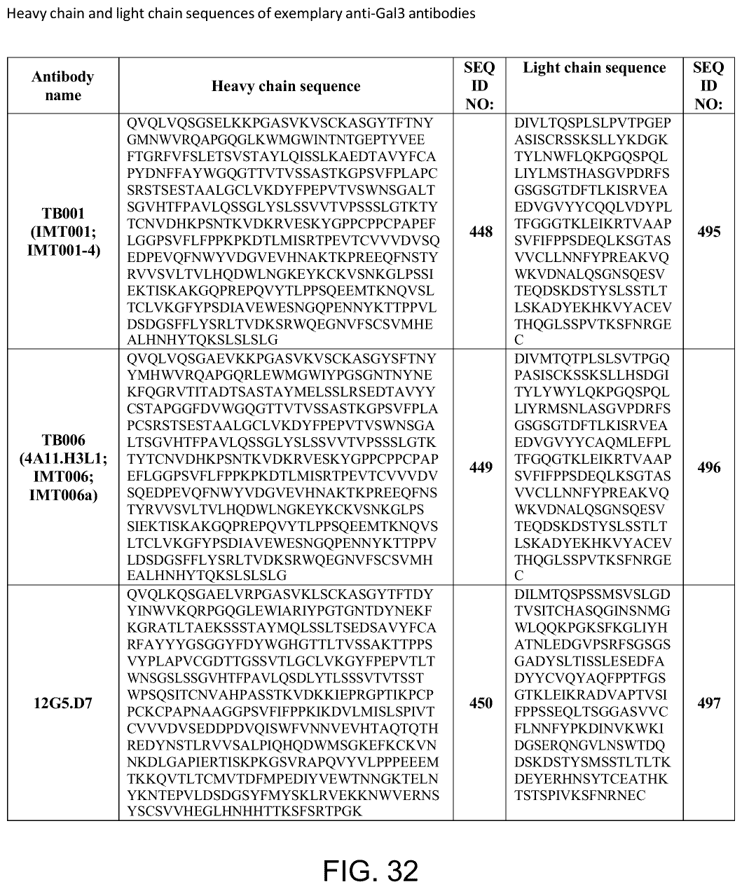

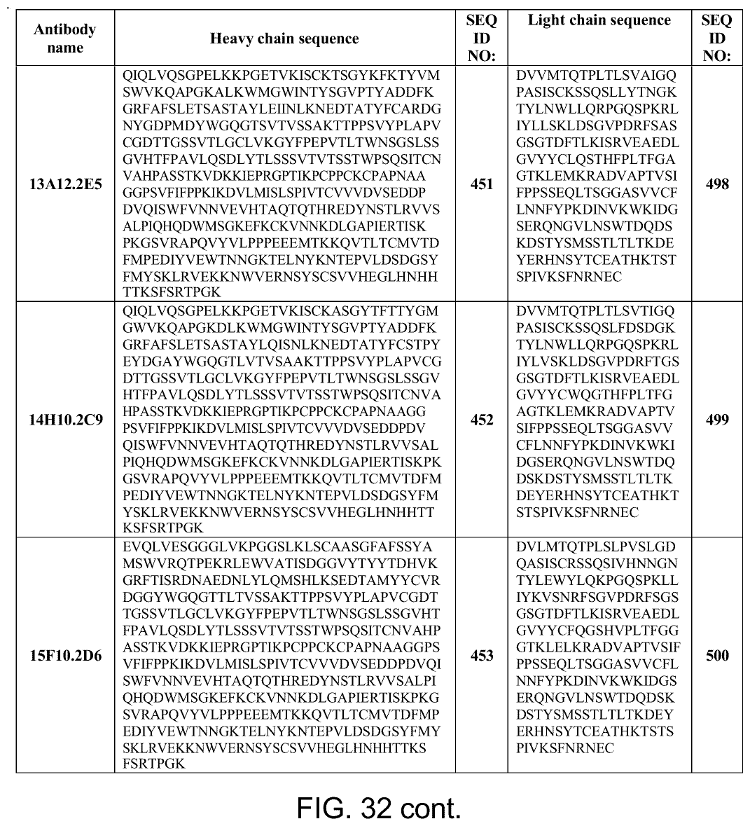

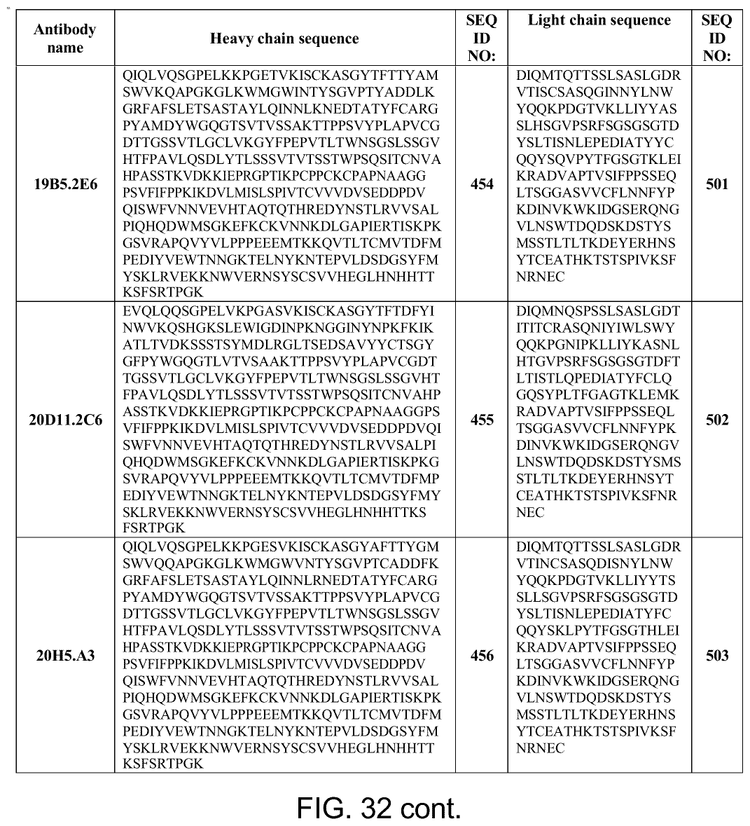

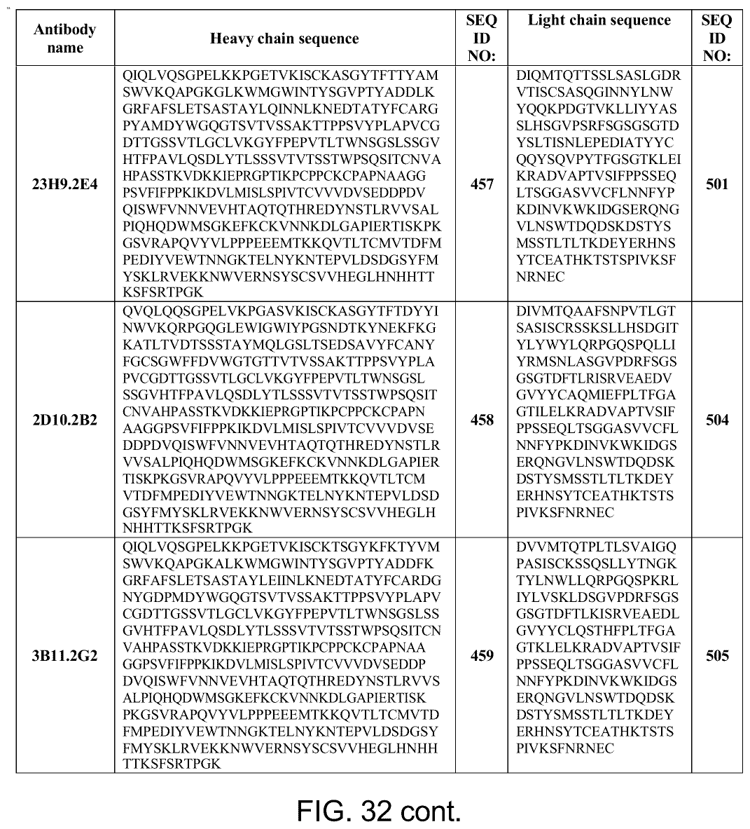

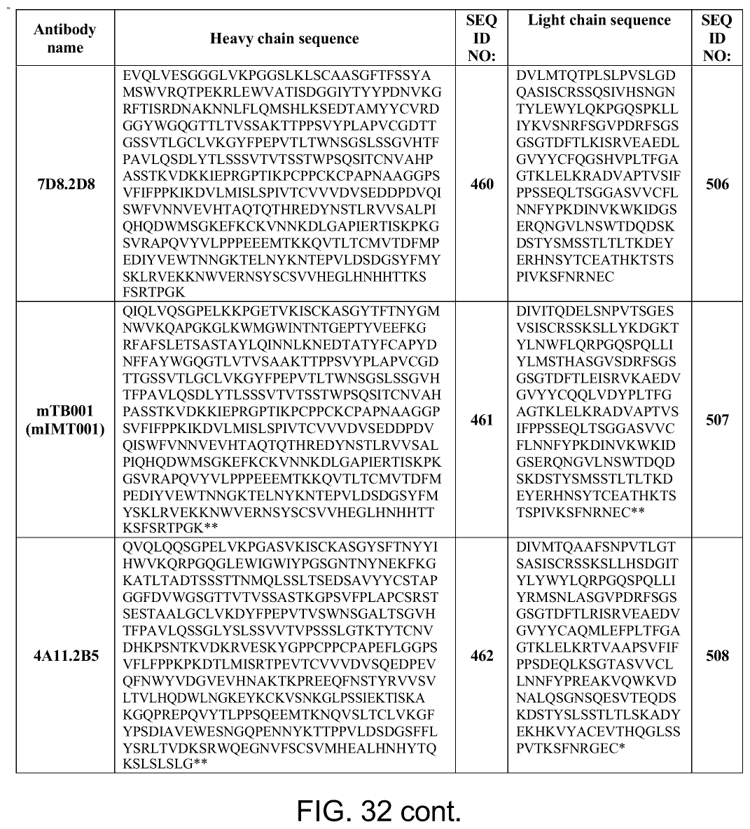

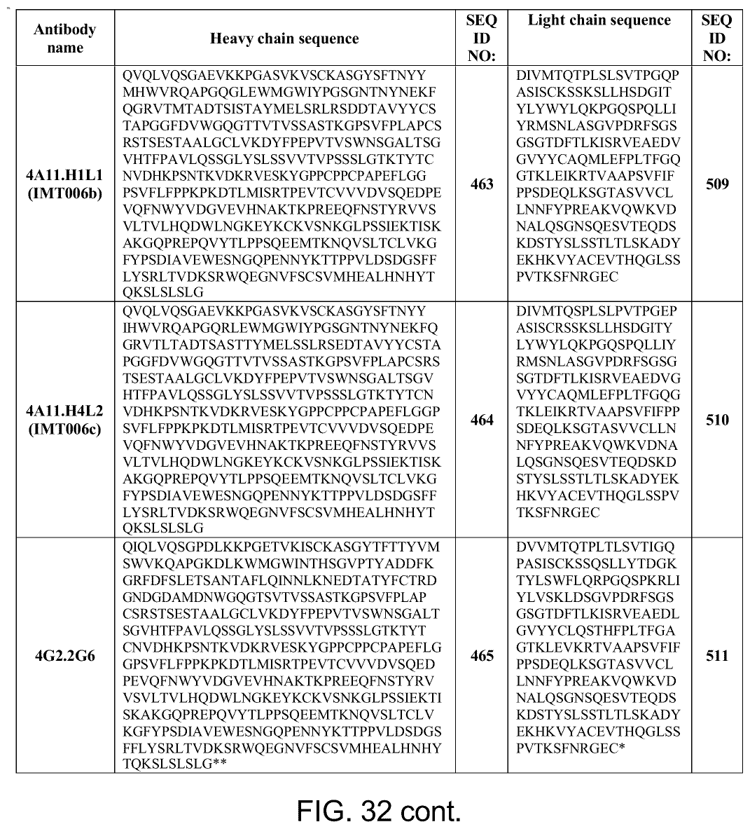

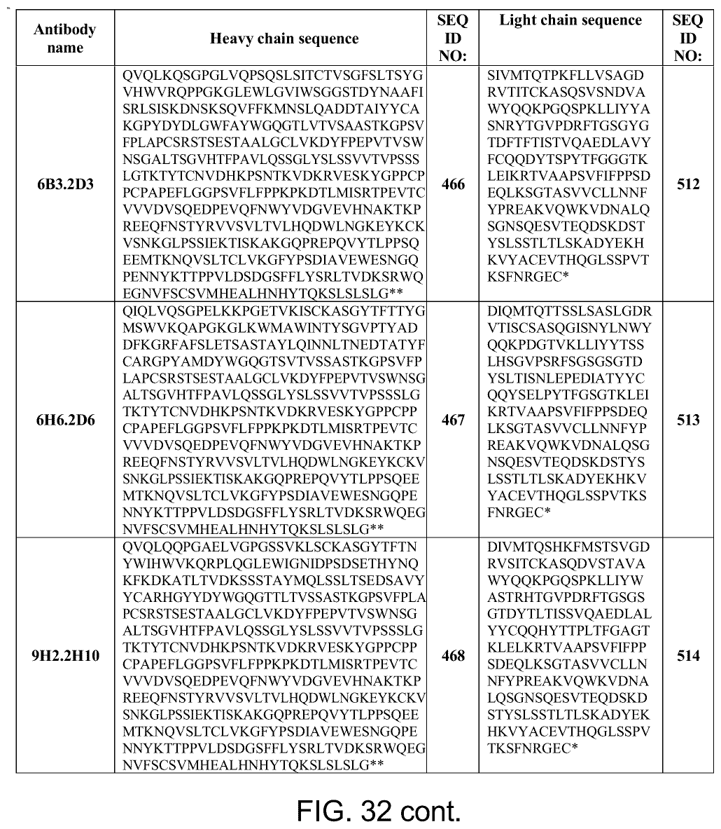

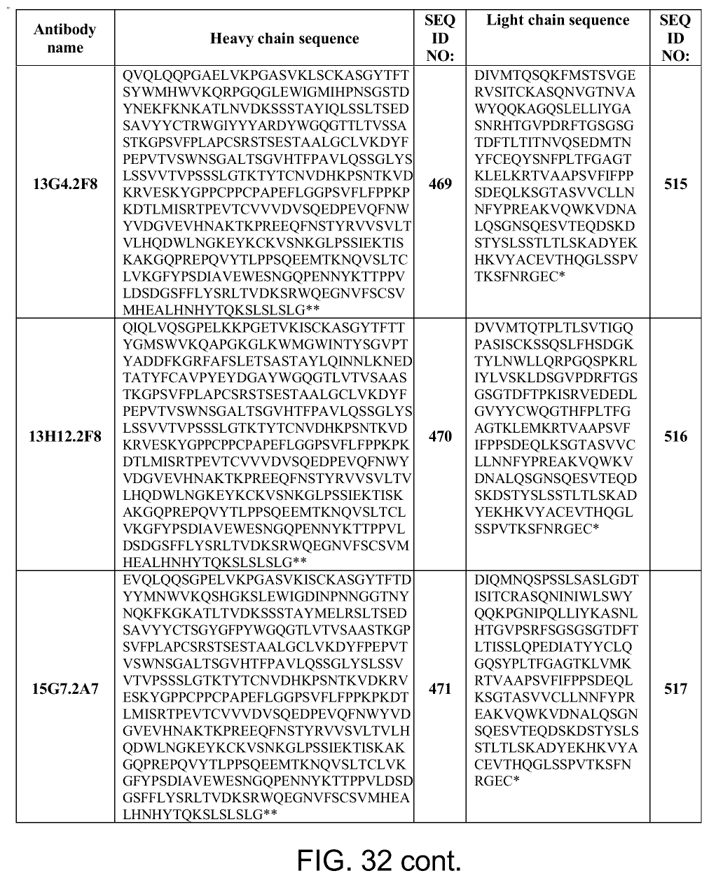

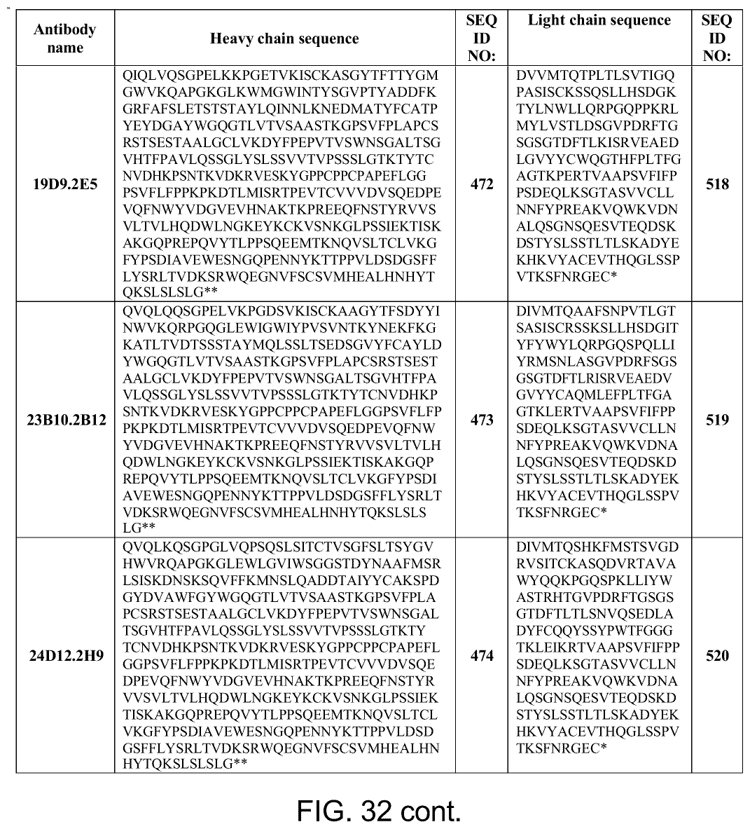

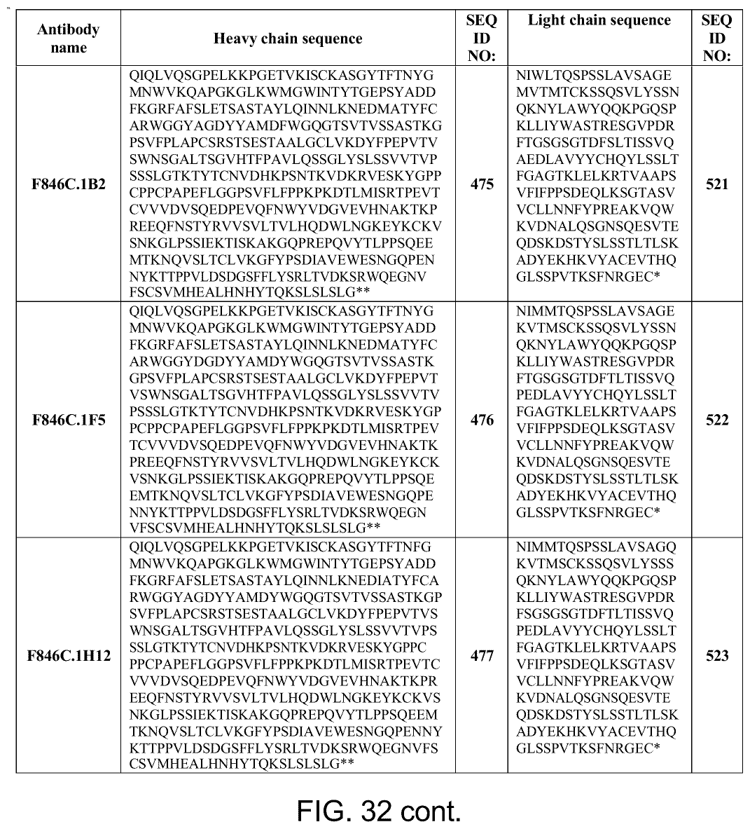

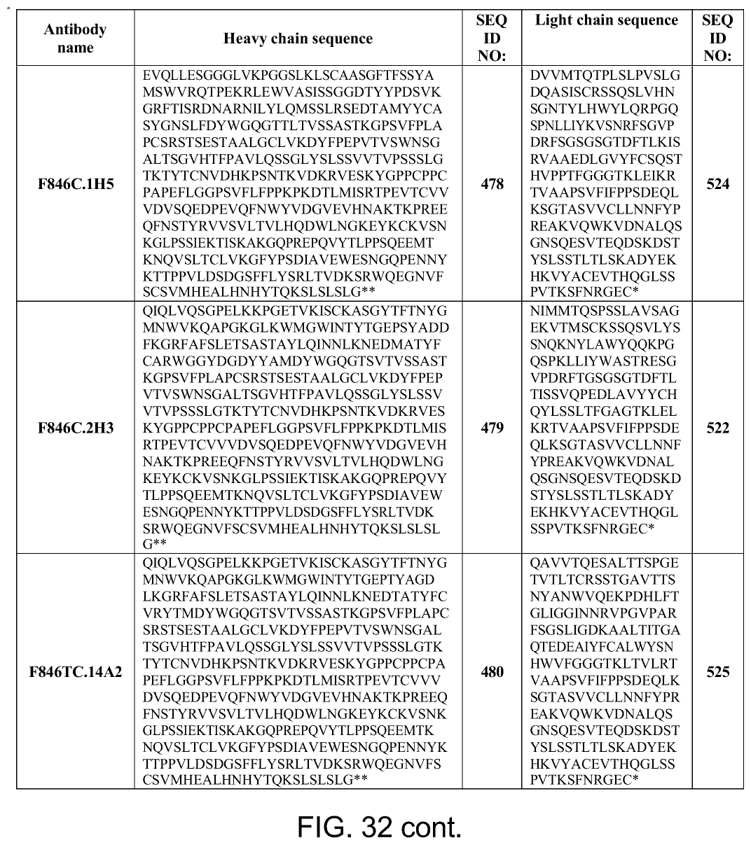

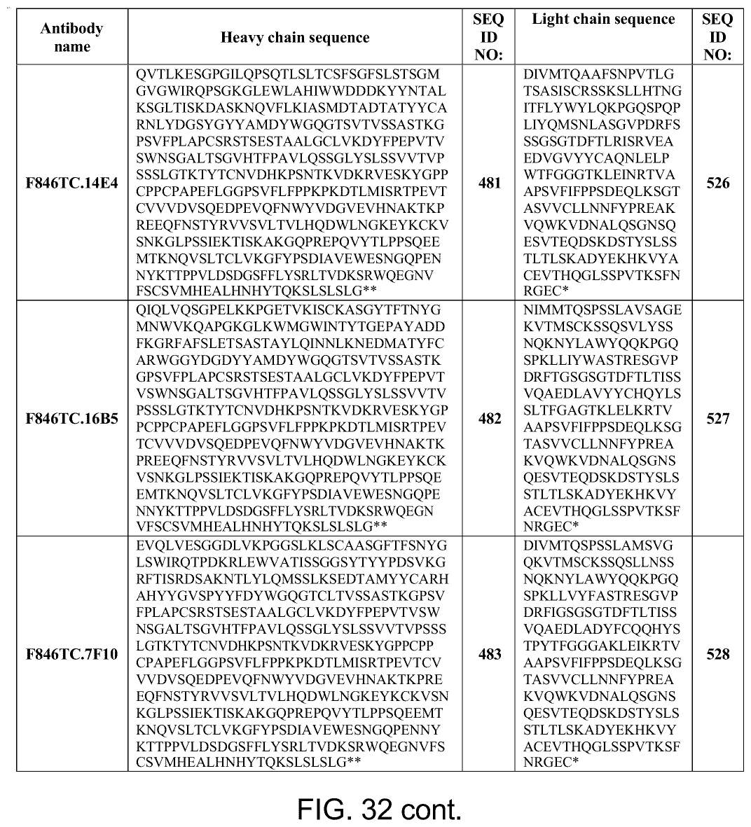

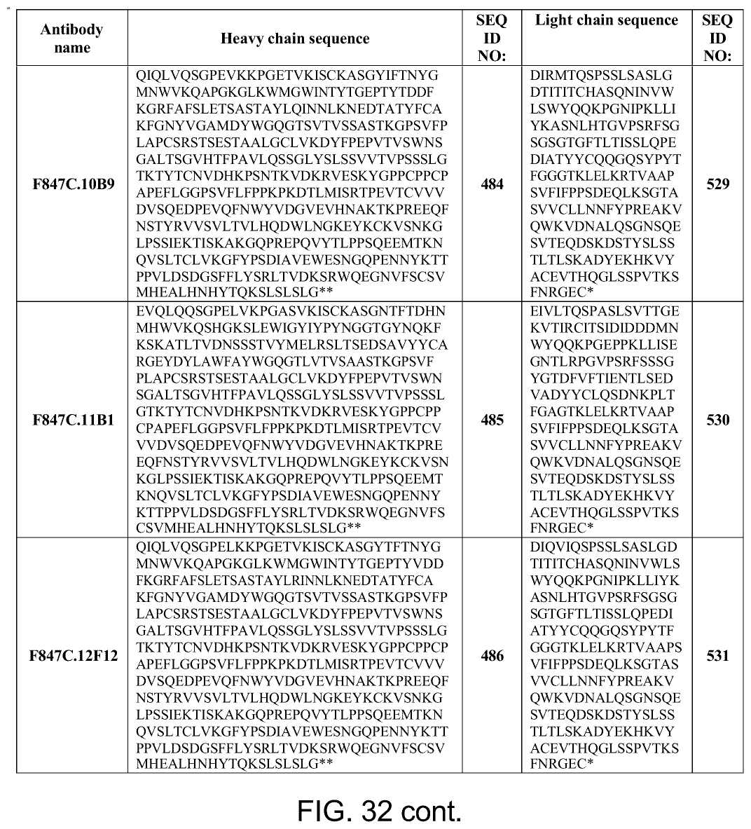

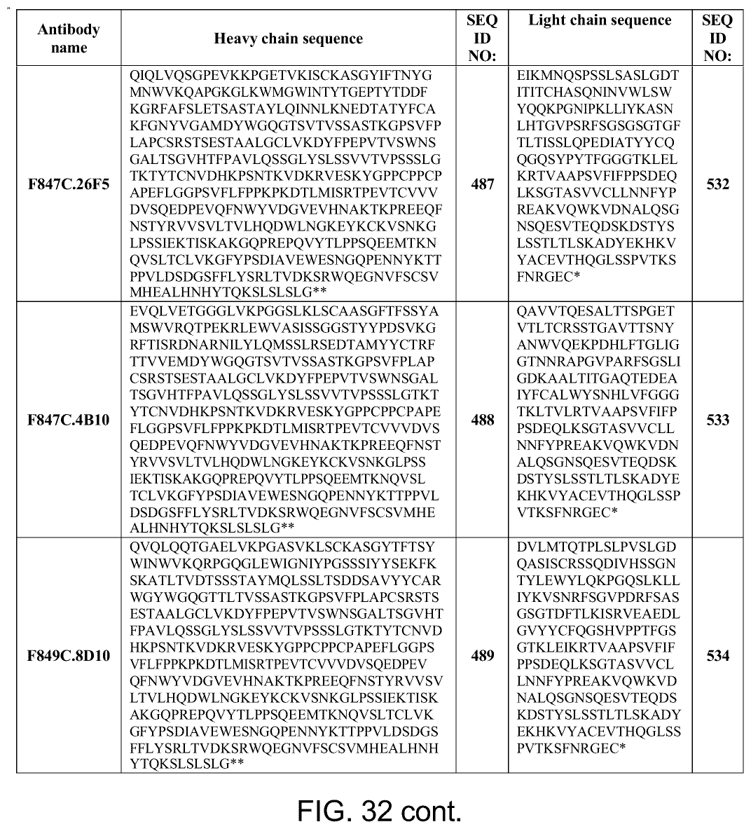

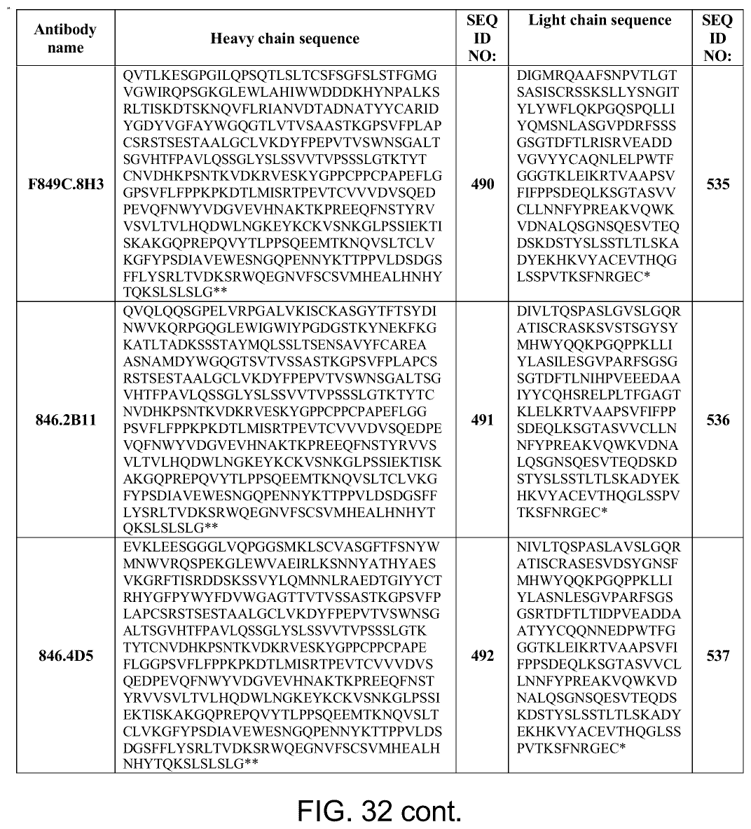

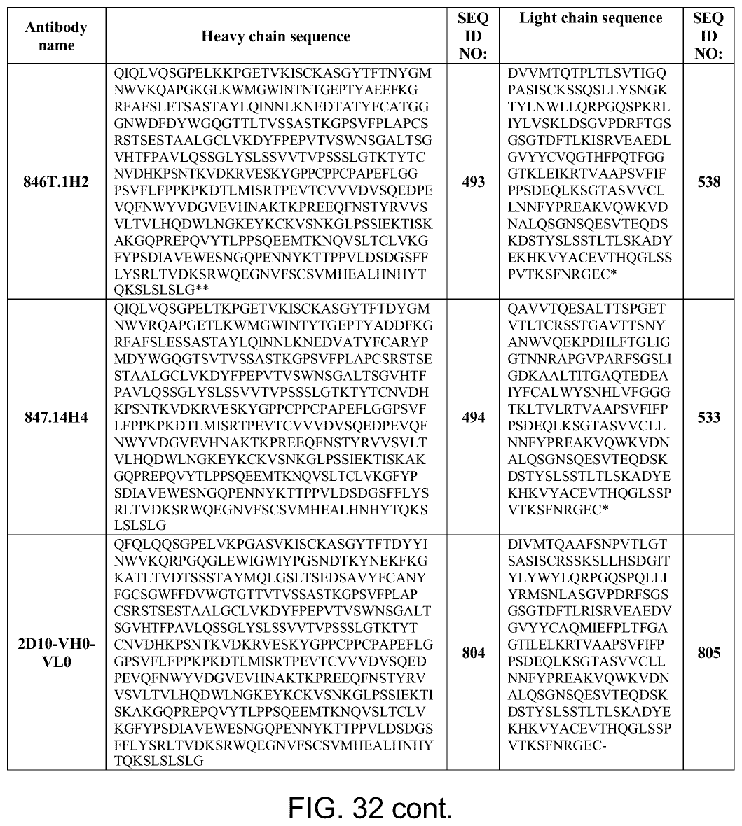

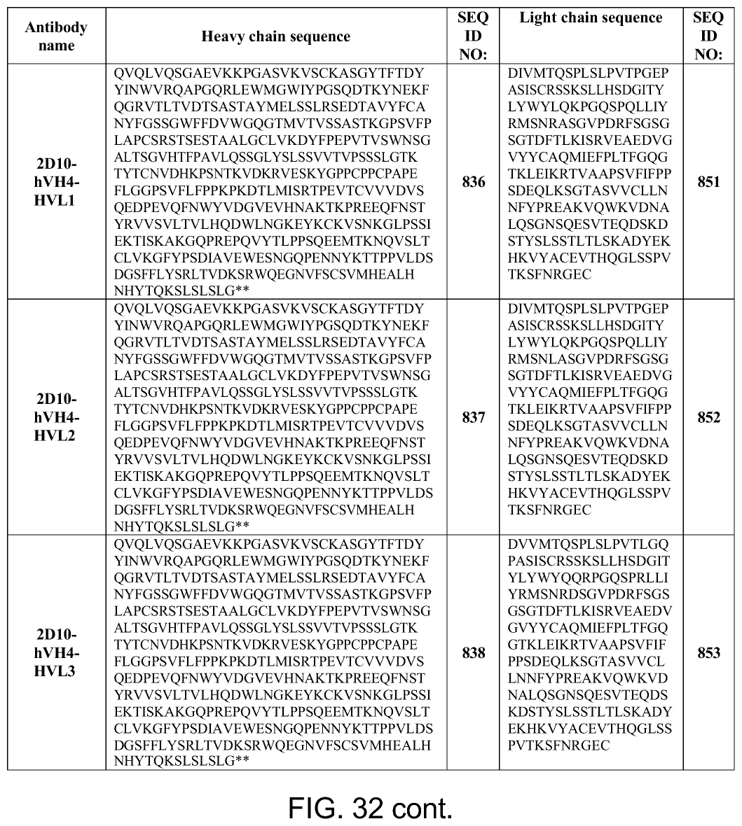

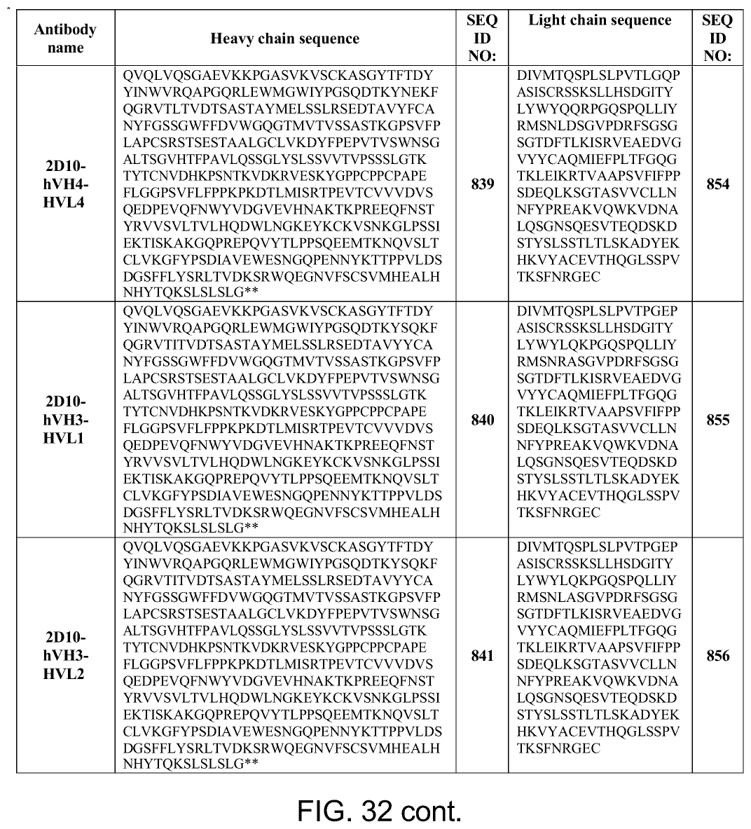

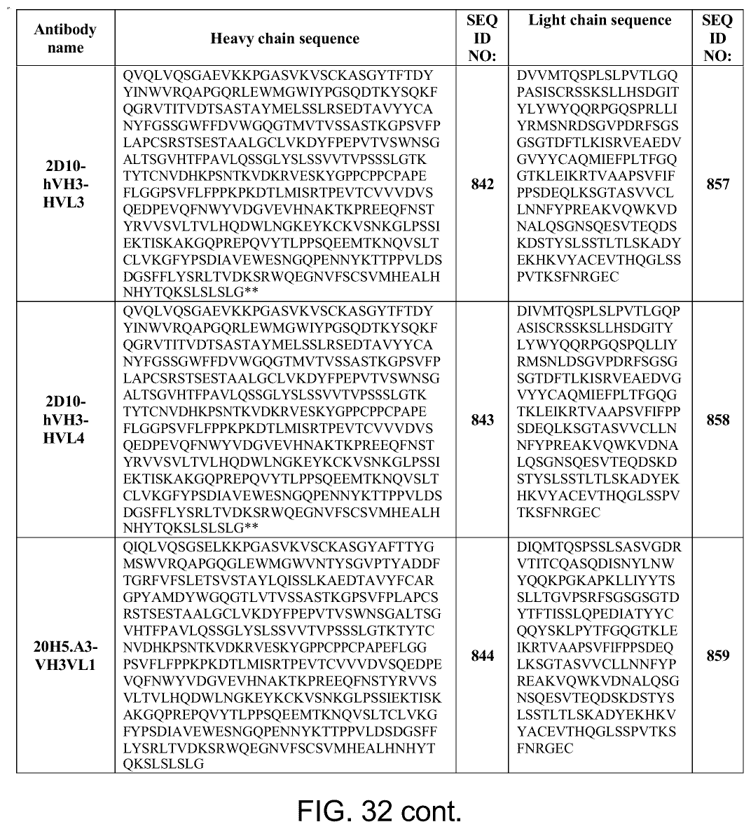

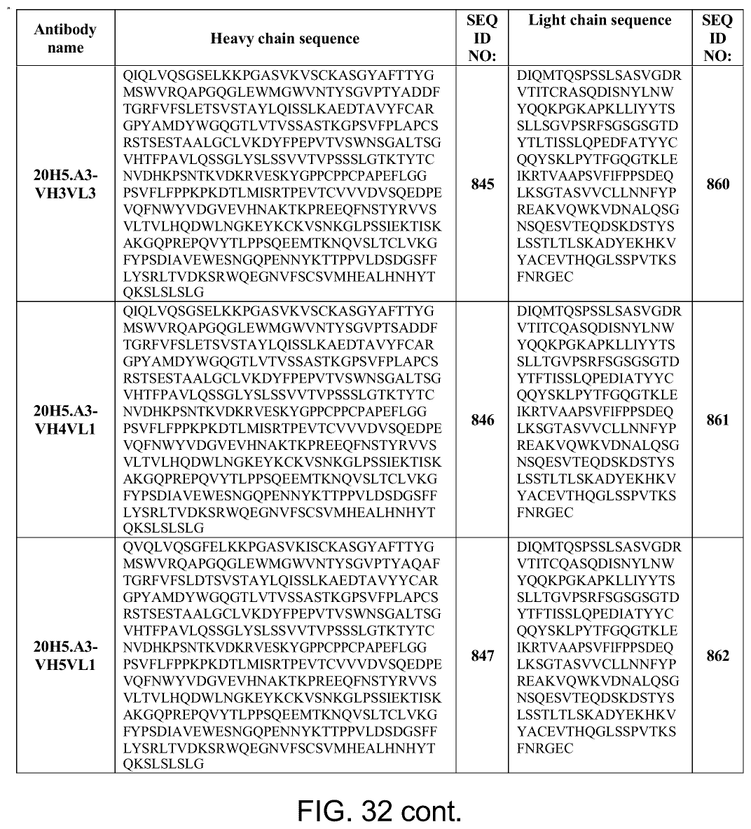

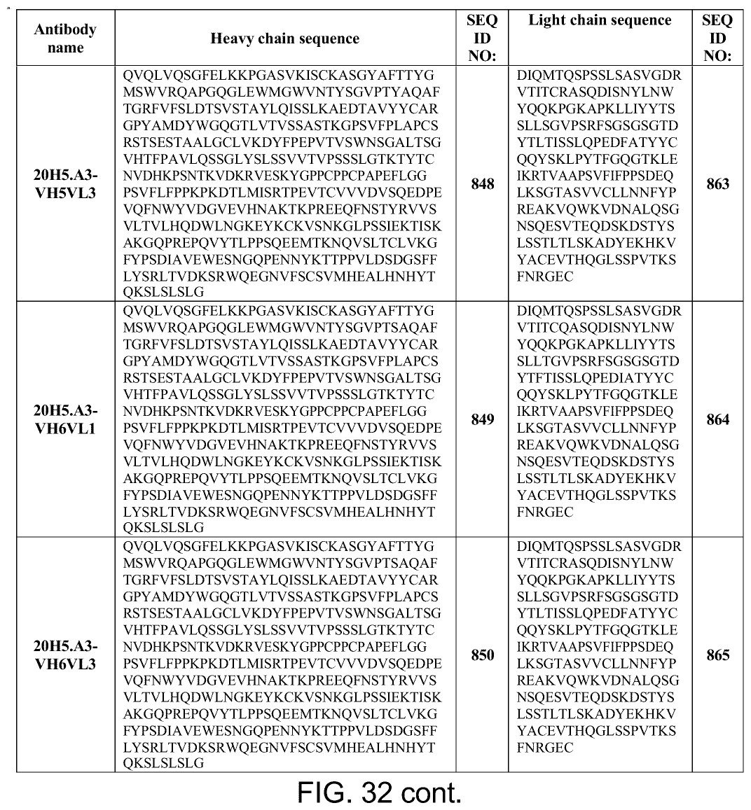

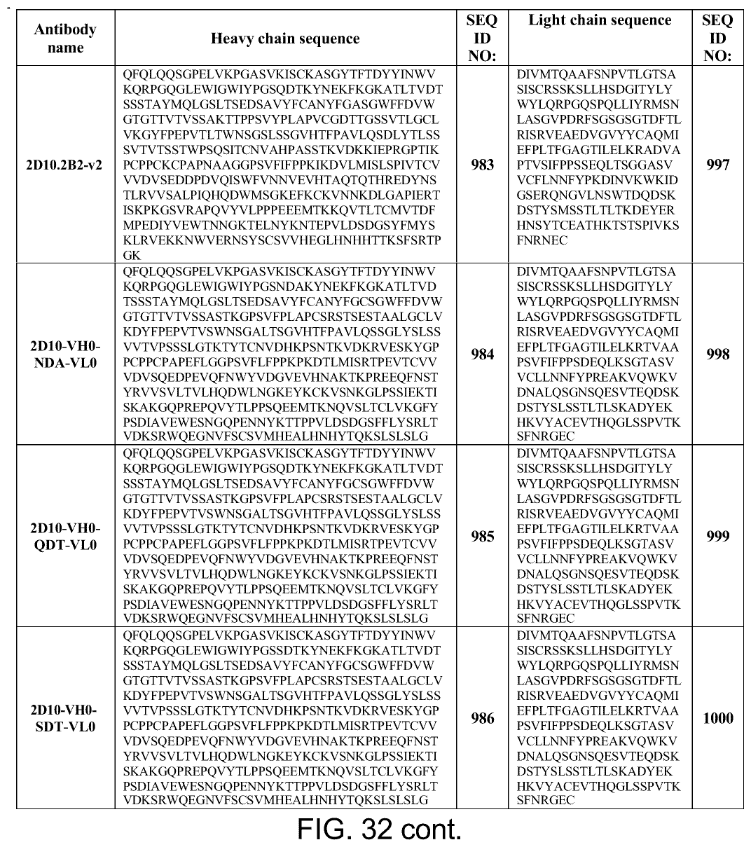

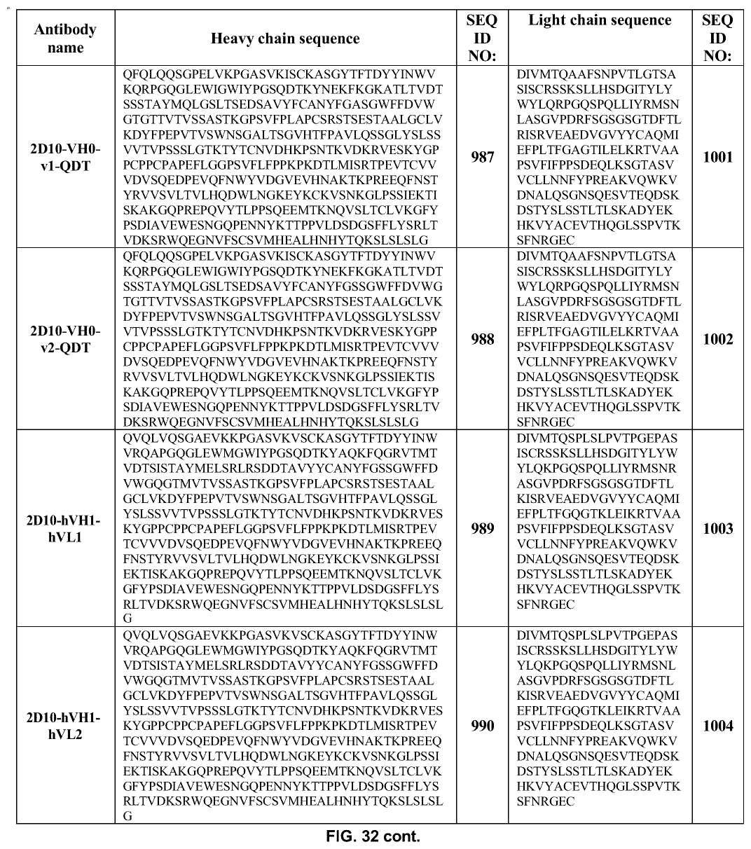

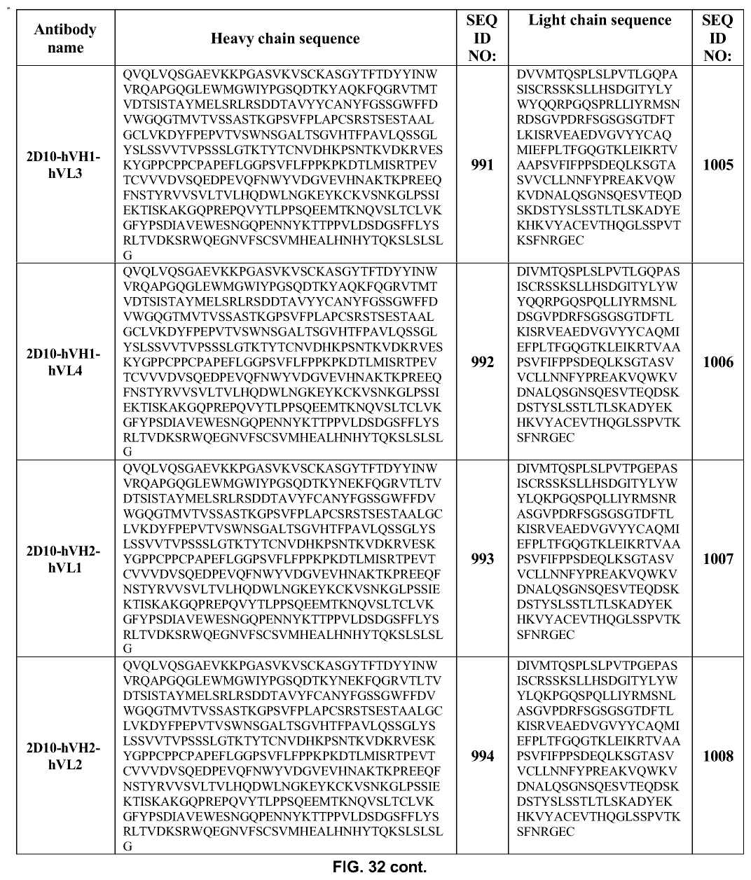

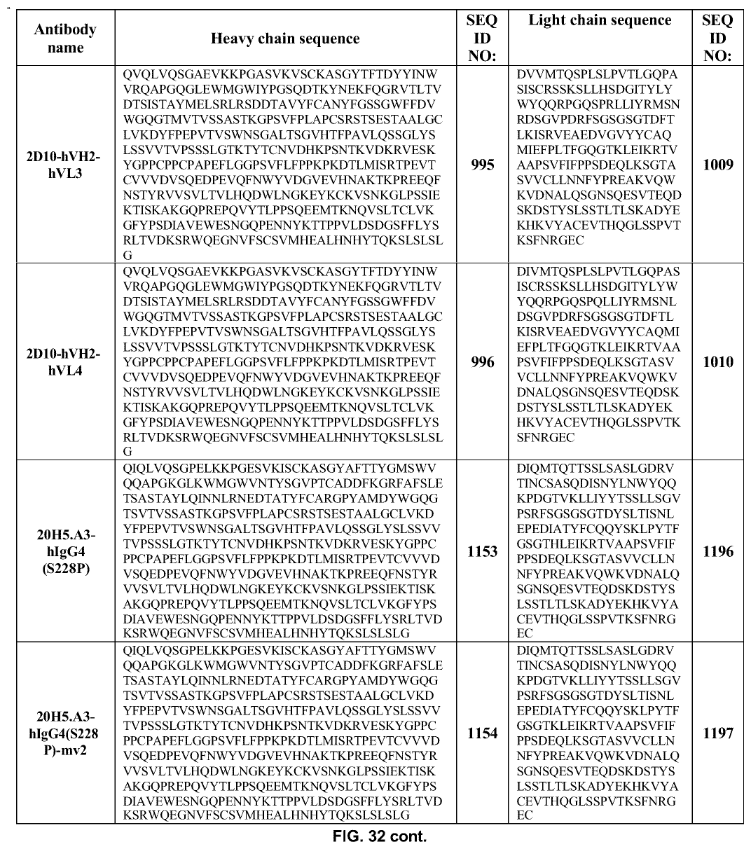

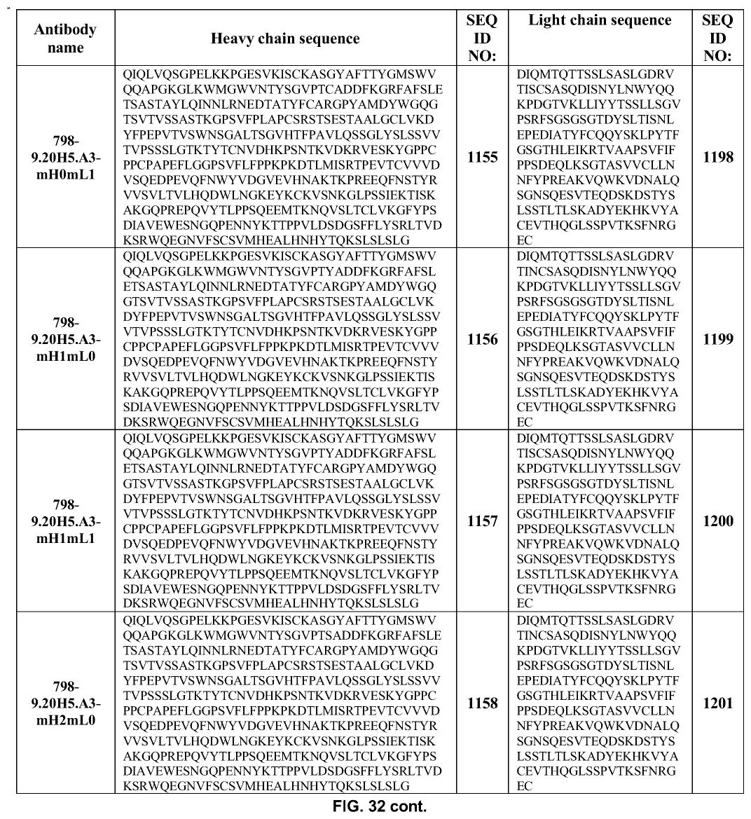

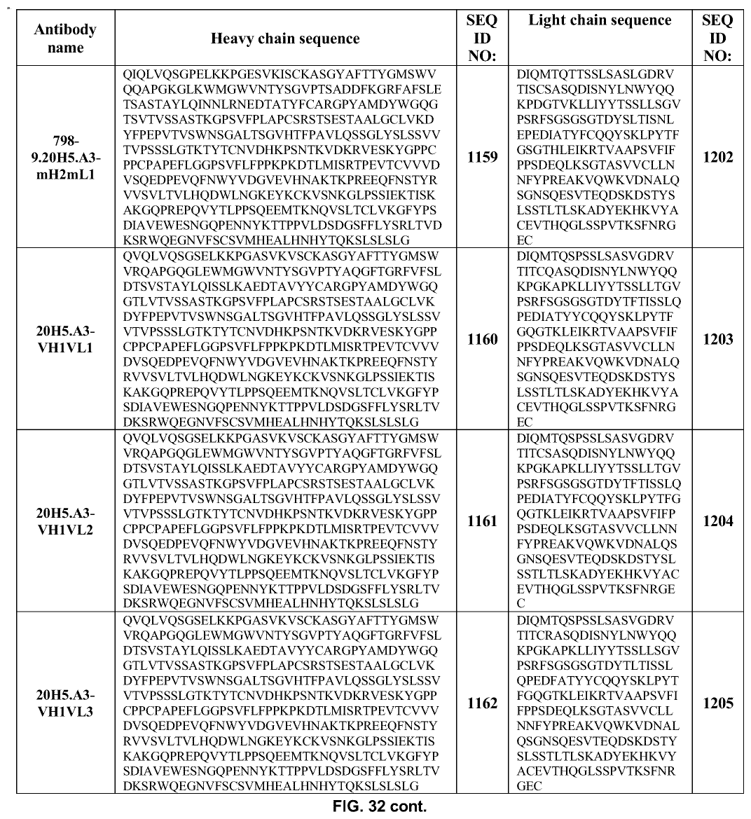

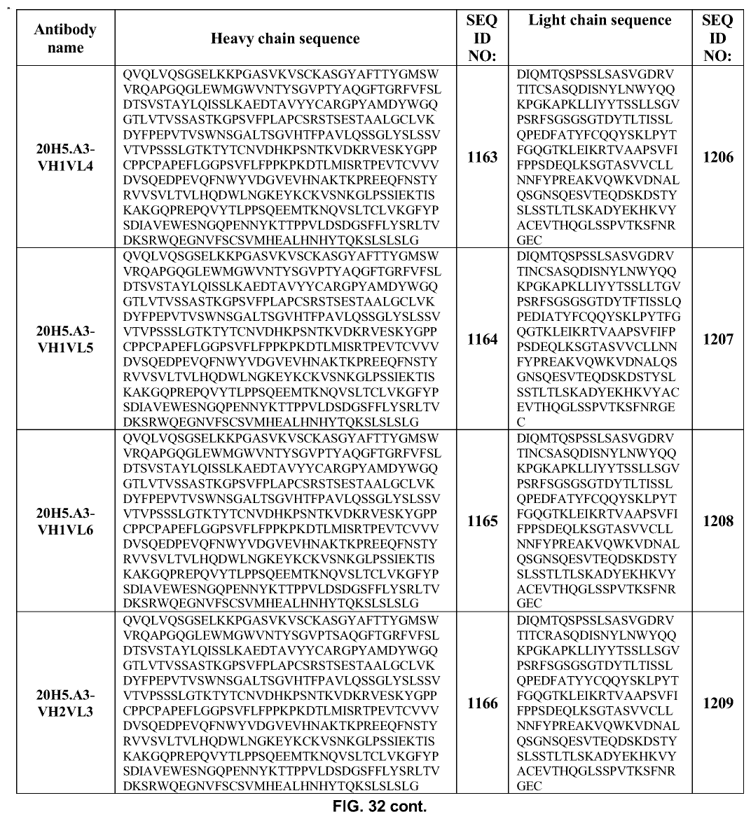

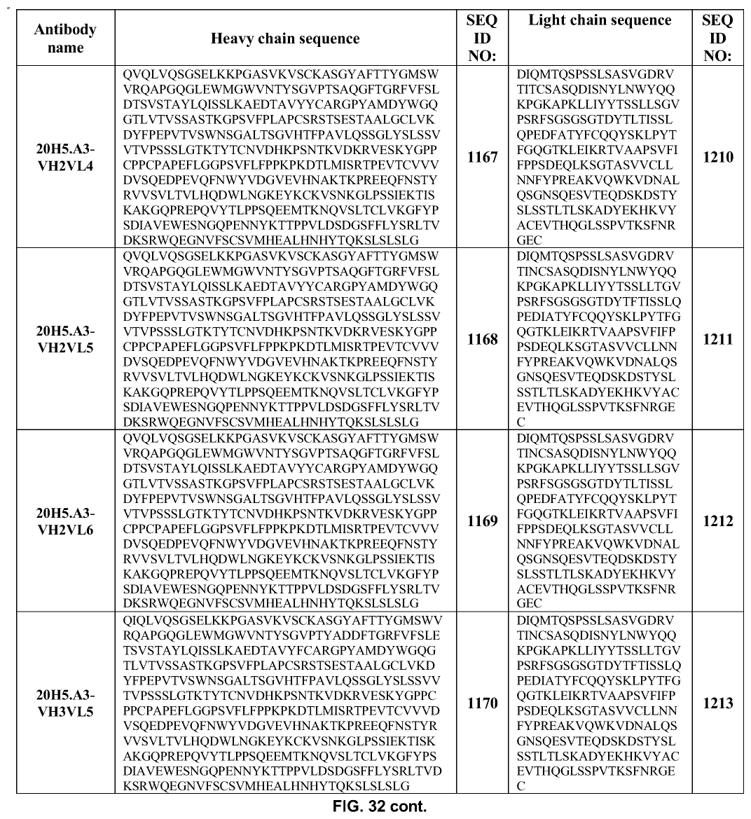

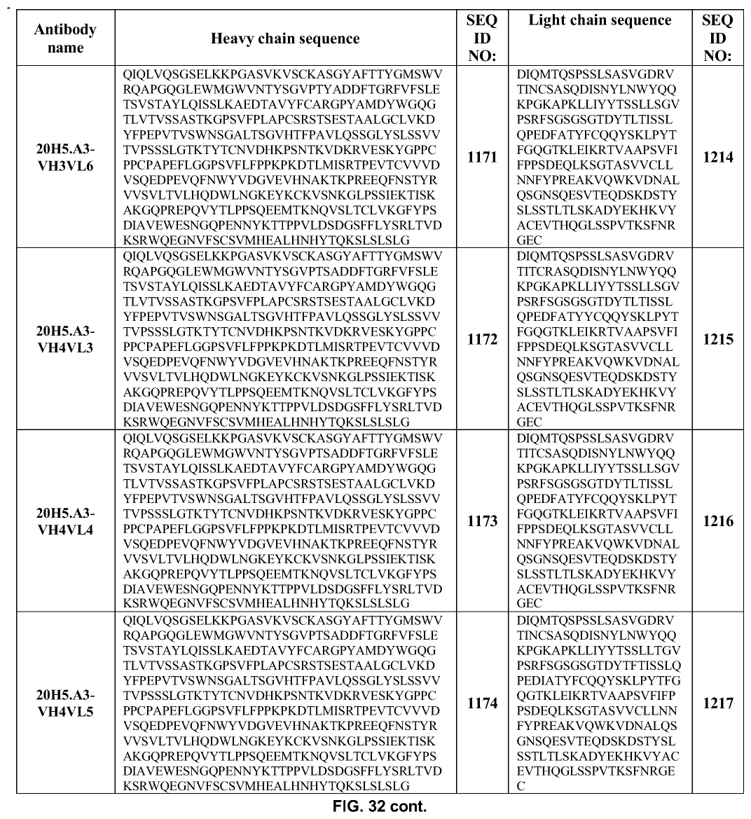

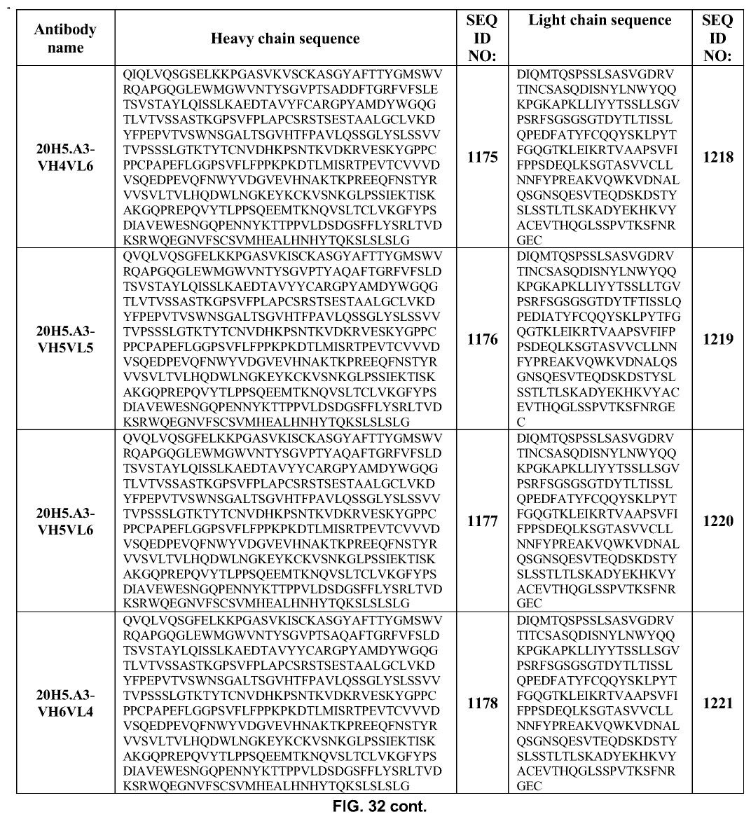

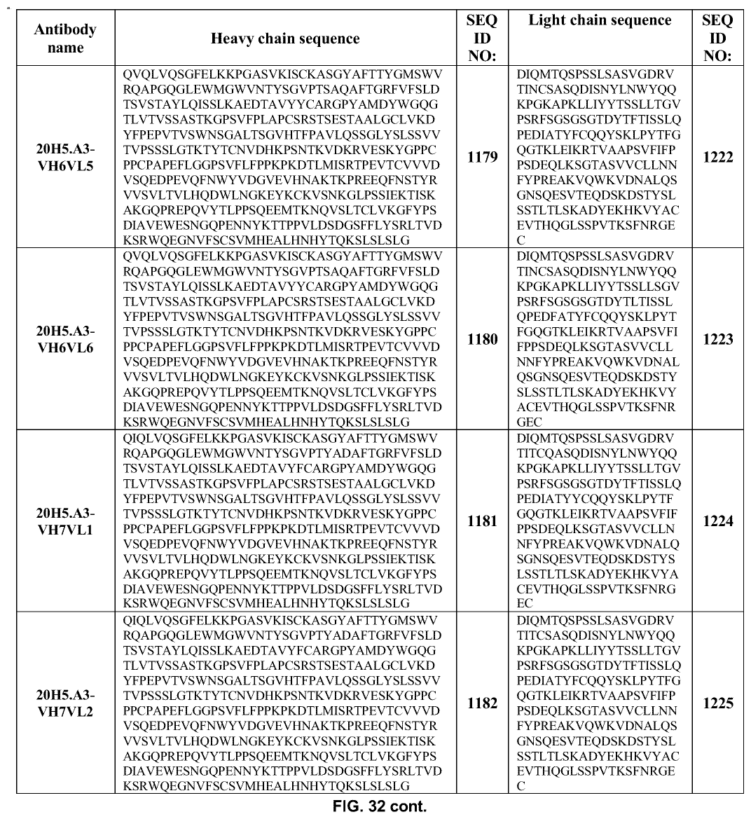

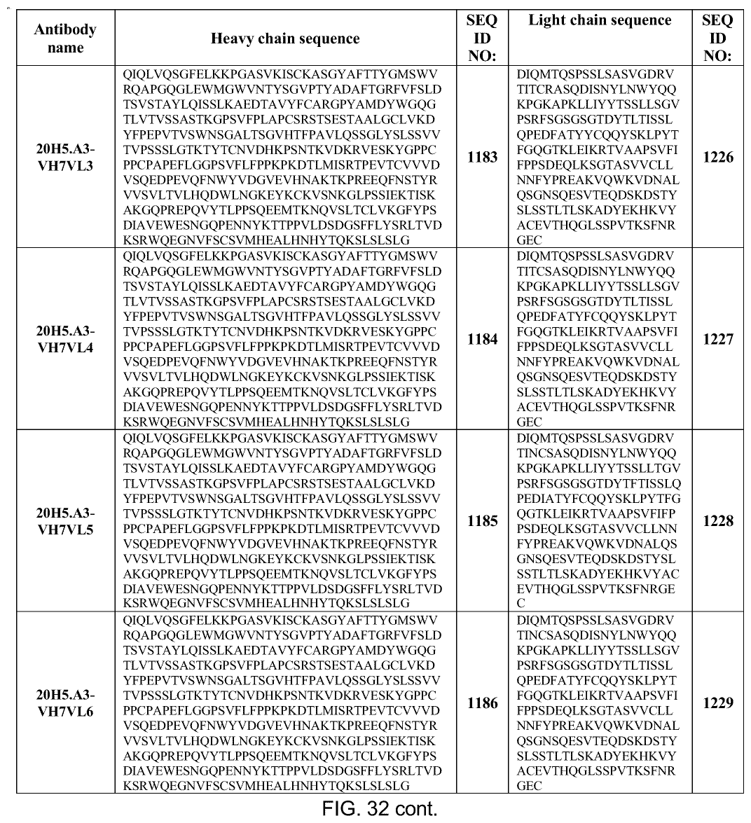

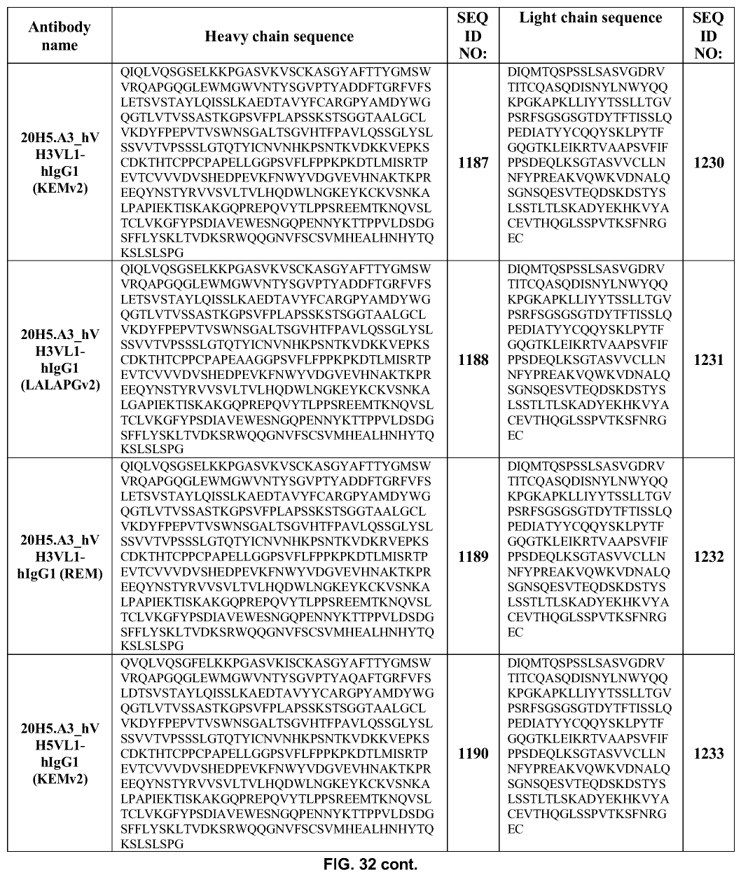

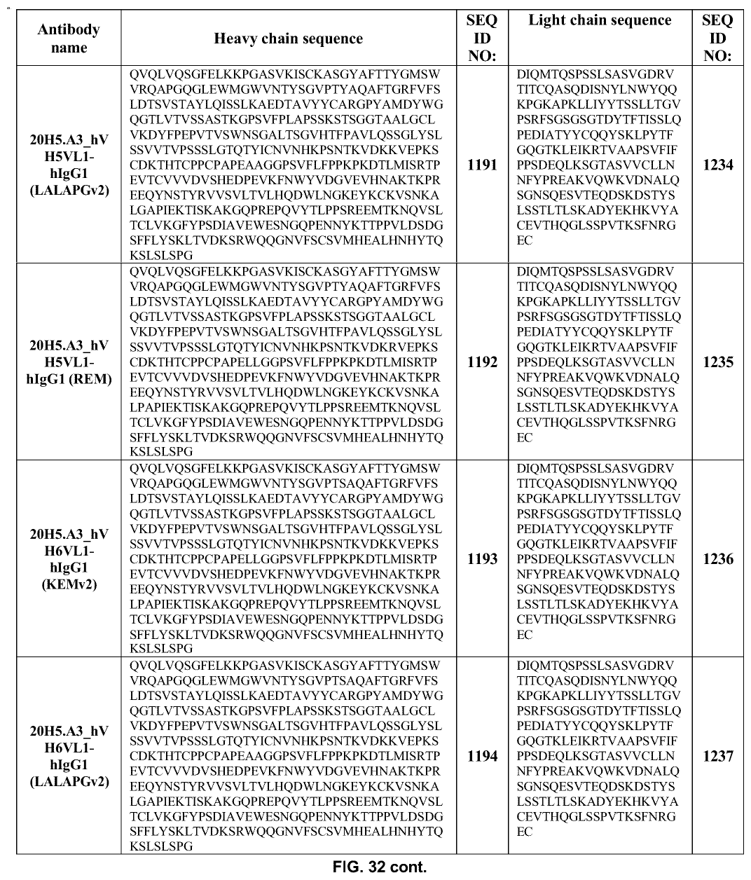

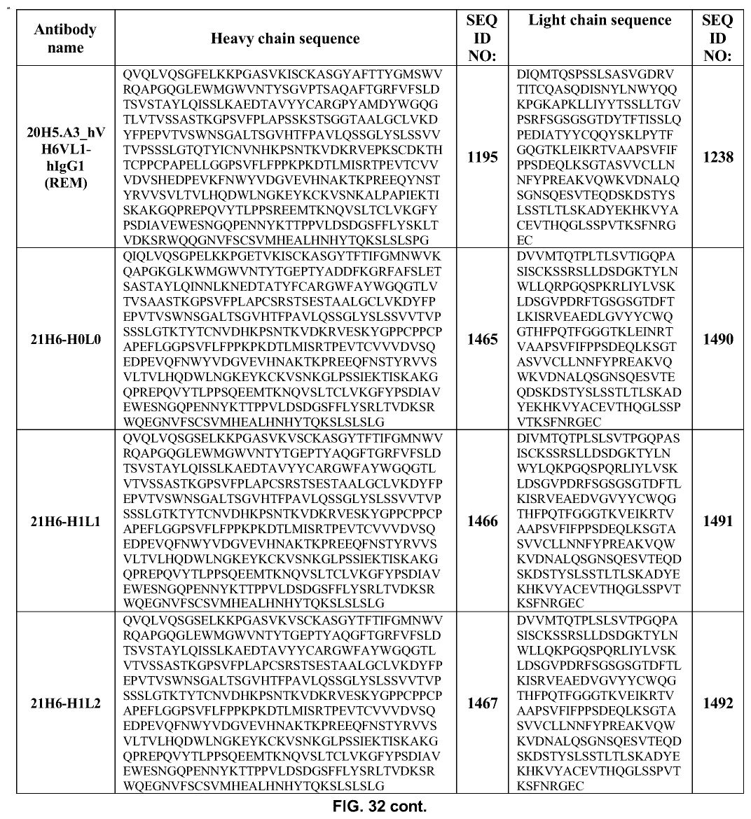

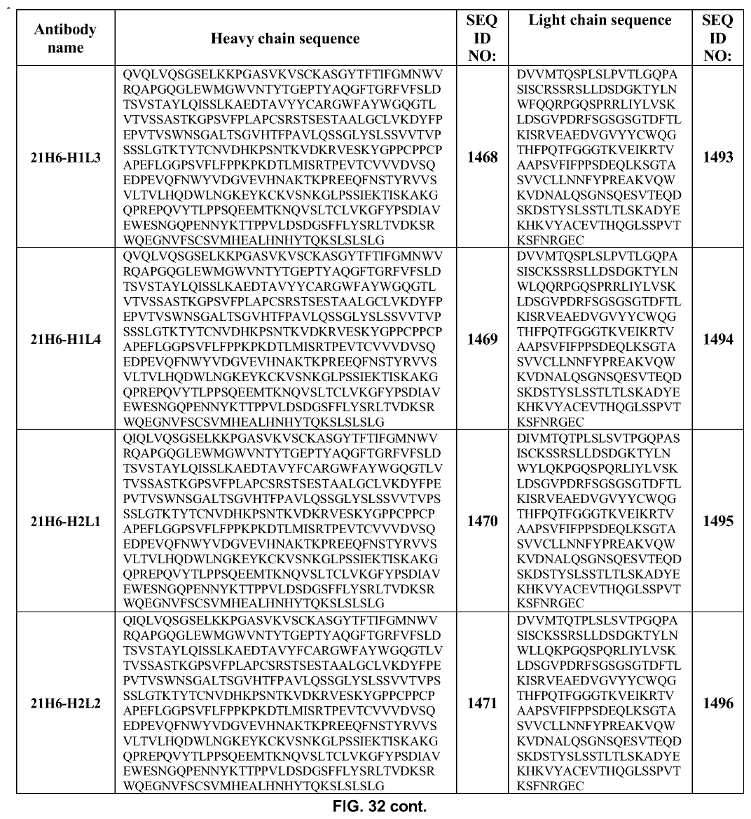

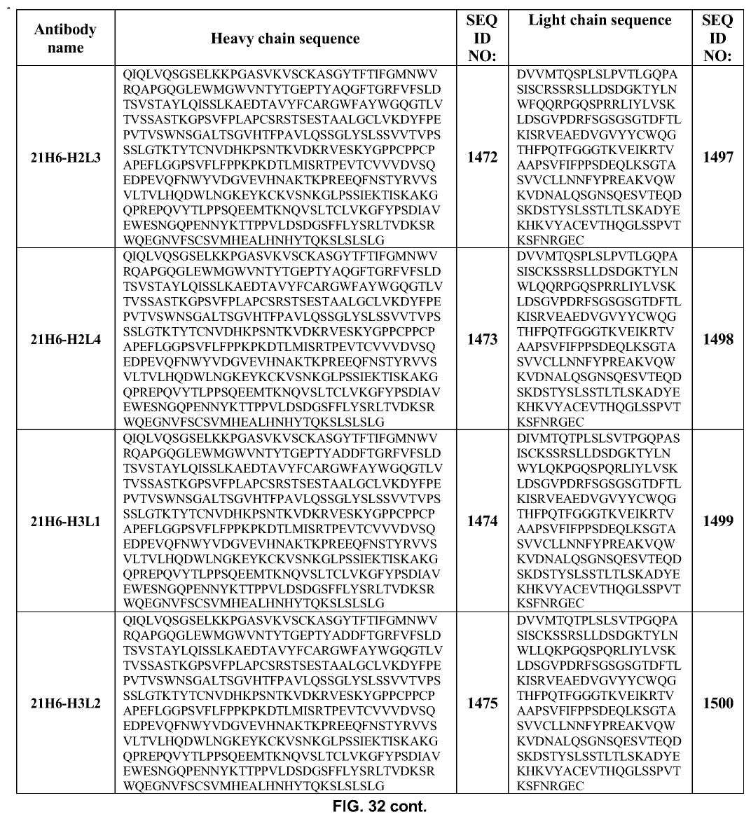

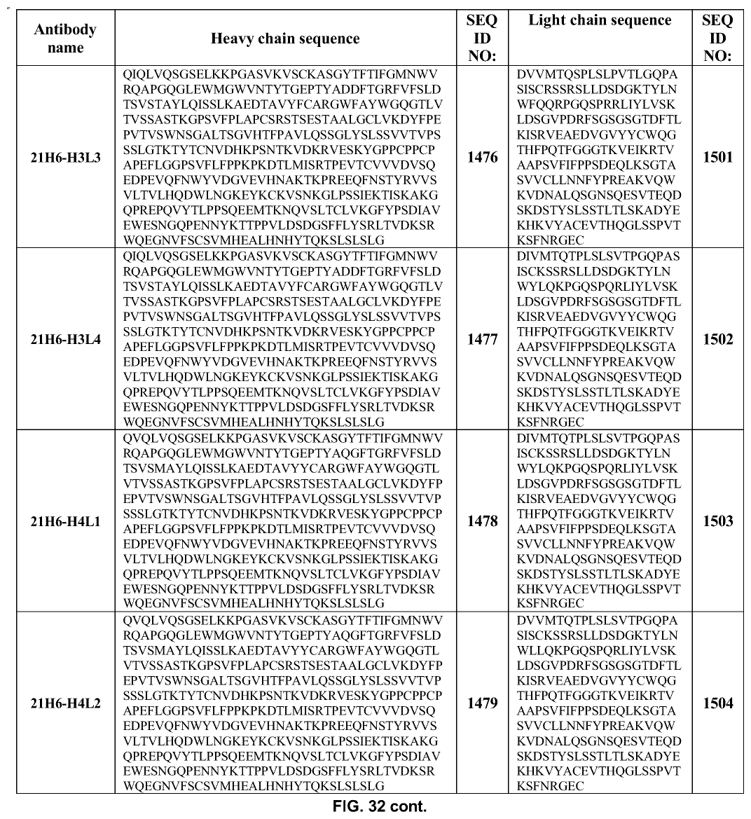

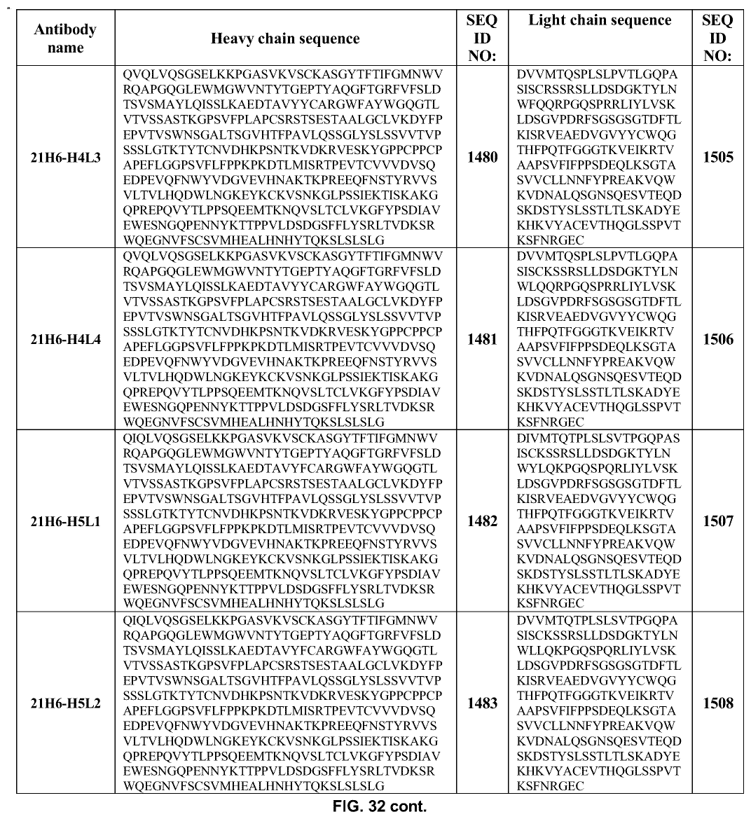

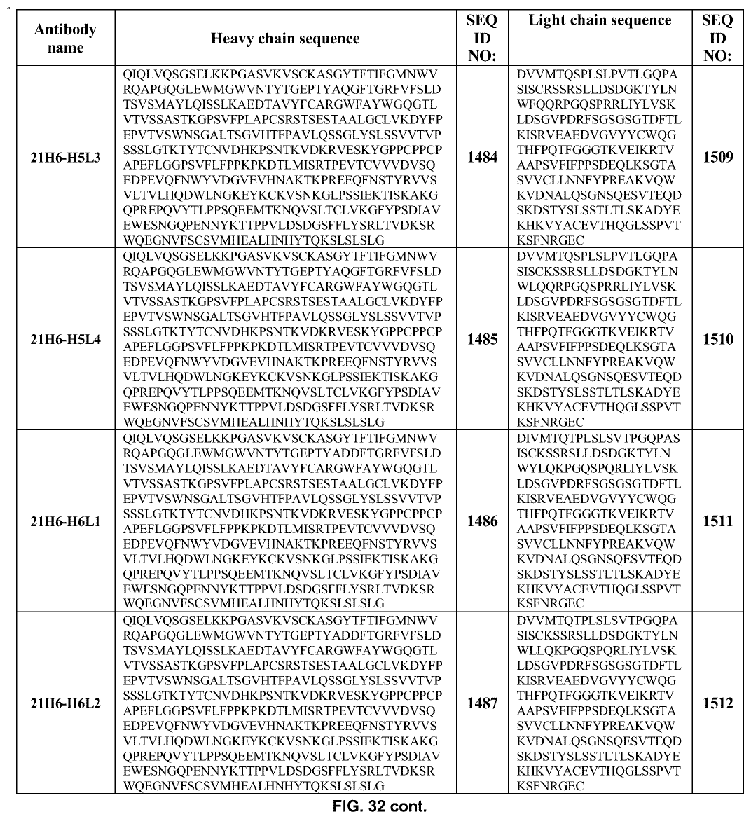

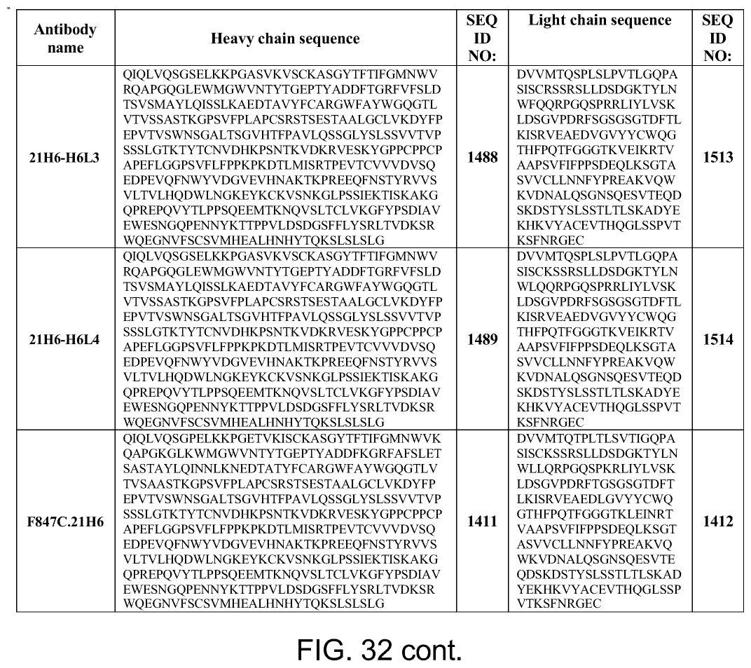

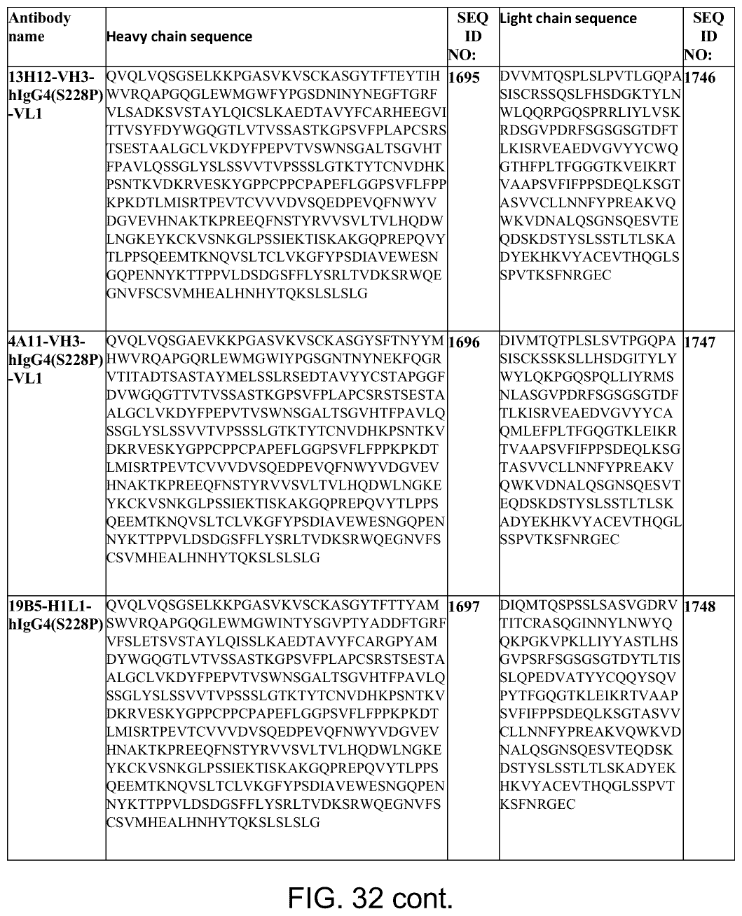

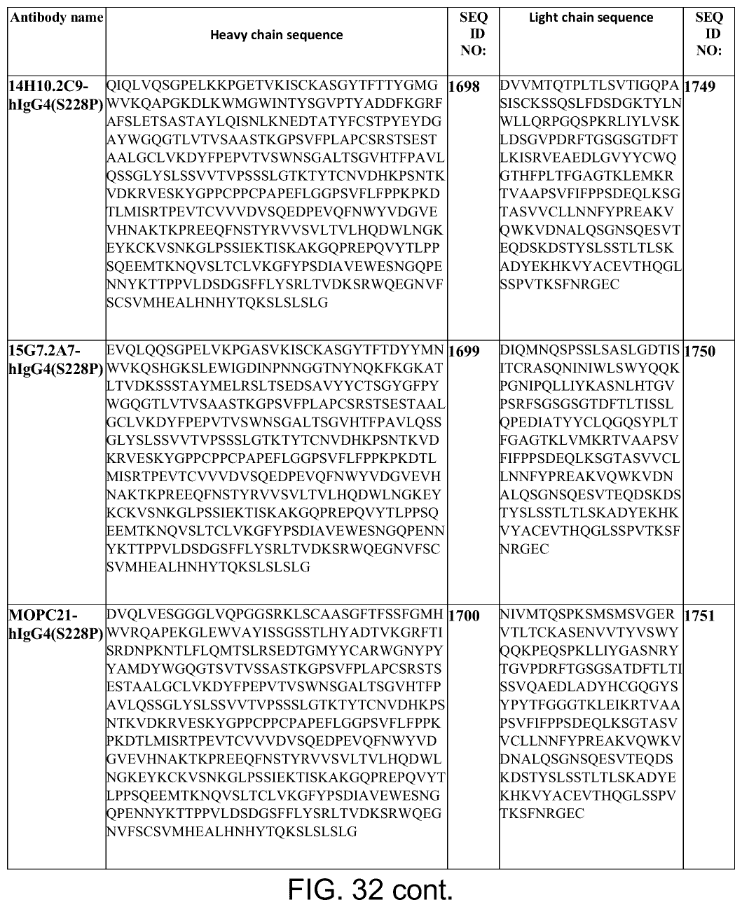

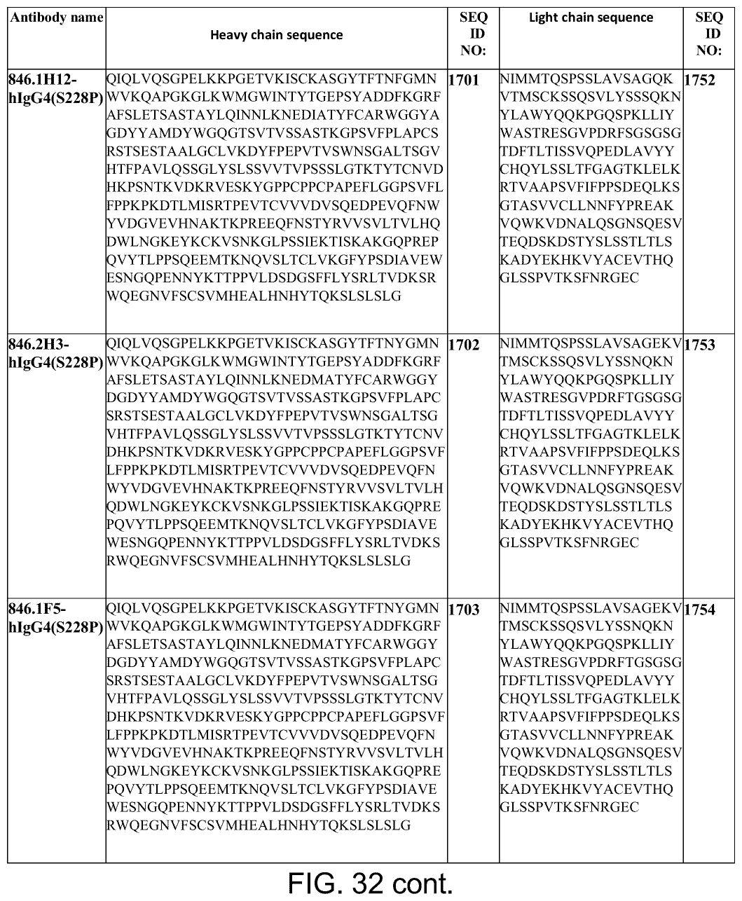

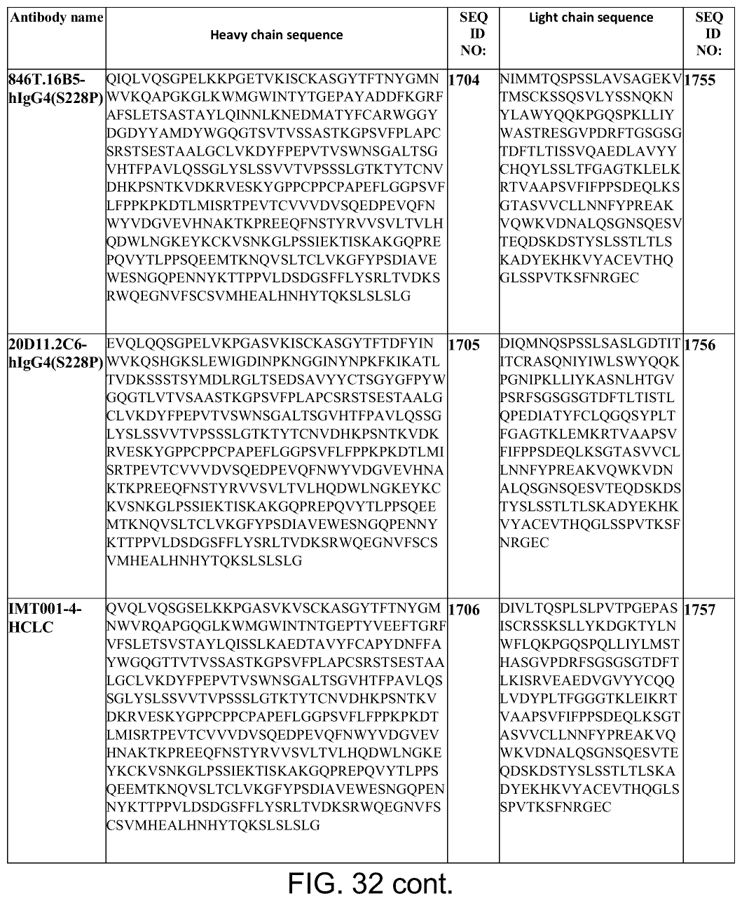

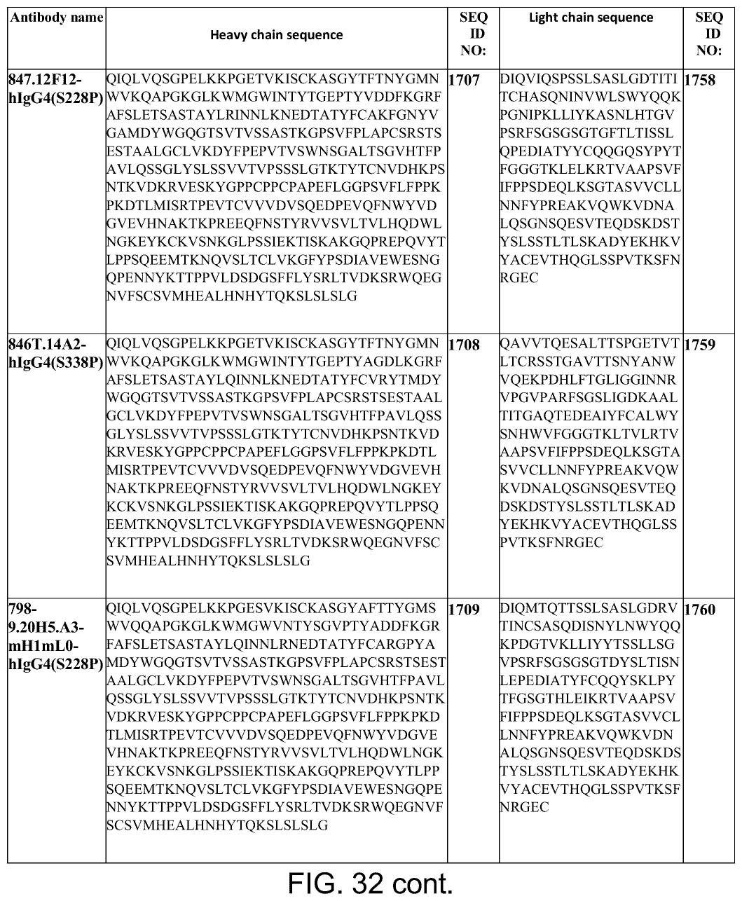

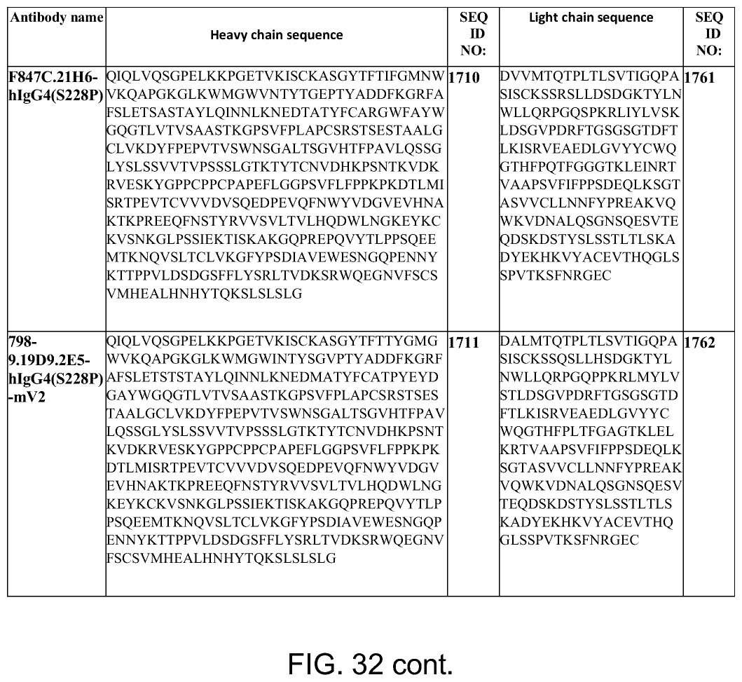

In some embodiments, the anti-Gal3 antibodies or binding fragments thereof comprise a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 27-70, 1763-1779. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 71-111, 801, 951, 952, 1780-1796. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 112-169, 802, 953, 954, 1797-1813. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 170-220, 1814-1830. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 211-247, 1831-1847. In some embodiments, the VL-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 248-296, 1848-1864. In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a VL sequence, a VH sequence, a VL/VH pairing, and/or V_{H}-CDR1, V_{H}-CDR2, V_{H}-CDR3, V_{L}-CDR1, V_{L}-CDR2, V_{L}-CDR3 (including 1, 2, 3, 4, or 5 amino acid substitutions of any one or more of these CDRs) set from the heavy chain and light chain sequences as depicted in FIG. 32.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 0, 1, 2, 3, 4, 5, or 6 substitutions relative to any amino acid sequence according to SEQ ID Nos: 27-70, 1763-1779. In some embodiments, the V_{H}-CDR2 comprises an amino acid sequence having at least 0, 1, 2, 3, 4, 5, or 6 substitutions relative to any amino acid sequence according to SEQ ID Nos: 71-111, 801, 951, 952, 1780-1796. In some embodiments, the V_{H}-CDR3 comprises an amino acid sequence having at least 0, 1, 2, 3, 4, 5, or 6 substitutions relative to any amino acid sequence according to SEQ ID Nos: 112-169, 802, 953, 954, 1797-1813. In some embodiments, the V_{L}-CDR1 comprises an amino acid sequence having at least 0, 1, 2, 3, 4, 5, or 6 substitutions relative to any amino acid sequence according to SEQ ID Nos: 170-220, 1814-1830. In some embodiments, the V_{L}-CDR2 comprises an amino acid sequence having at least 0, 1, 2, 3, 4, 5, or 6 substitutions relative to any amino acid sequence according to SEQ ID Nos: 211-247, 1831-1847. In some embodiments, the V_{L}-CDR3 comprises an amino acid sequence having at least 0, 1, 2, 3, 4, 5, or 6 substitutions relative to any amino acid sequence according to SEQ ID Nos: 248-296, 1848-1864.

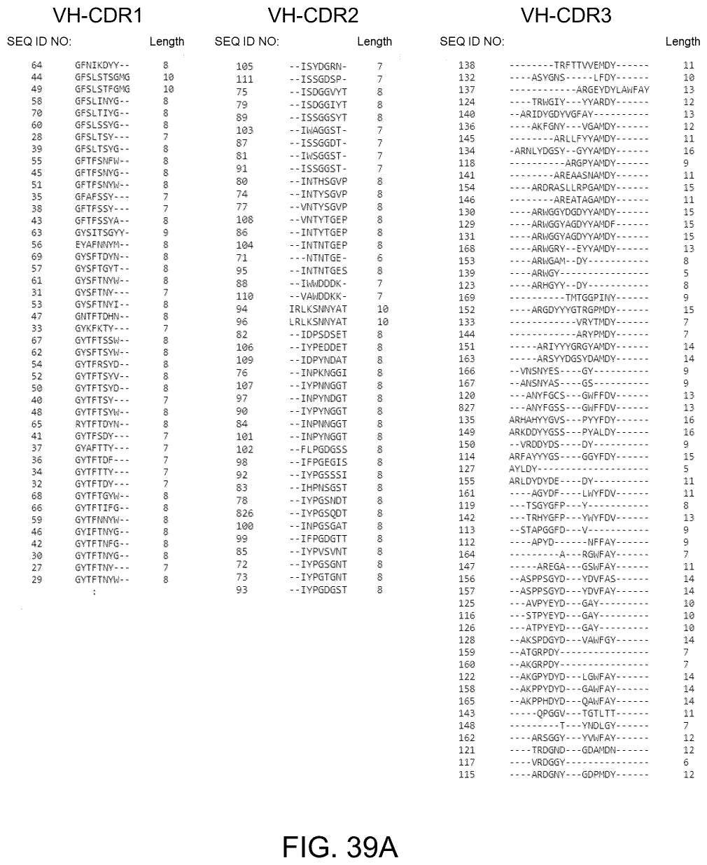

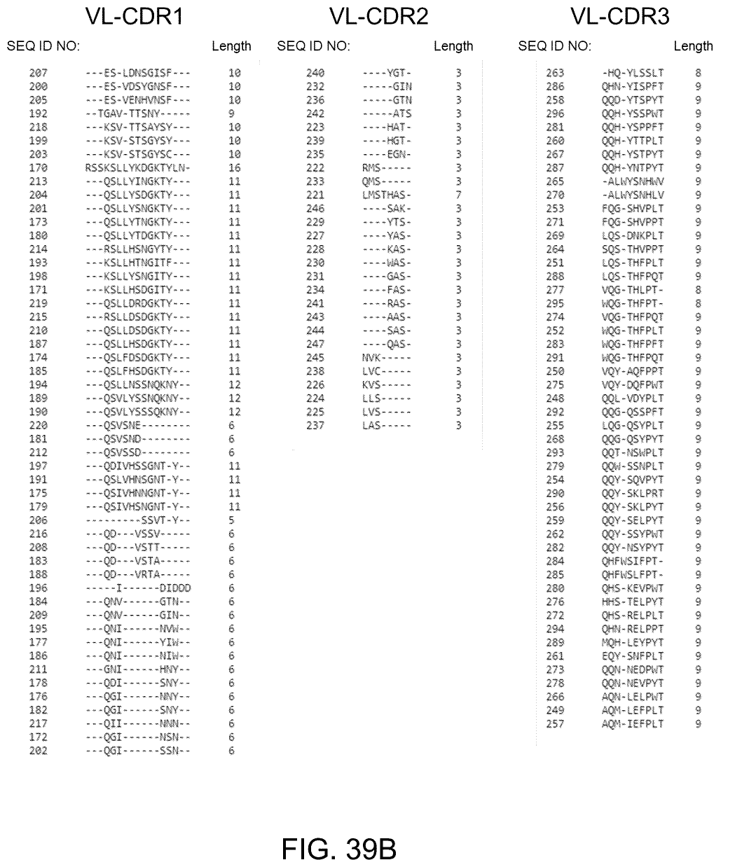

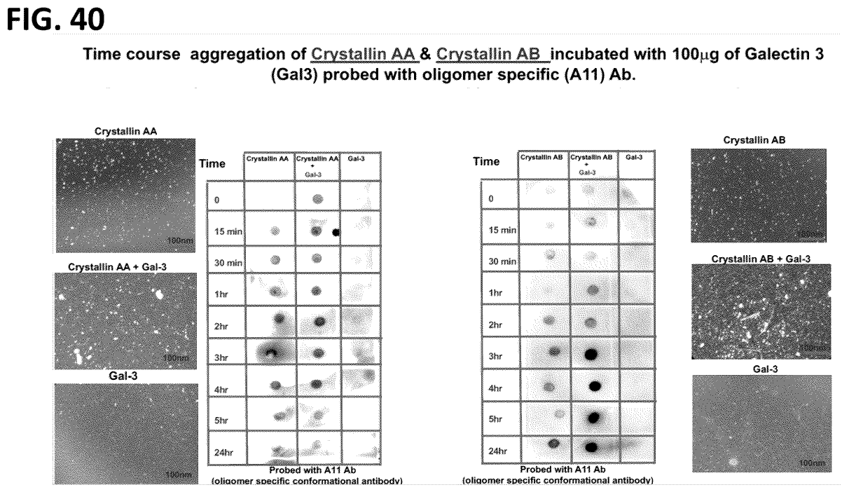

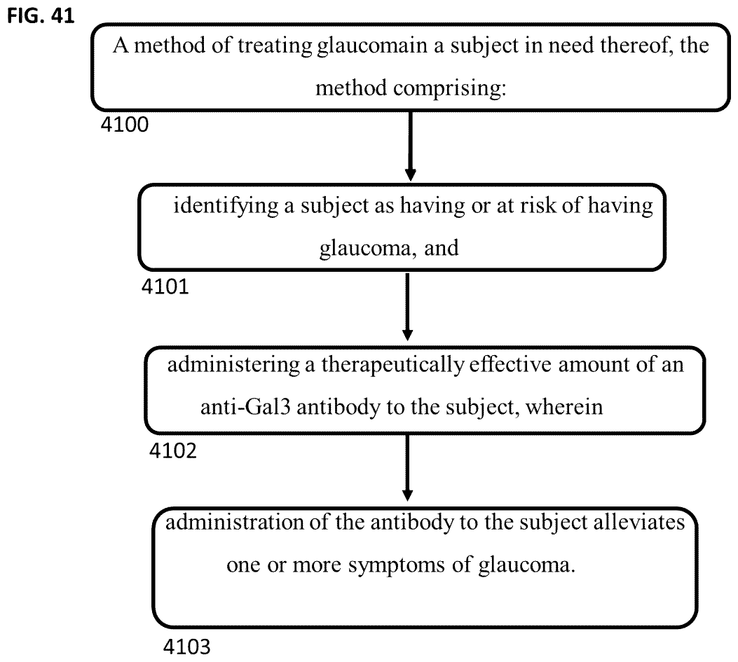

In some embodiments, the antibody or binding fragment thereof comprises a combination of a V_{H}-CDR1, a V_{H}-CDR2, a V_{H}-CDR3, V_{L}-CDR1, a V_{L}-CDR2, and a V_{L}-CDR3, where one or more of these CDRs is defined by a consensus sequence. The consensus sequences provided herein have been derived from the alignments of CDRs depicted in FIG. 39A-B. However, it is envisioned that alternative alignments may be done (e.g., using global or local alignment, or with different algorithms, such as Hidden Markov Models, seeded guide trees, Needleman-Wunsch algorithm, or Smith-Waterman algorithm) and as such, alternative consensus sequences can be derived.

In some embodiments, the V_{H}-CDR1 is defined by the formula X₁X₂X₃X₄X₅X₆X₇X₈X₉X₁₀ (SEQ ID NO. 1915), where X₁ is E, G, or R; X₂ is F, N, or Y; X₃ is A, I, K, N, S, or T; X₄ is F, I, or L; X₅ is I, K, N, R, S, or T; X₆ is D, G, I, N, S, or T; X₇ is F, G, H, S, or Y; X₈ is no amino acid, A, D, G, I, M, N, T, V, W, or Y; X₉ is no amino acid, M, or Y; X₁₀ is no amino acid or G; In some embodiments, the V_{H}-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR1 comprises a sequence having 0, 1, 2, 3, 4, 5, or 6 substitutions from this consensus sequence.

In some embodiments, the VH-CDR2 is defined by the formula X₁X₂X₃X₄X₅X₆X₇X₈X₉X₁₀ (SEQ ID NO. 1916), where X₁ is no amino acid, I, or L; X₂ is no amino acid or R; X₃ is no amino acid, F, I, L, or V; X₄ is A, D, F, H, K, L, N, S, W, or Y; X₅ is A, D, P, S, T, W, or Y; X₆ is D, E, G, H, K, N, S, V, or Y; X₇ is D, E, G, N, S, or T; X₈ is D, G, I, K, N, Q, R, S, V, or Y; X₉ is A, D, E, G, I, K, N, P, S, T, V, or Y; X₁₀ is no amino acid, I, P, S, or T. In some embodiments, the V_{H}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR2 comprises a sequence having 0, 1, 2, 3, 4, 5, or 6 substitutions from this consensus sequence.

In some embodiments, the V_{H}-CDR3 is defined by the formula X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅ (SEQ ID NO. 1917), where X₁ is no amino acid or A; X₂ is no amino acid, A, R, or Y; X₃ is no amino acid, A, F, H, K, L, R, S, or V; X₄ is no amino acid, A, D, K, N, R, S, or T; X₅ is no amino acid, A, D, G, H, I, L, N, P, R, S, T, V, or Y; X₆ is no amino acid, A, D, G, H, K, N, P, Q, R, S, or Y; X₇ is no amino acid, D, F, G, H, P, R, S, W, or Y; X₈ is no amino acid, A, D, E, G, I, R, or S; X₉ is no amino acid, A, C, D, E, F, G, I, N, R, S, T, V, or Y; X₁₀ is no amino acid, A, D, M, P, R, S, T, V, or Y; X₁₁ is no amino acid, A, D, E, F, L, T, V, or Y; X₁₂ is no amino acid, A, G, L, M, R, or T; X₁₃ is no amino acid, A, D, E, F, G, R, S, T, or V; X₁₄ is no amino acid, A, D, G, L, P, Q, R, S, T, V, or Y; X₁₅ is no amino acid, A, D, G, N, S, V, W, or Y; X₁₆ is no amino acid, A, D, E, F, L, P, T, V, W, or Y; X 17 is no amino acid, F, I, L, M, R, or Y; X₁₈ is no amino acid, A, D, G, N, or T; X₁₉ is no amino acid, F, N, S, T, V, or Y; X₂₀ is no amino acid or L; X₂₁ is no amino acid or A; X₂₂ is no amino acid or W; X₂₃ is no amino acid or F; X₂₄ is no amino acid or A; X₂₅ is no amino acid or Y. In some embodiments, the V_{H}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR3 comprises a sequence having 0, 1, 2, 3, 4, 5, or 6 substitutions from this consensus sequence.

In some embodiments, the VL-CDR1 is defined by the formula X₁X₂X₃X₄X₅X₆X₇X₈X₉X₁₀X₁₁X₁₂X₁₃X₁₄X₁₅X₁₆X₁₇ (SEQ ID NO. 1918), where X₁ is no amino acid or R; X₂ is no amino acid or S; X₃ is no amino acid, S, or T; X₄ is no amino acid, E, G, K, Q, or R; X₅ is no amino acid, A, D, G, I, N, or S; X₆ is no amino acid, I, L, or V; X₇ is no amino acid, F, L, S, or V; Xx is no amino acid, D, E, H, N, S, T, or Y; X₉ is no amino acid, D, E, I, K, N, R, S, T, or V; X₁₀ is no amino acid, D, H, N, R, S, or Y; X₁₁ is no amino acid, A, G, N, S, T, or V; X₁₂ is no amino acid, A, I, K, N, Q, T, V, or Y; X₁₃ is no amino acid, D, G, H, K, N, S, T, or Y; X₁₄ is no amino acid, C, F, I, N, S, T, V, or Y; X₁₅ is no amino acid, D, L, N, W, or Y; X₁₆ is no amino acid, N, or D; X₁₇ is no amino acid or D. In some embodiments, the V_{L}-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR1 comprises a sequence having 0, 1, 2, 3, 4, 5, or 6 substitutions from this consensus sequence.

In some embodiments, the V_{L}-CDR2 is defined by the formula X₁X₂X₃X₄X₅X₆X₇X₈ (SEQ ID NO. 1919), where X₁ is no amino acid, K, L, N, Q, or R; X₂ is no amino acid, A, L, M, or V; X₃ is no amino acid, C, K, or S; X₄ is no amino acid or T; X₅ is no amino acid, A, E, F, G, H, K, Q, R, S, W, or Y; X₆ is no amino acid, A, G, or T; X₇ is no amino acid, I, K, N, S, or T; Xx is no amino acid, N, or S. In some embodiments, the V_{L}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR2 comprises a sequence having 0, 1, 2, 3, 4, 5, or 6 substitutions from this consensus sequence.

In some embodiments, the V_{L}-CDR3 is defined by the formula X₁X₂X₃X₄X₅X₆X₇X₈X₉X₁₀ (SEQ ID NO. 1920), where X₁ is no amino acid, A, E, F, H, L, M, Q, S, V, or W; X₂ is A, H, or Q; X₃ is D, F, G, H, L, M, N, Q, S, T, W, or Y; X₄ is no amino acid or W; X₅ is A, D, I, K, L, N, Q, R, S, T, V, or Y; X₆ is D, E, H, I, K, L, N, Q, S, or T; X₇ is D, F, K, L, N, P, S, T, V, W, or Y; X₈ is H, P, or S; X₉ is F, L, P, Q, R, T, W, or Y; X₁₀ is no amino acid, T, or V. In some embodiments, the V_{L}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR3 comprises a sequence having 0, 1, 2, 3, 4, 5, or 6 substitutions from this consensus sequence.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID Nos: 297-373, 803, 806-820, 955-968, 1067-1109, 1415-1439, 1882-1898. In some embodiments, the light chain variable region of the antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID Nos: 374-447, 821-835, 941-943, 969-982, 1110-1152, 1440-1464, 1899-1914, 1921. In some embodiments, the antibodies or binding fragments thereof are anti-Gal3 antibodies or binding fragments thereof.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID Nos: 297-373, 803, 806-820, 955-968, 1067-1109, 1415-1439, 1882-1898. In some embodiments, the light chain variable region of the antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID Nos: 374-447, 821-835, 941-943, 969-982, 1110-1152, 1440-1464, 1899-1914, 1921. In some embodiments, the antibodies or binding fragments thereof are anti-Gal3 antibodies or binding fragments thereof.

In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a V_{L} sequence, a V_{H} sequence, a VL/VH pairing, and/or V_{L}-CDR1, V_{L}-CDR2, V_{L}-CDR3, V_{H}-CDR1, V_{H}-CDR2, V_{H}-CDR3 (including 1, 2, 3, 4, or 5 amino acid substitutions of any one or more of these CDRs) set from the heavy chain and light chain sequences as depicted in FIG. 32.

In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises one of the amino acid sequences of SEQ ID Nos: 27-70, 1763-1779, the V_{H}-CDR2 comprises one of the amino acid sequences of SEQ ID Nos: 71-111, 801, 951, 952, 1780-1796, the V_{H}-CDR3 comprises one of the amino acid sequences of SEQ ID NO: 112-169, 802, 953, 954, 1797-1813, the V_{L}-CDR1 comprises one of the amino acid sequences of SEQ ID Nos: 170-220, 1814-1830, the V_{L}-CDR2 comprises one of the amino acid sequences of SEQ ID Nos: 211-247, 1831-1847, the V_{L}-CDR3 comprises one of the amino acid sequences of SEQ ID Nos: 248-296, 1848-1864, the heavy chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID Nos: 297-373, 803, 806-820, 955-968, 1067-1109, 1415-1439, 1882-1898, and the light chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID Nos: 374-447, 821-835, 941-943, 969-982, 1110-1152, 1440-1464, 1899-1914, 1921.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID Nos: 448-494, 804, 836-850, 983-996, 1153-1195, 1465-1489, 1695-1711. In some embodiments, the heavy chain further comprises a signal peptide having at least 90% identity to SEQ ID NO: 1922. In some embodiments, the heavy chain further comprises a signal peptide that is SEQ ID NO: 1922. In some embodiments, the antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID Nos: 495-538, 805, 851-865, 997-1010, 1196-1238, 1490-1514, 1746-1762. In some embodiments, the light chain further comprises a signal peptide. In some embodiments, the light chain further comprises a signal peptide having at least 90% identity to SEQ ID NO: 1923 or SEQ ID NO: 1924. In some embodiments, the light chain further comprises a signal peptide that is SEQ ID NO: 1923. In some embodiments, the light chain further comprises a signal peptide that is SEQ ID NO: 1924.

In some embodiments, the anti-Gal3 antibody or binding fragment thereof comprises a heavy chain, wherein the heavy chain is encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a sequence selected from SEQ ID NOs: 703-749, 799, 896-910, 1039-1052, 1325-1367, 1413, and 1565-1589. In some embodiments, the heavy chain further comprises a signal peptide encoded by a nucleic acid sequence having at least 90% identity to SEQ ID NO: 1925. In some embodiments, the antibody or binding fragment thereof comprises a light chain, wherein the light chain is encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 750-796, 911-925, 1053-1066, 1368-1410, 1414, and 1590-1614. In some embodiments, the light chain further comprises a signal peptide. In some embodiments, the light chain further comprises a signal peptide encoded by a nucleic acid sequence having at least 90% identity to SEQ ID NO: 1926 or SEQ ID NO: 1927. In some embodiments, the light chain further comprises a signal peptide encoded by a nucleic acid sequence that is SEQ ID NO: 1926. In some embodiments, the light chain further comprises a signal peptide that is SEQ ID NO: 1927.

In some embodiments, the antibodies or binding fragments thereof are anti-Gal3 antibodies or binding fragments thereof.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 448-494, 804, 836-850, 983-996, 1153-1195, 1465-1489, 1695-1711. In some embodiments, the heavy chain further comprises a signal peptide. In some embodiments, the heavy chain further comprises a signal peptide having at least 90% identity to SEQ ID NO: 1922. In some embodiments, the antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% similarity to the sequence selected from SEQ ID NOs: 495-538, 805, 851-865, 997-1010, 1196-1238, 1490-1514, 1746-1762. In some embodiments, the light chain further comprises a signal peptide. In some embodiments, the light chain further comprises a signal peptide having at least 90% identity to SEQ ID NO: 1923 or SEQ ID NO: 1924. In some embodiments, the light chain further comprises a signal peptide that is SEQ ID NO: 1923. In some embodiments, the light chain further comprises a signal peptide that is SEQ ID NO: 1924. In some embodiments, the antibodies or binding fragments thereof are anti-Gal3 antibodies or binding fragments thereof.

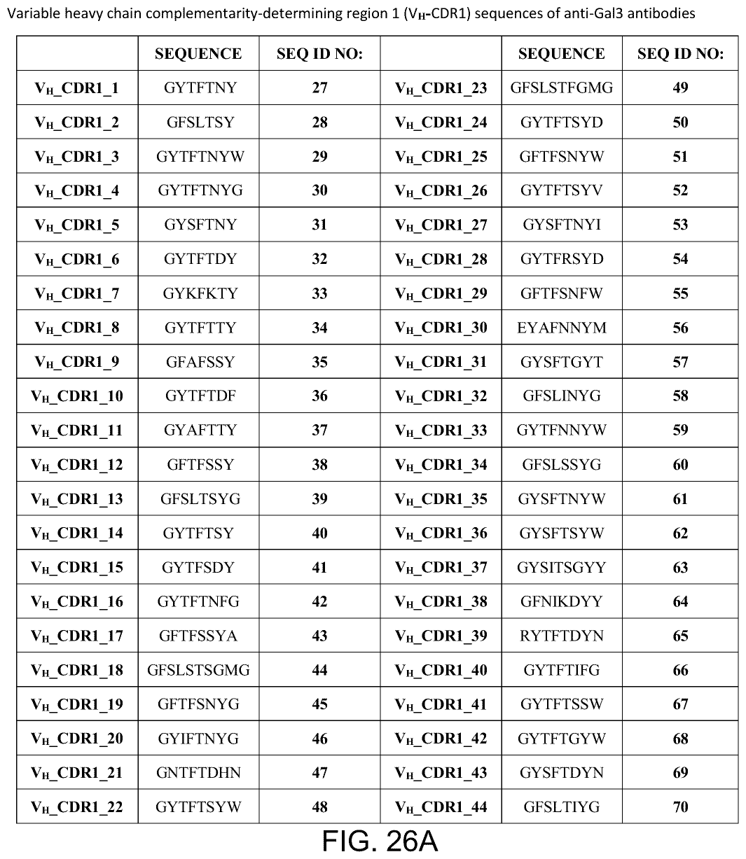

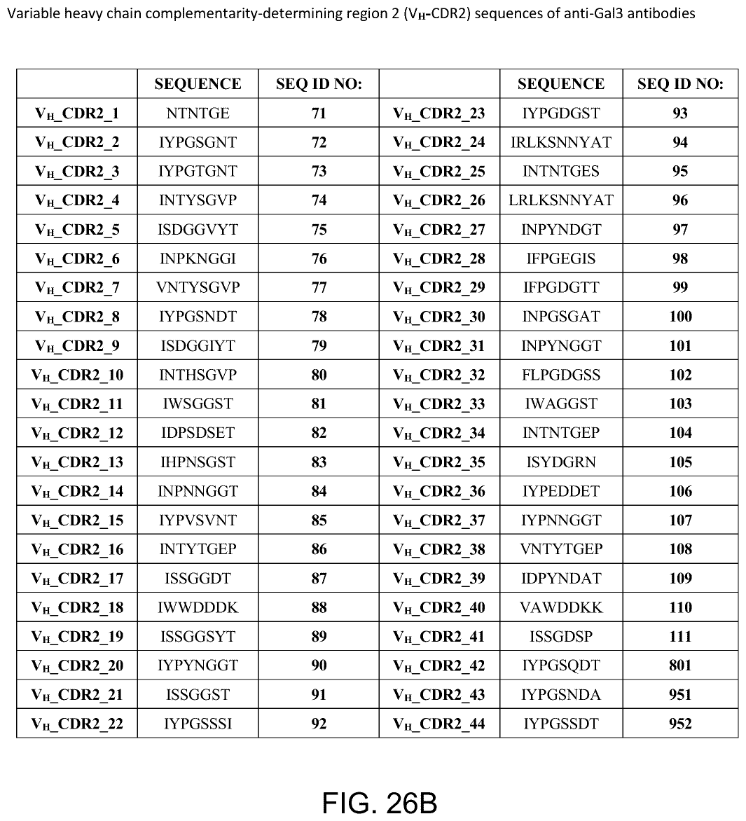

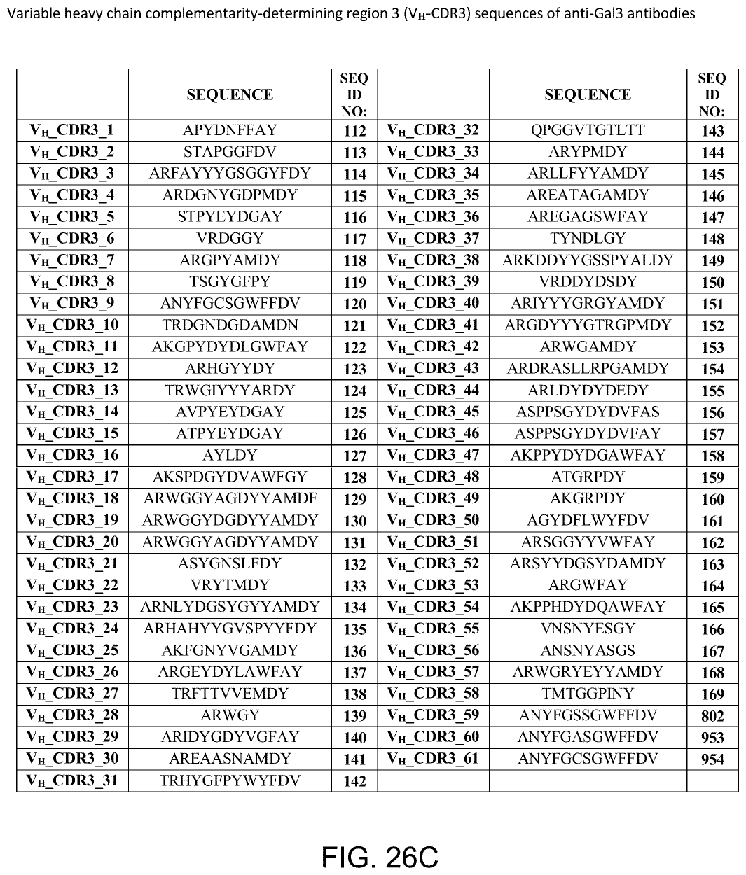

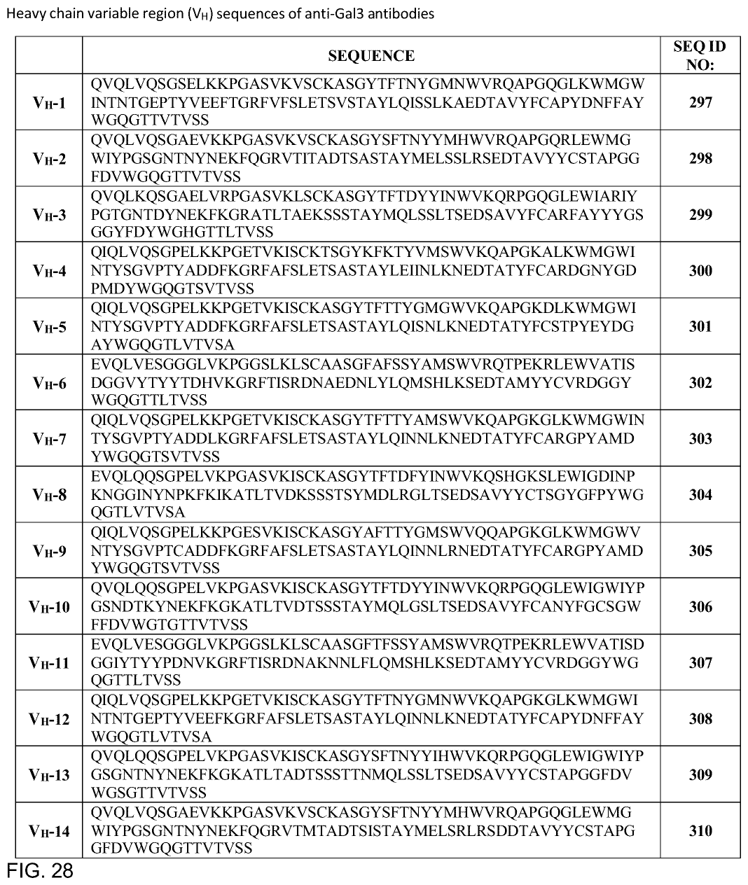

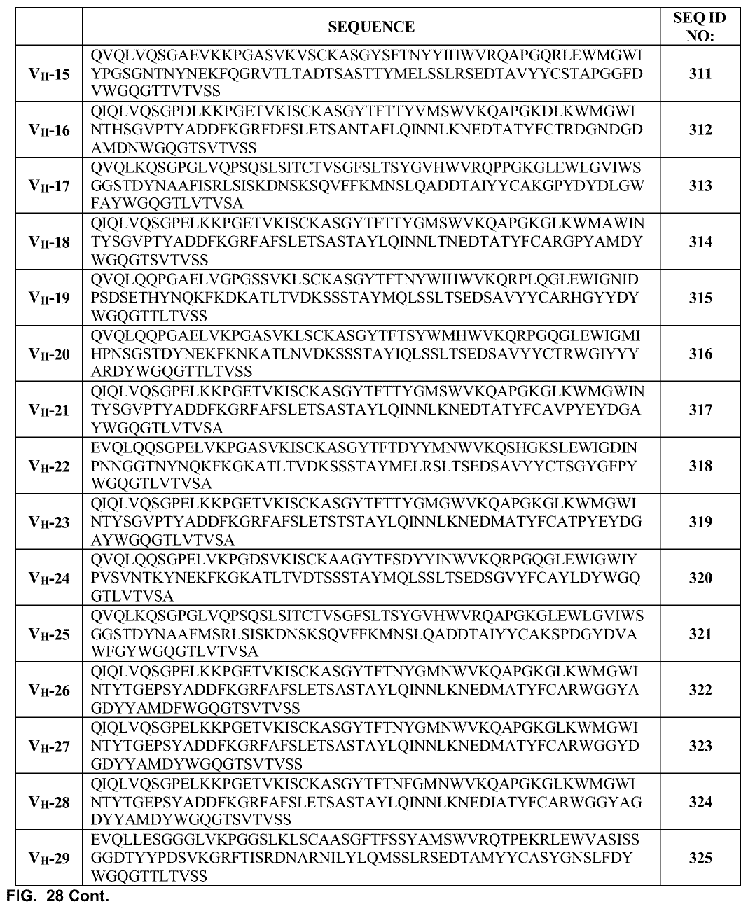

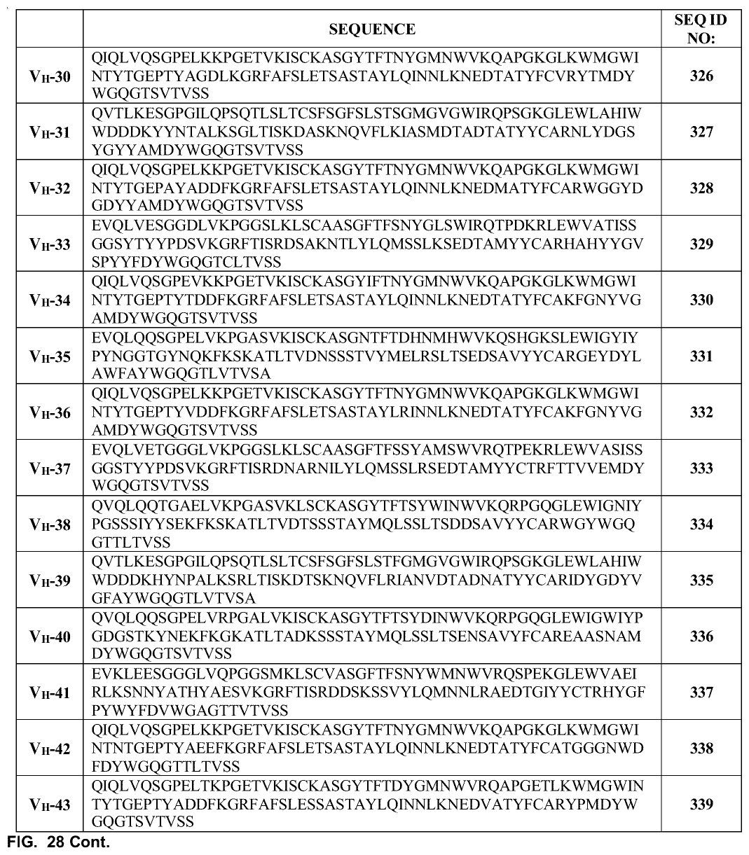

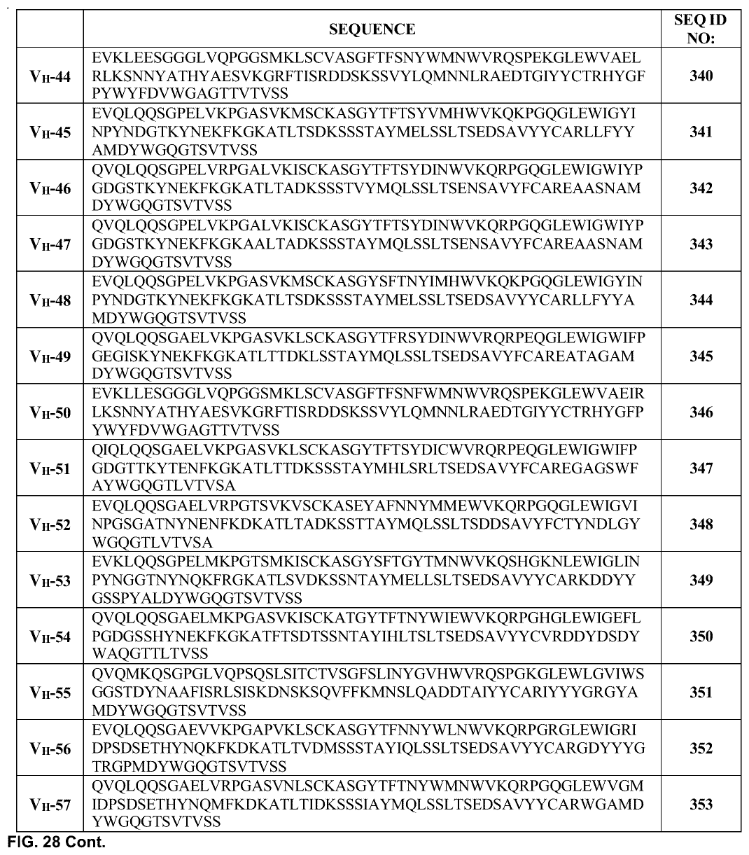

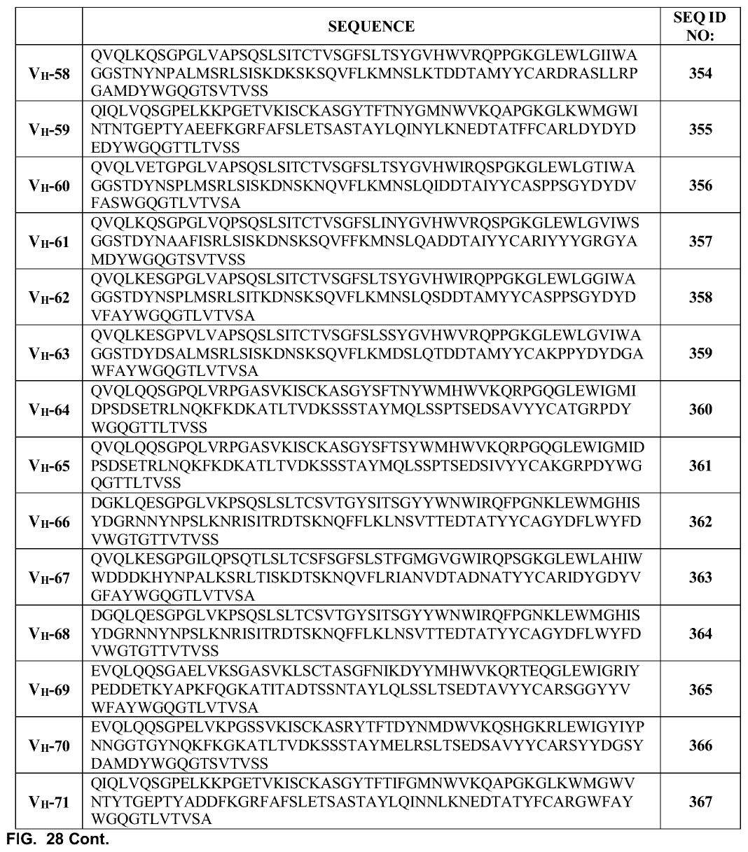

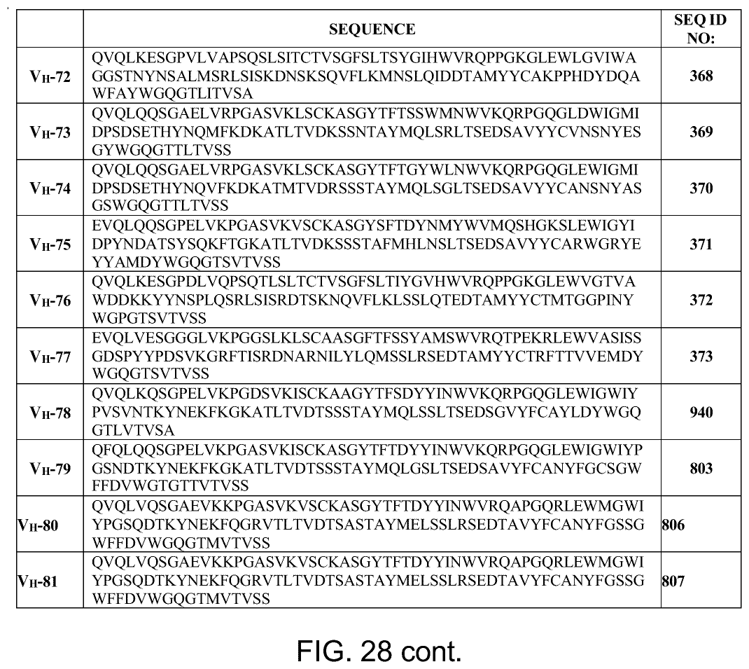

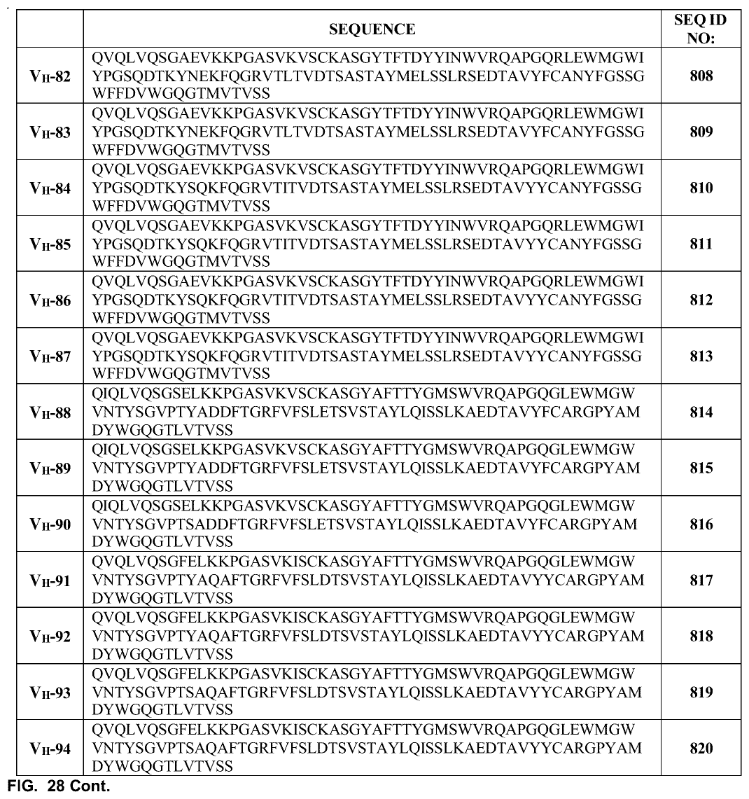

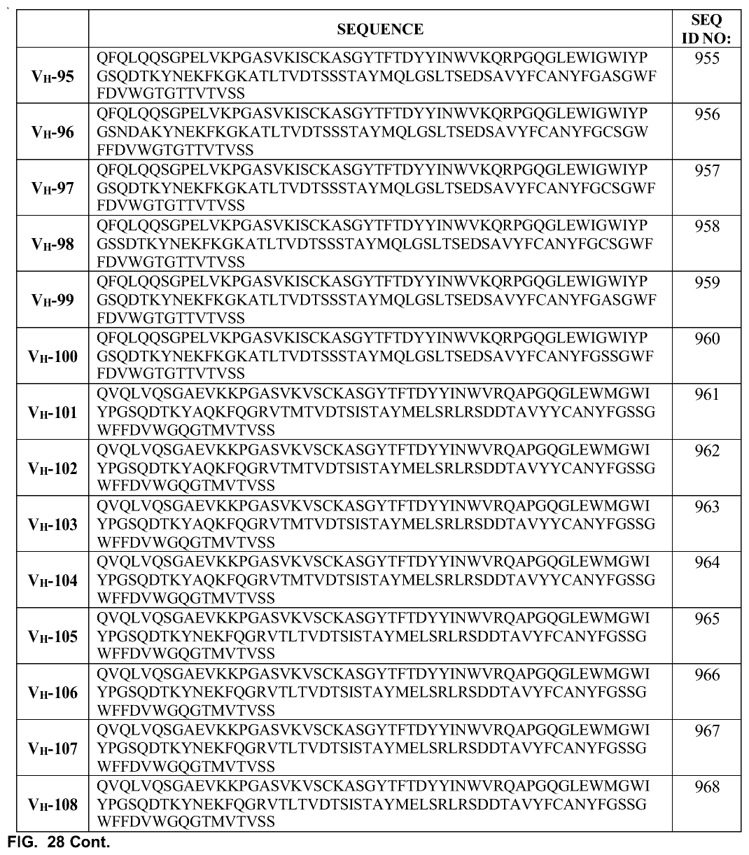

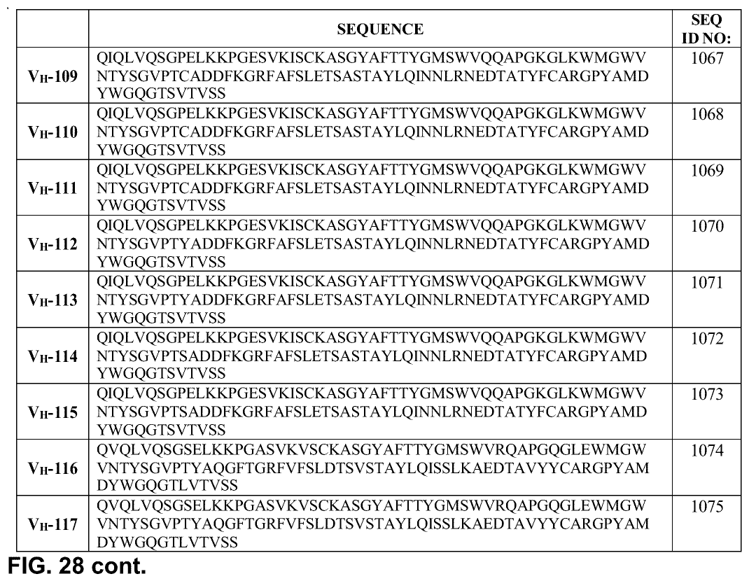

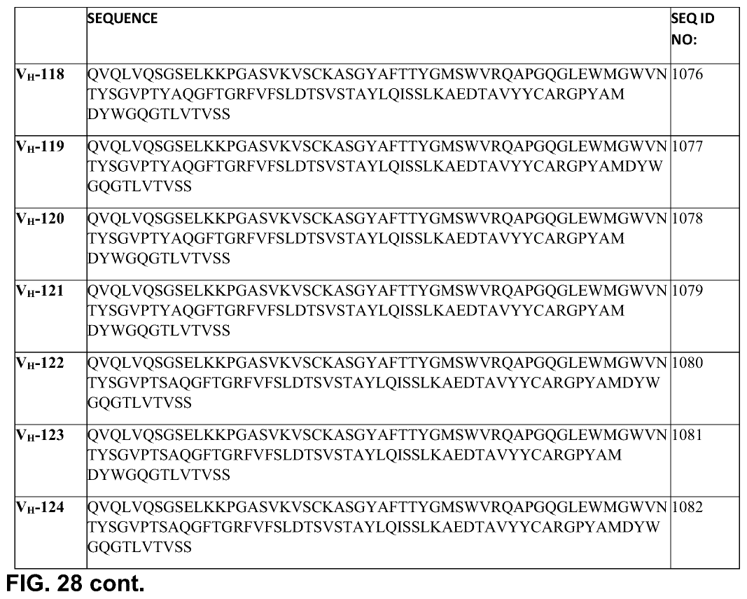

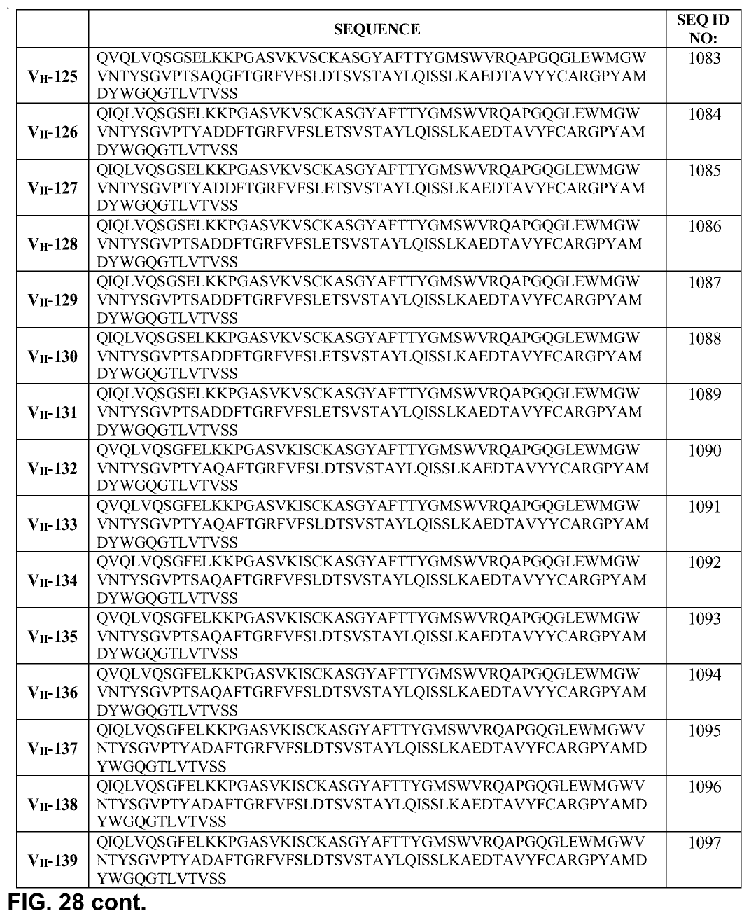

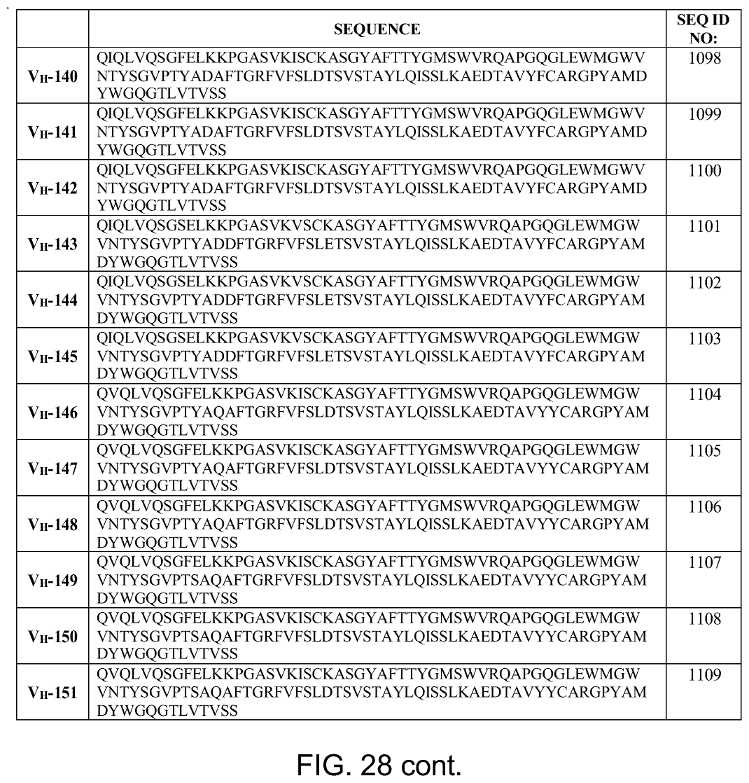

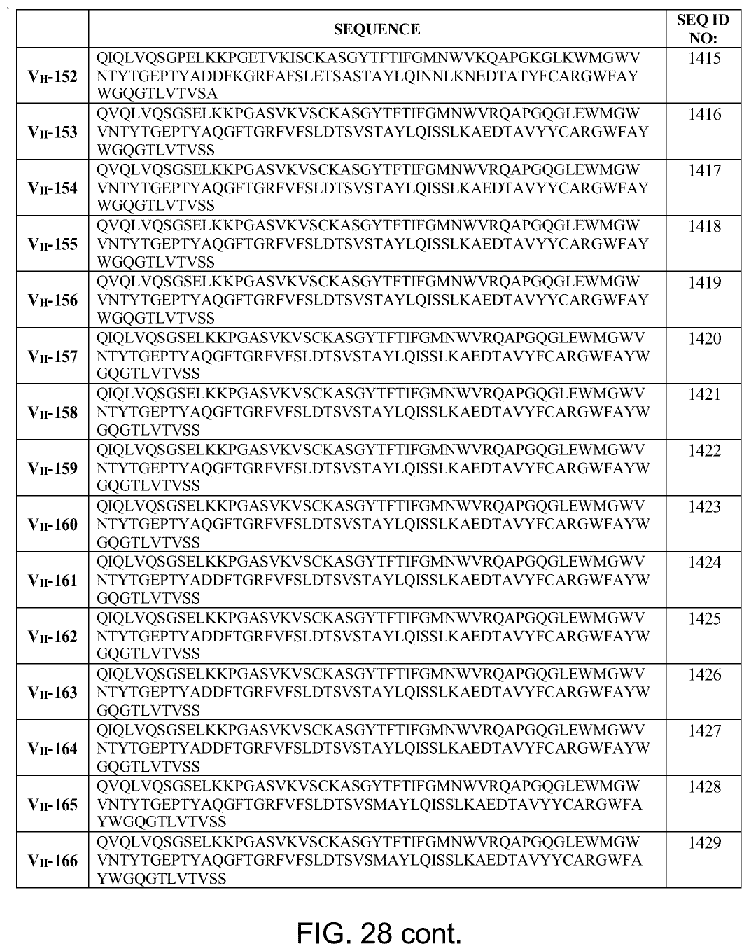

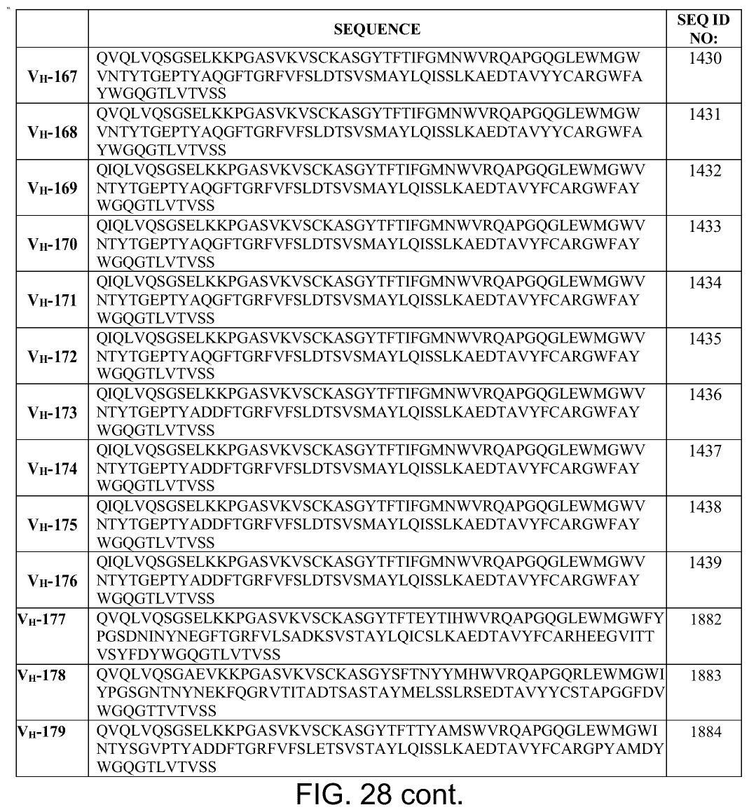

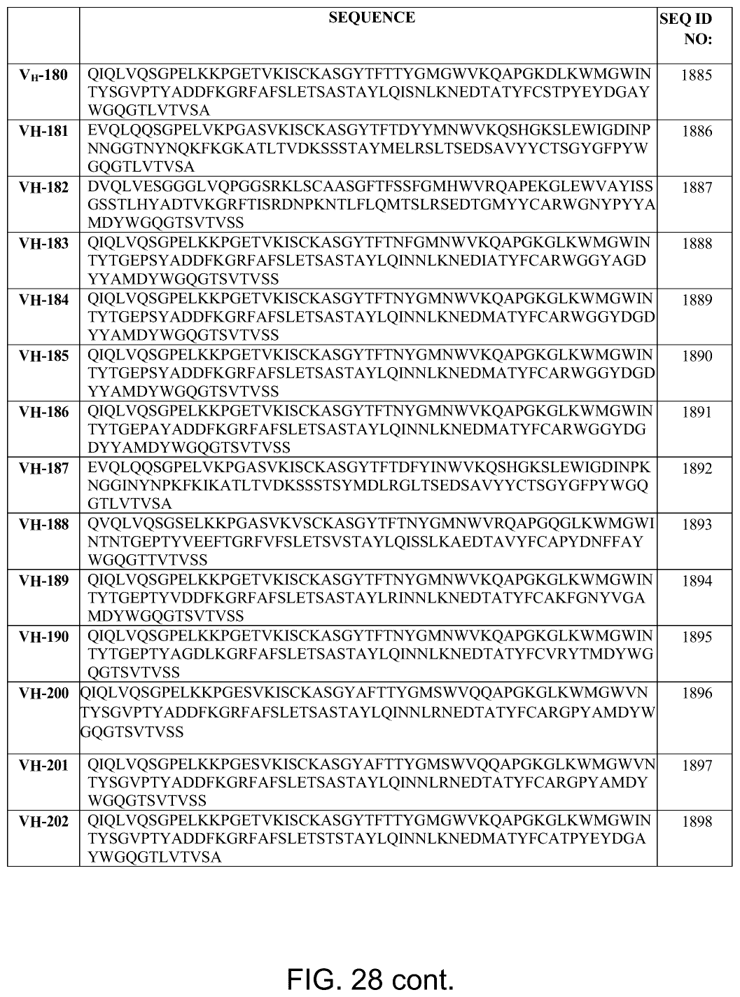

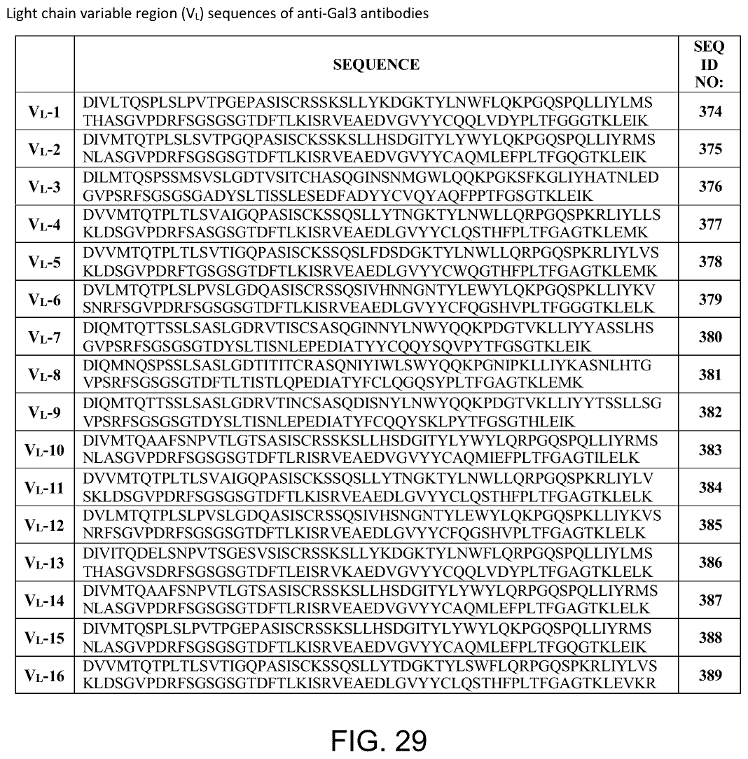

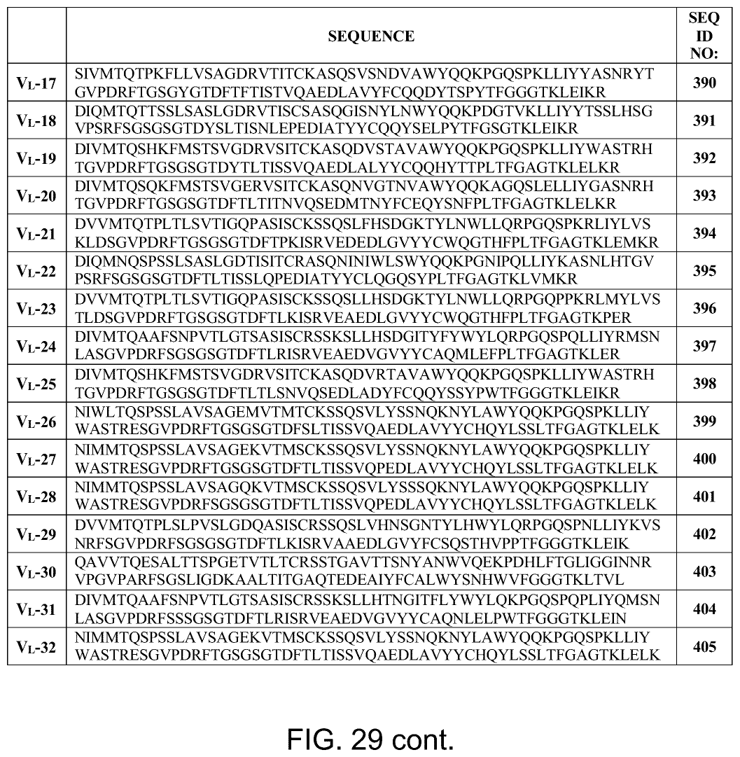

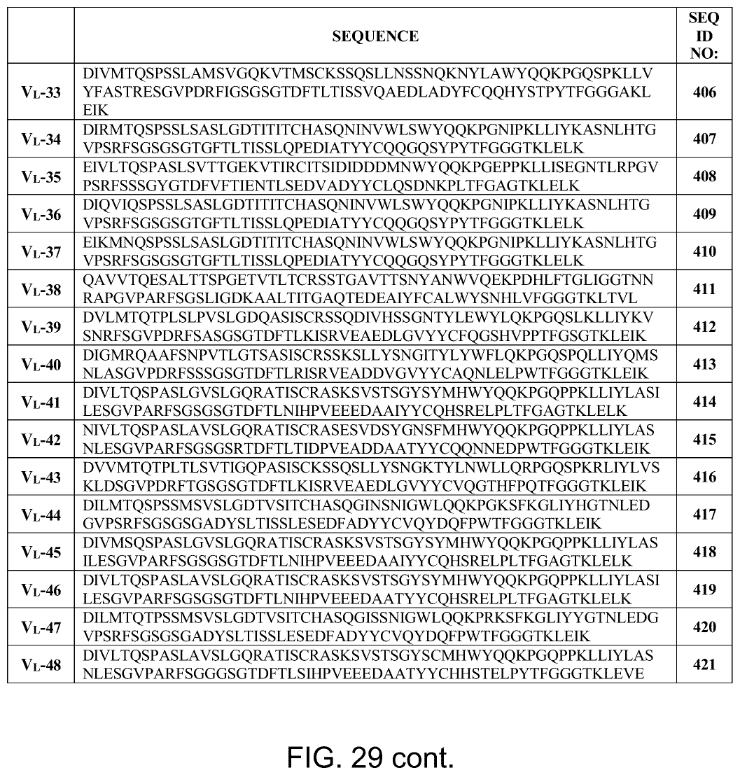

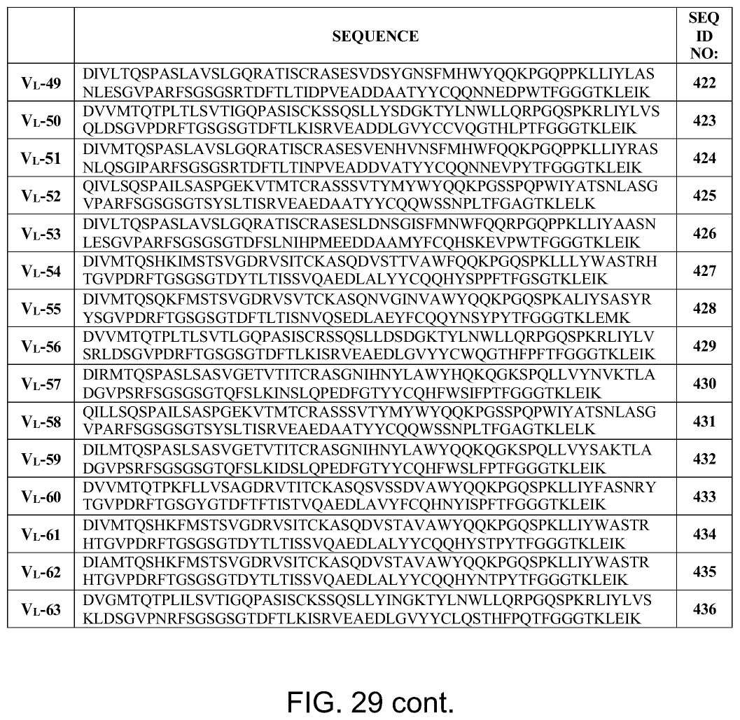

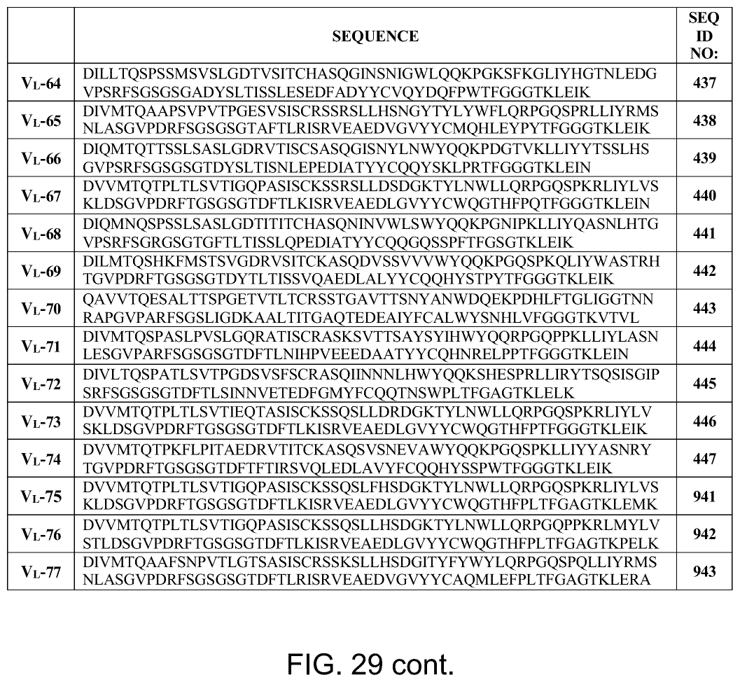

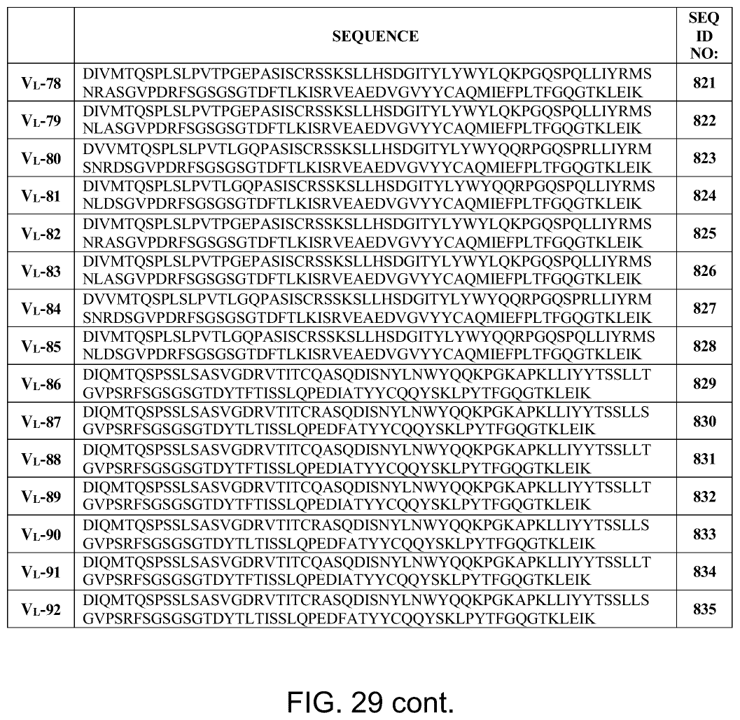

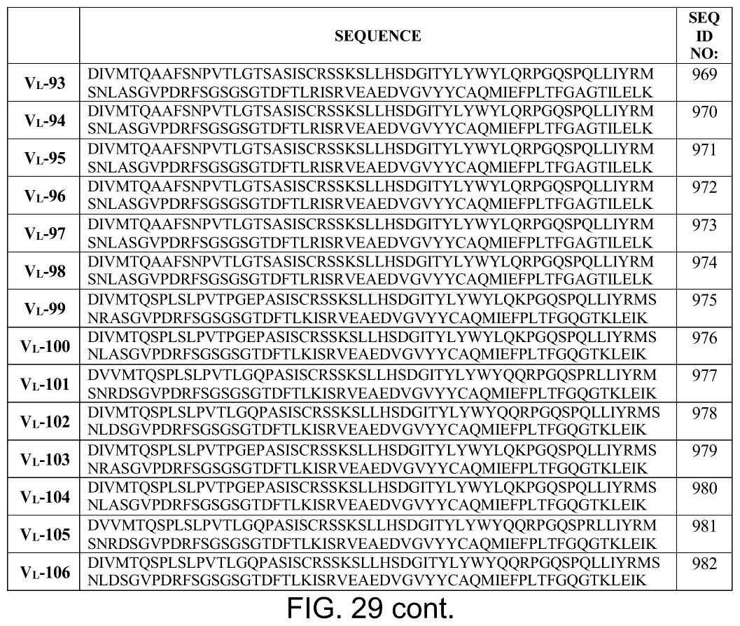

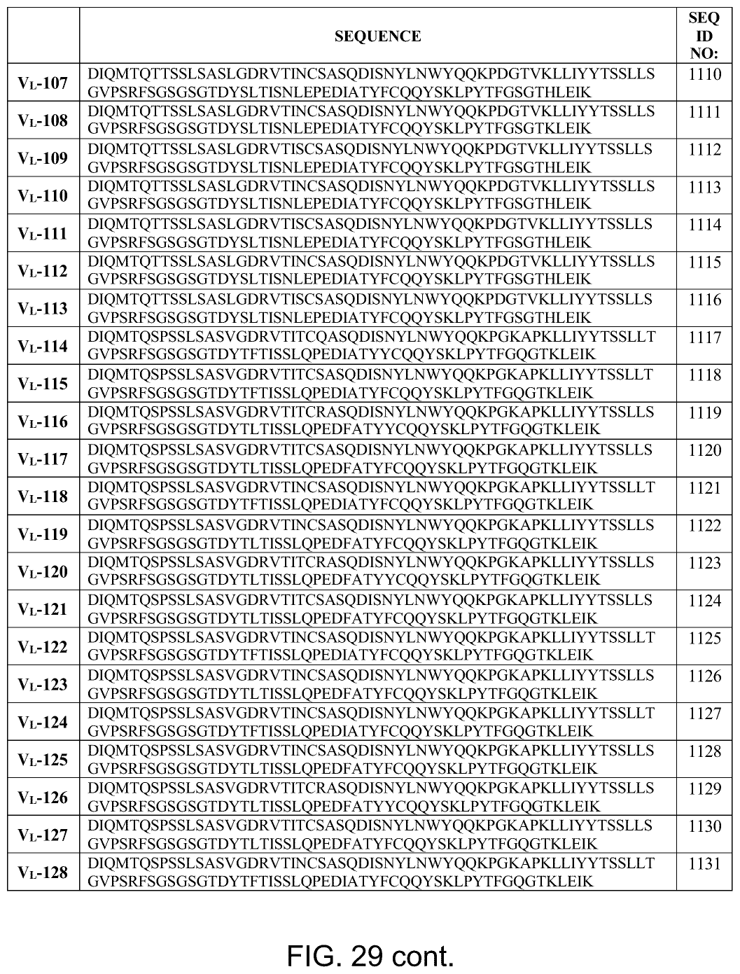

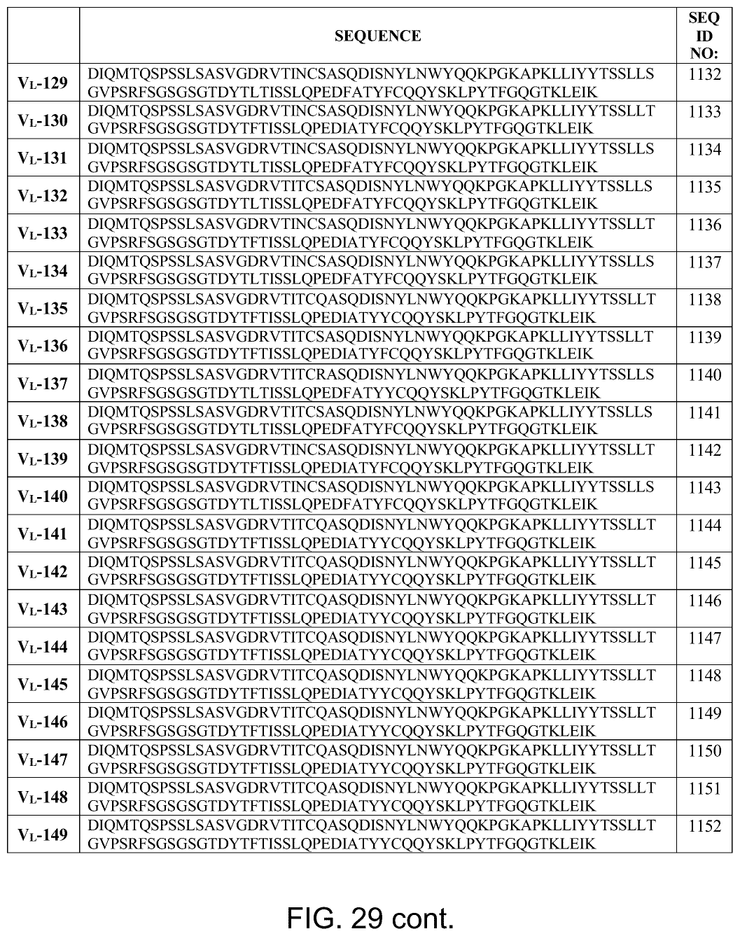

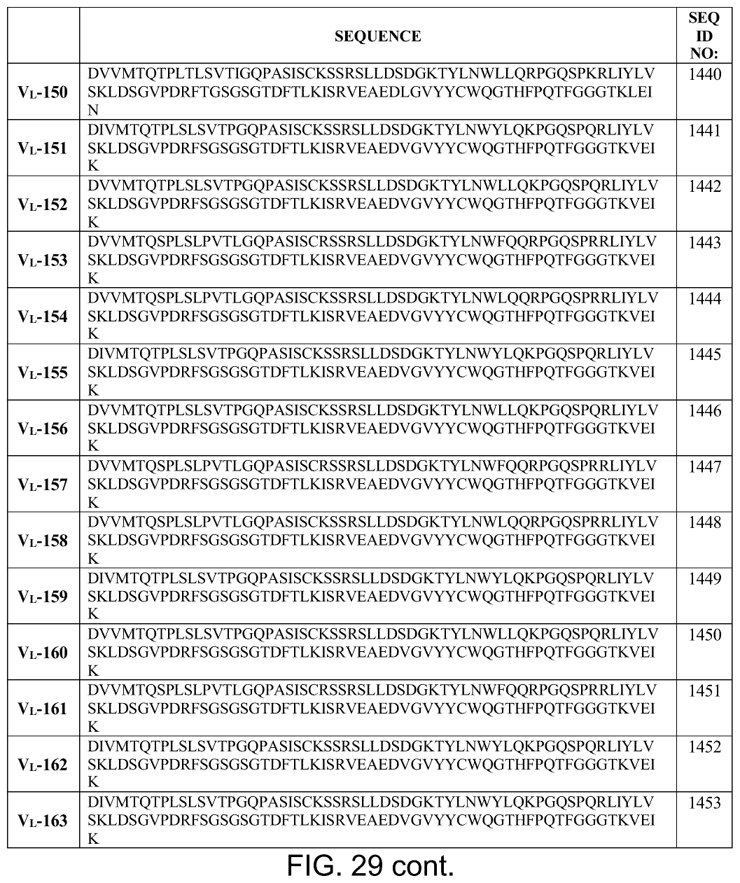

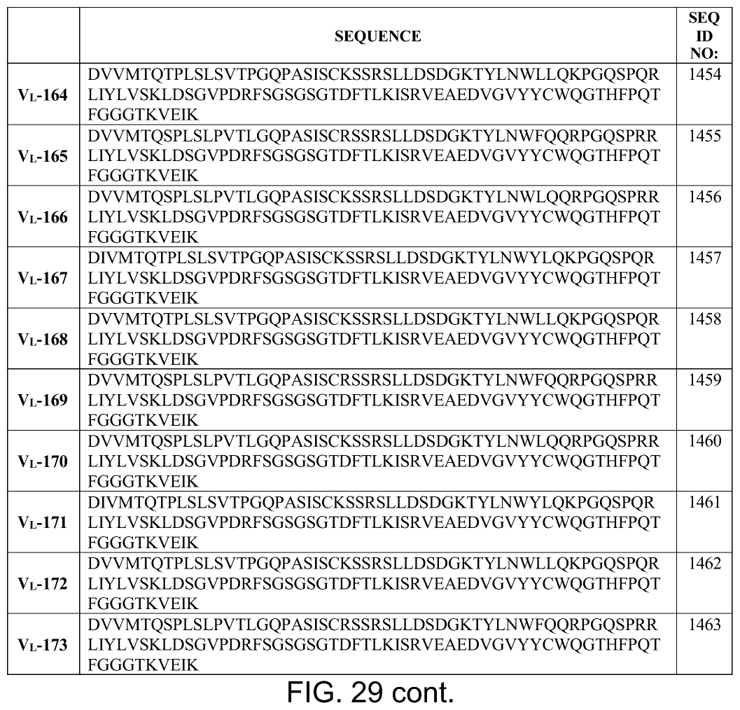

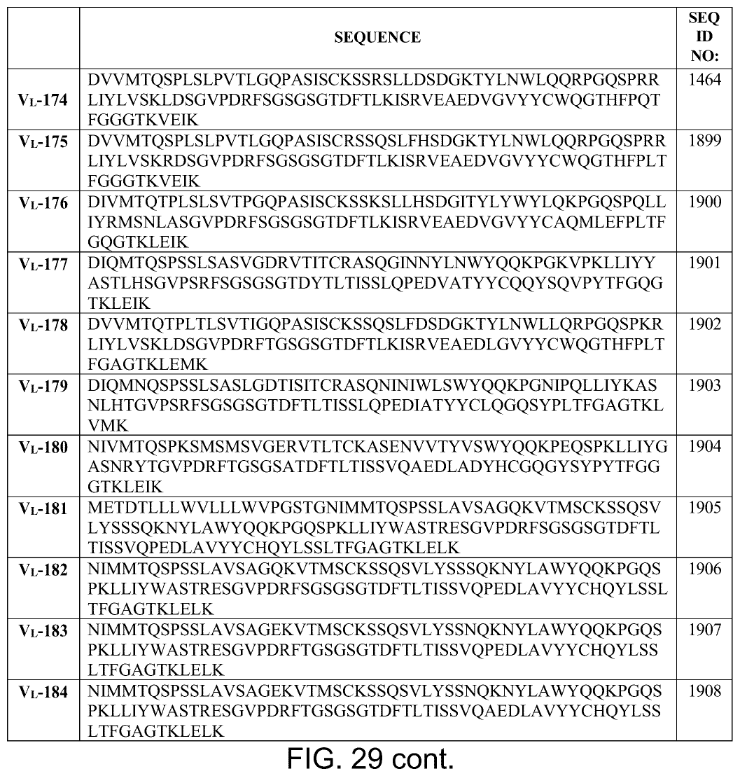

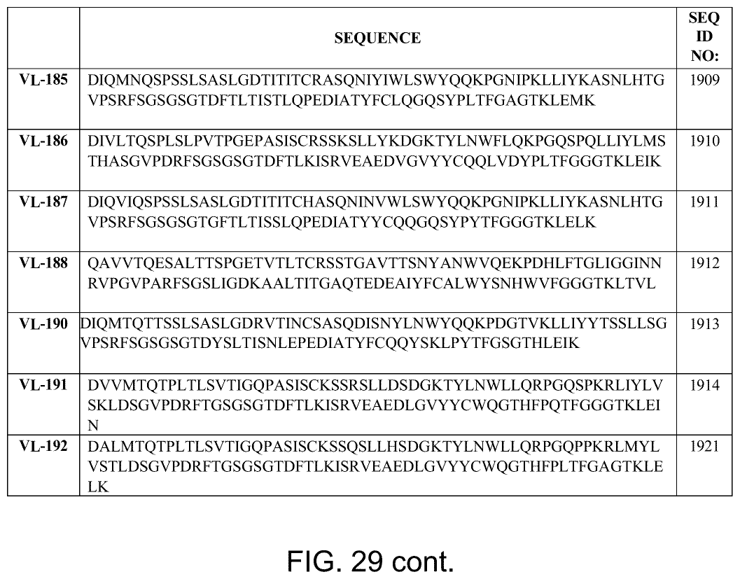

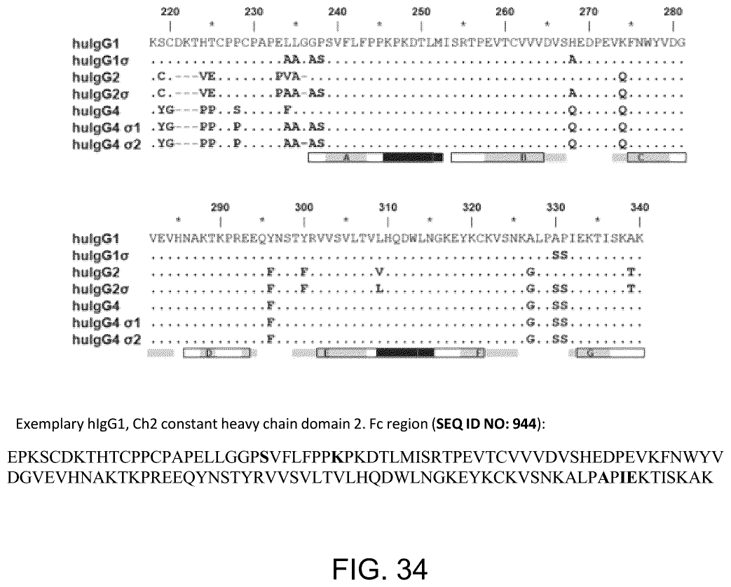

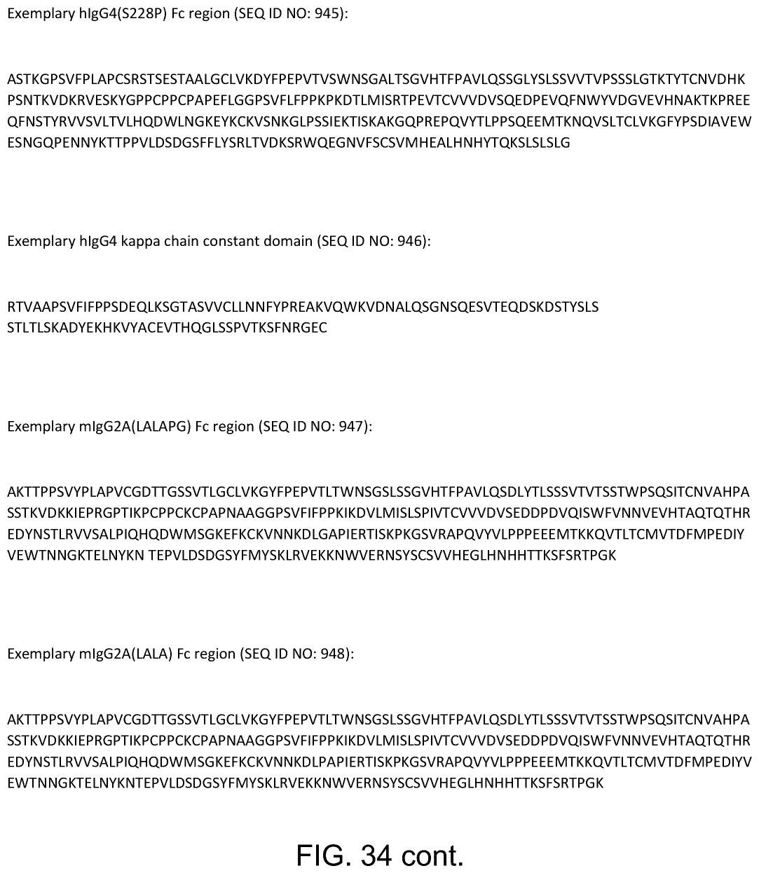

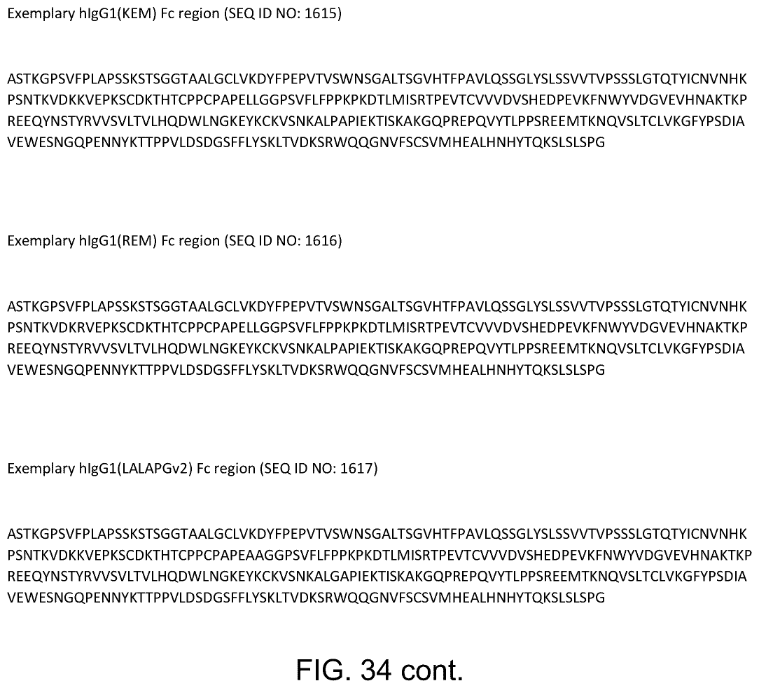

In some embodiments, the anti-Gal3 antibodies or binding fragments thereof comprise a heavy chain variable region and a light chain variable region. In some embodiments, the heavy chain variable region is paired with an IgG4 heavy chain constant domain or an IgG2 heavy chain constant domain. In some embodiments, the IgG4 heavy chain constant domain or IgG2 heavy chain constant domain are human or murine. In some embodiments, the IgG4 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 931. In some embodiments, the IgG4 heavy chain constant domain is an S228P mutant. In some embodiments, the IgG2 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 933 or SEQ ID NO: 934. In some embodiments, the IgG2 heavy chain constant domain is a LALAPG or a LALA mutant. In some embodiments, the light chain variable region is paired with an IgG4 kappa chain constant domain. In some embodiments, the IgG4 kappa chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 932. Exemplary heavy chain and light chain constant domains can be found in FIG. 34. In some embodiments, the light chain variable region and/or heavy chain variable region may be selected from those depicted in FIGS. 28 and 29 and/or the combinations of light chain variable region and heavy chain variable region as depicted in FIG. 34. In some embodiments, the light chain variable region and/or heavy chain variable regions comprise one or more CDRs depicted in FIGS. 26A-C, 27A-C and/or the combinations of CDRs depicted in FIG. 30.

In some embodiments, the antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, the antibody or binding fragment thereof further comprises a signal peptide.

In some embodiments, the antibody or binding fragment thereof comprises a sequence (e.g. CDR, VL, VH, LC, HC) having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, the antibody or binding fragment thereof further comprises a signal peptide.

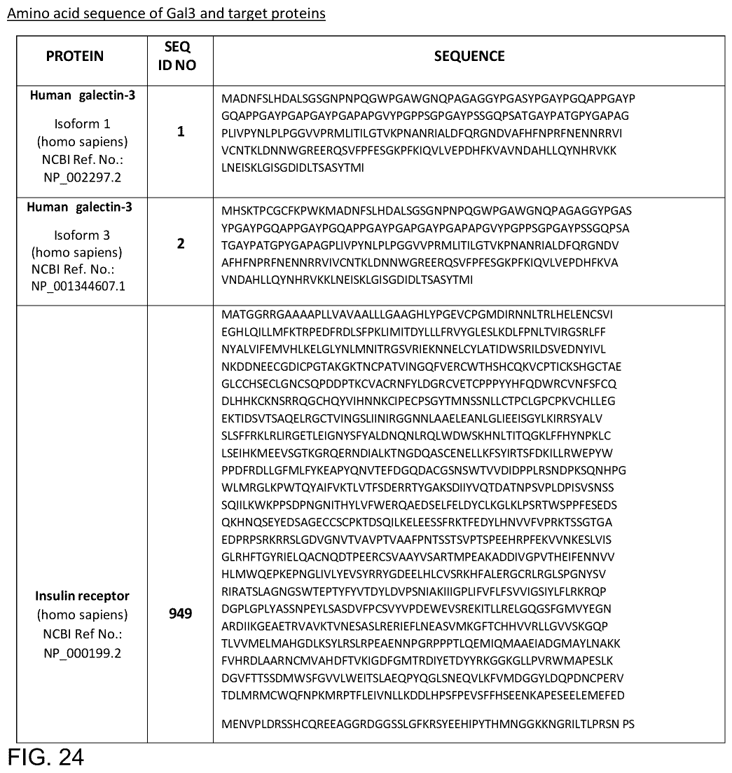

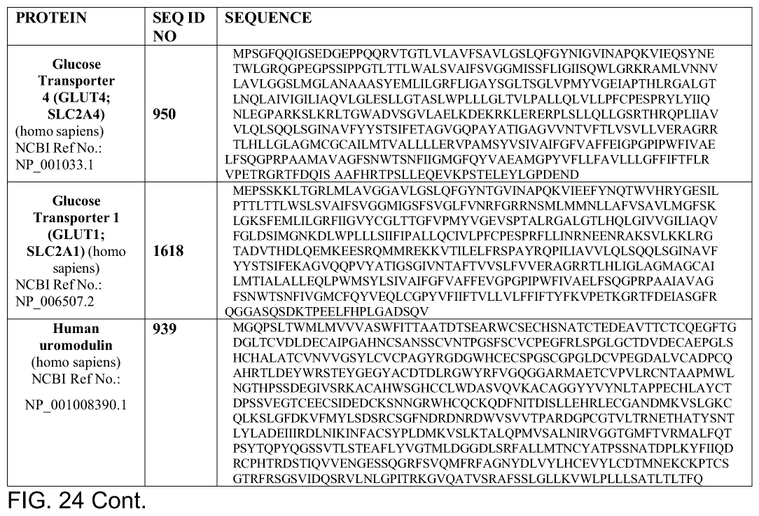

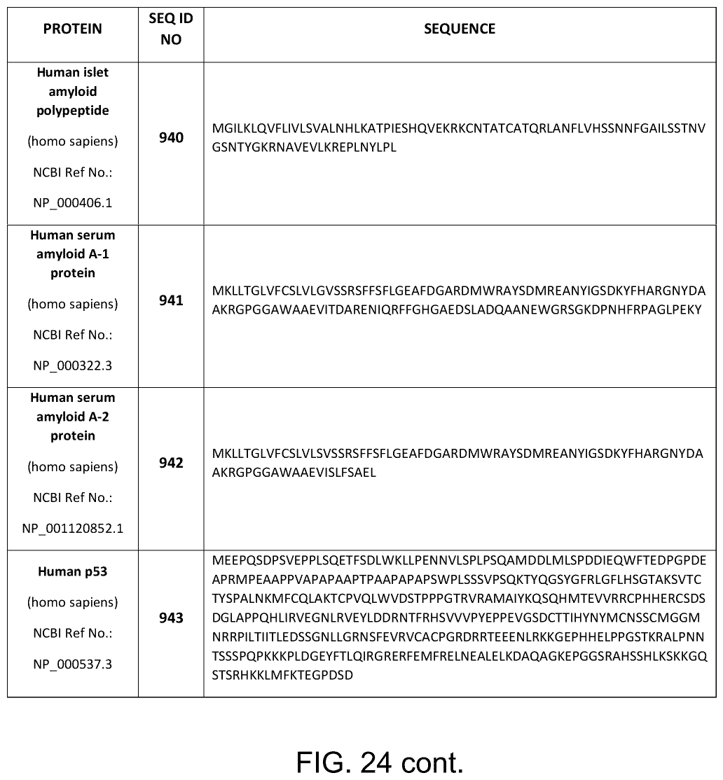

In some embodiments, the anti-Gal3 antibody or binding fragment thereof binds to specific epitopes within a Gal3 protein. In some embodiments, the anti-Gal3 antibody or binding fragment thereof binds to a specific epitope within a Gal3 protein having an amino acid sequence according to SEQ ID NO: 1-2, provided in FIG. 24.

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within a peptide illustrated in FIG. 25 (SEQ ID NOs: 3-26).

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 1-20 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 31-50 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 51-70 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 61-80 of SEQ ID NO: 1-2.

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3), Peptide 4 (SEQ ID NO: 6), Peptide 6 (SEQ ID NO: 8), or Peptide 7 (SEQ ID NO: 9). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 4 (SEQ ID NO: 6). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 6 (SEQ ID NO: 8). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 7 (SEQ ID NO: 9). In some embodiments, the anti-Gal3 antibody or binding fragment thereof binds to an epitope present within a region of Gal3 defined by Peptide 1 (SEQ ID NO: 3). In some embodiments, the anti-Gal3 antibody or binding fragment thereof binds to an epitope present within a region of Gal3 defined by Peptide 4 (SEQ ID NO: 6). In some embodiments, the anti-Gal3 antibody or binding fragment thereof binds to an epitope present within a region of Gal3 defined by Peptide 6 (SEQ ID NO: 8). In some embodiments, the anti-Gal3 antibody or binding fragment thereof binds to an epitope present within a region of Gal3 defined by Peptide 7 (SEQ ID NO: 9). In some embodiments, the antibody is one that binds to 1, 2, or all 3 of peptides 1, 6, and/or 7.

In some embodiments, the anti-Gal3 antibody or binding fragment thereof further comprises one or more signal peptides. In some embodiments, the anti-Gal3 antibody or binding fragment thereof heavy chain further comprises a signal peptide. In some embodiments, the heavy chain further comprises a signal peptide having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 1922. In some embodiments, the heavy chain further comprises a signal peptide that is SEQ ID NO: 1922. In some embodiments, the heavy chain further comprises a signal peptide encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 1925. In some embodiments, the heavy chain further comprises a signal peptide encoded by a nucleic acid sequence that is SEQ ID NO: 1925. In some embodiments, the anti-Gal3 antibody or binding fragment thereof light chain further comprises a signal peptide. In some embodiments, the light chain further comprises a signal peptide having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 1923 or SEQ ID NO: 1924. In some embodiments, the light chain further comprises a signal peptide that is SEQ ID NO: 1923. In some embodiments, the light chain further comprises a signal peptide that is SEQ ID NO: 1924. In some embodiments, the light chain further comprises a signal peptide encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 1926 or SEQ ID NO: 1927. In some embodiments, the light chain further comprises a signal peptide encoded by a nucleic acid sequence that is SEQ ID NO: 1926. In some embodiments, the light chain further comprises a signal peptide encoded by a nucleic acid sequence that is SEQ ID NO: 1927.

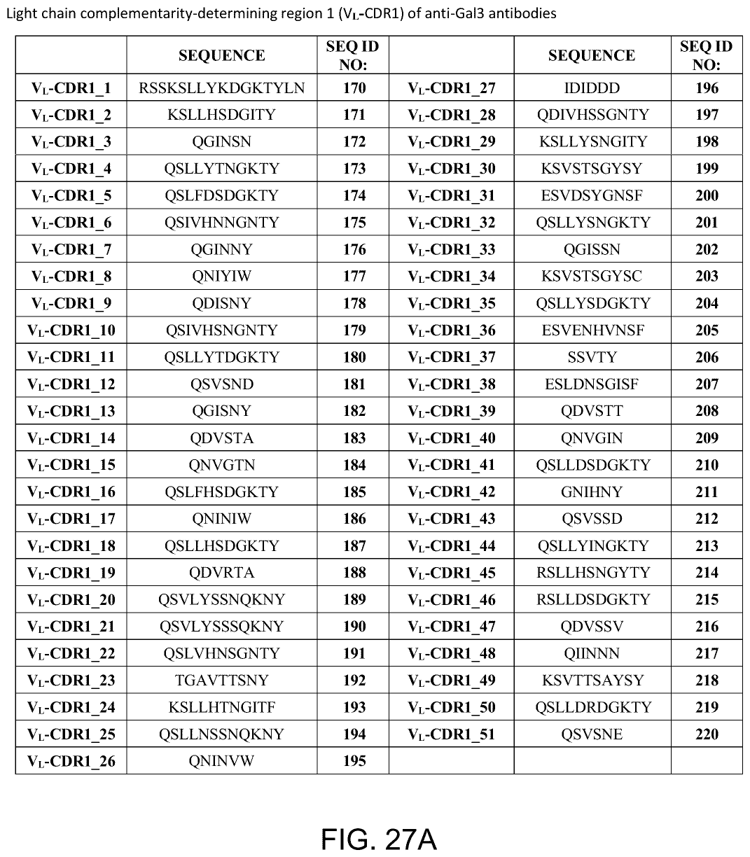

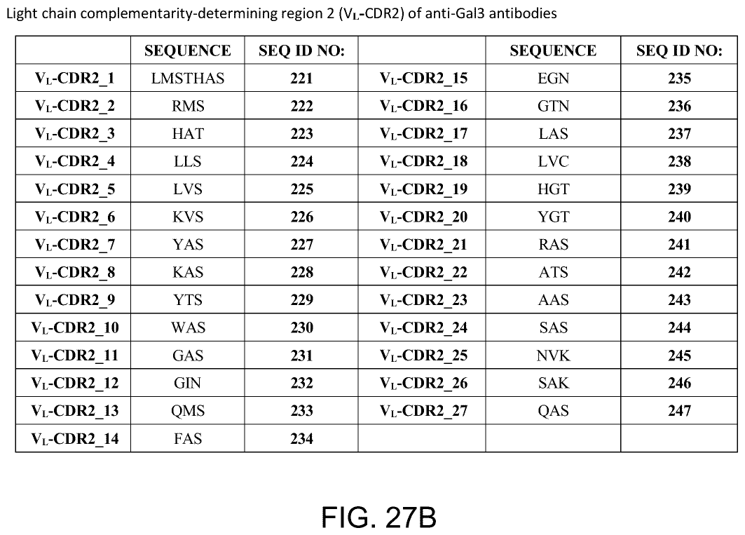

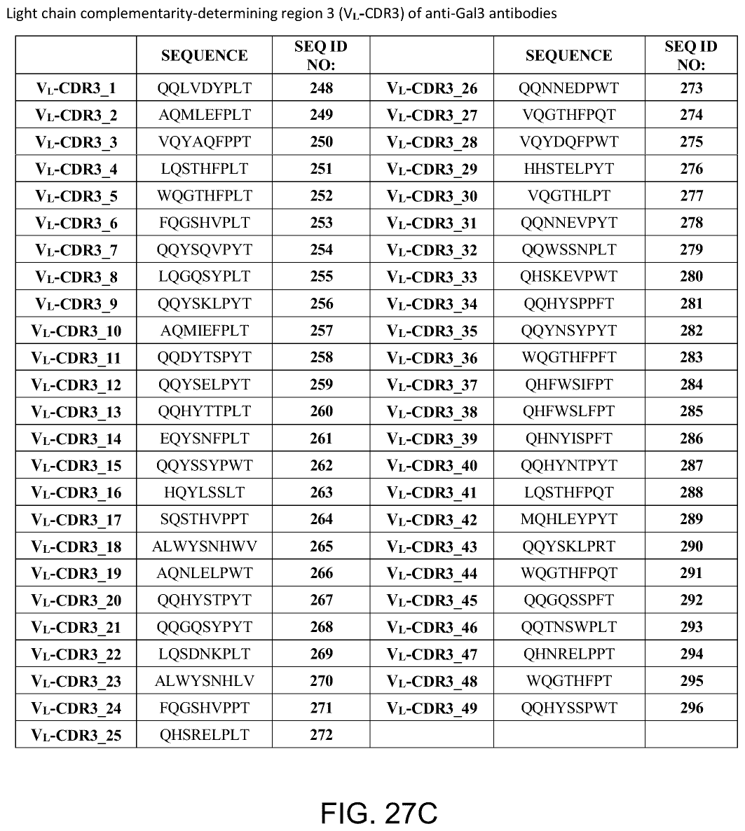

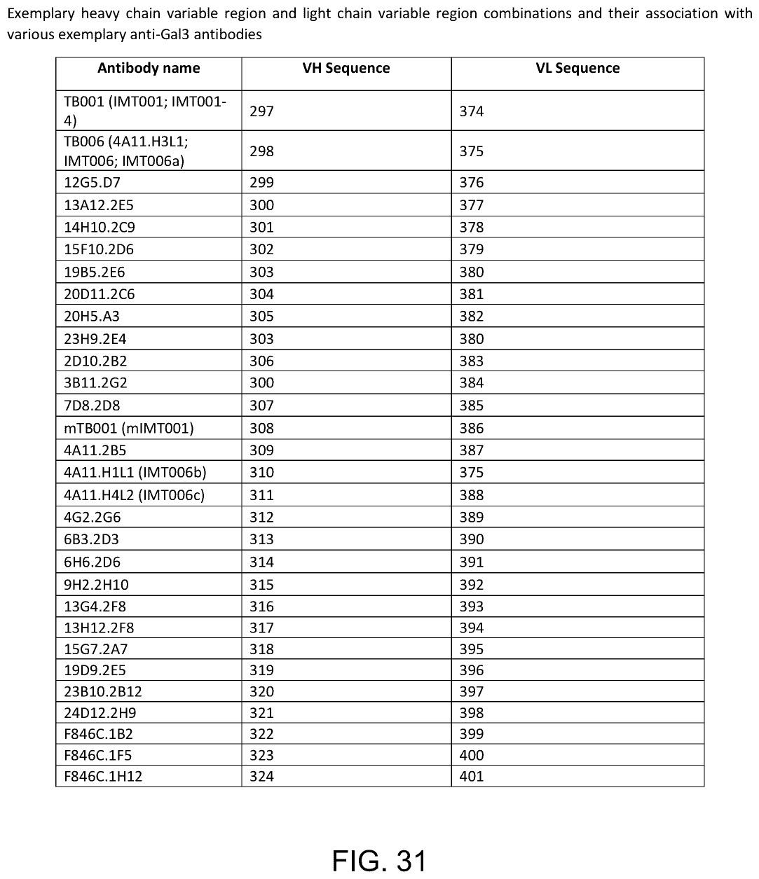

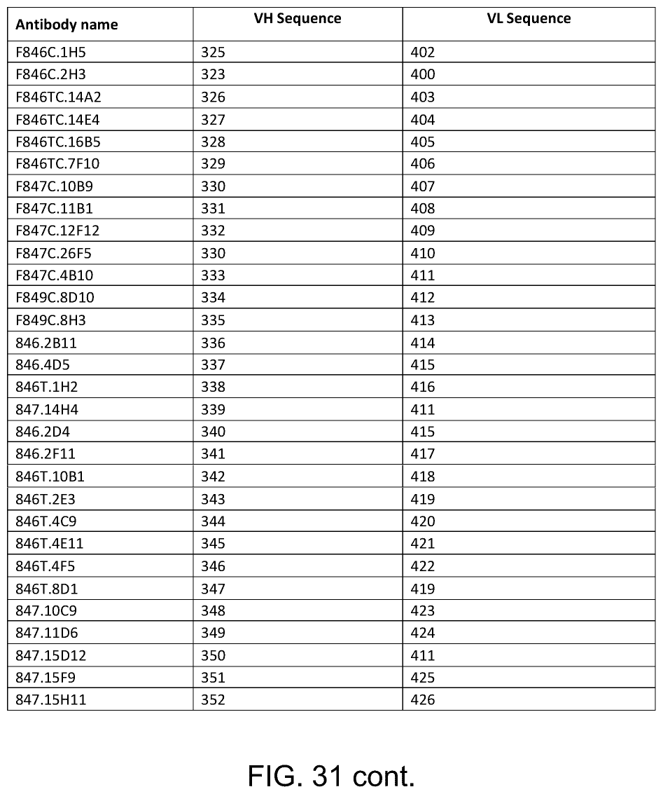

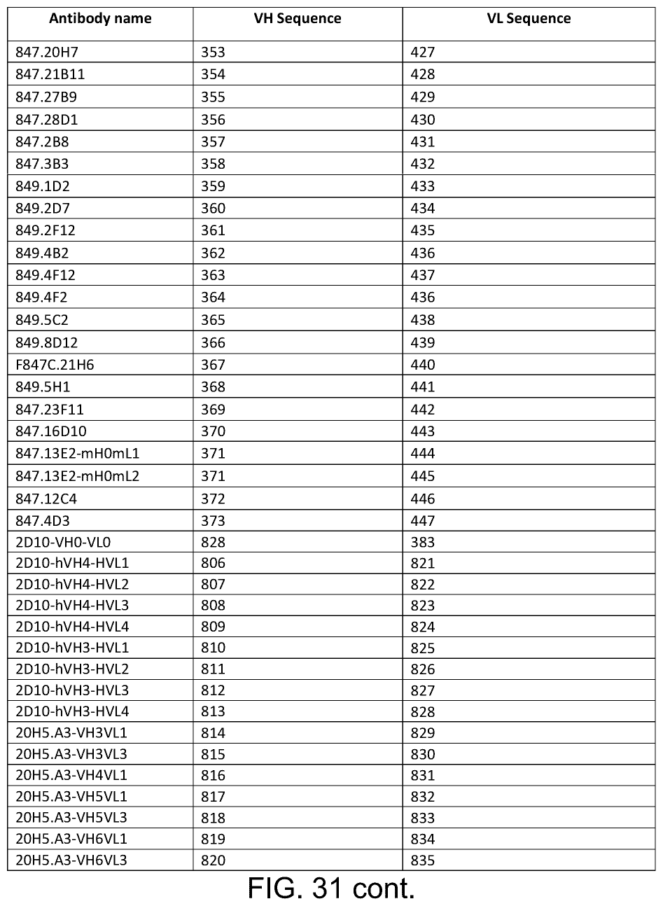

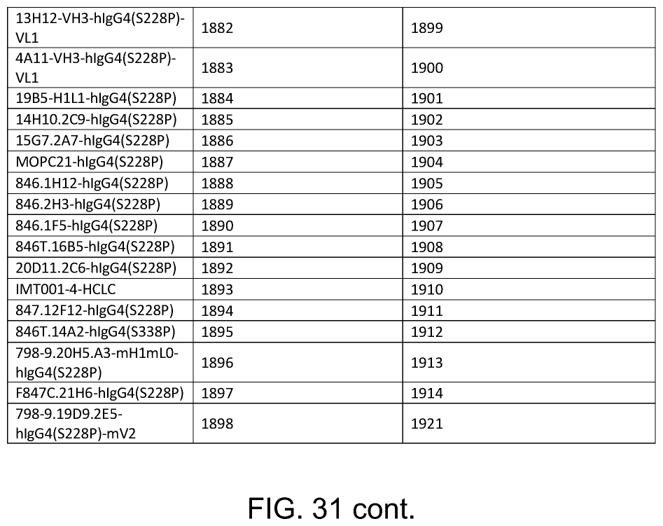

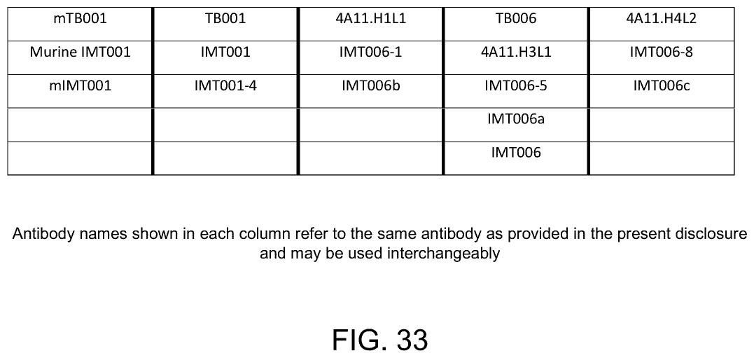

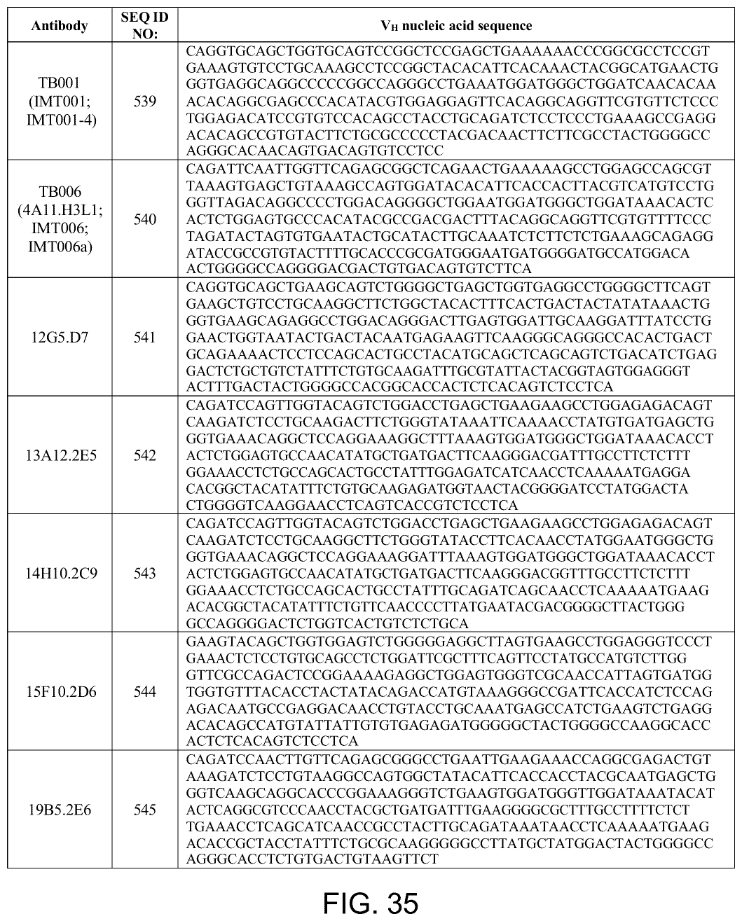

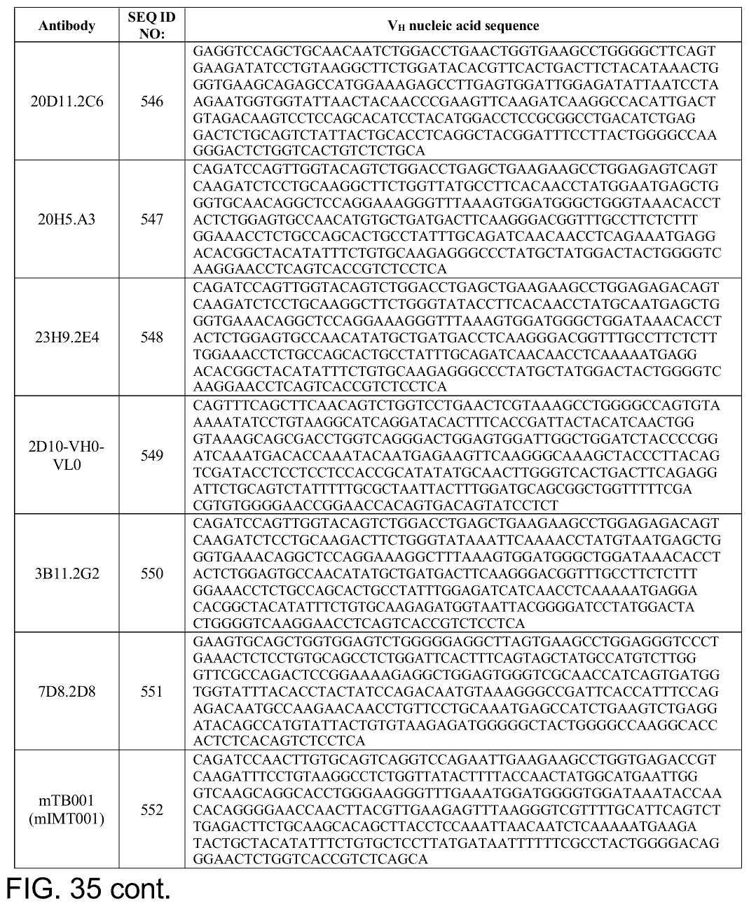

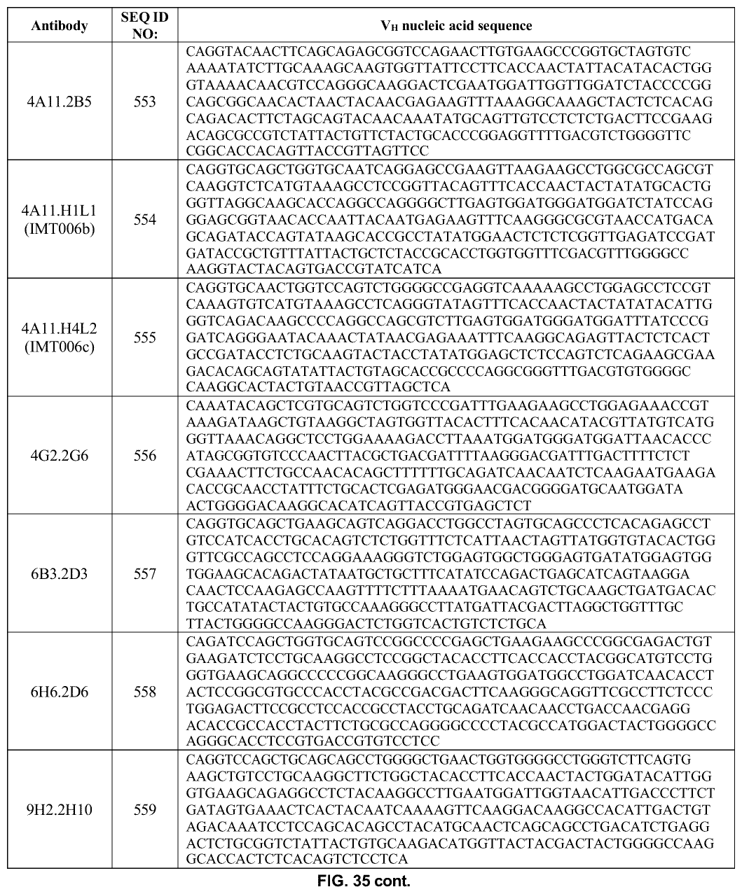

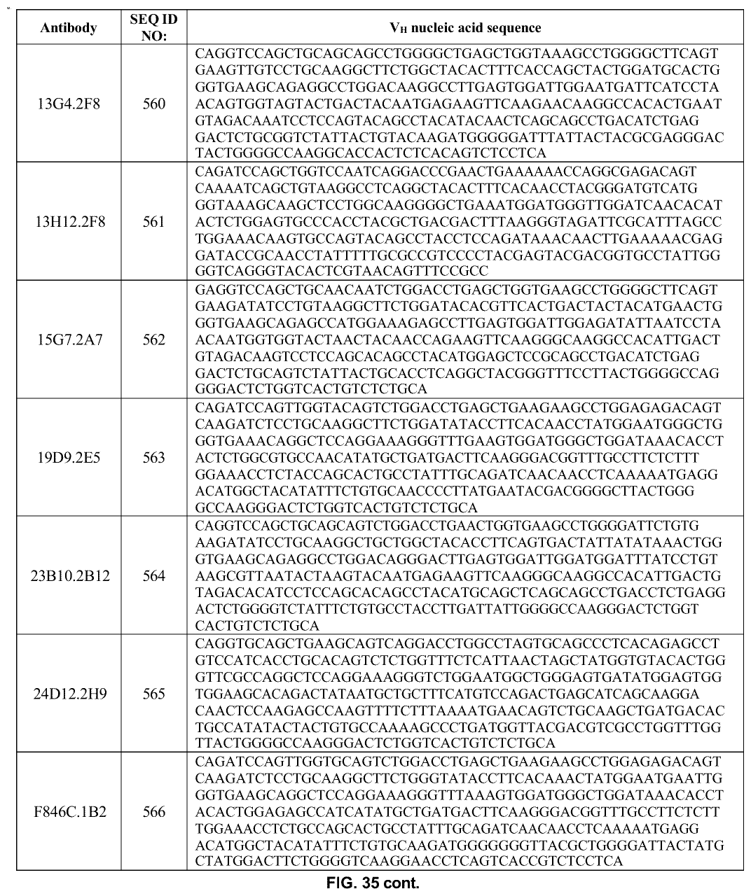

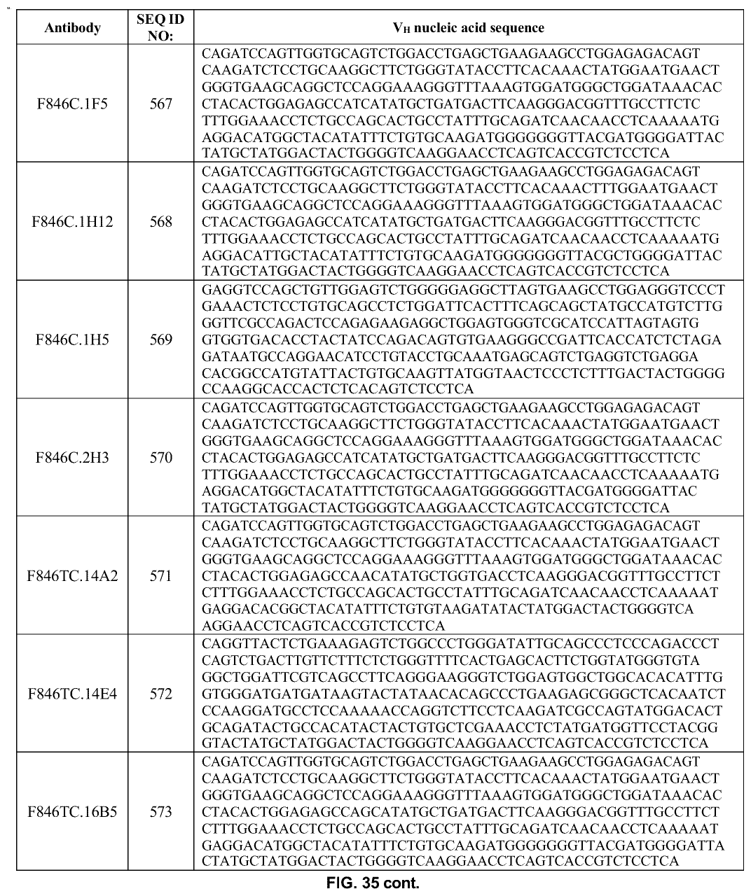

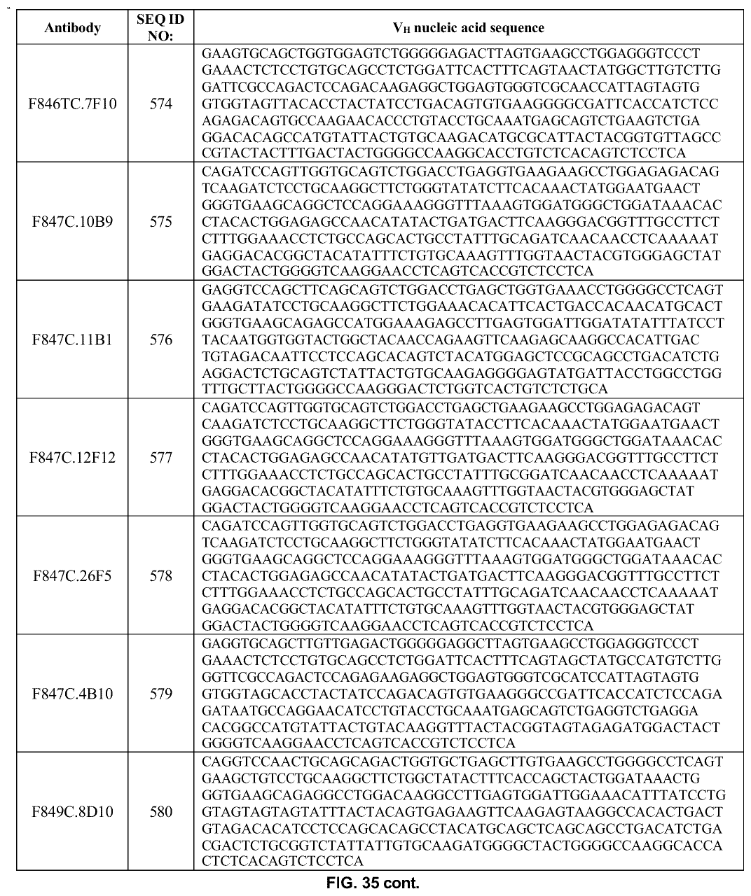

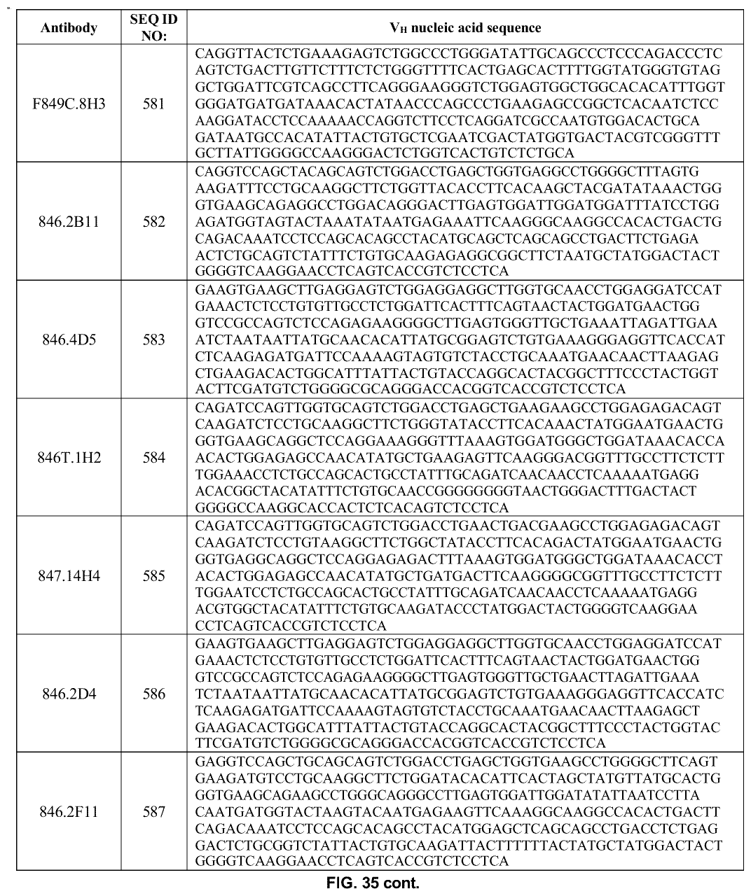

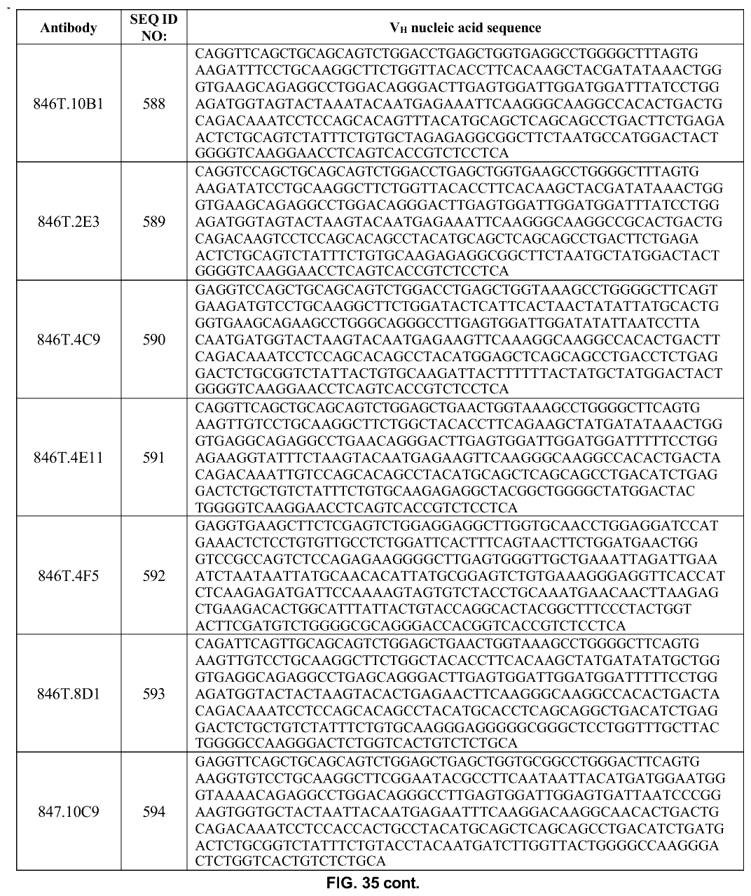

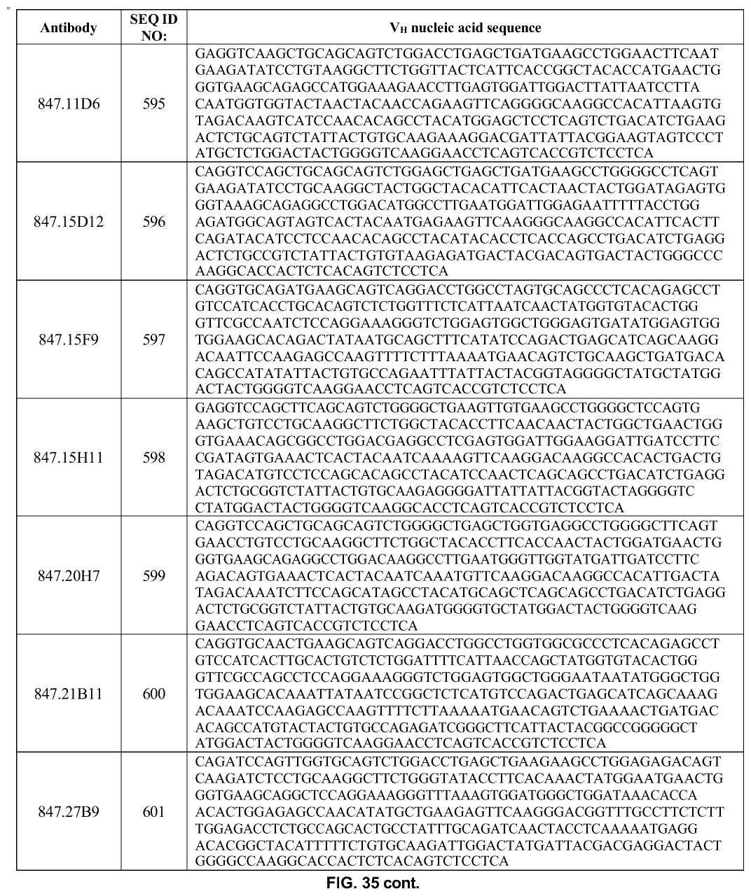

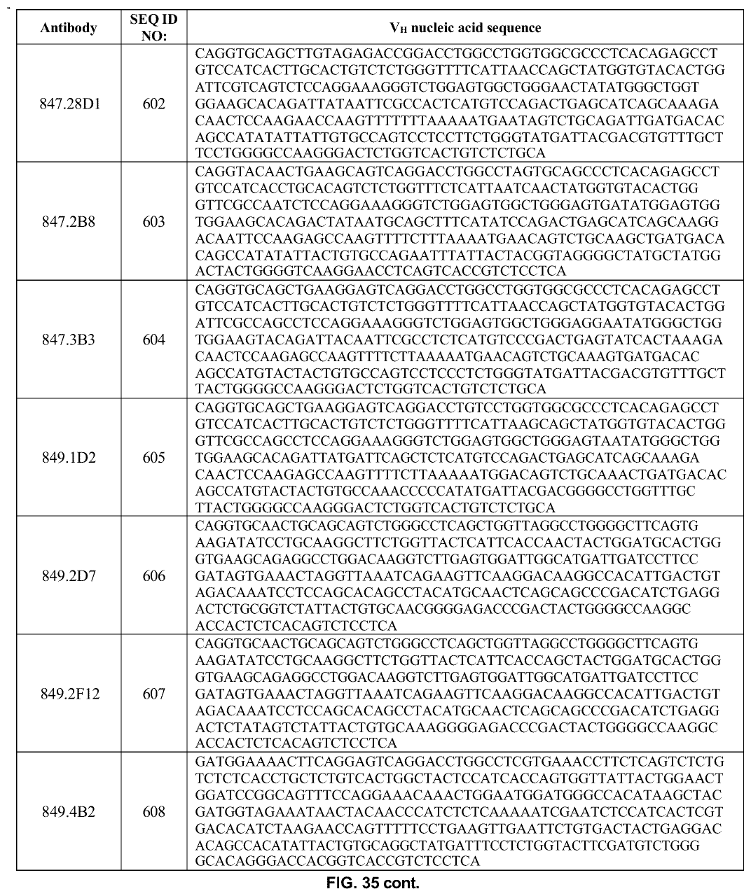

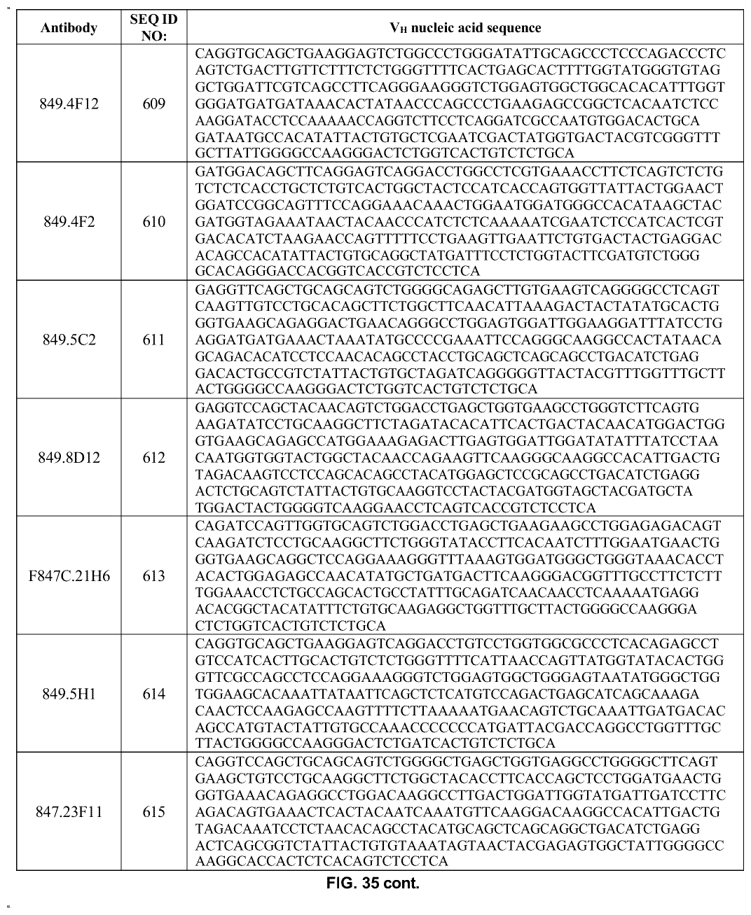

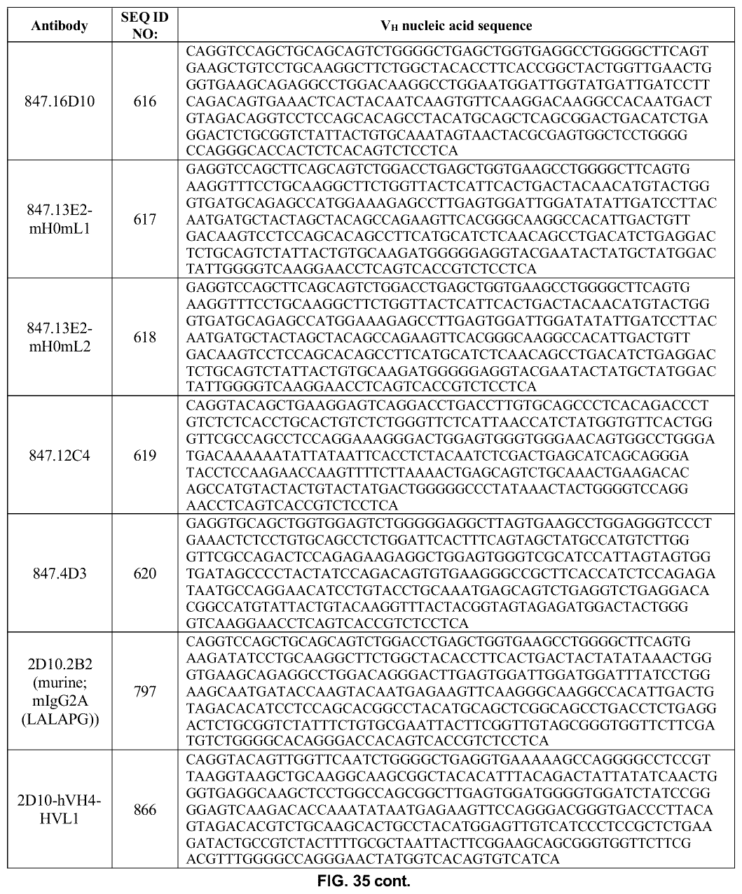

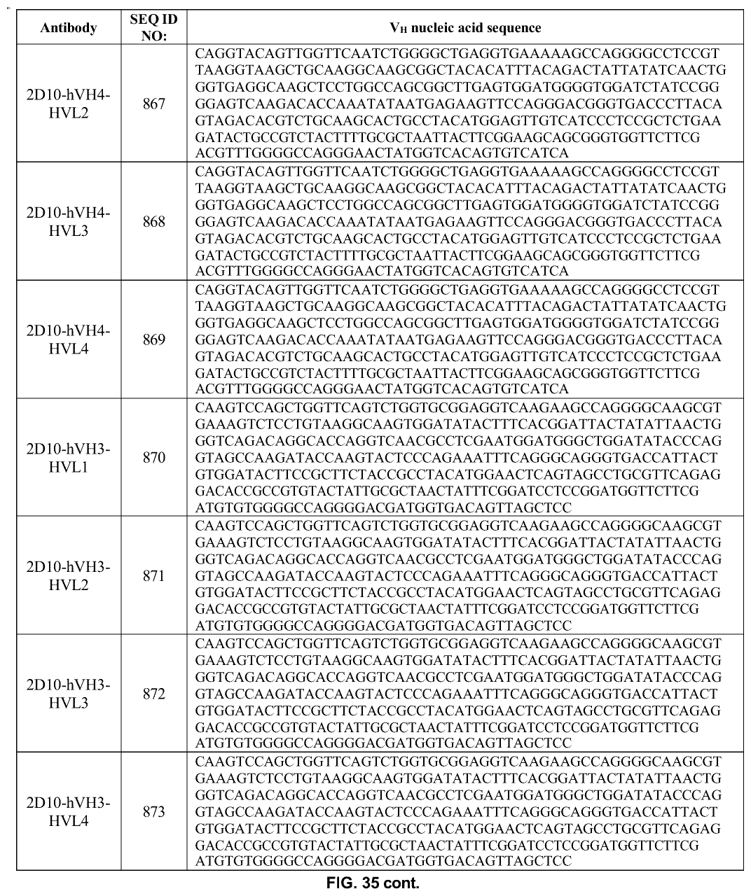

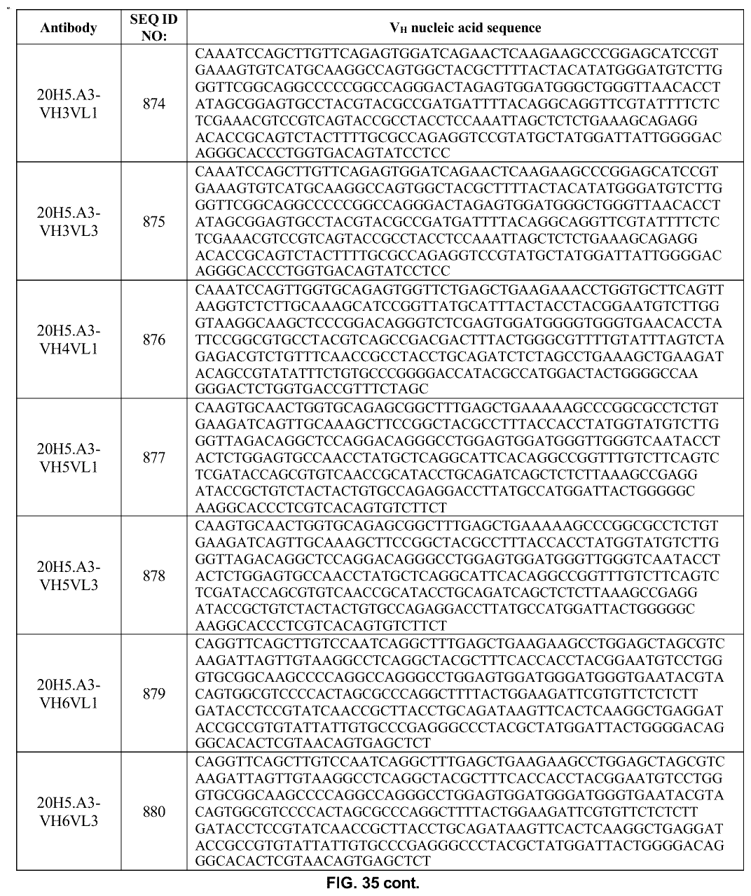

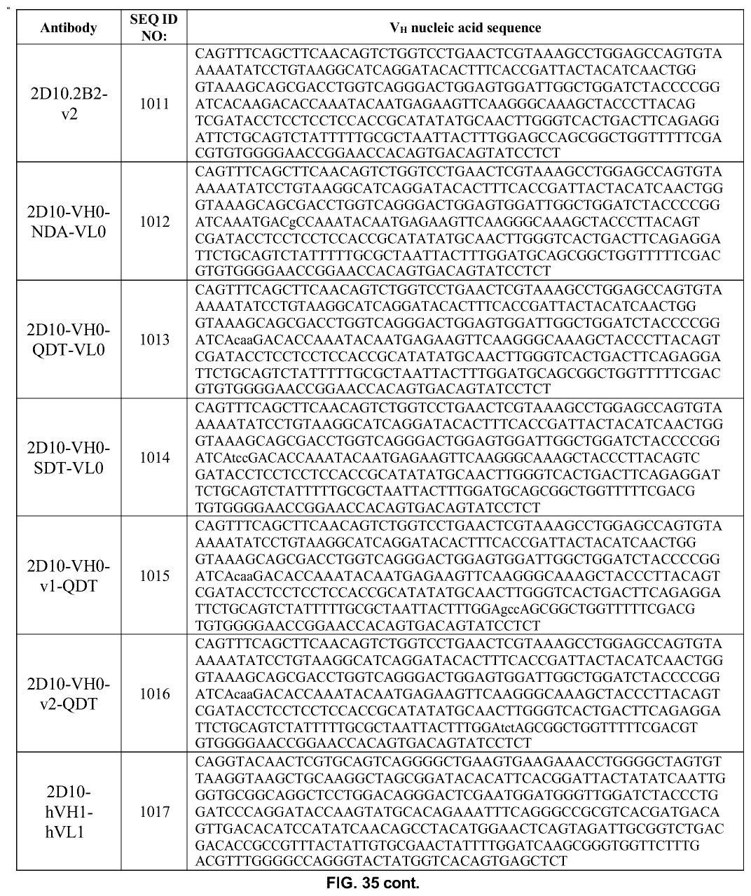

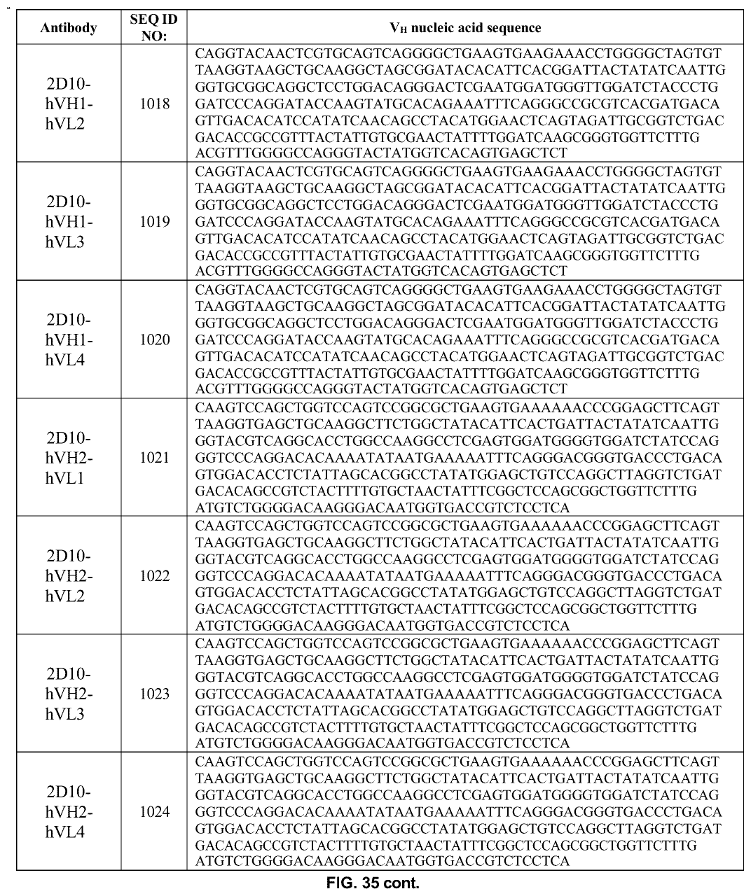

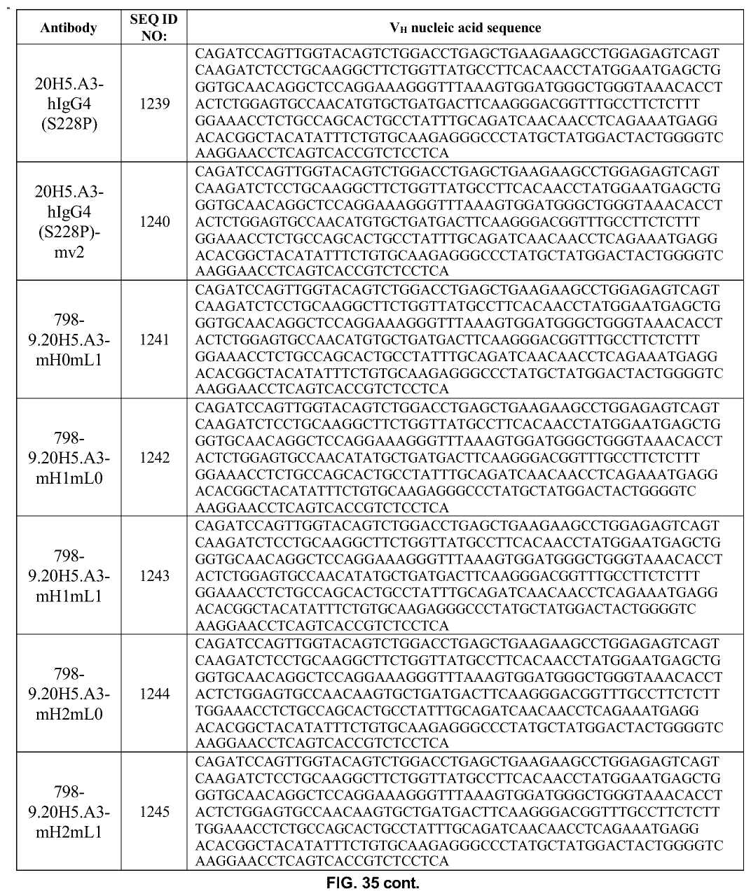

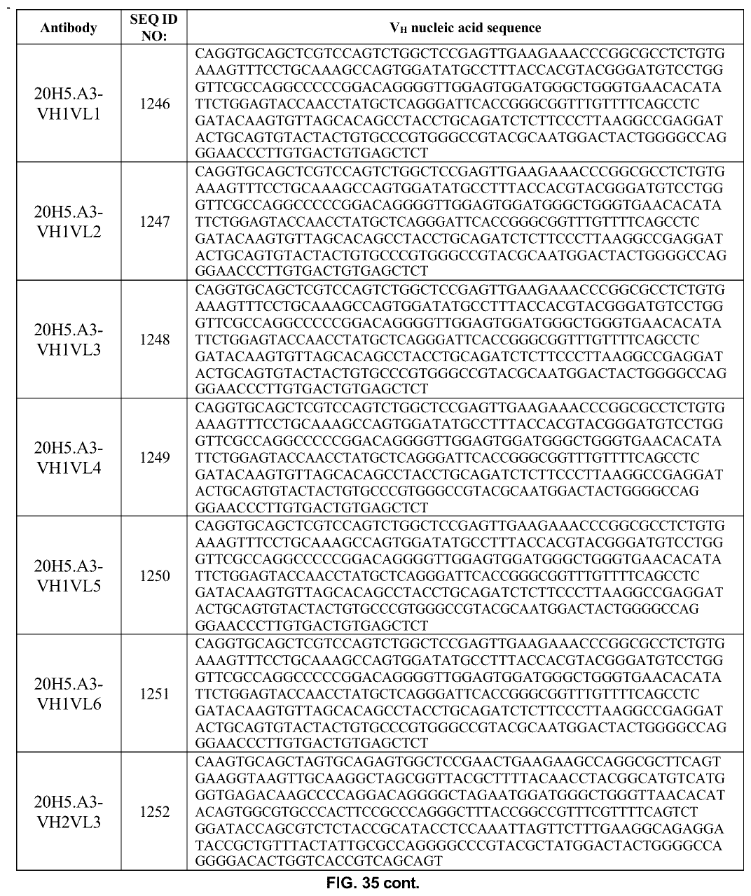

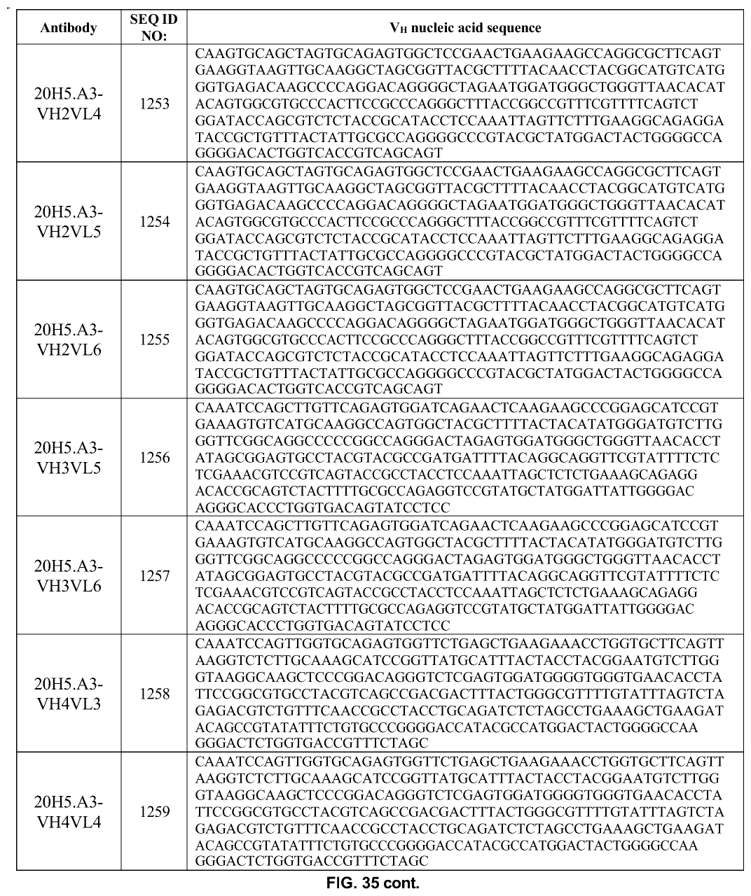

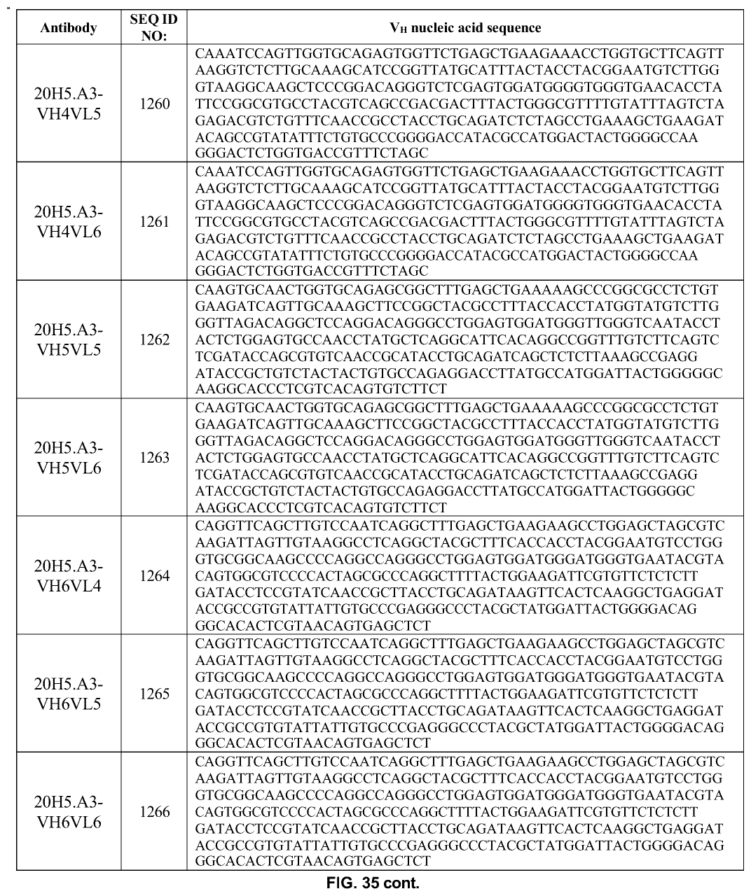

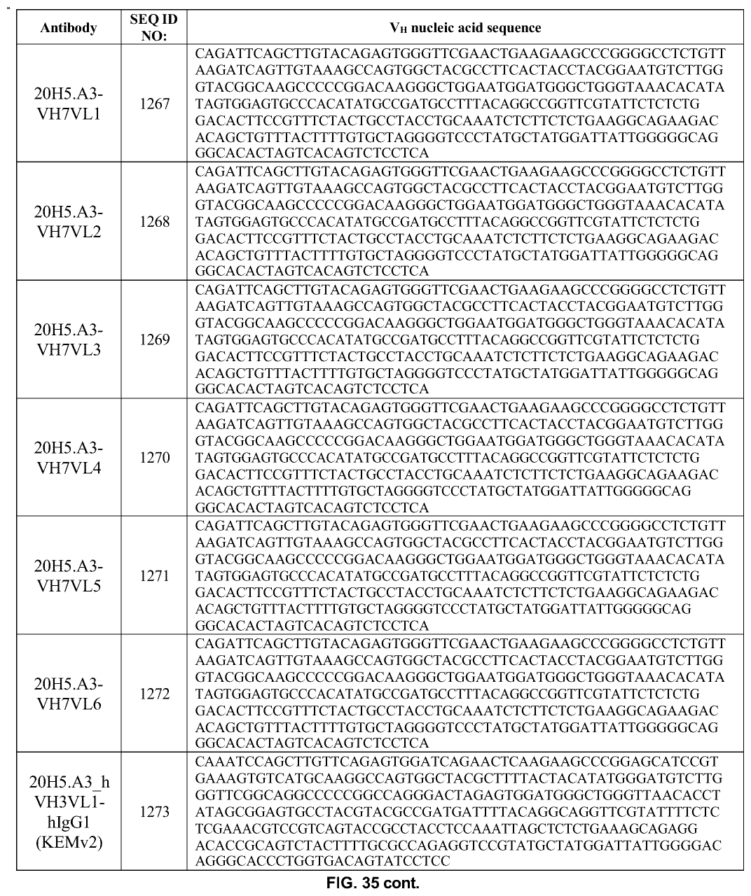

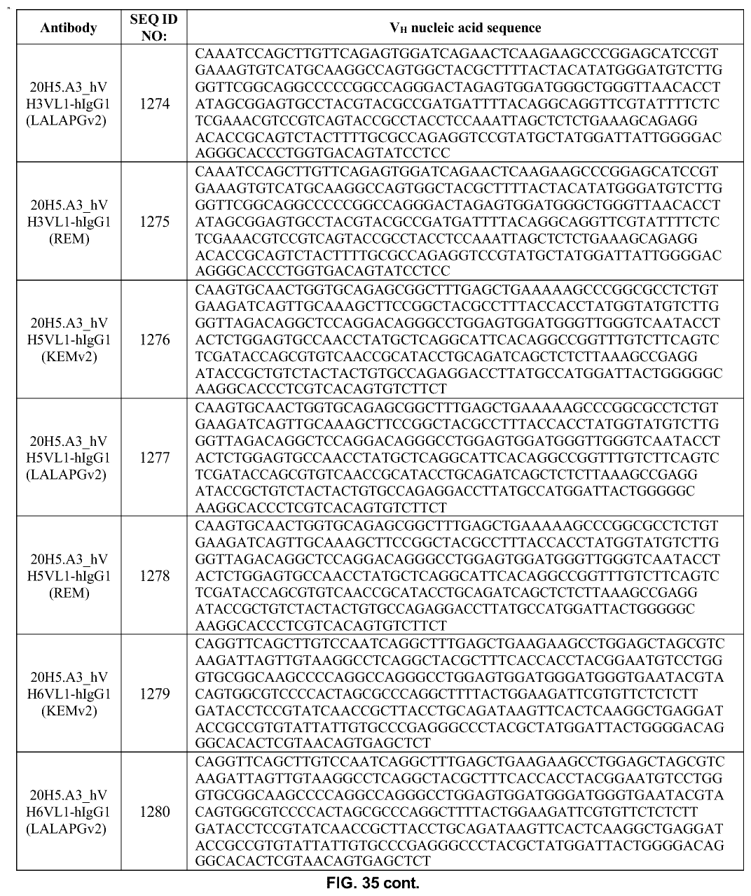

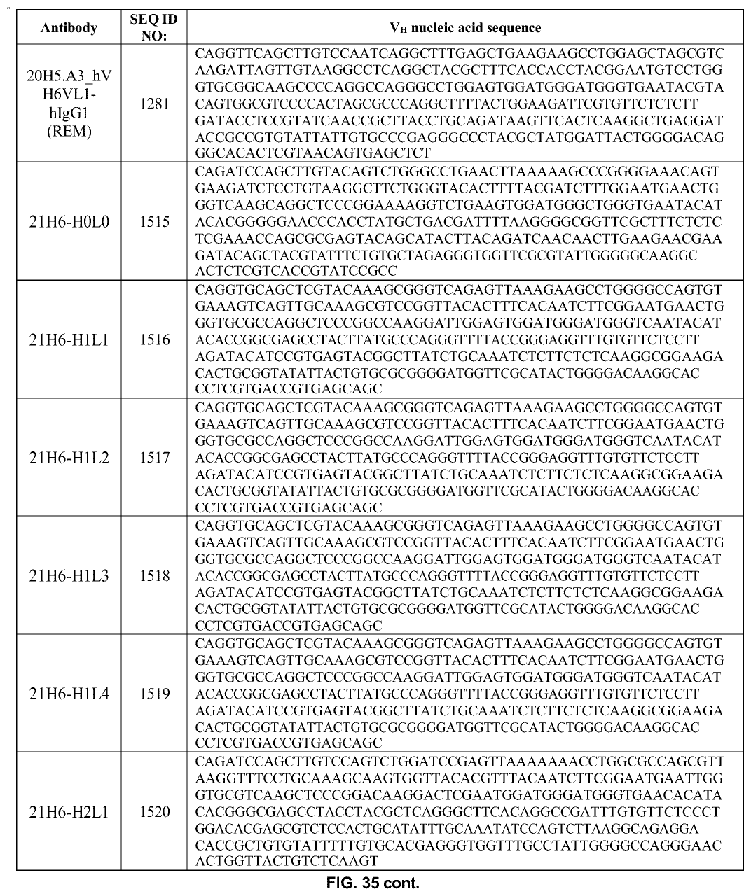

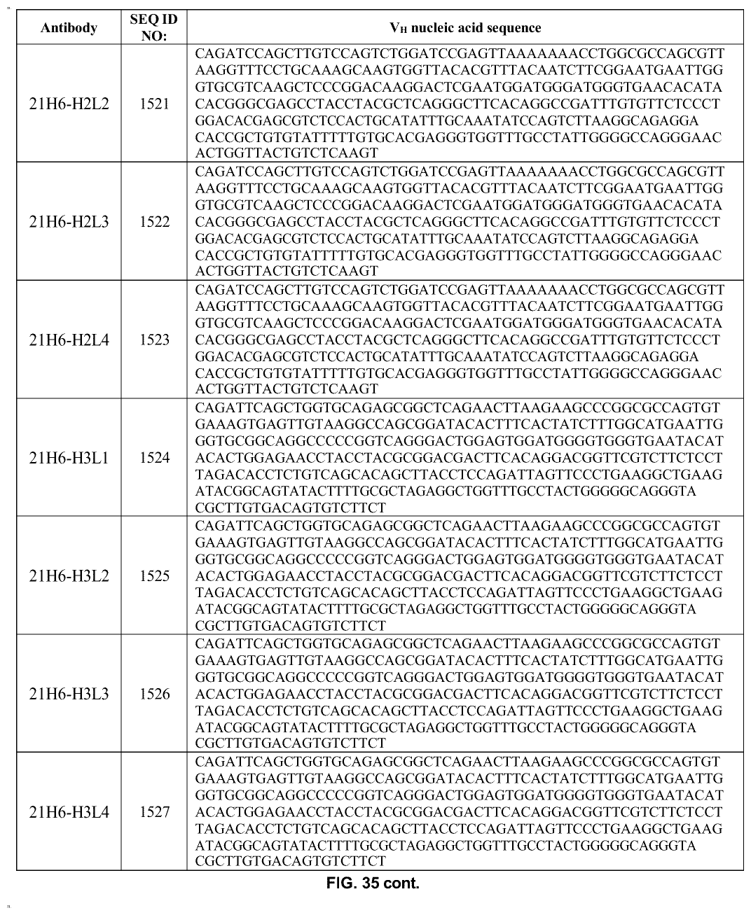

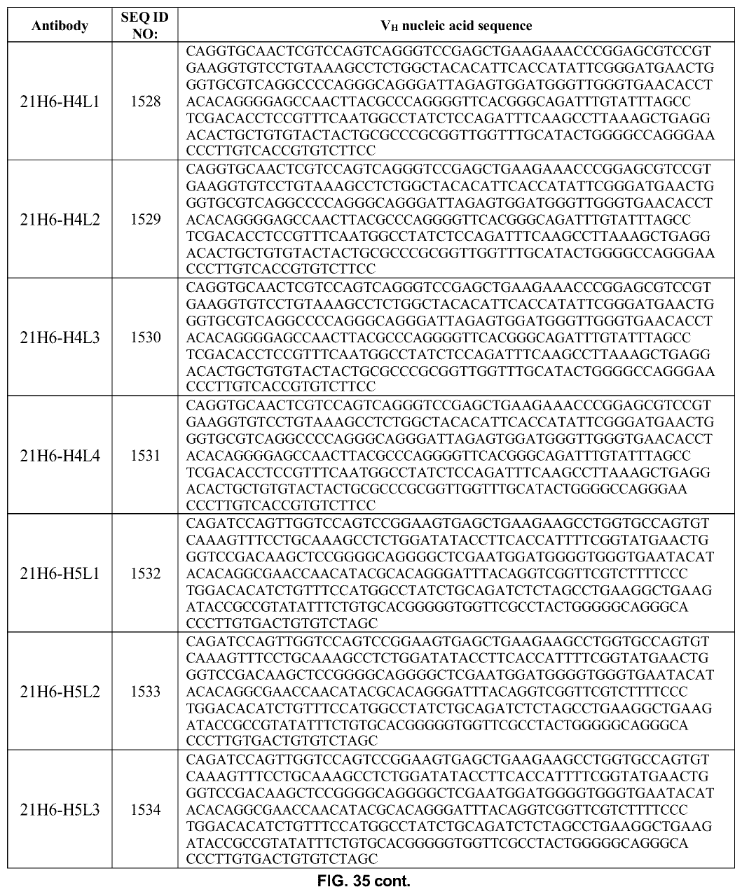

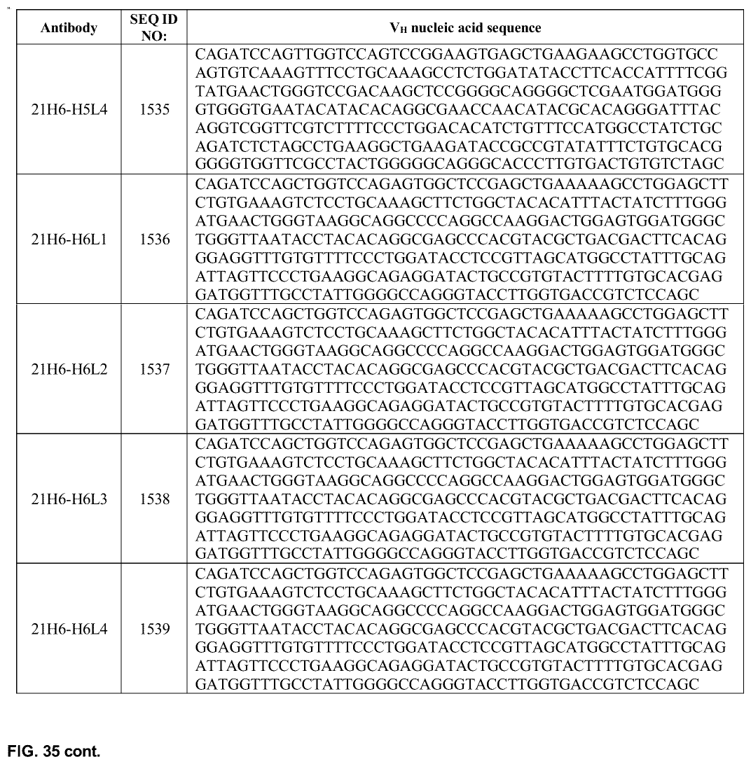

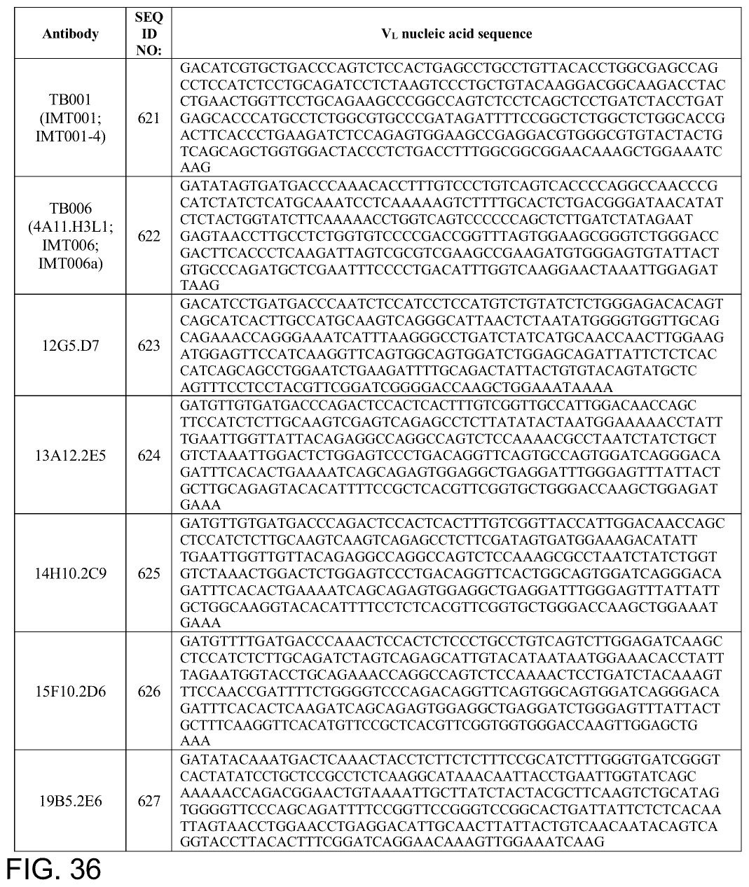

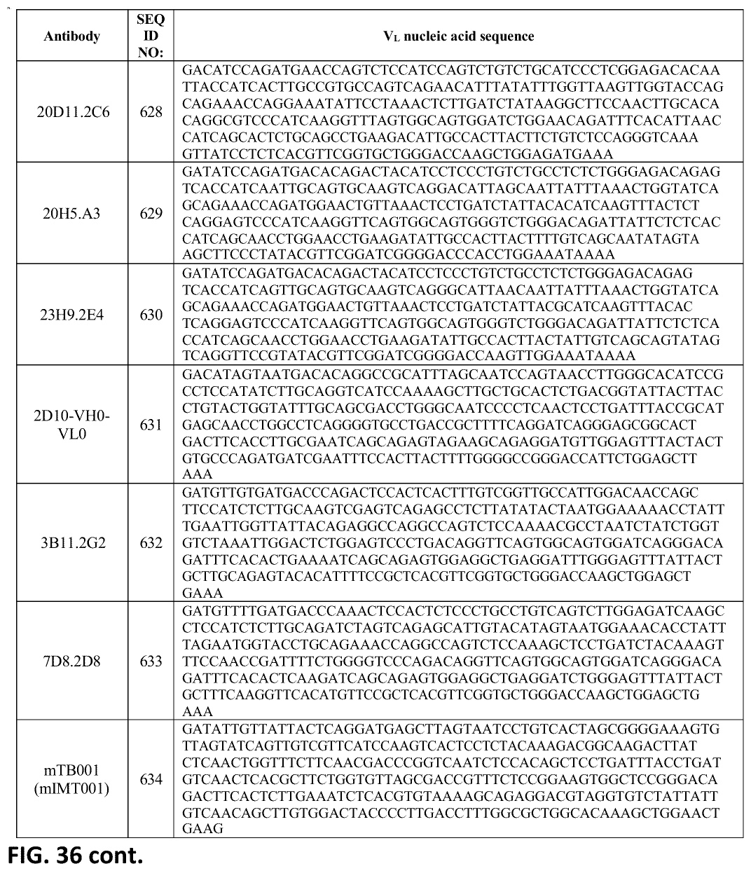

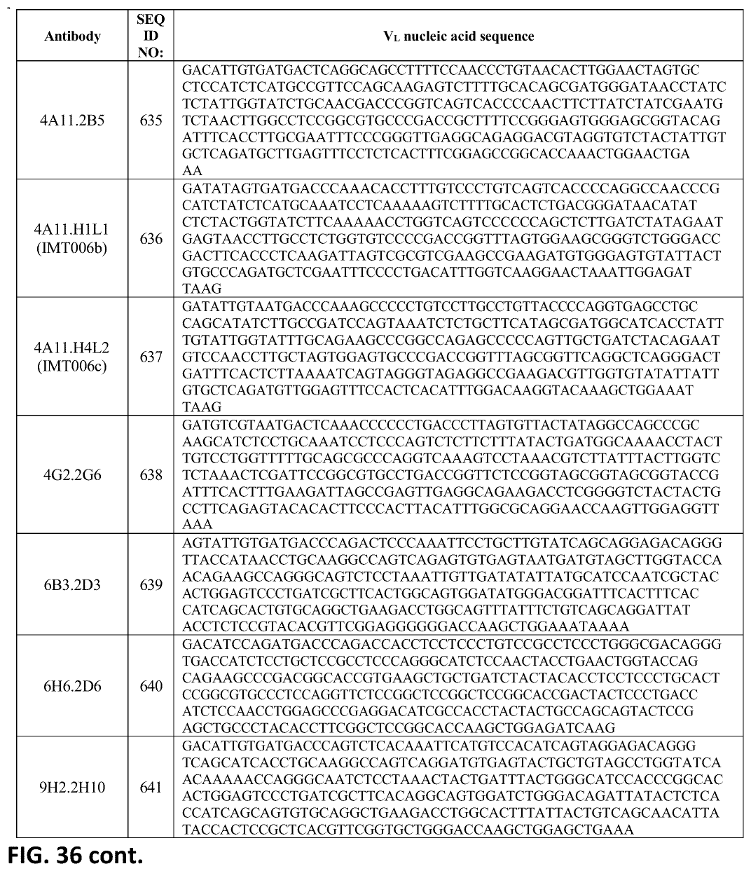

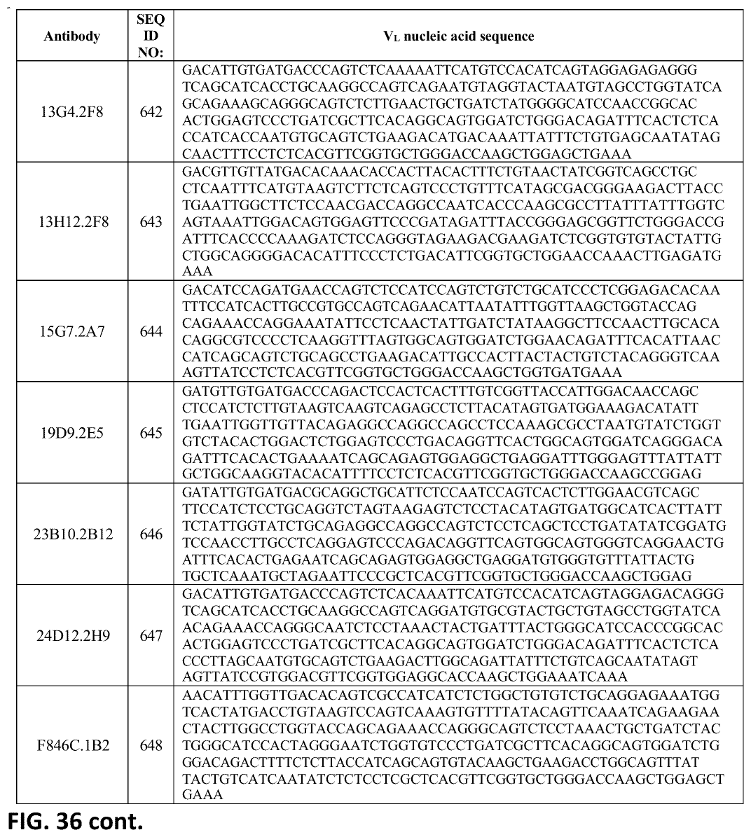

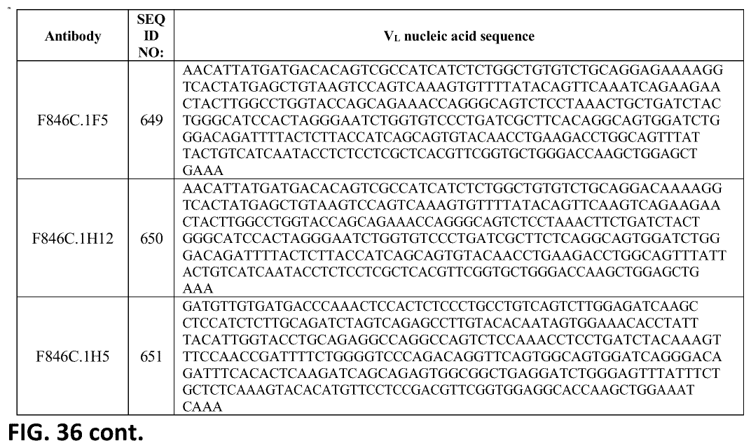

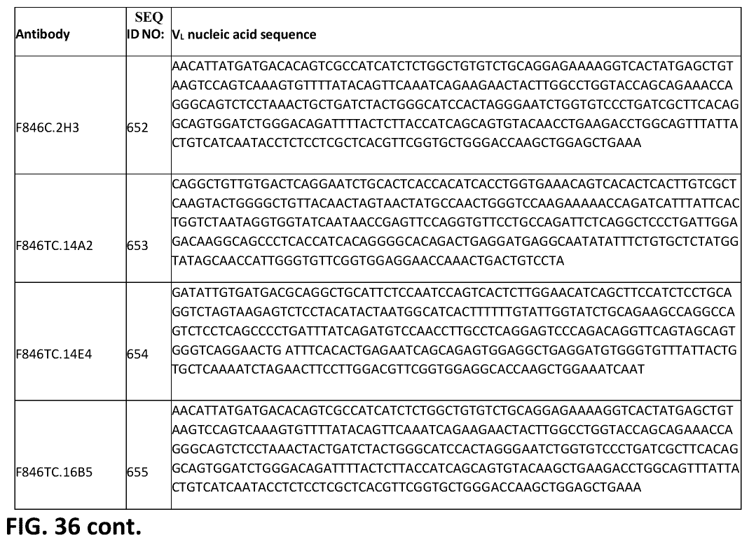

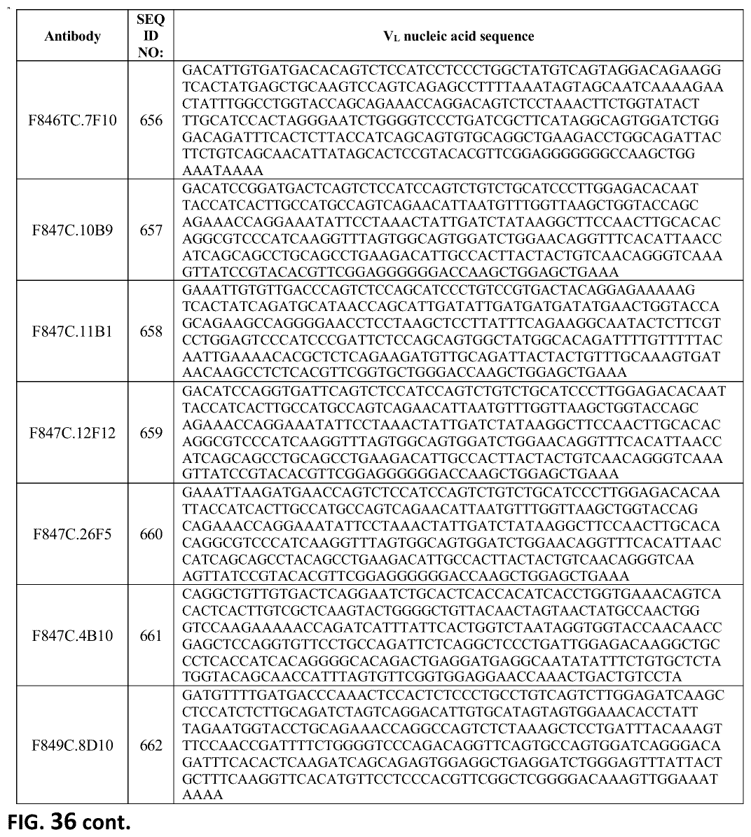

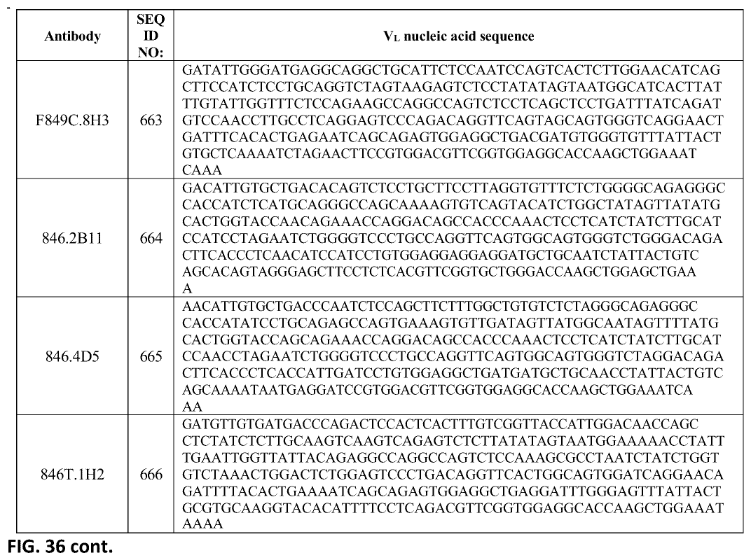

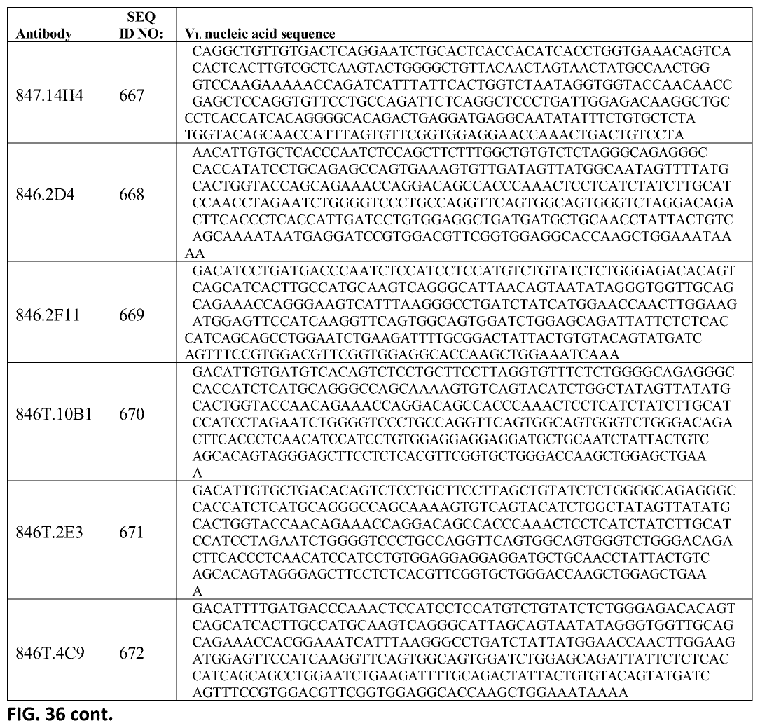

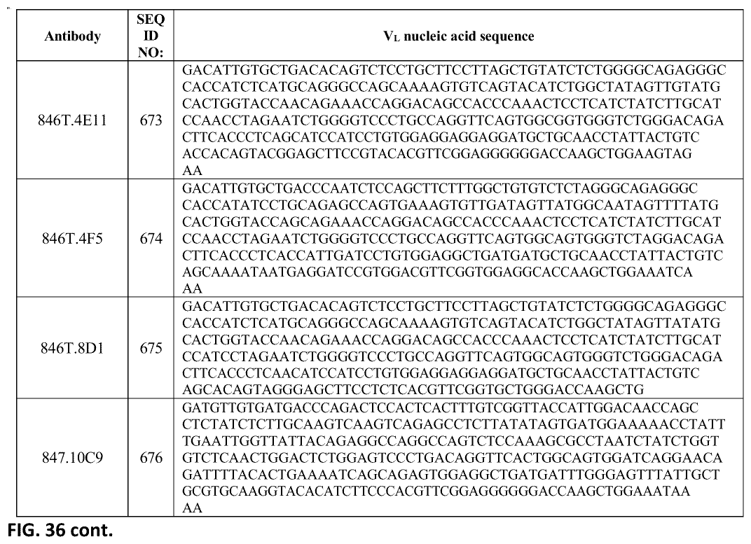

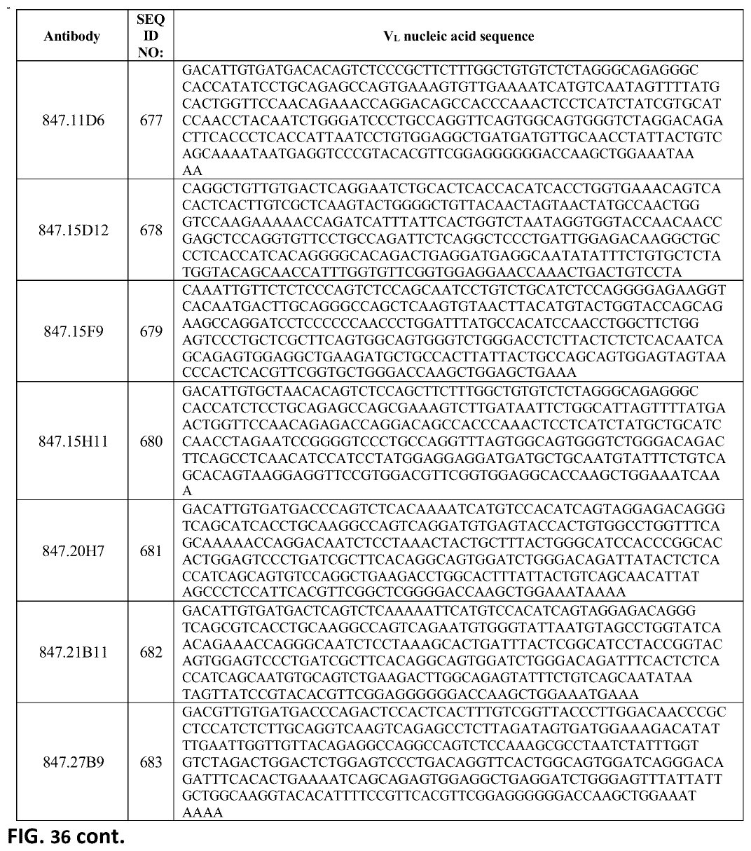

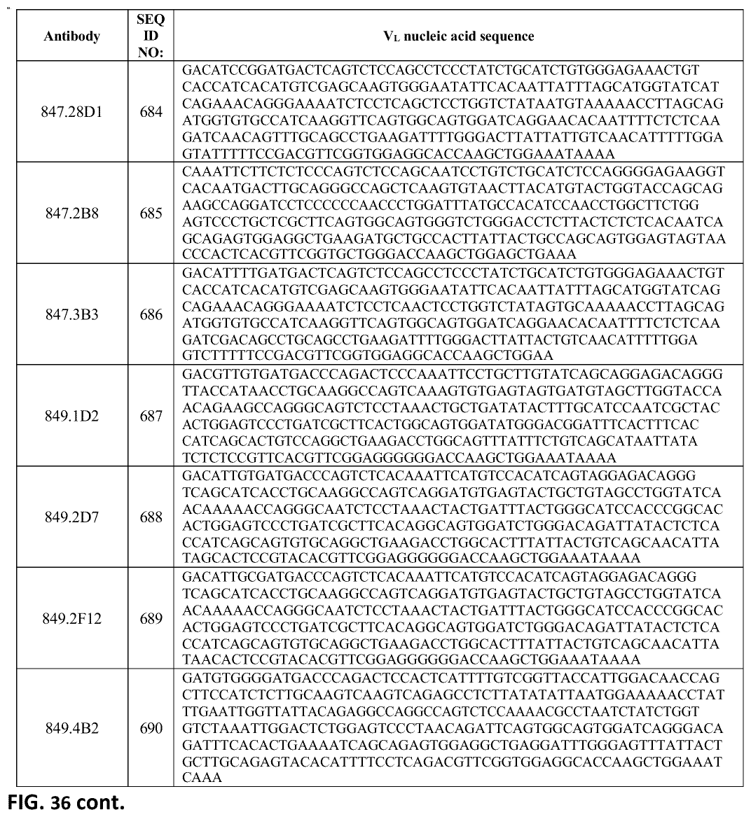

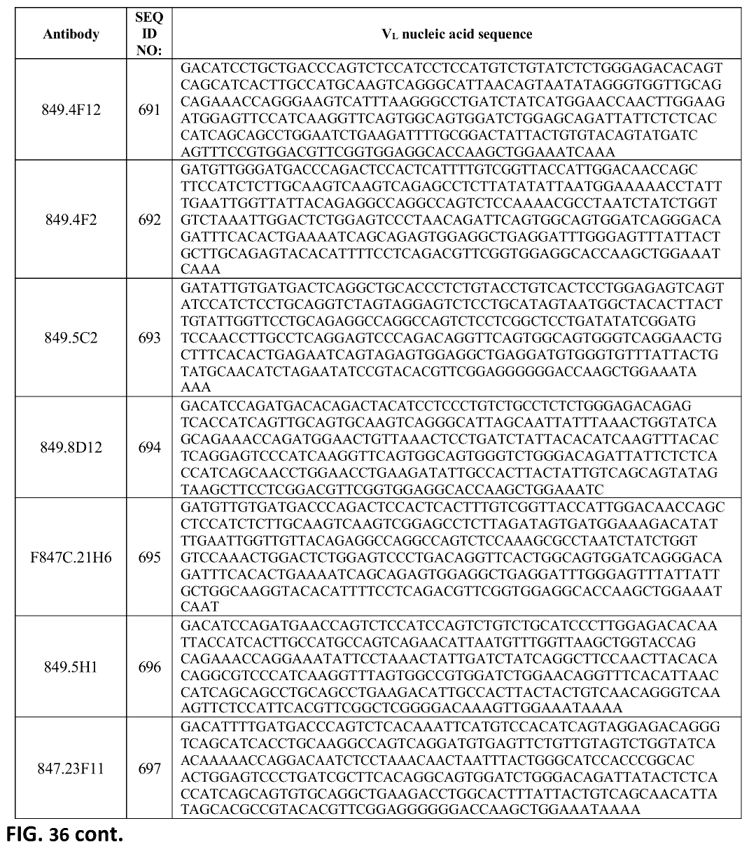

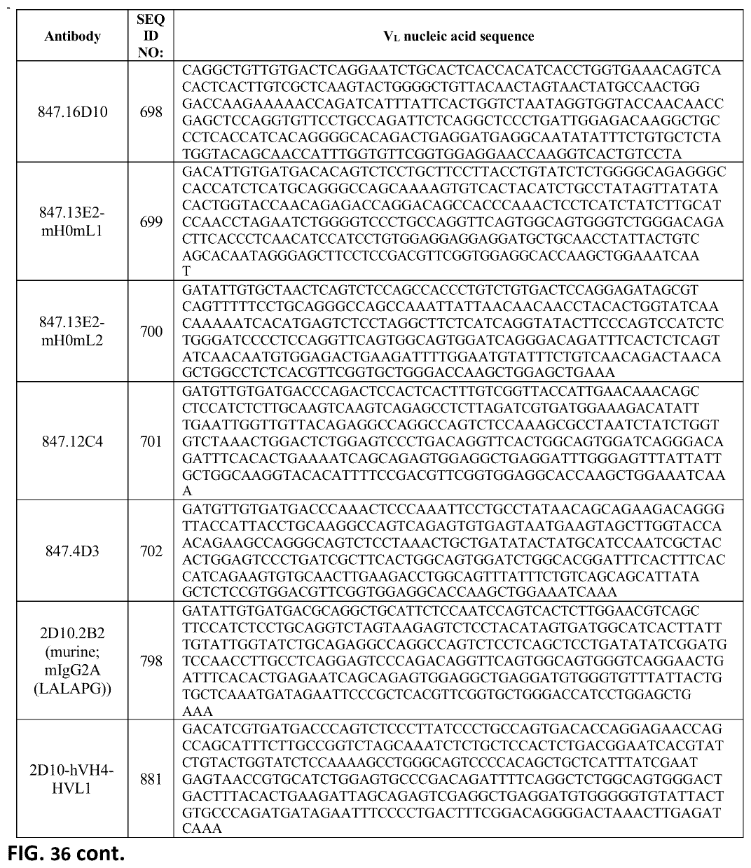

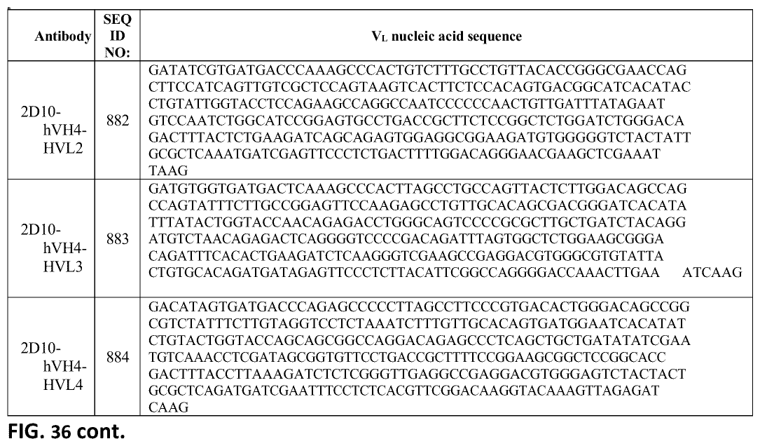

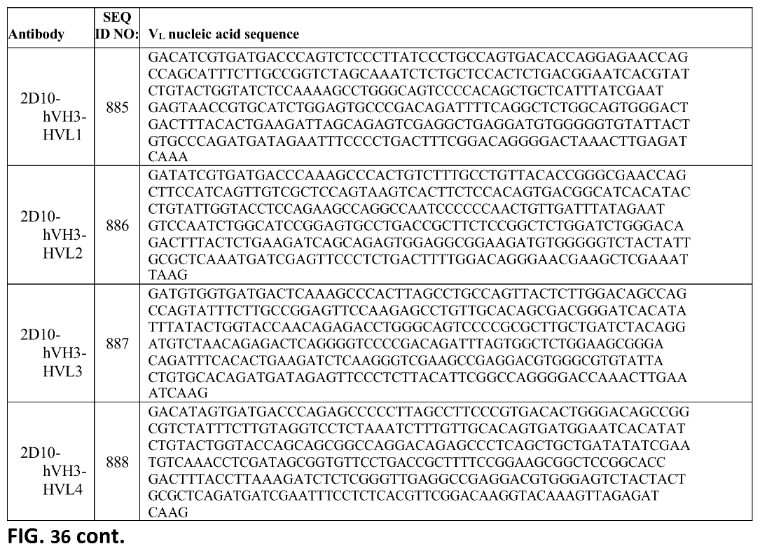

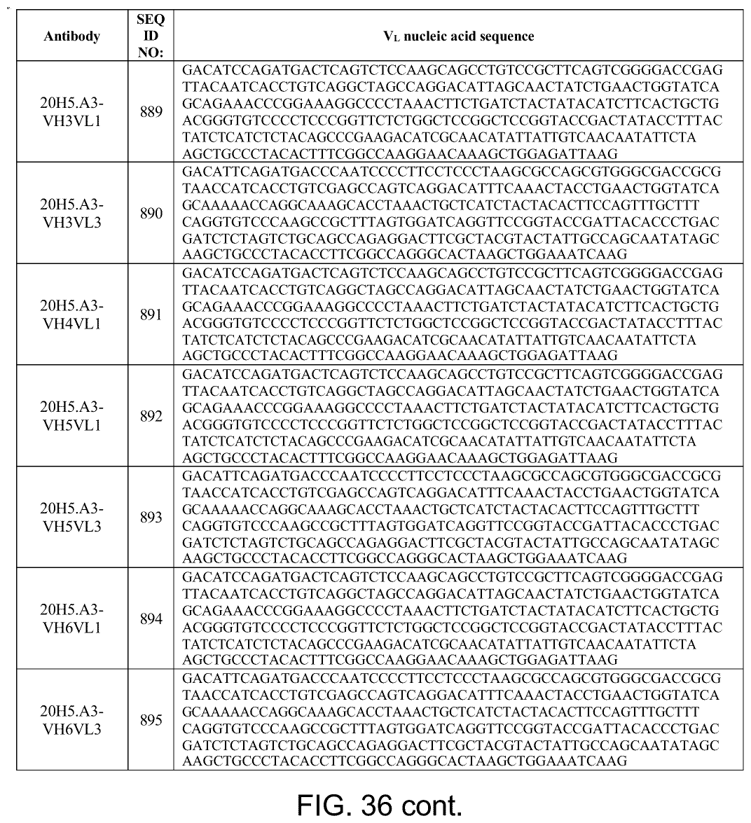

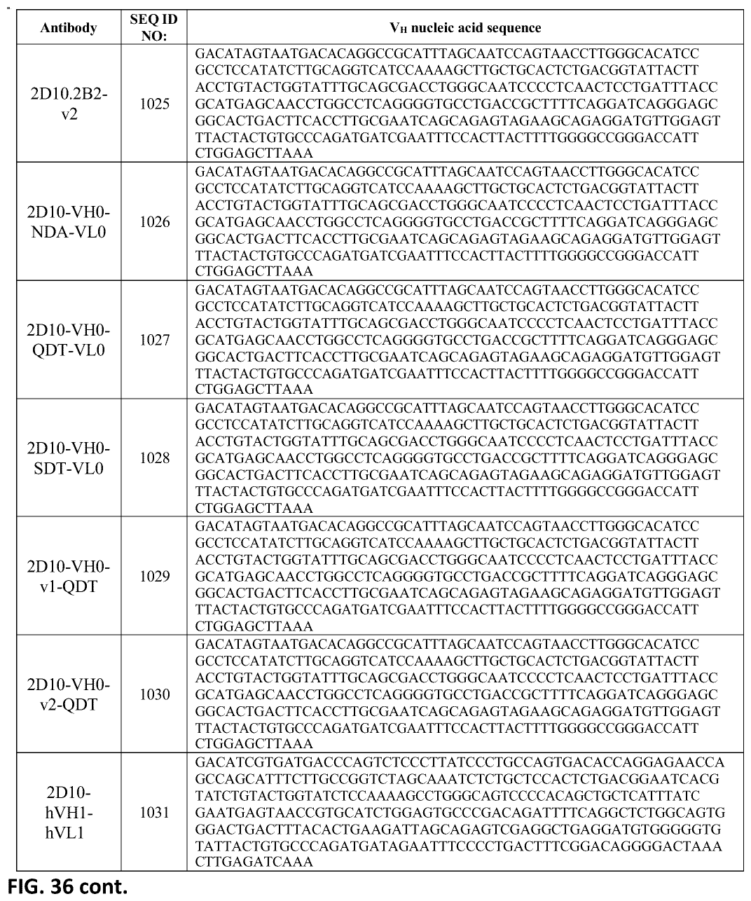

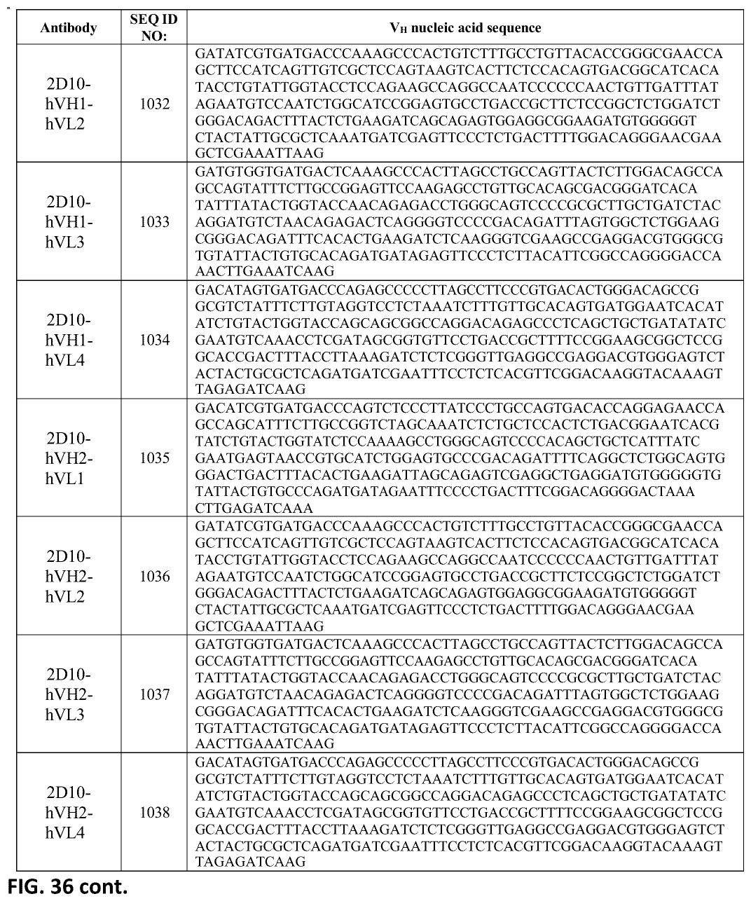

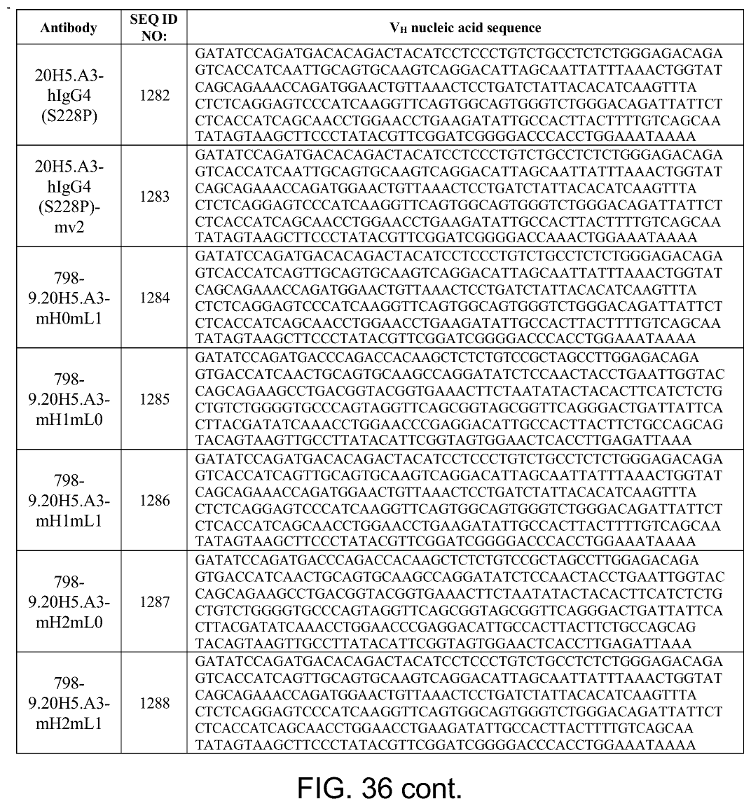

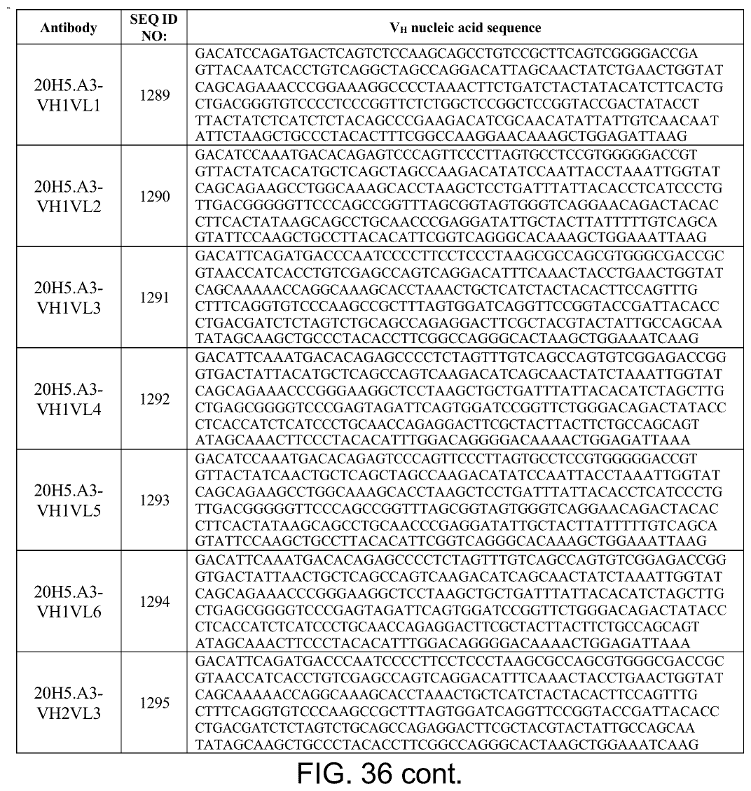

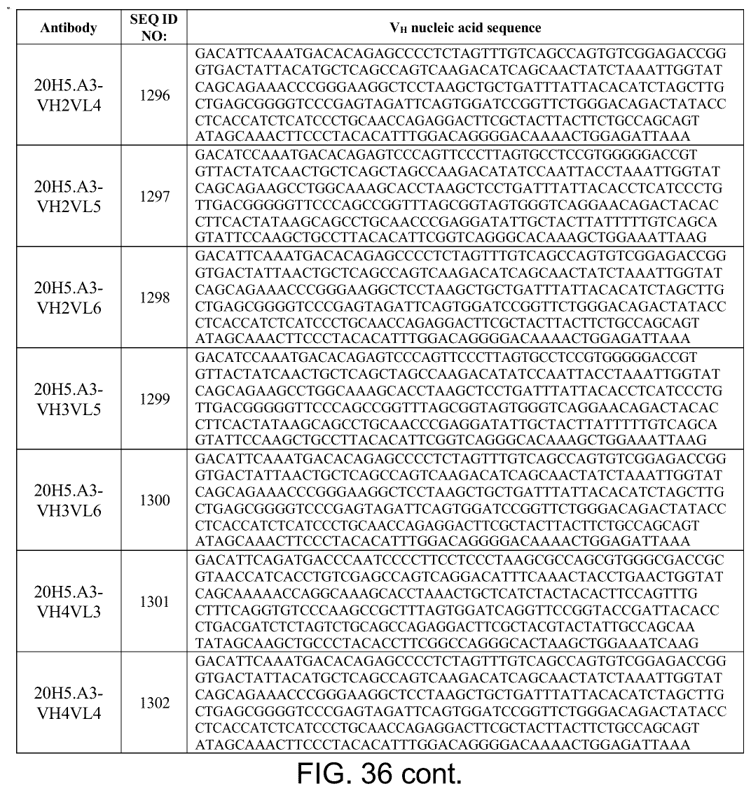

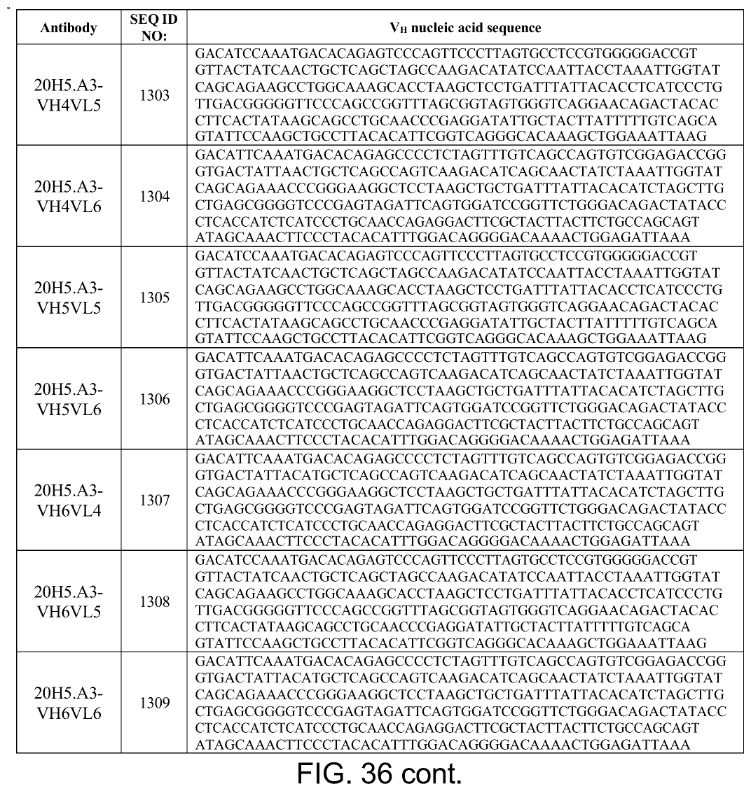

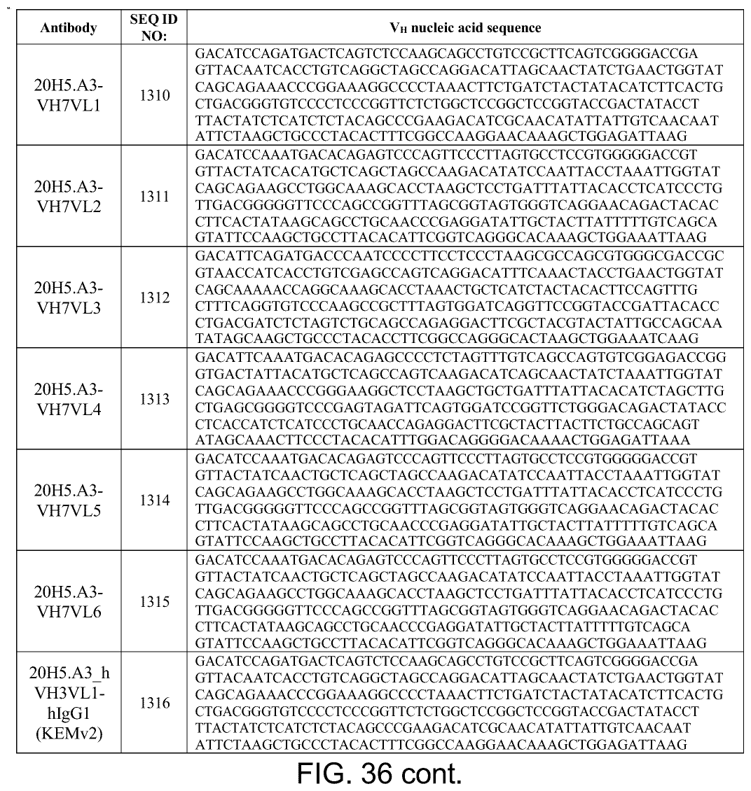

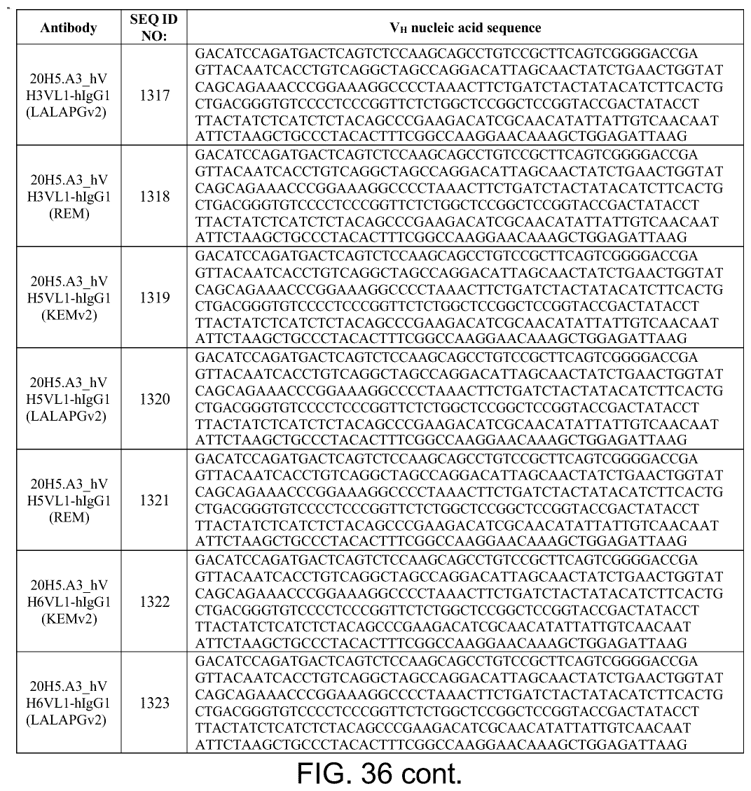

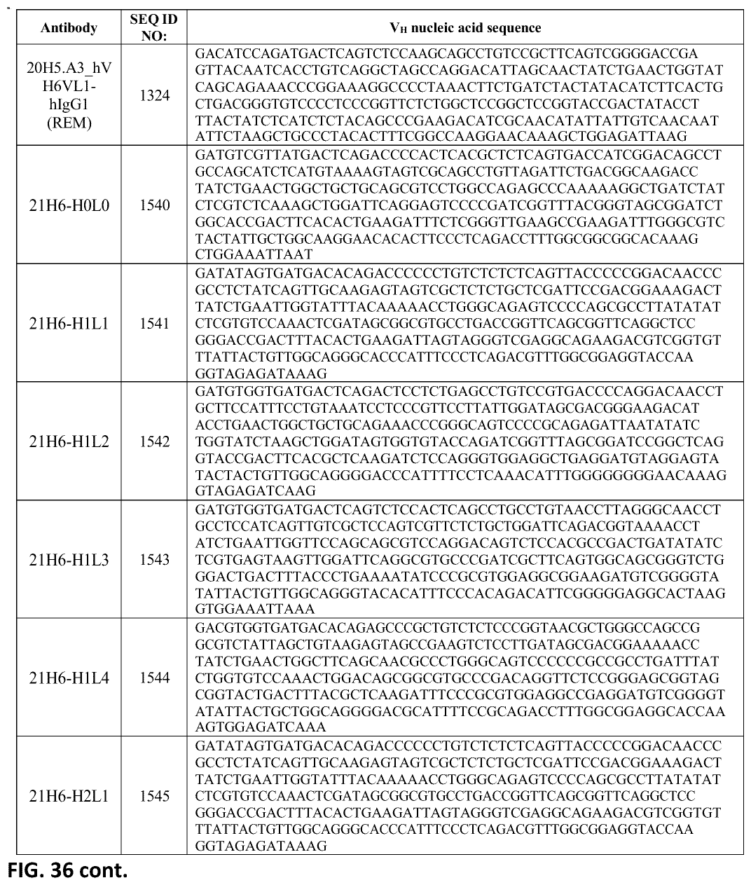

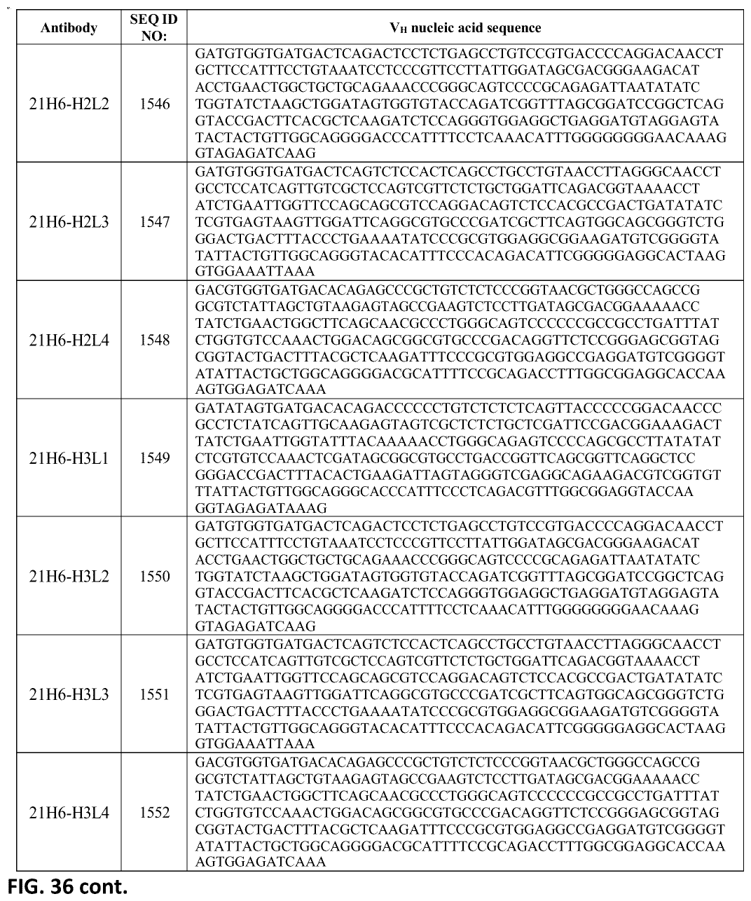

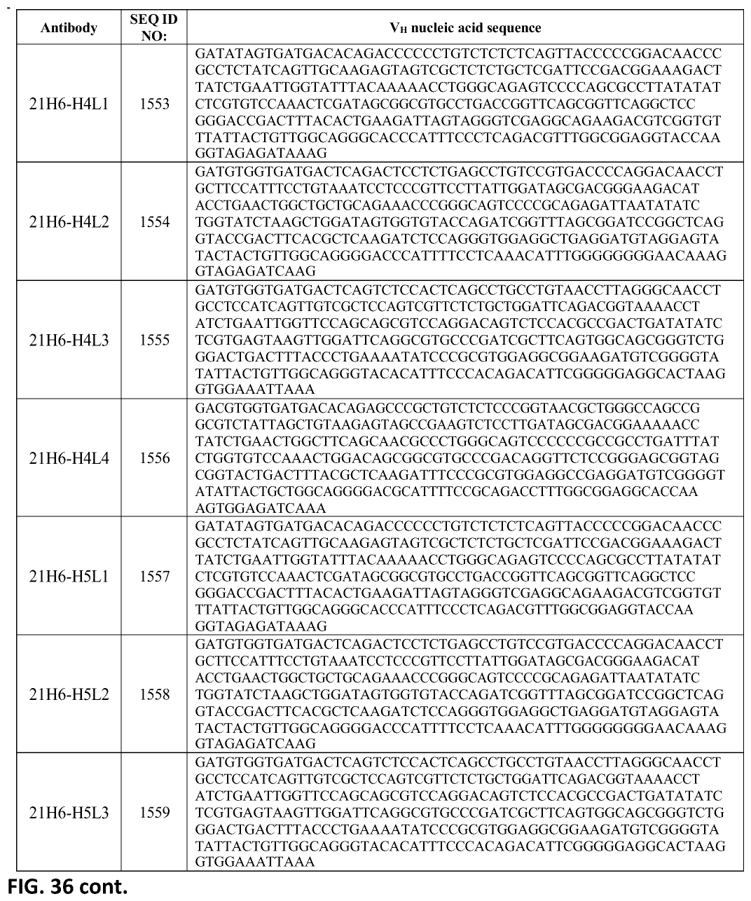

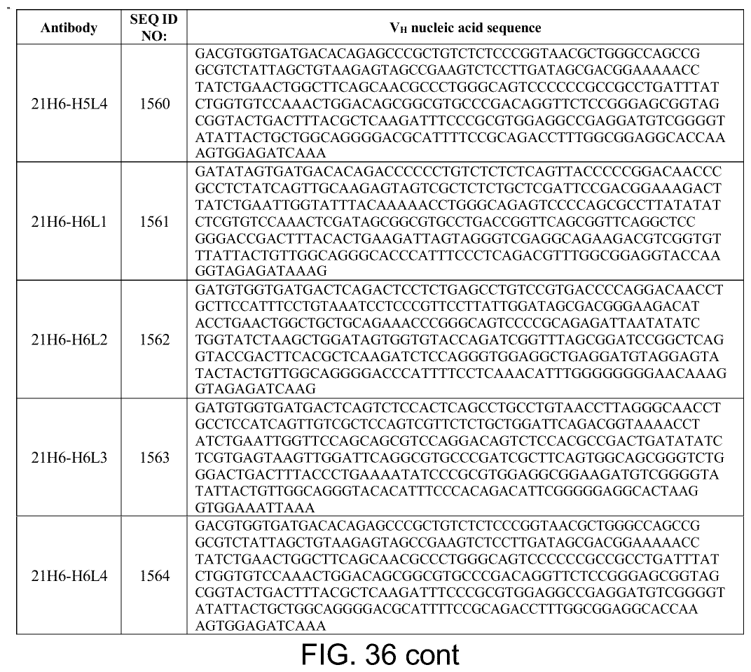

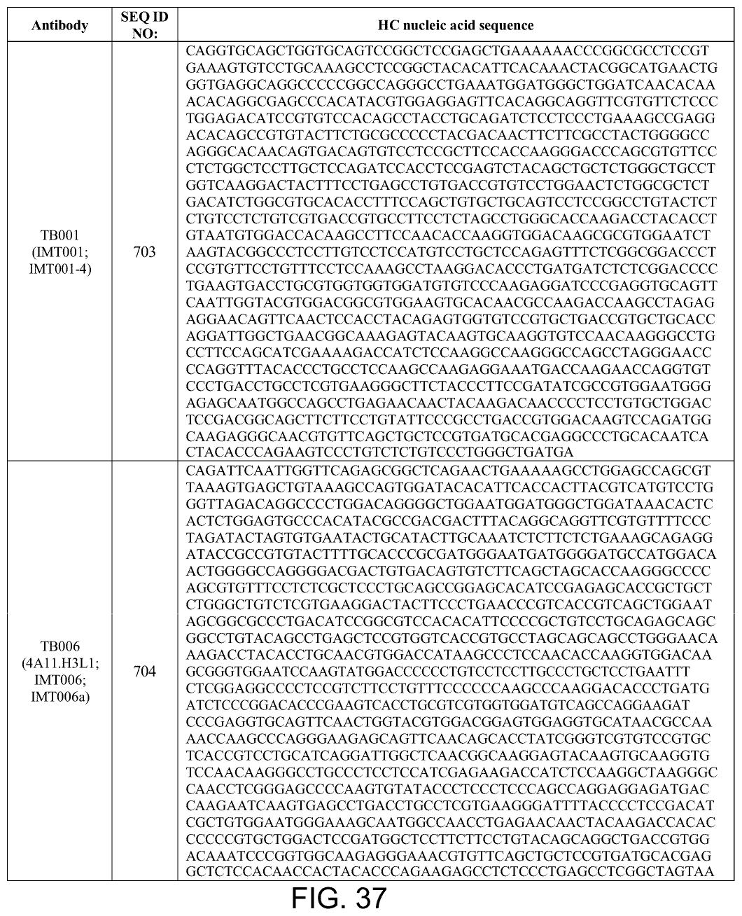

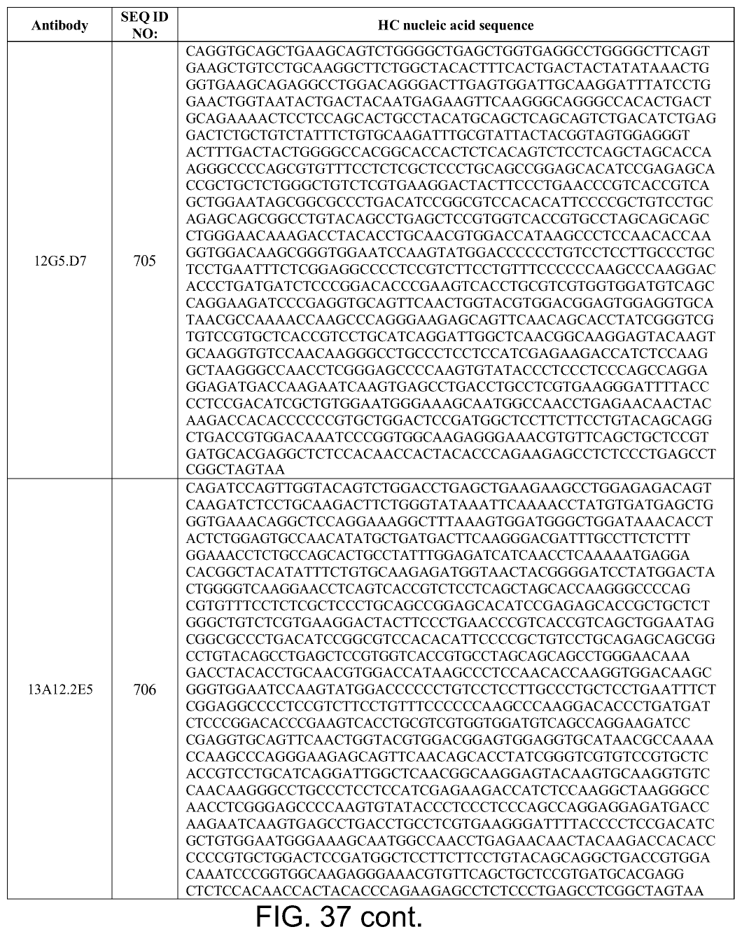

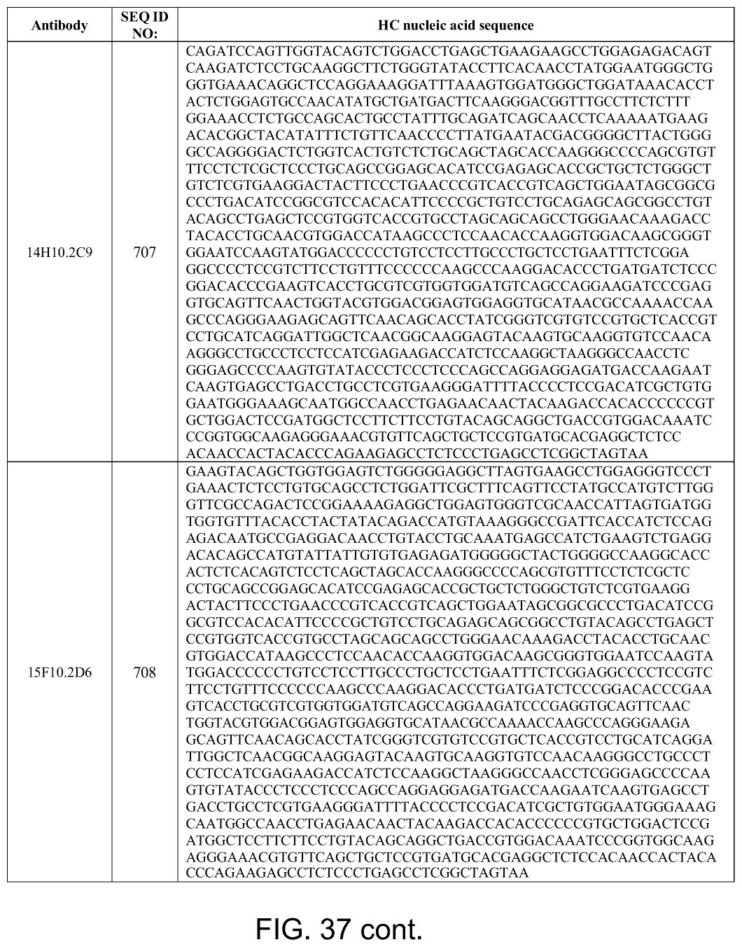

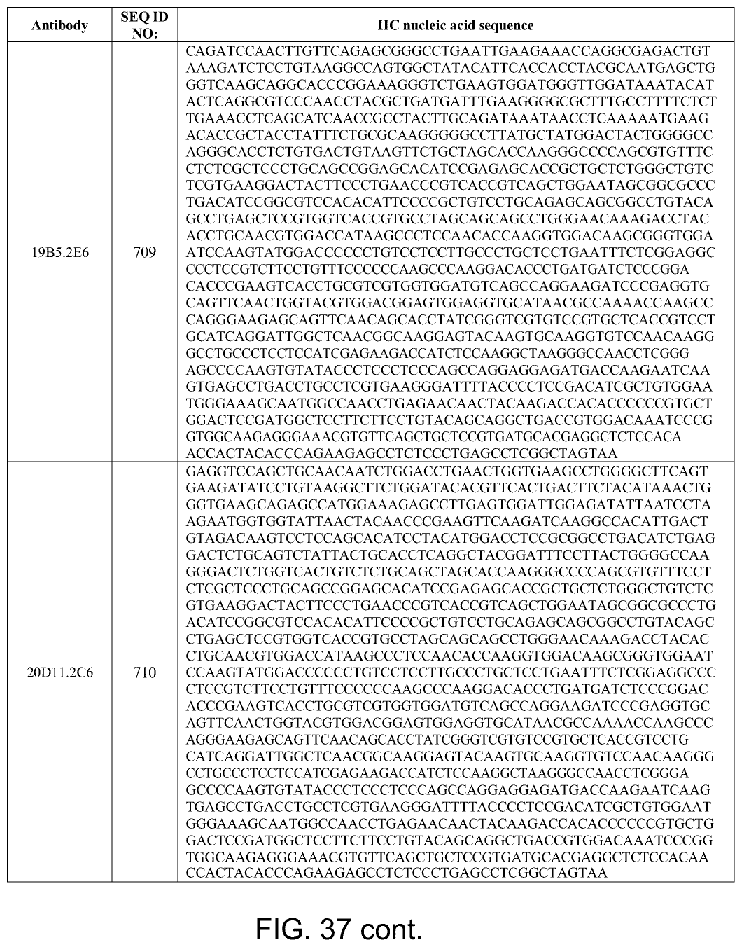

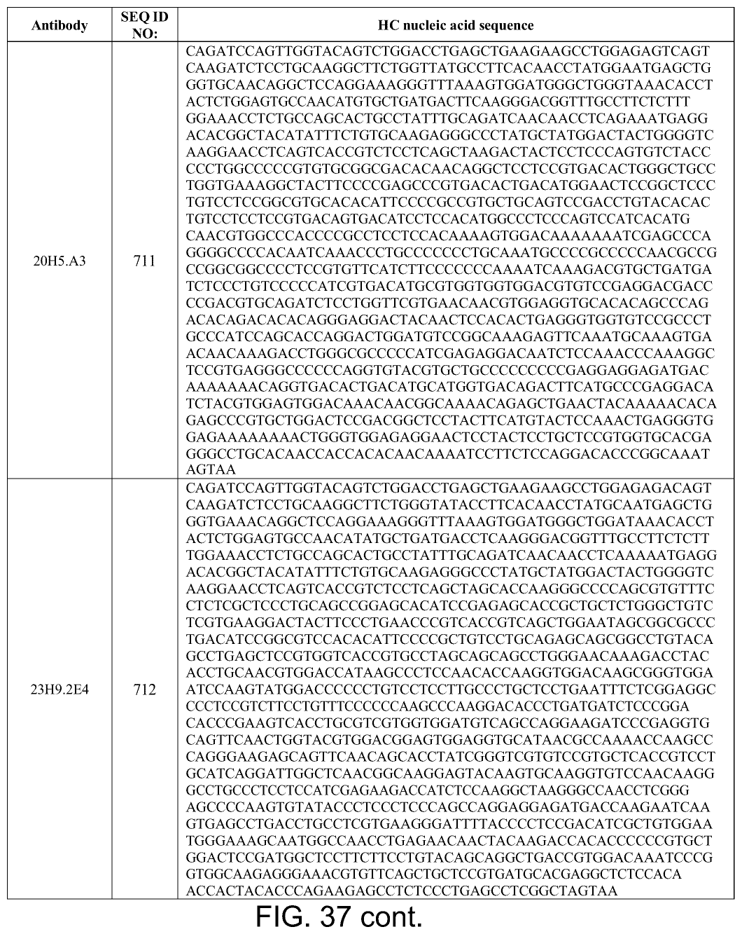

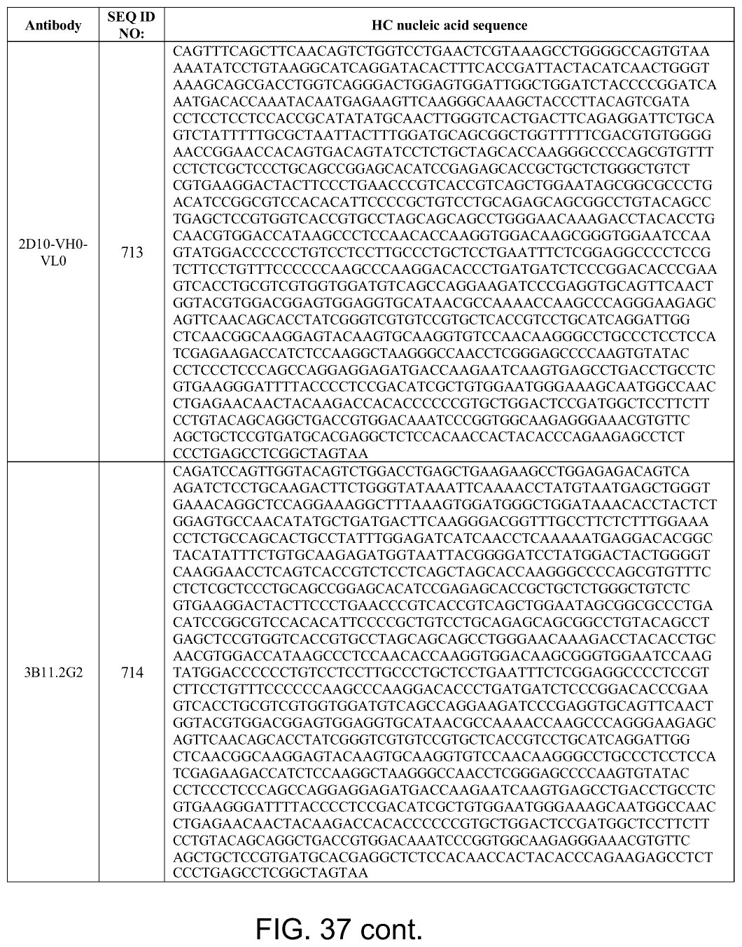

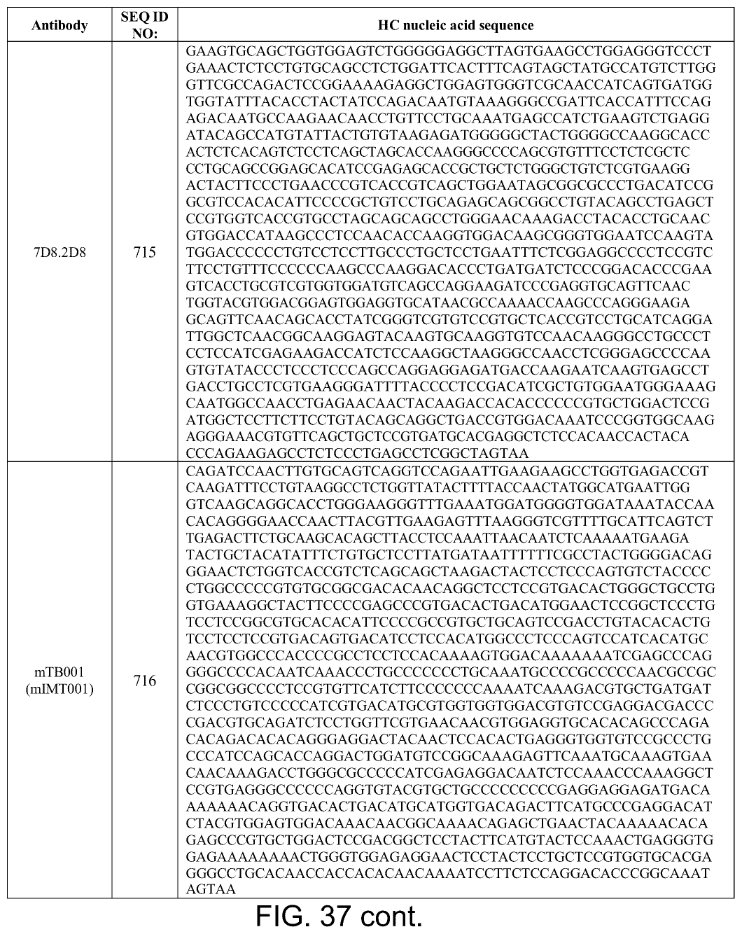

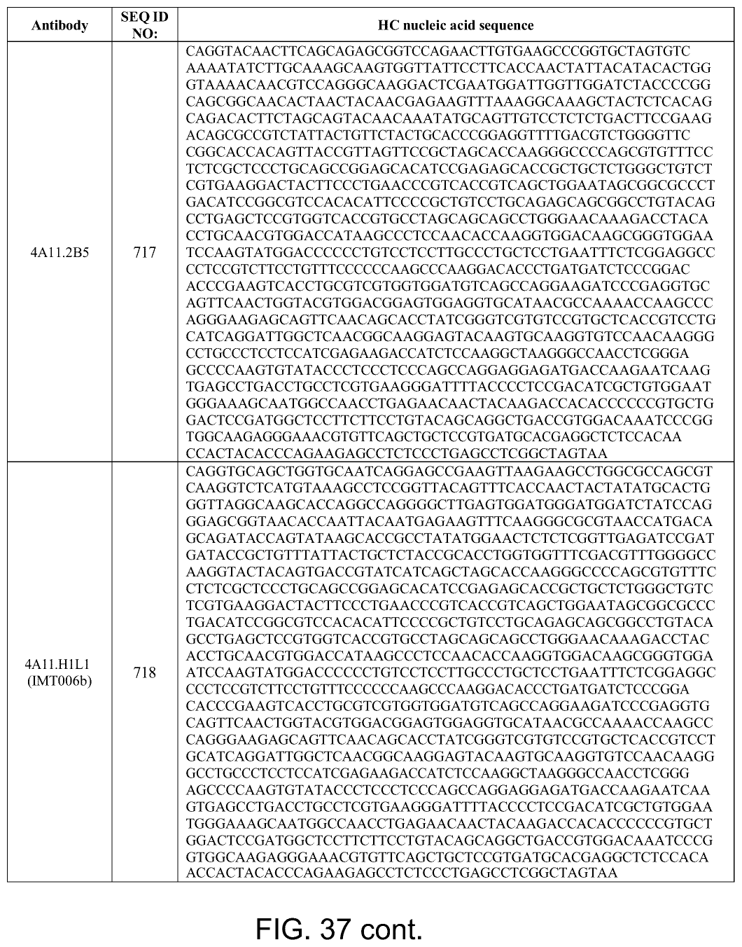

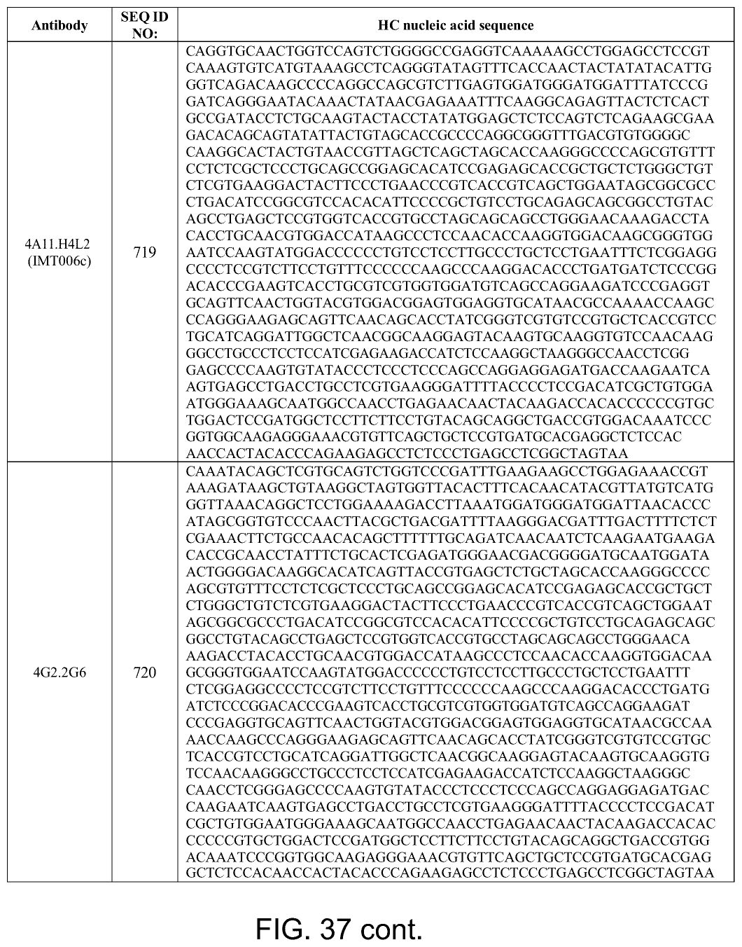

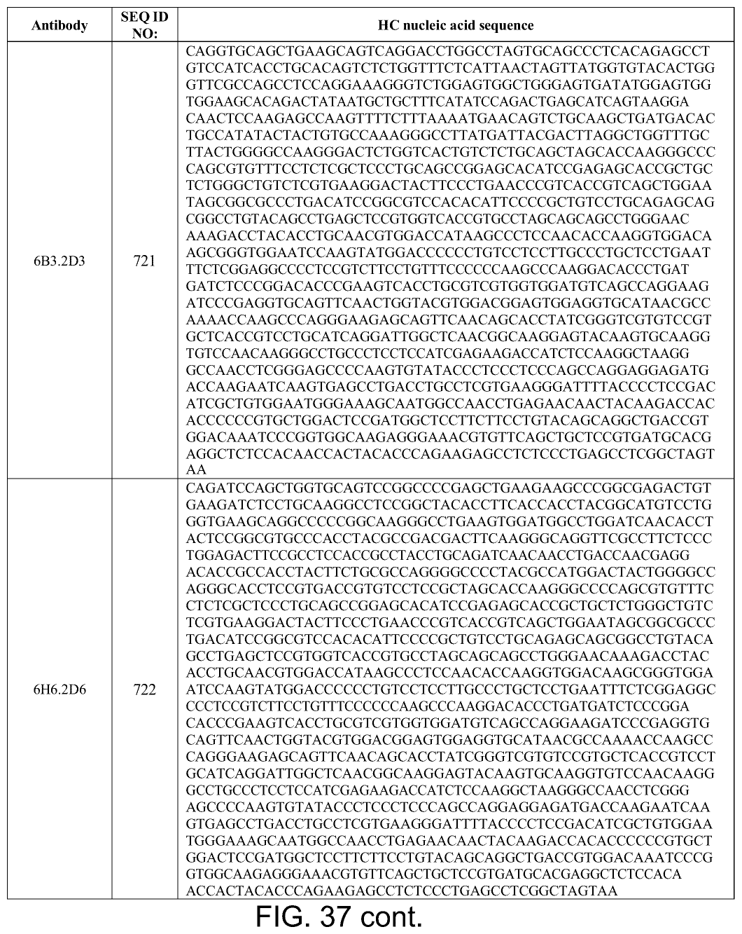

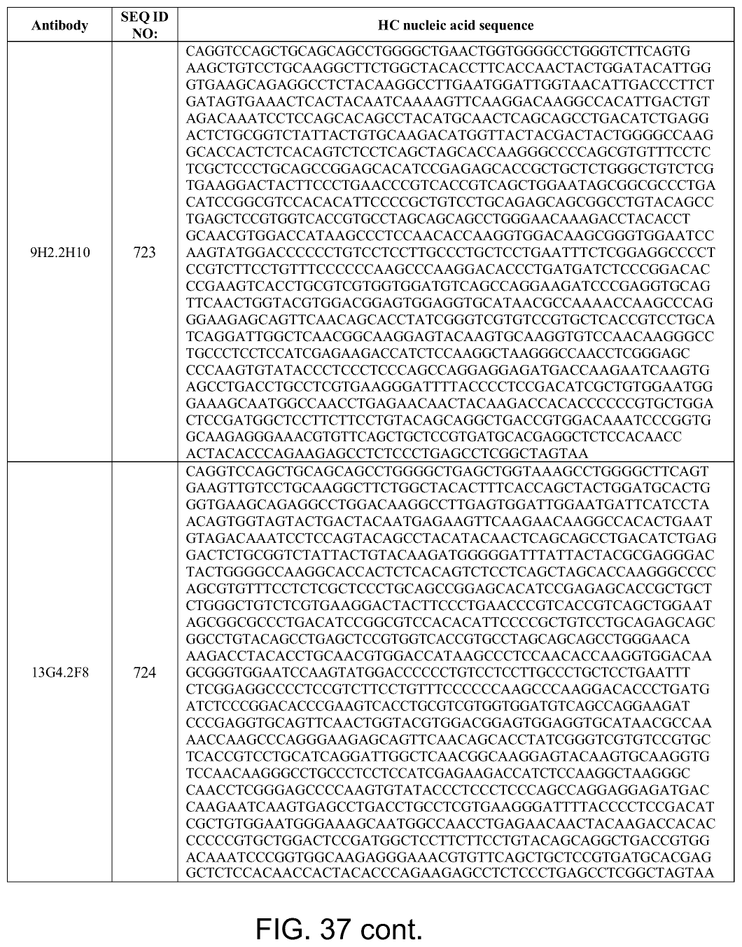

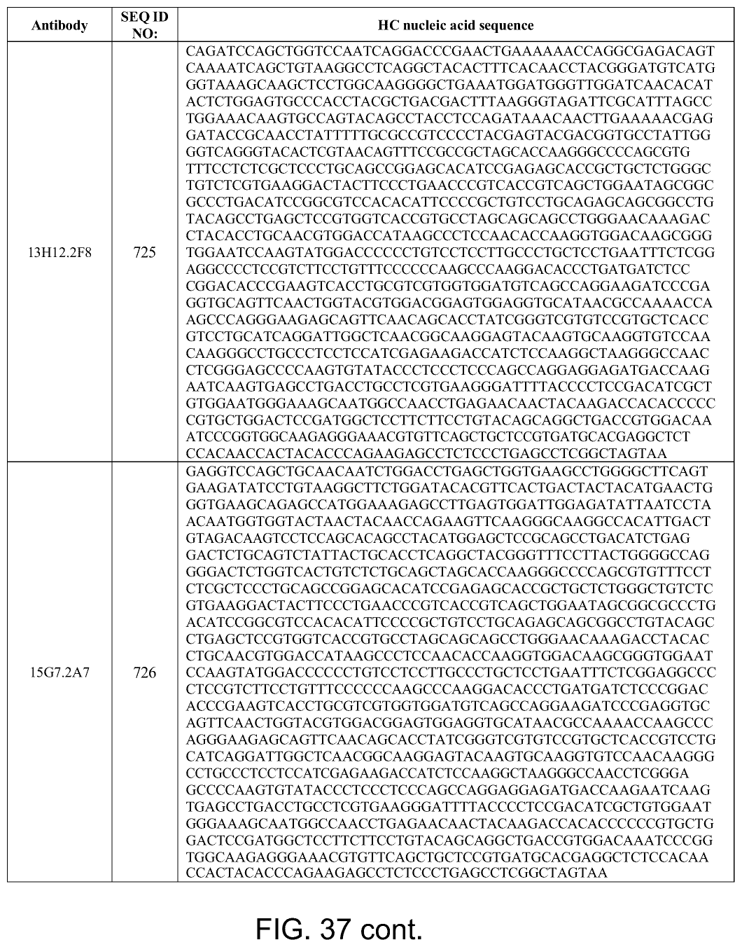

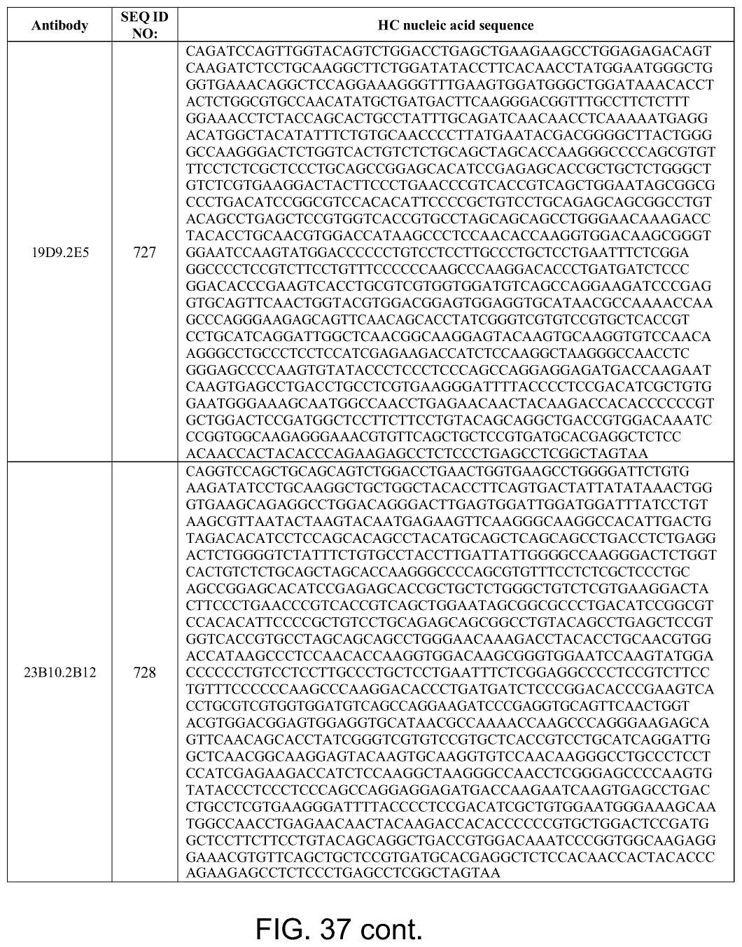

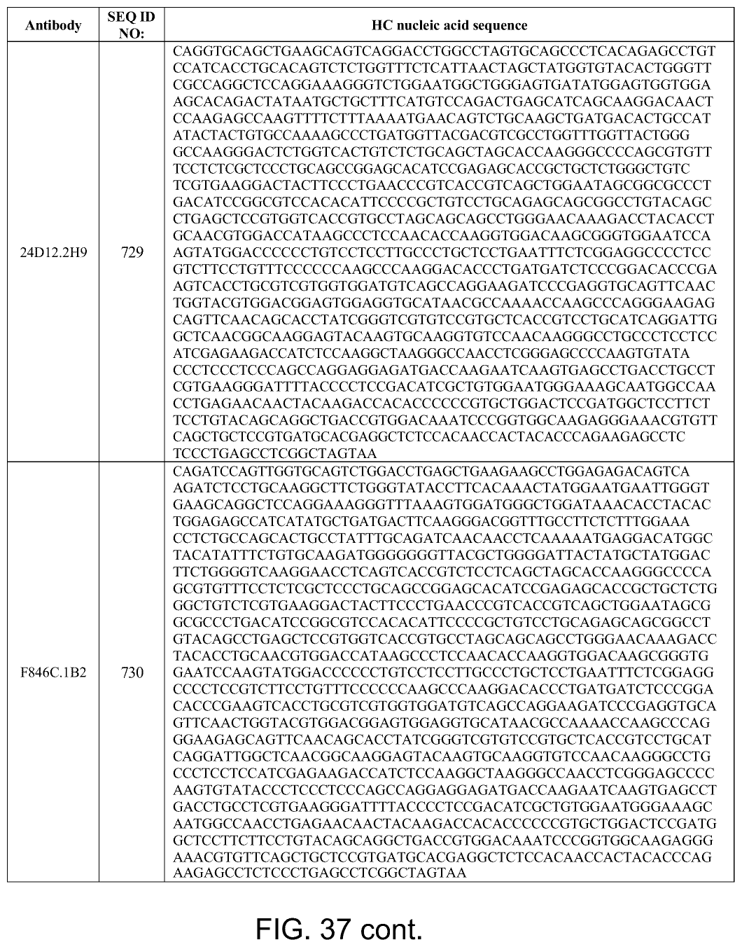

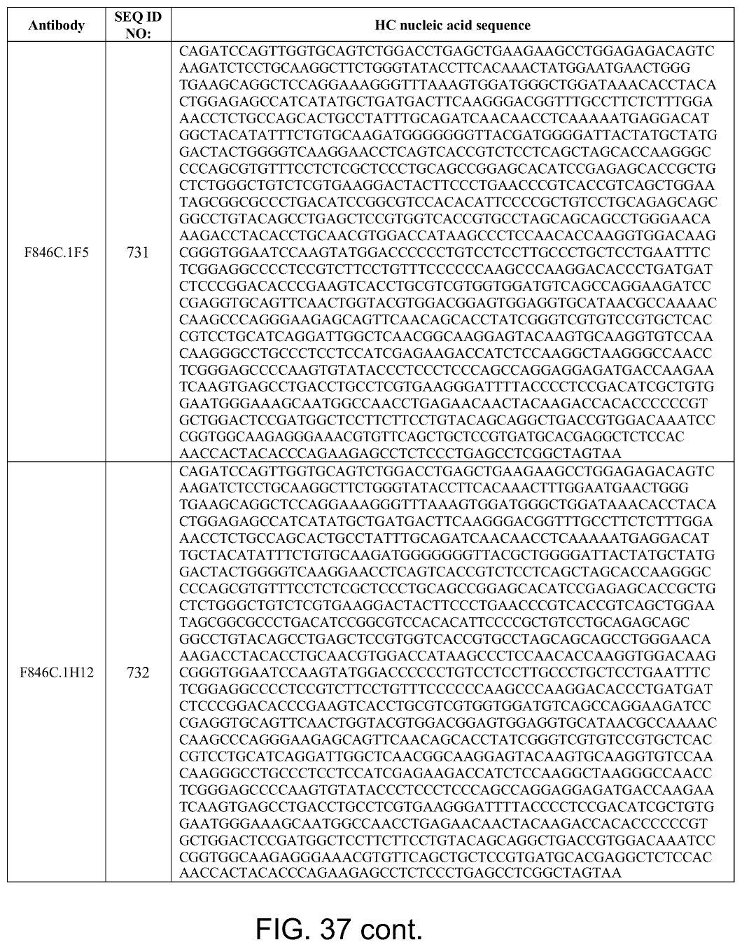

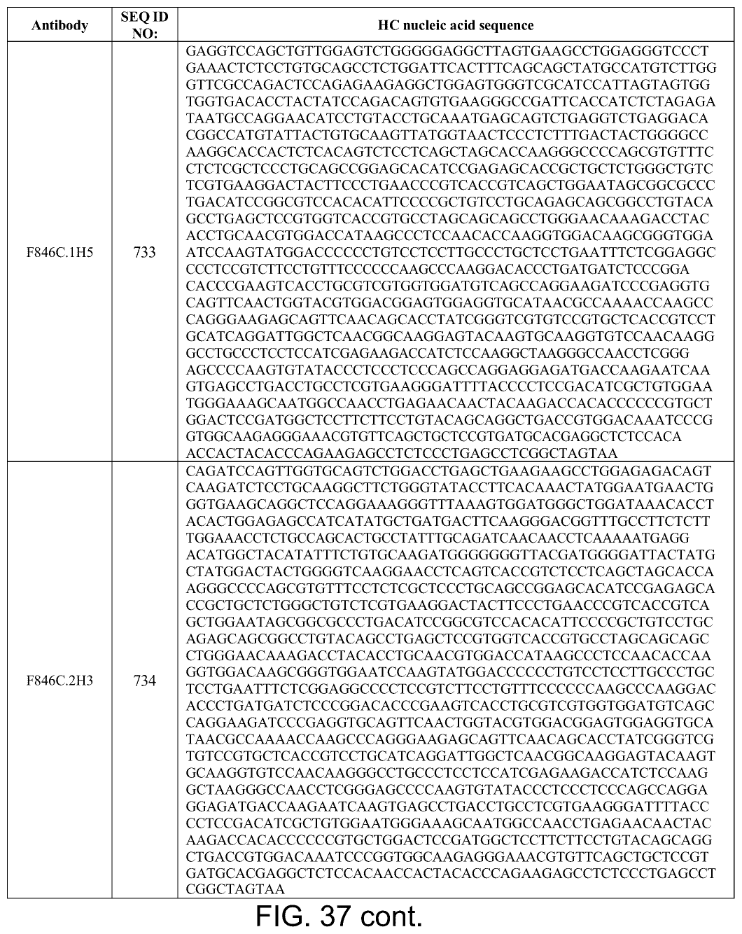

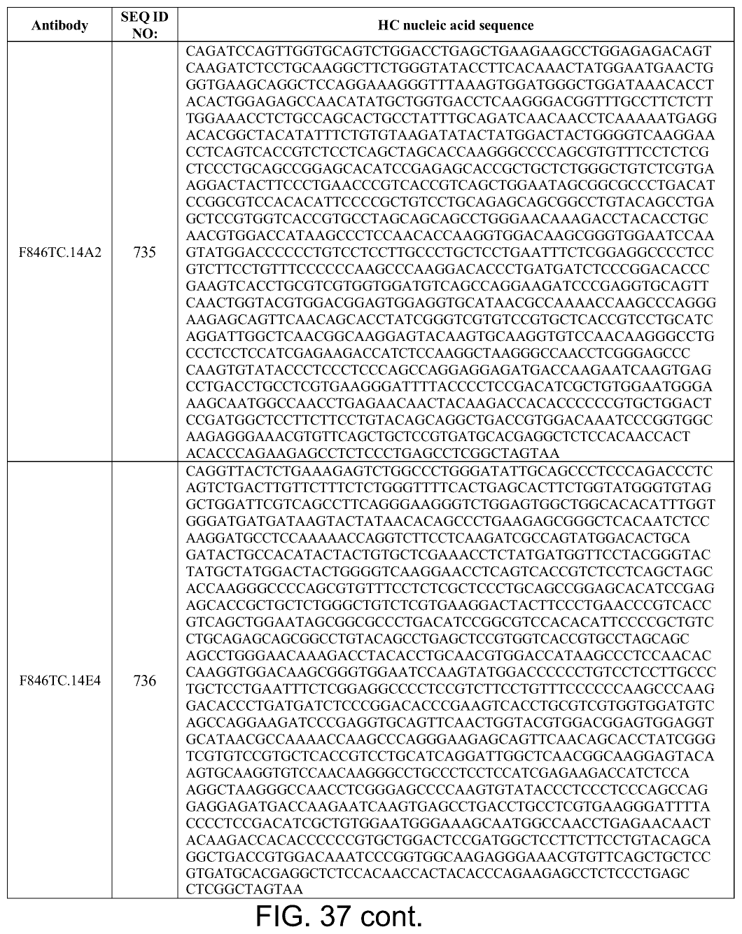

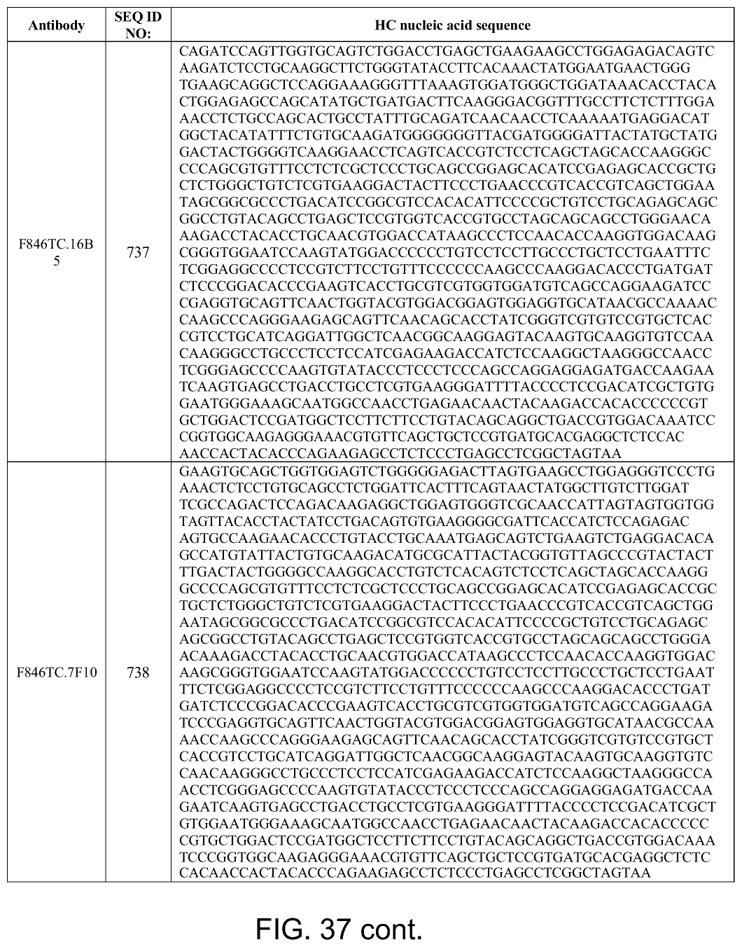

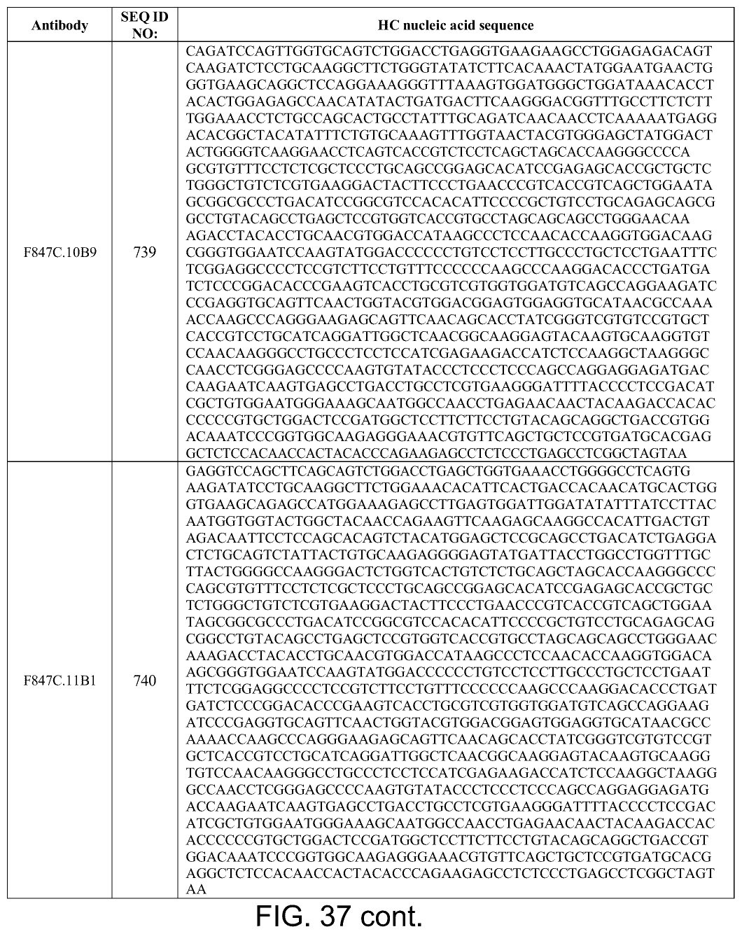

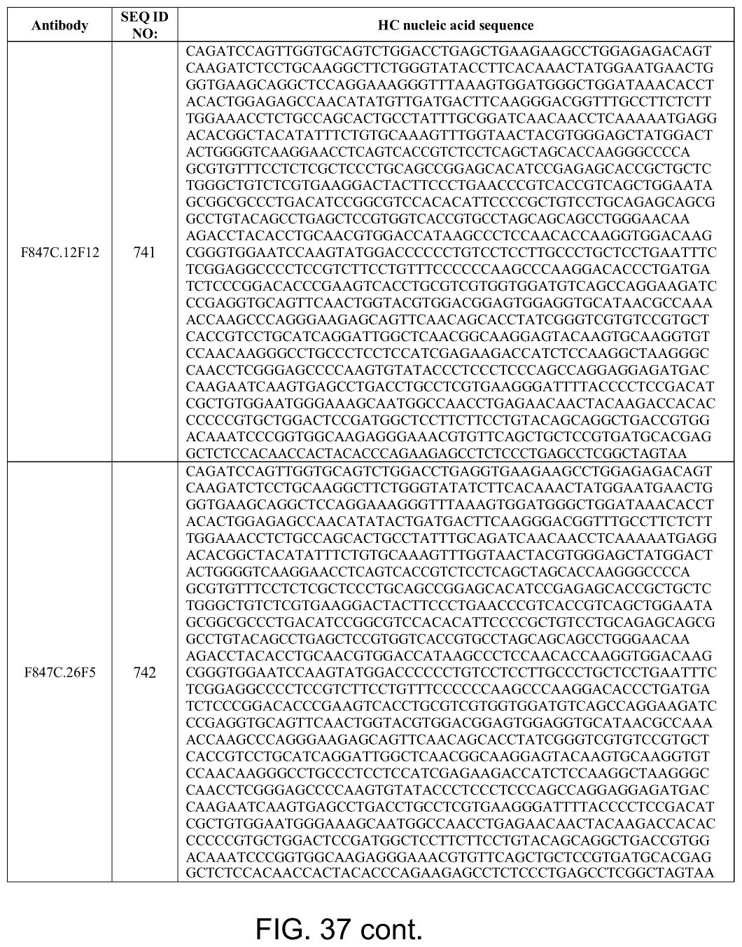

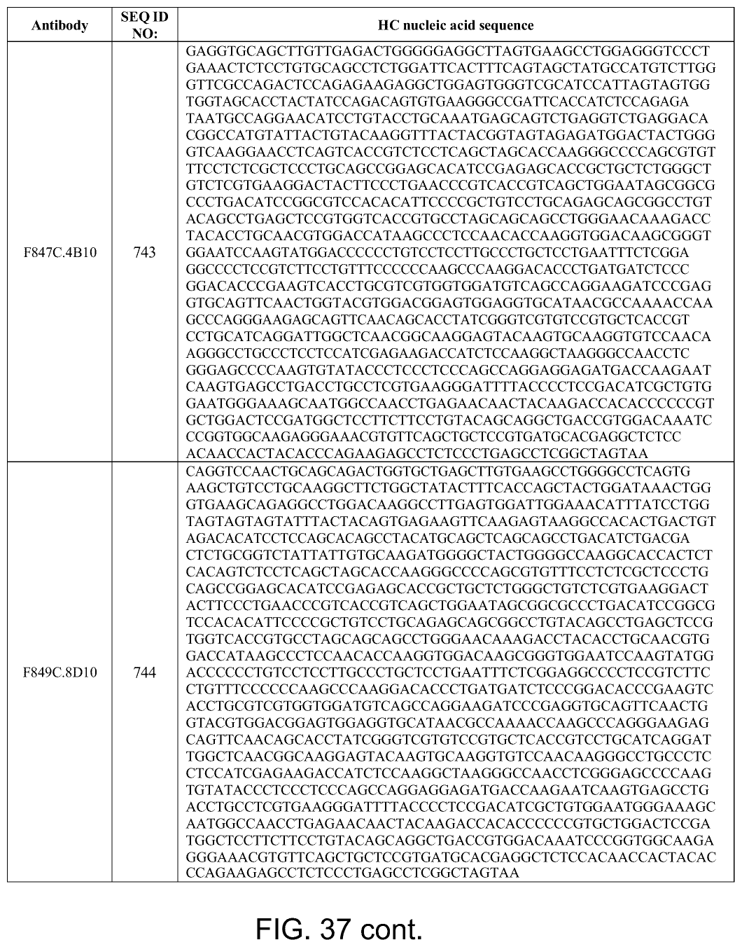

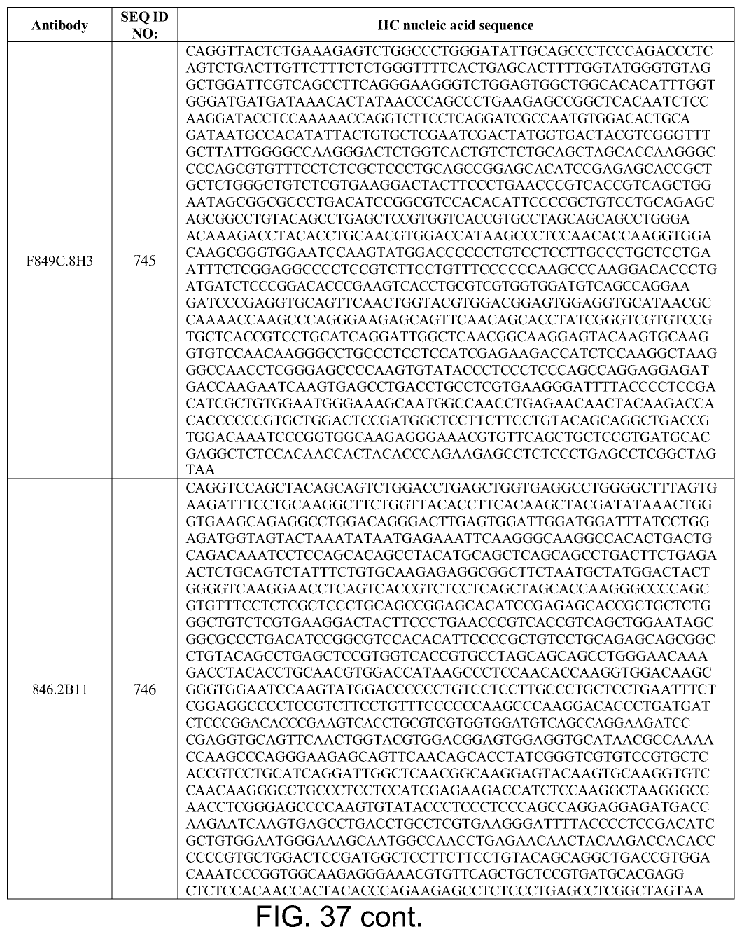

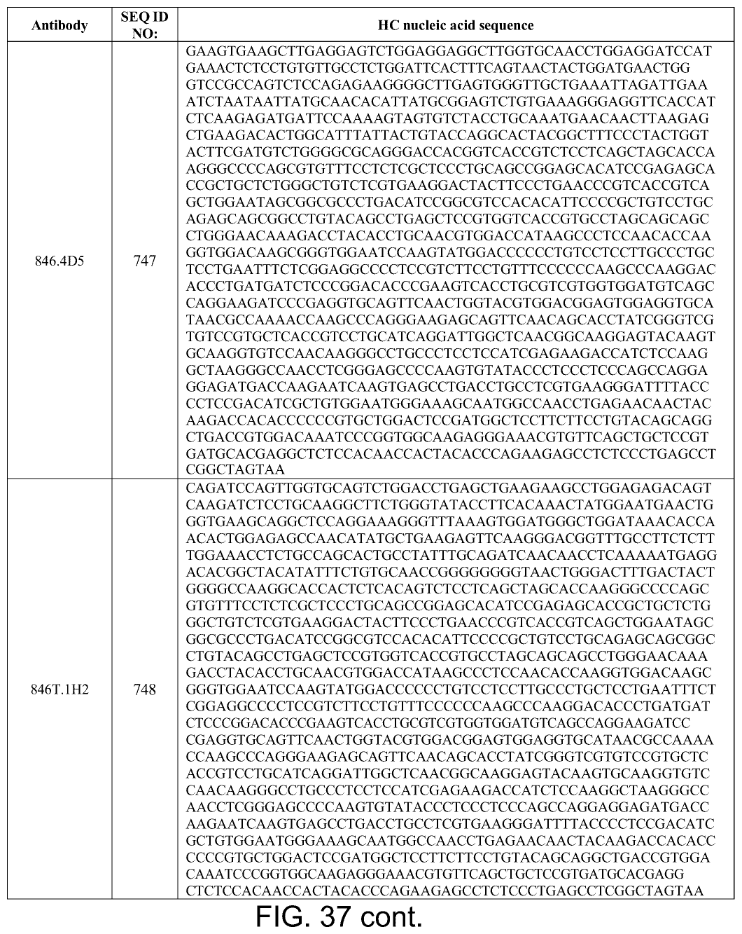

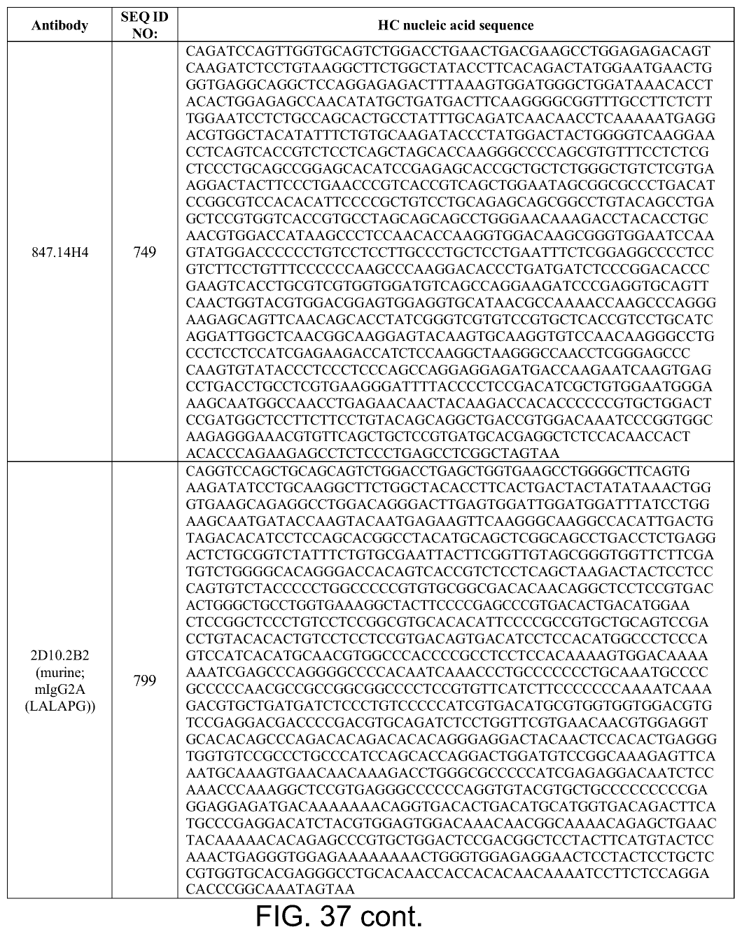

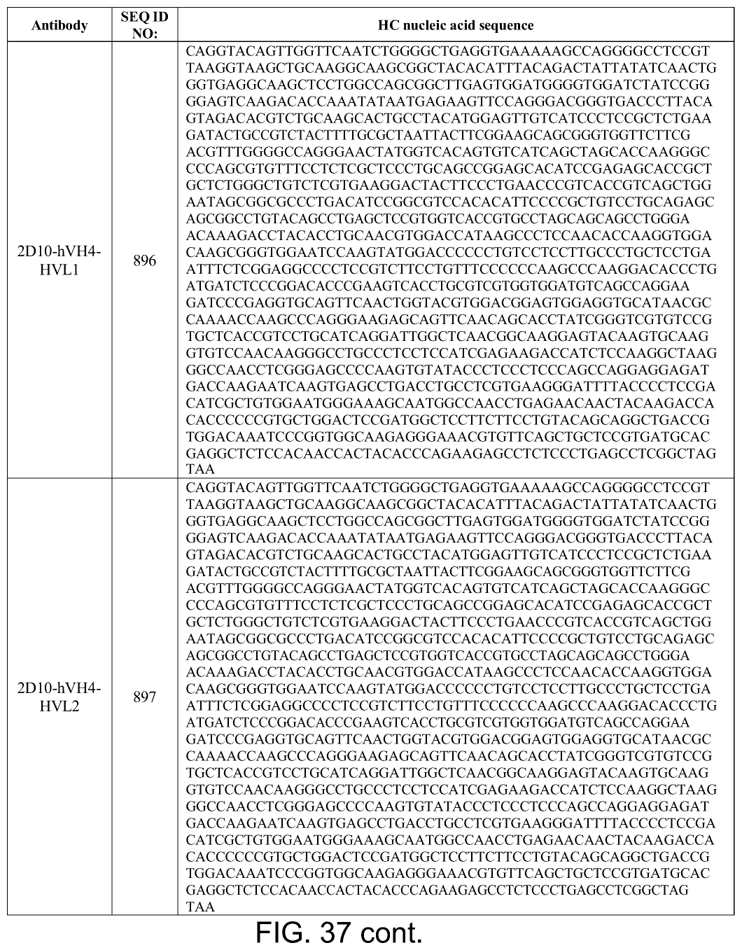

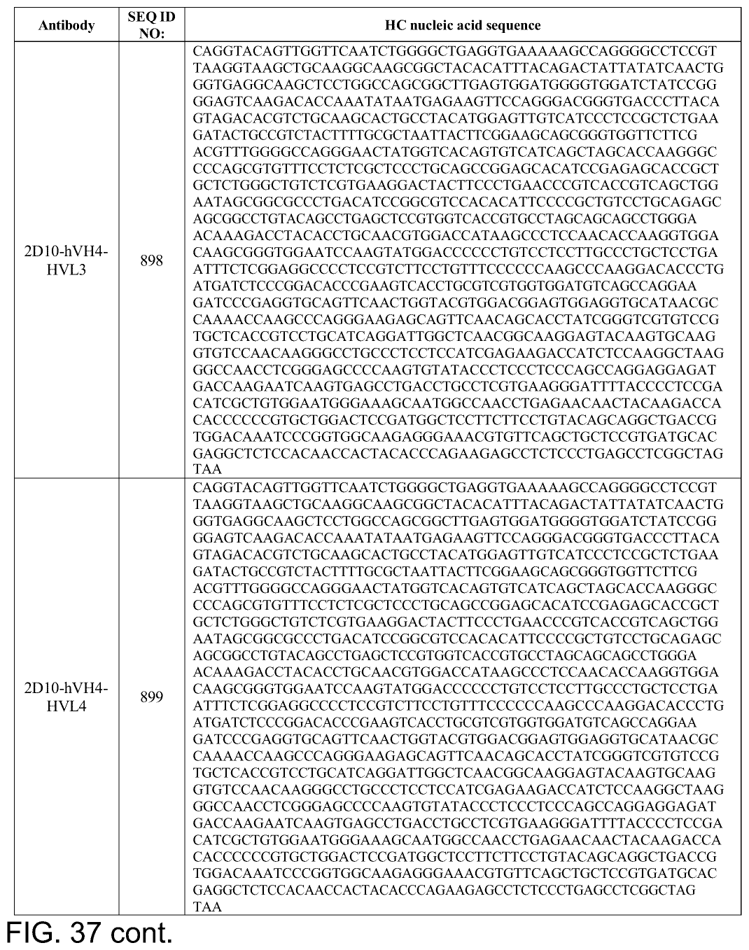

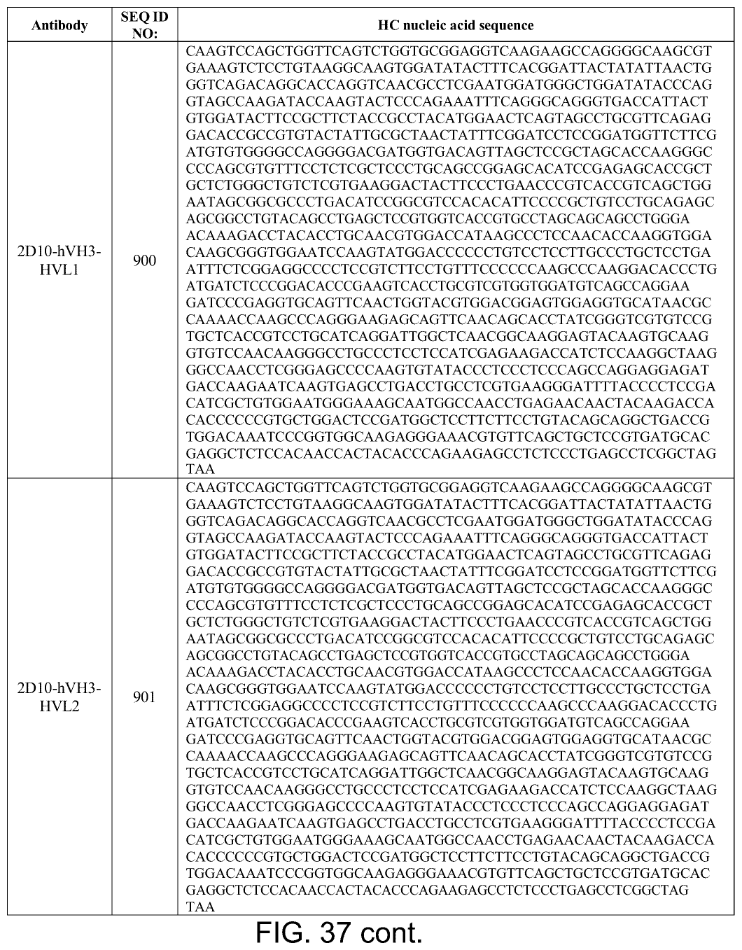

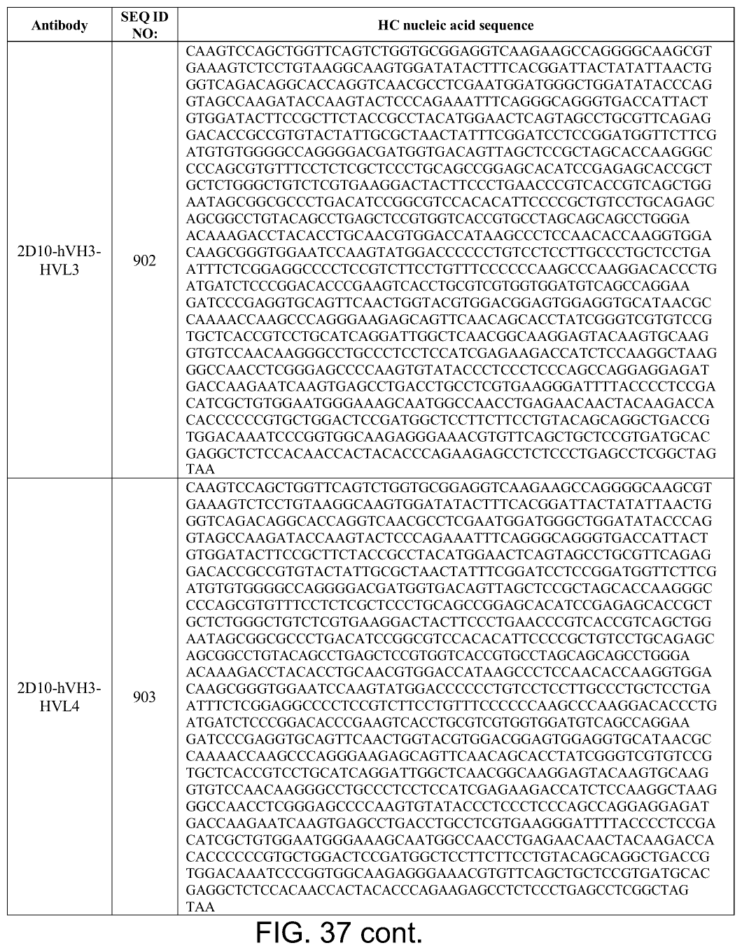

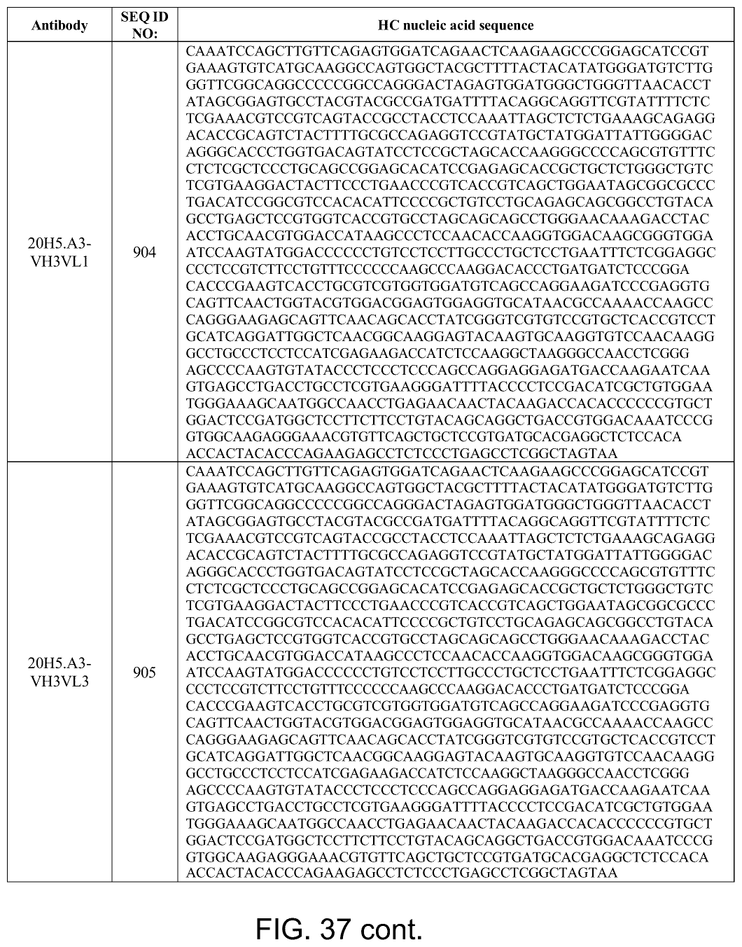

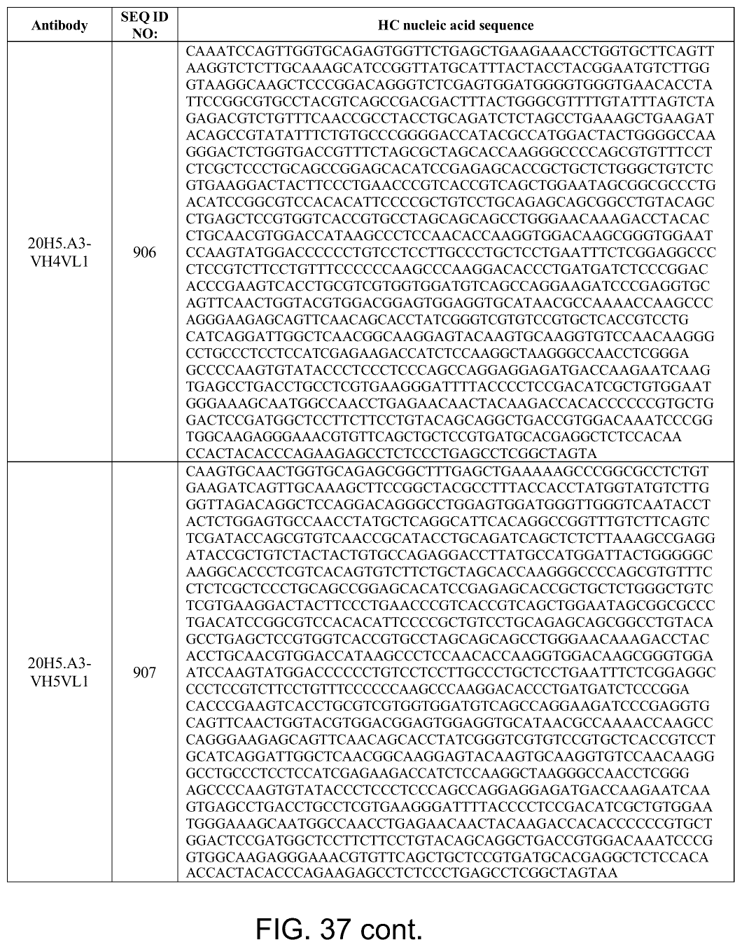

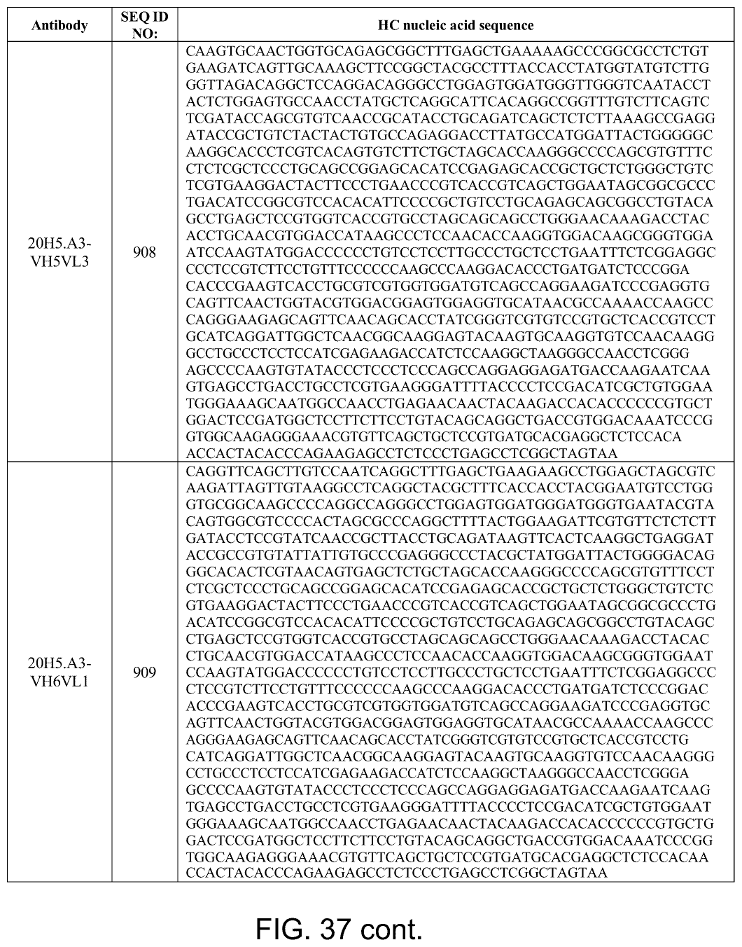

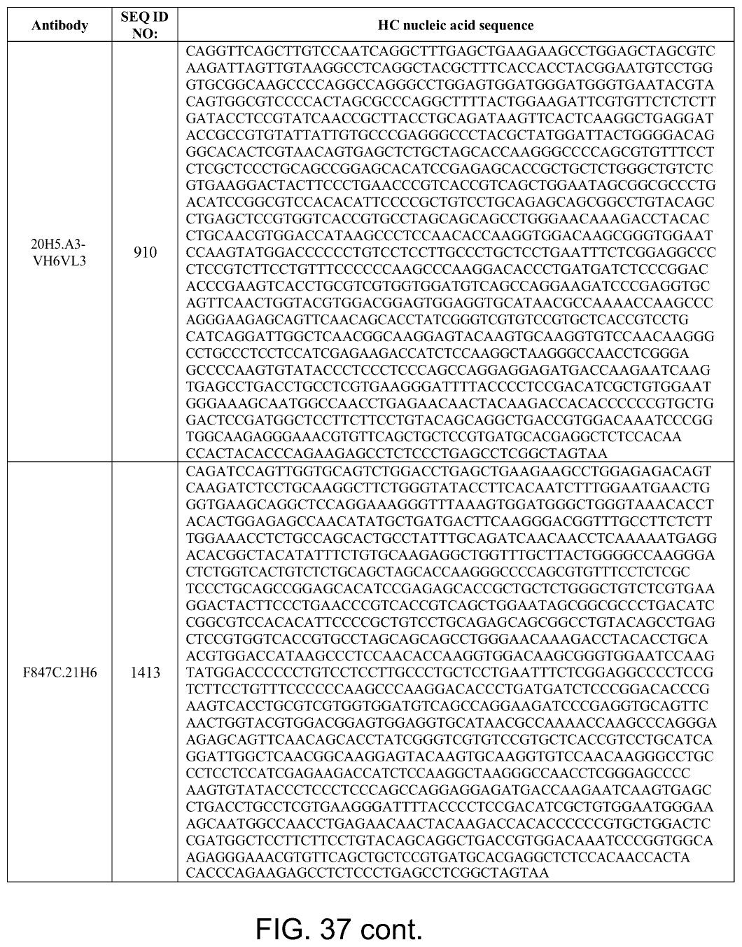

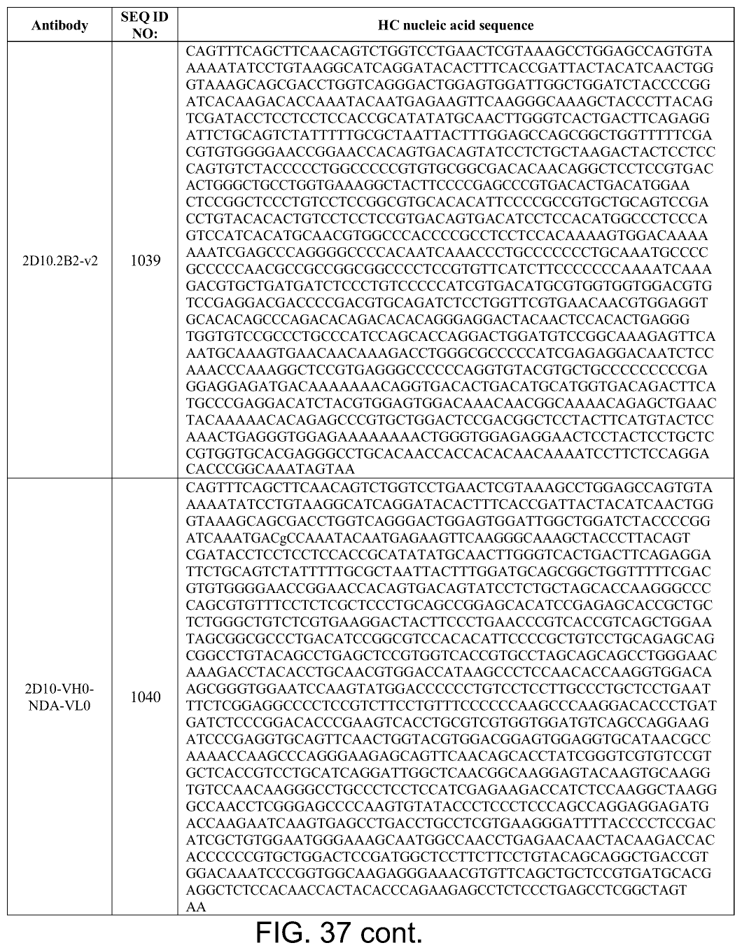

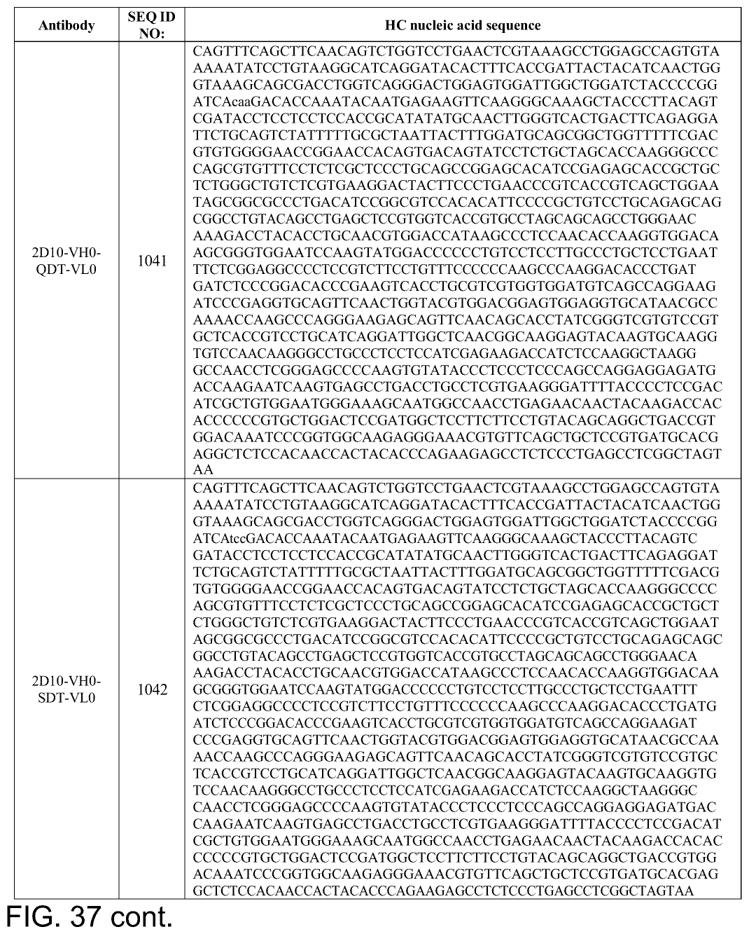

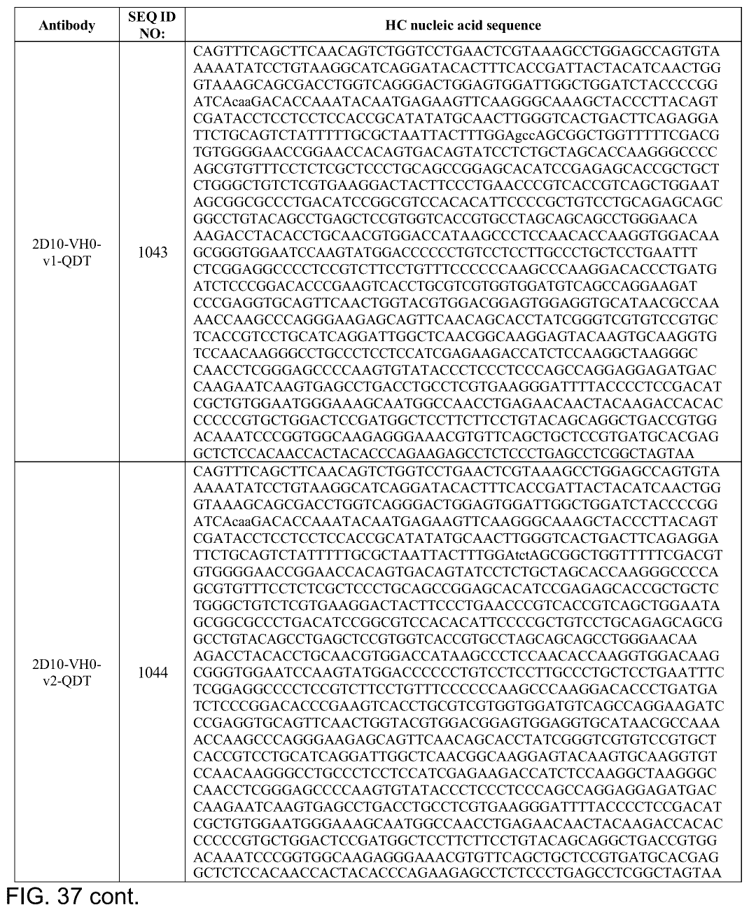

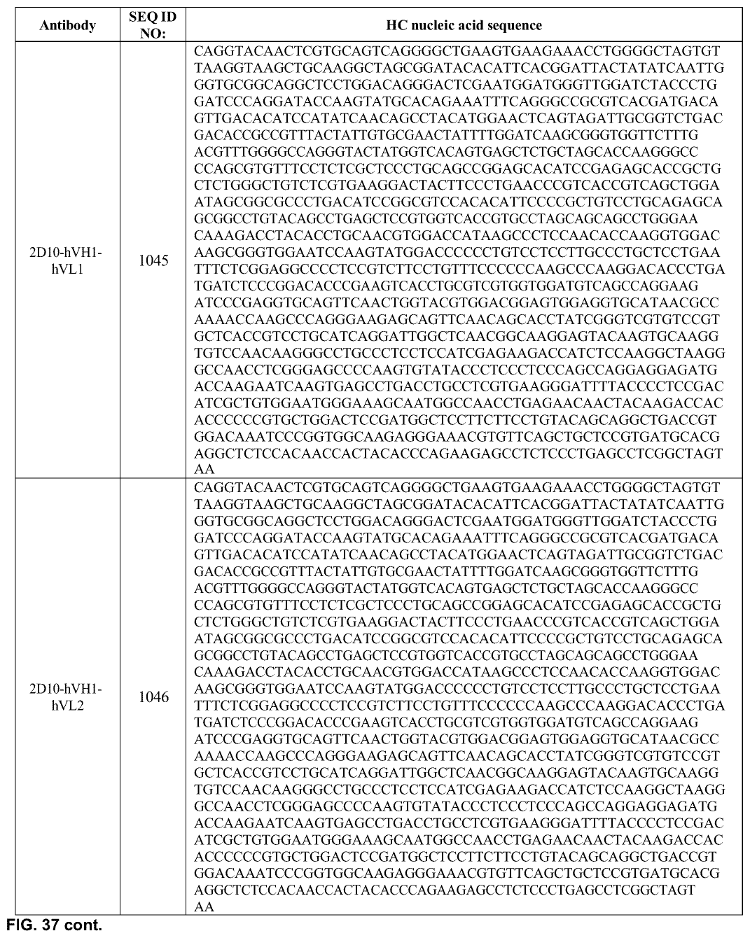

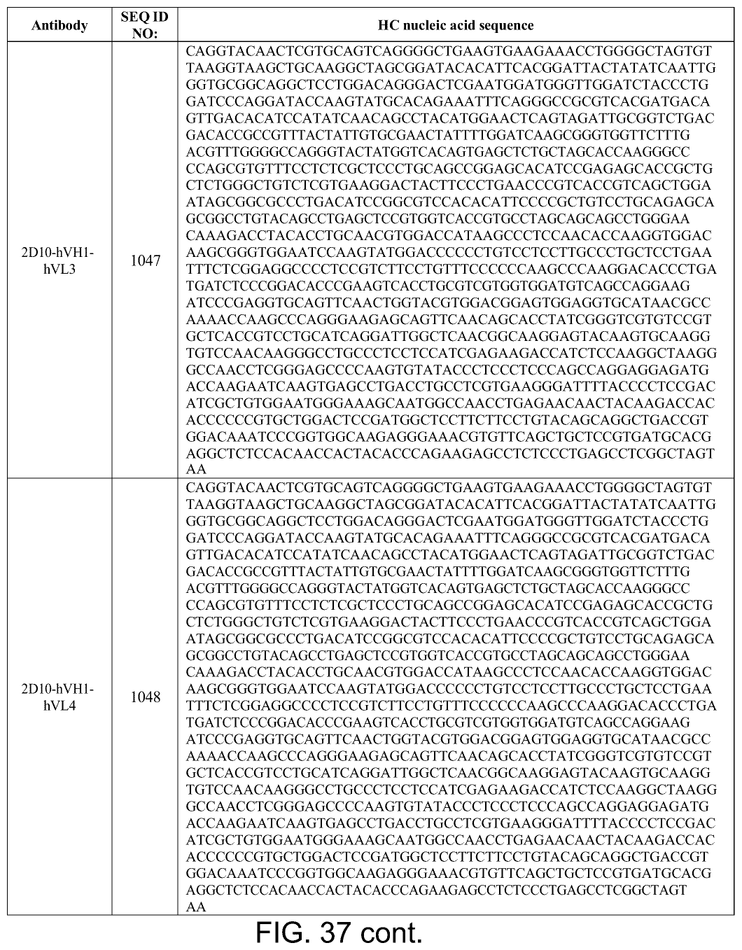

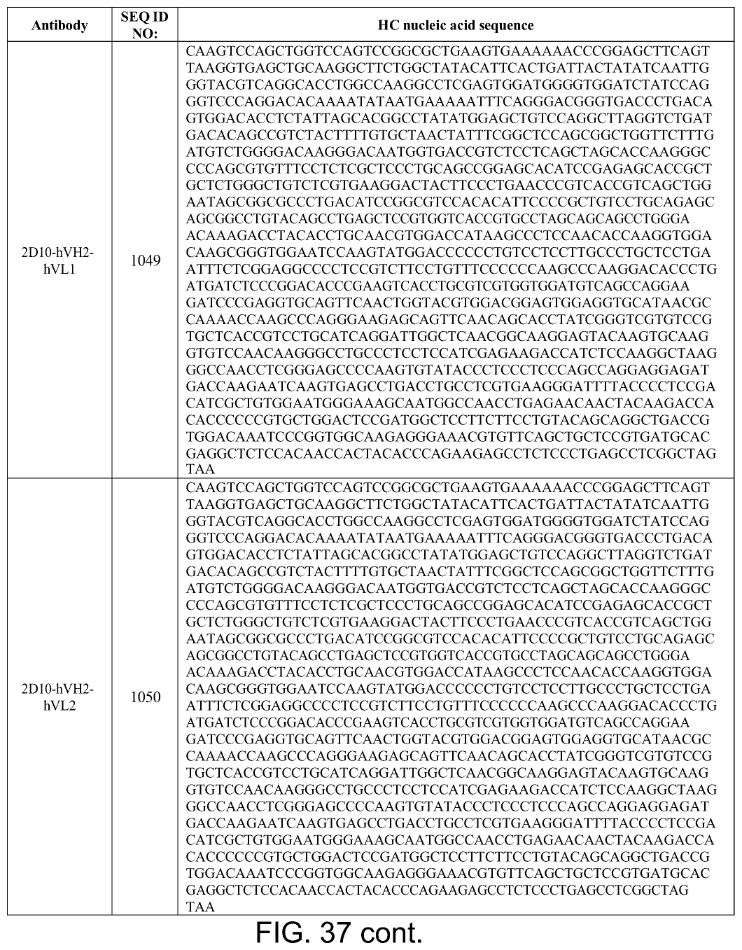

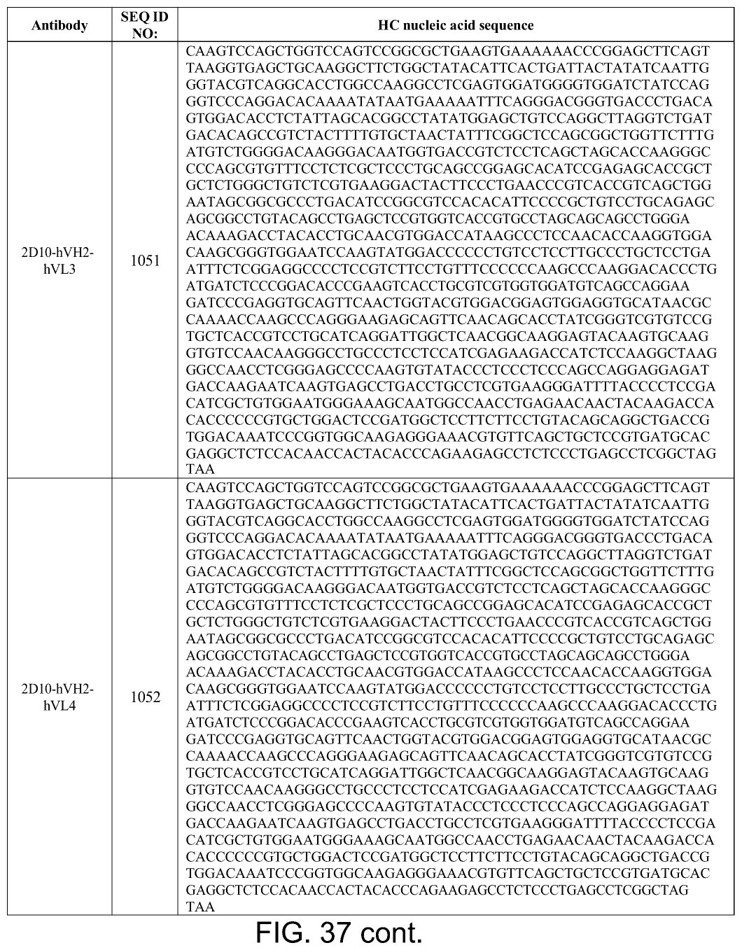

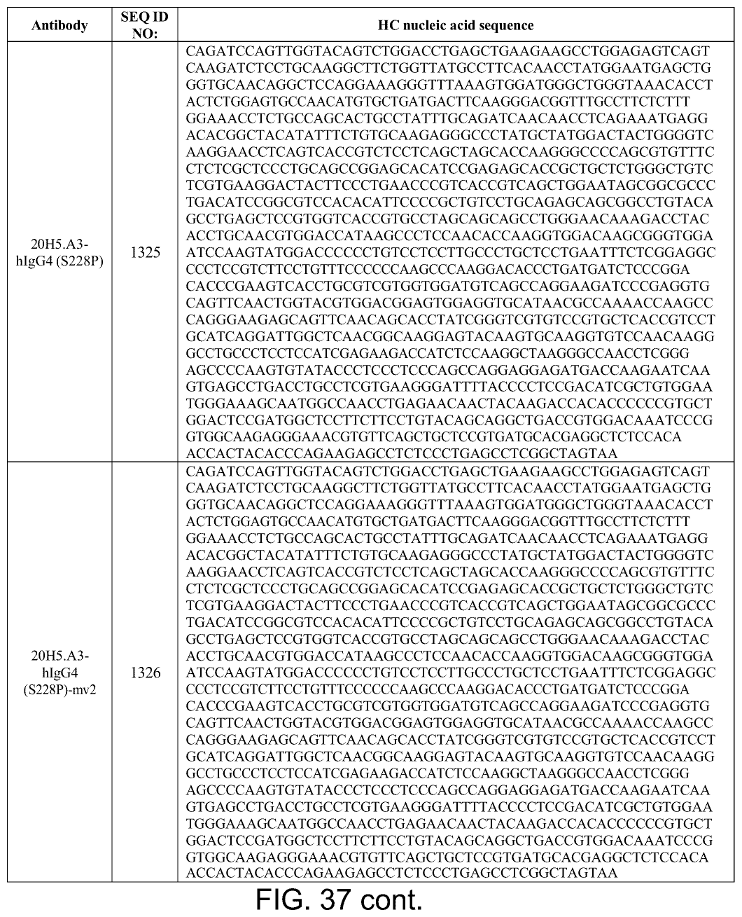

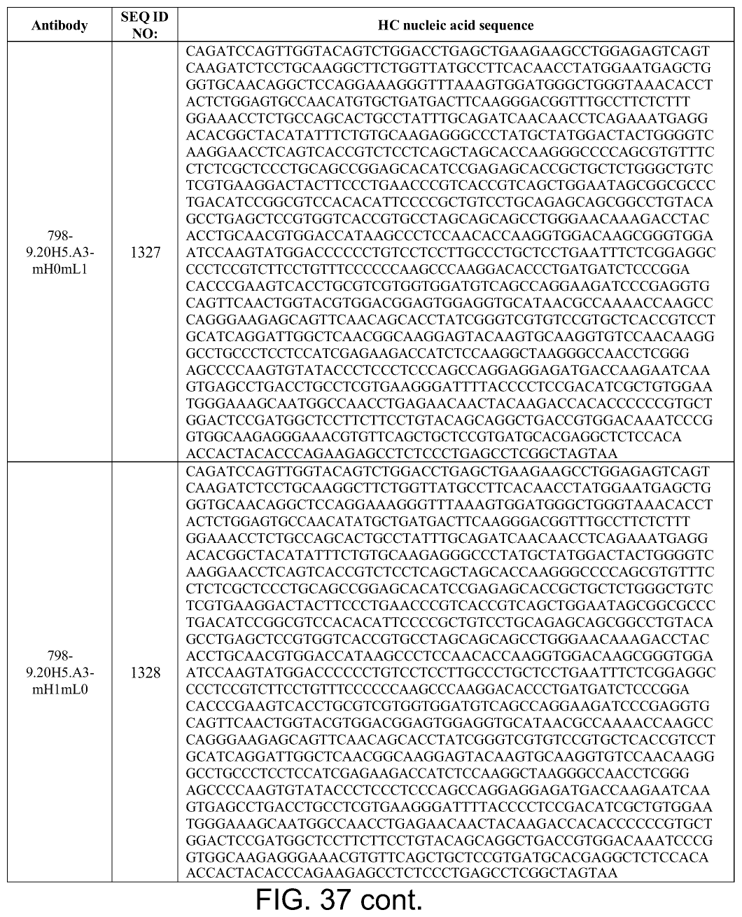

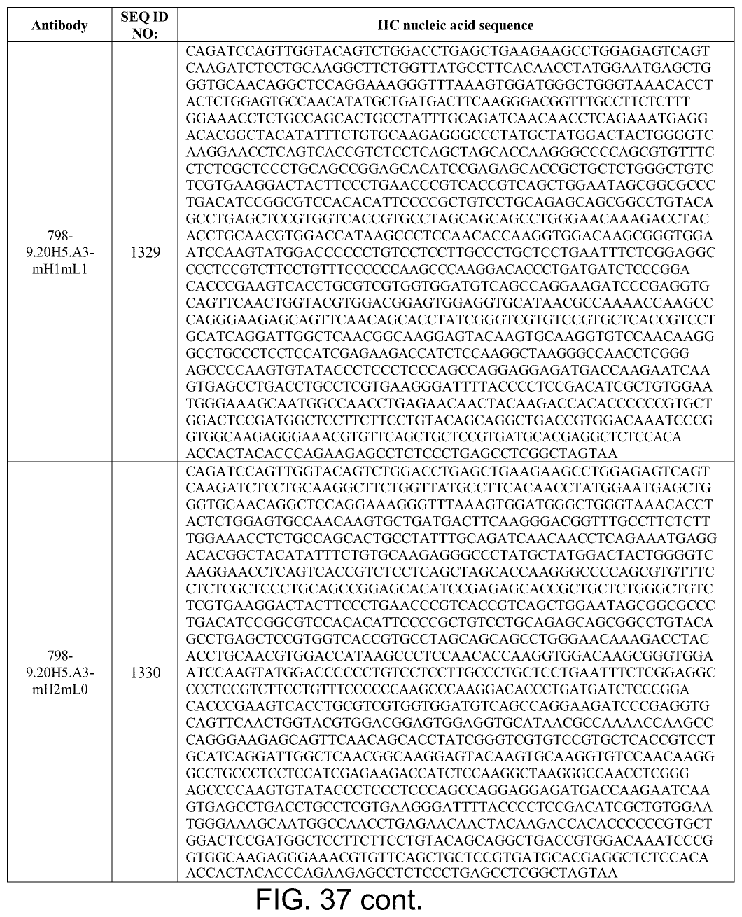

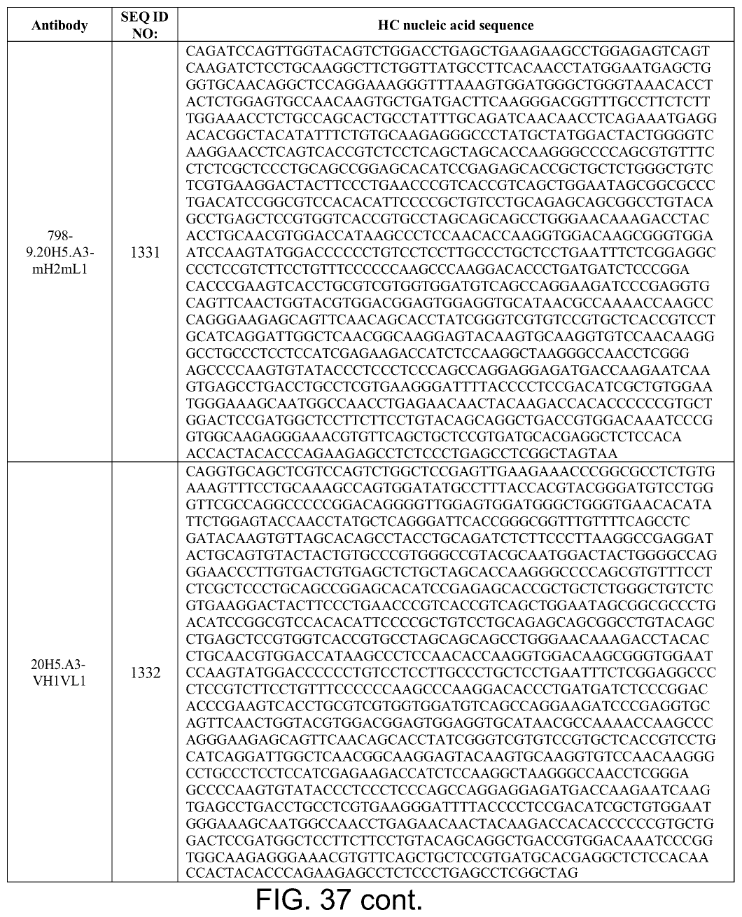

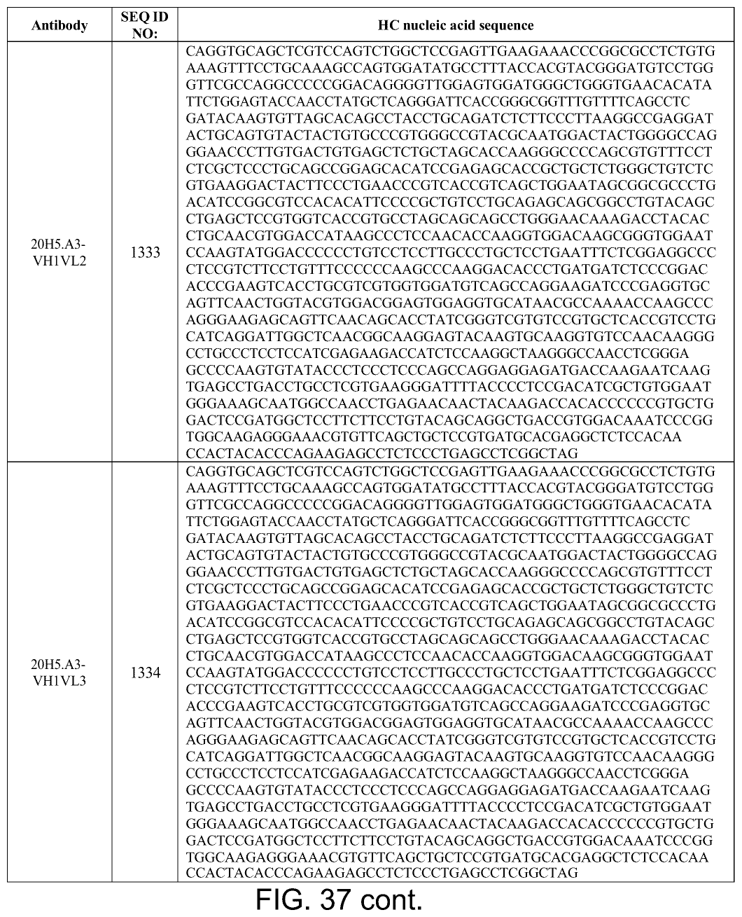

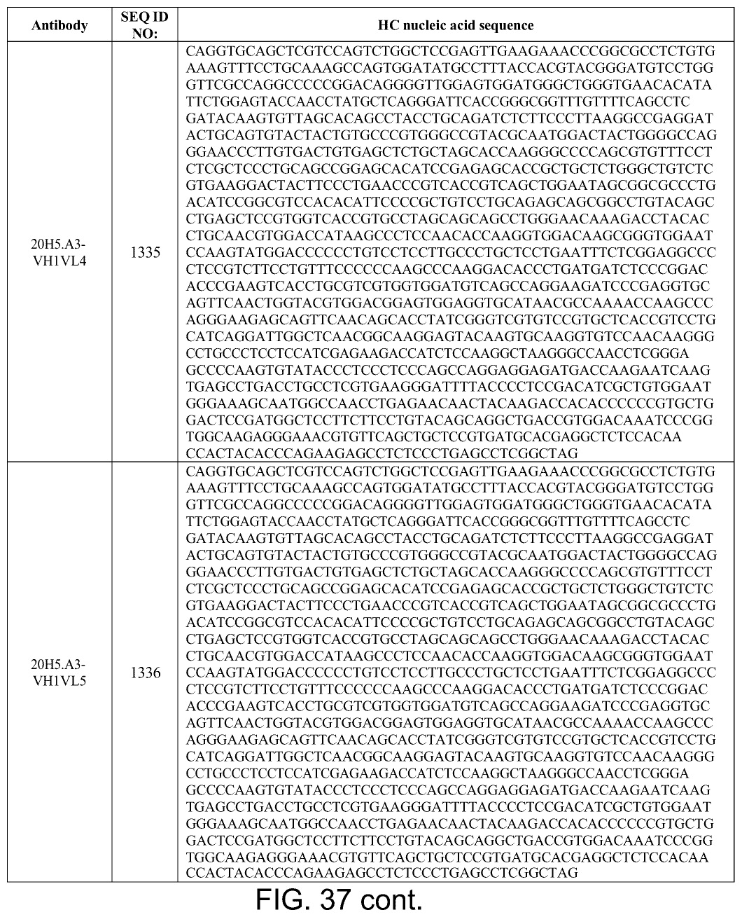

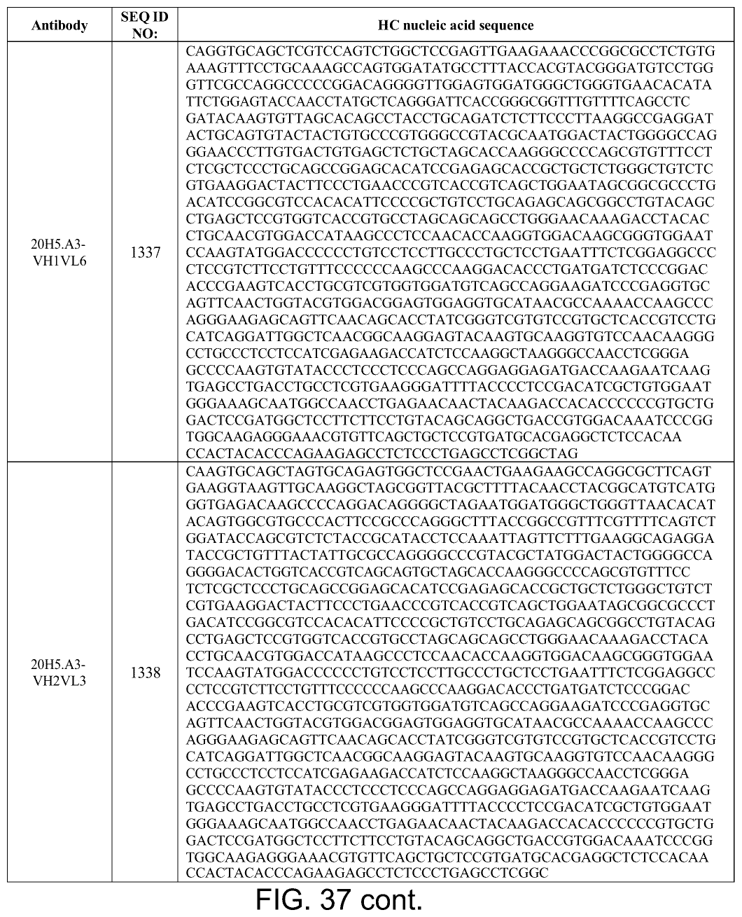

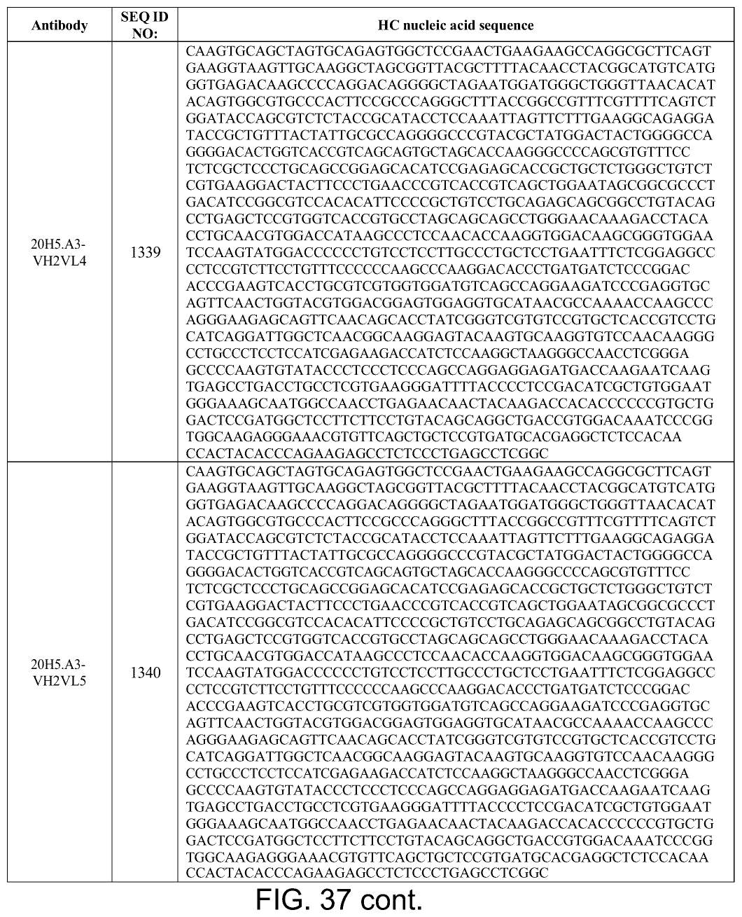

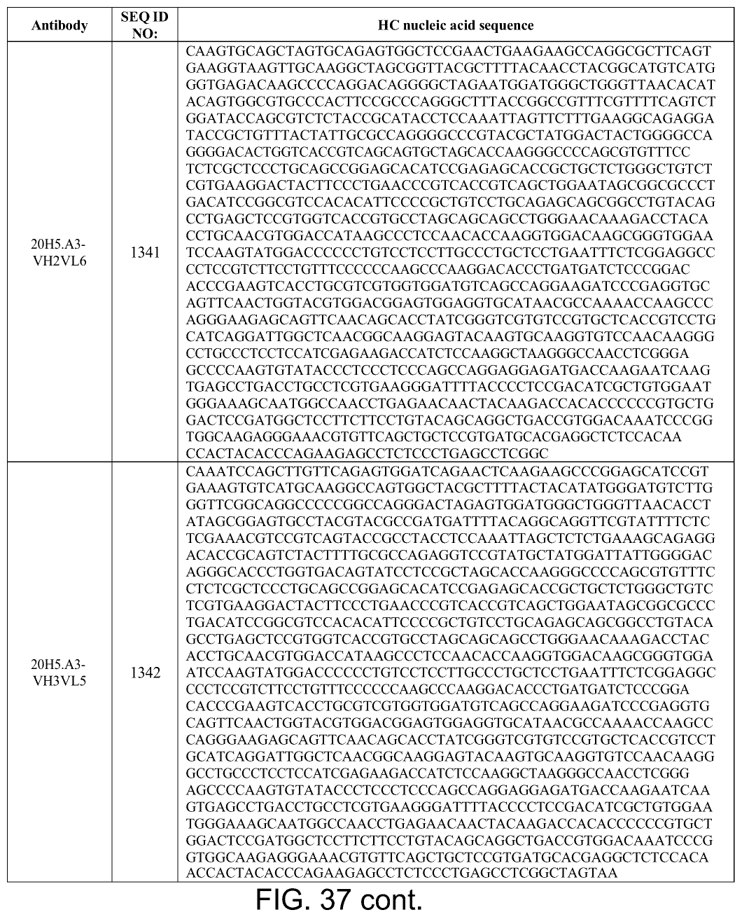

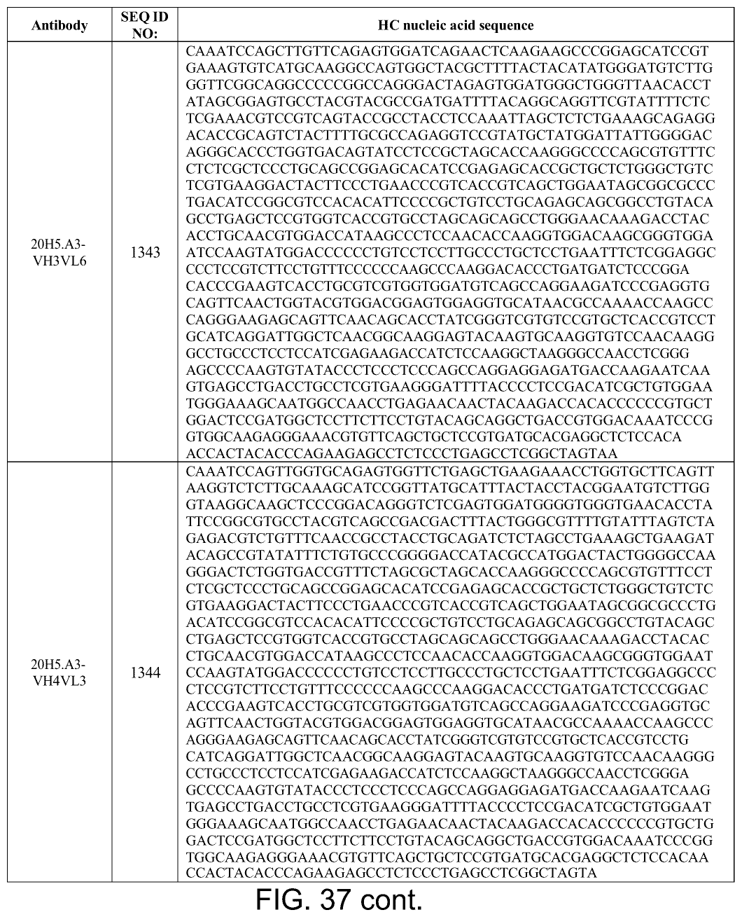

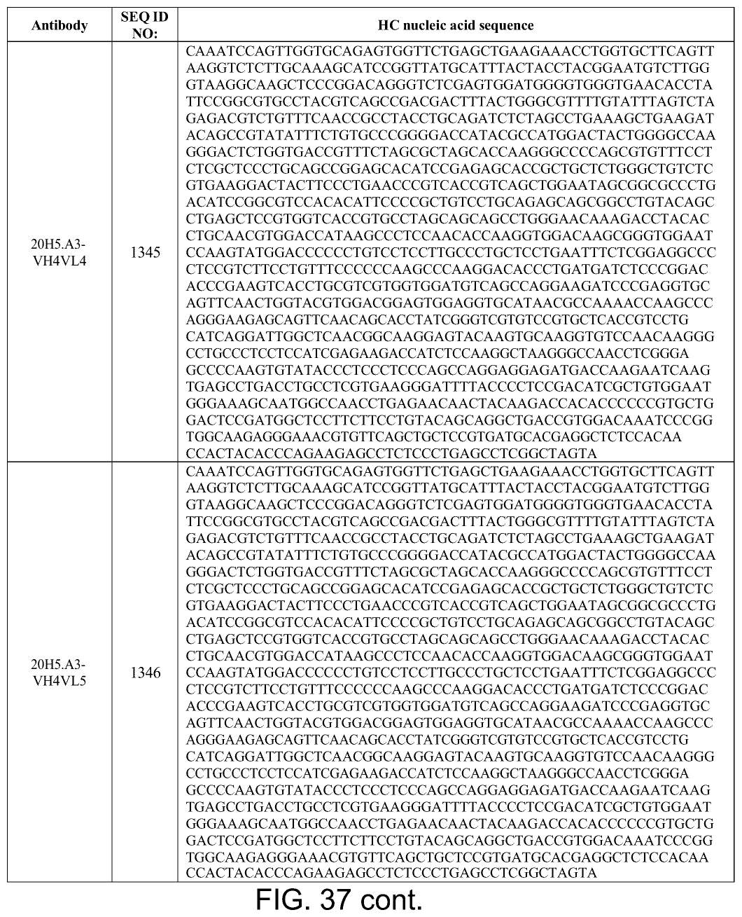

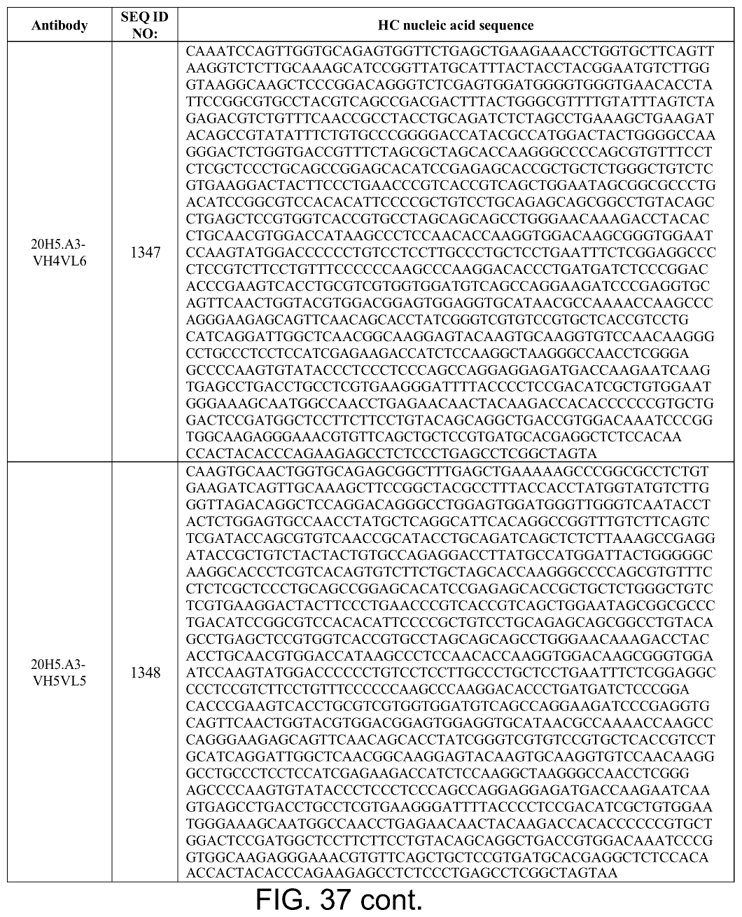

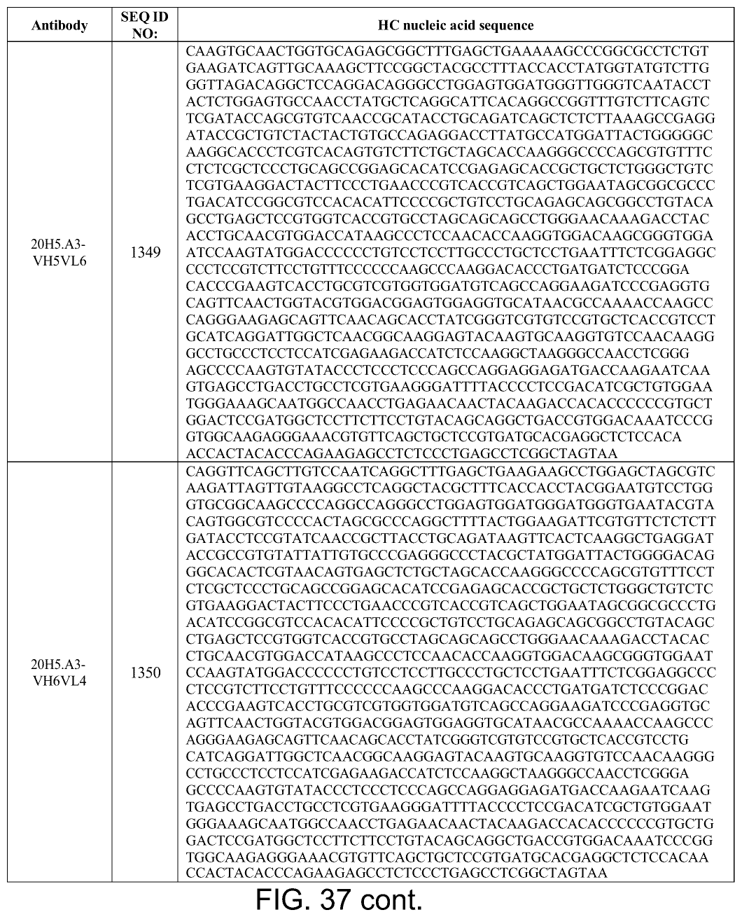

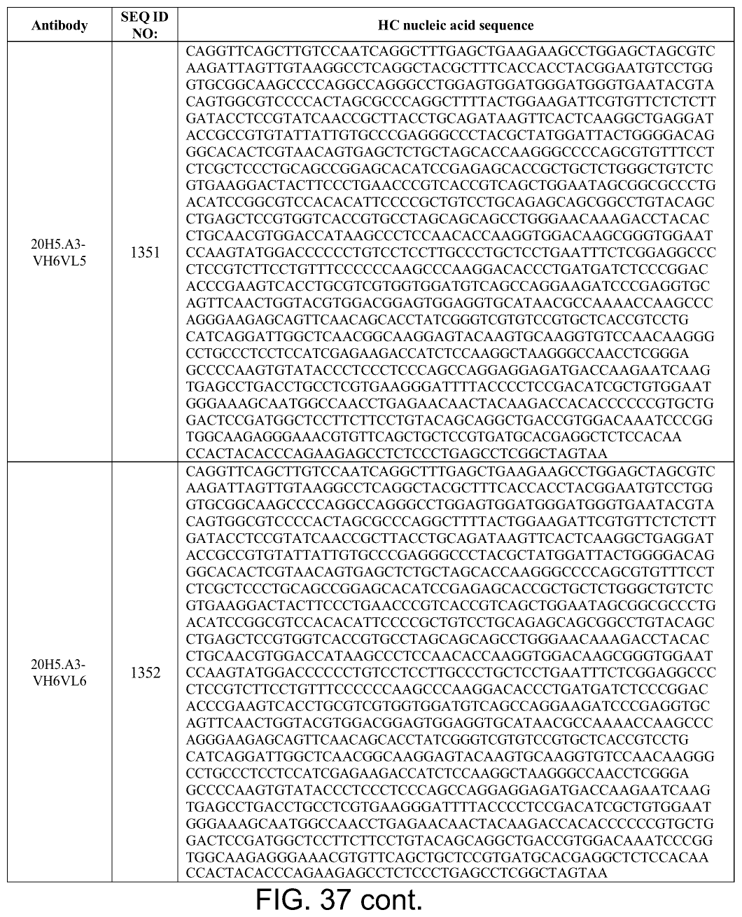

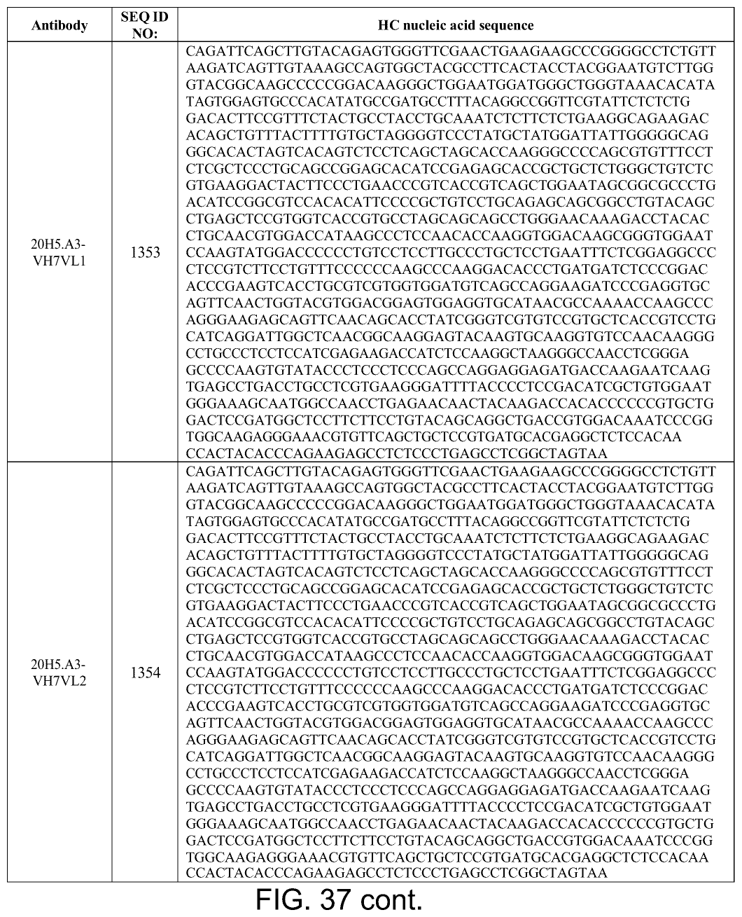

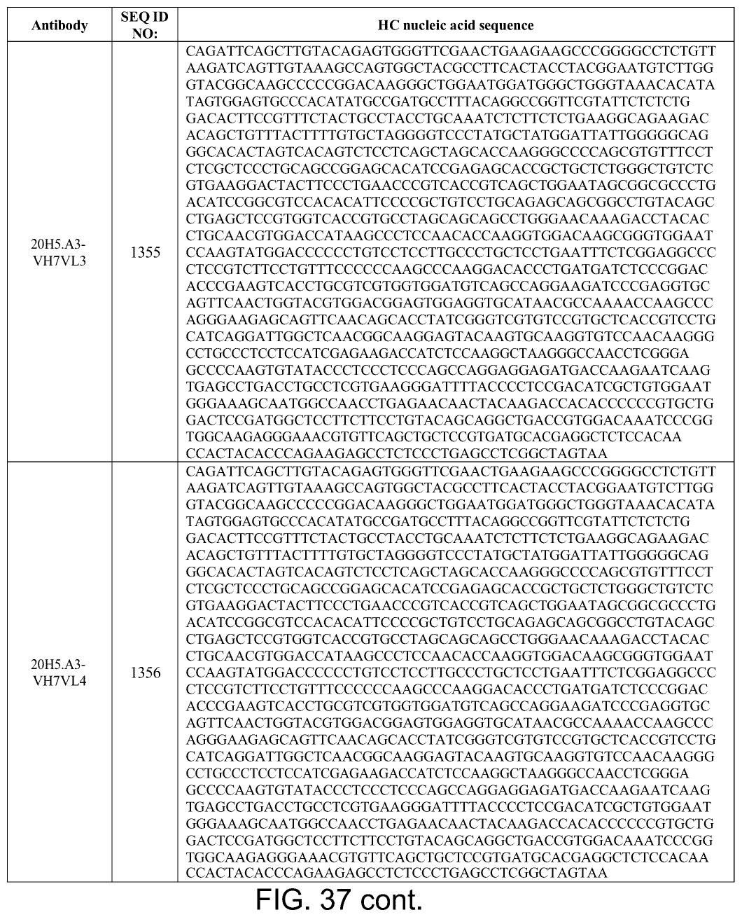

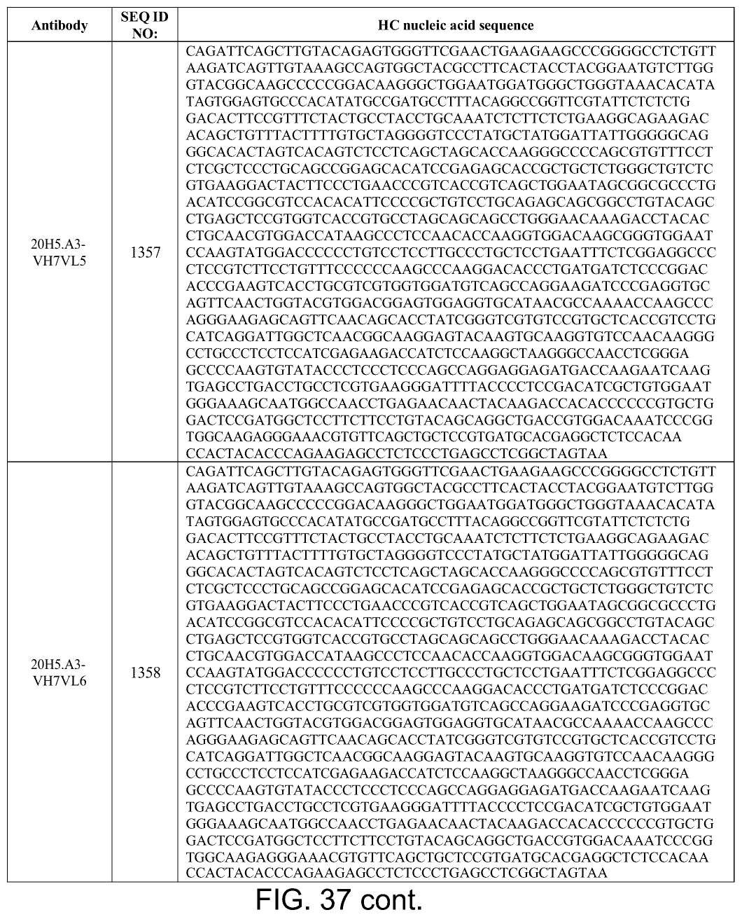

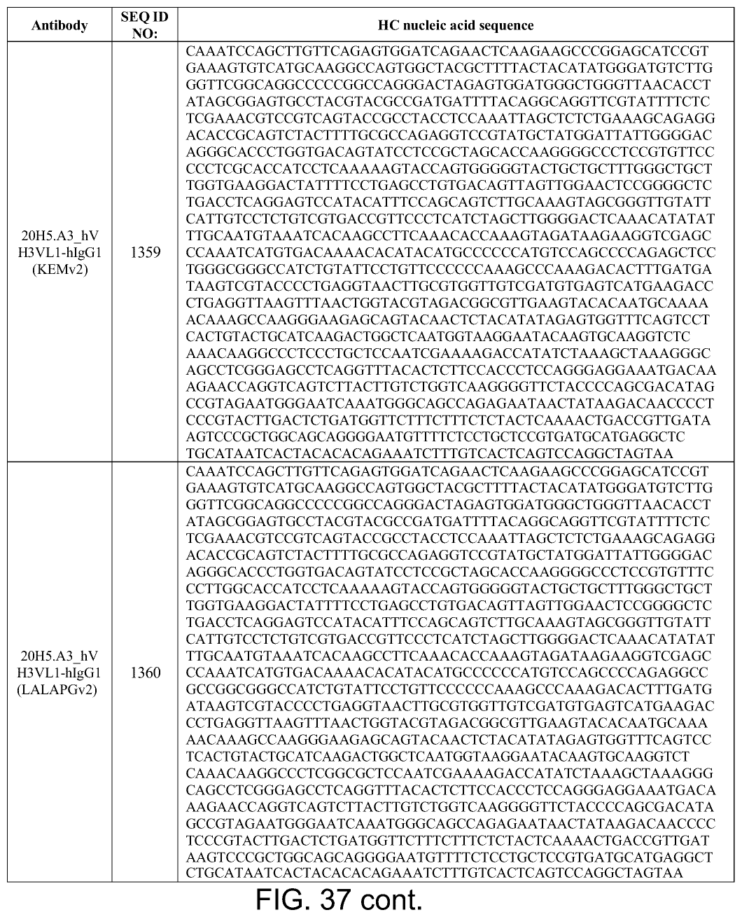

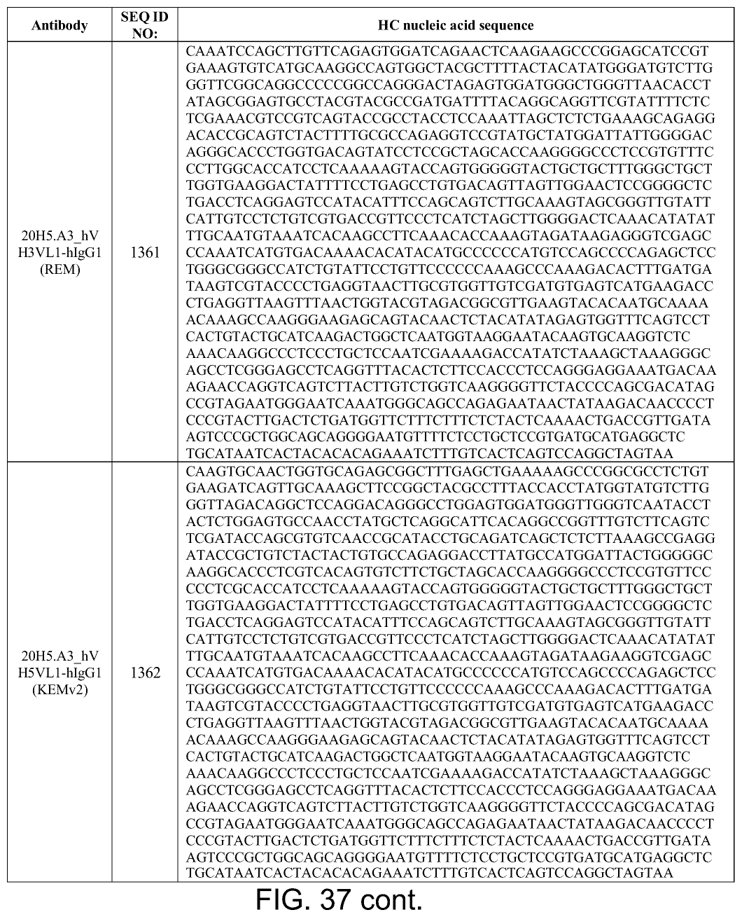

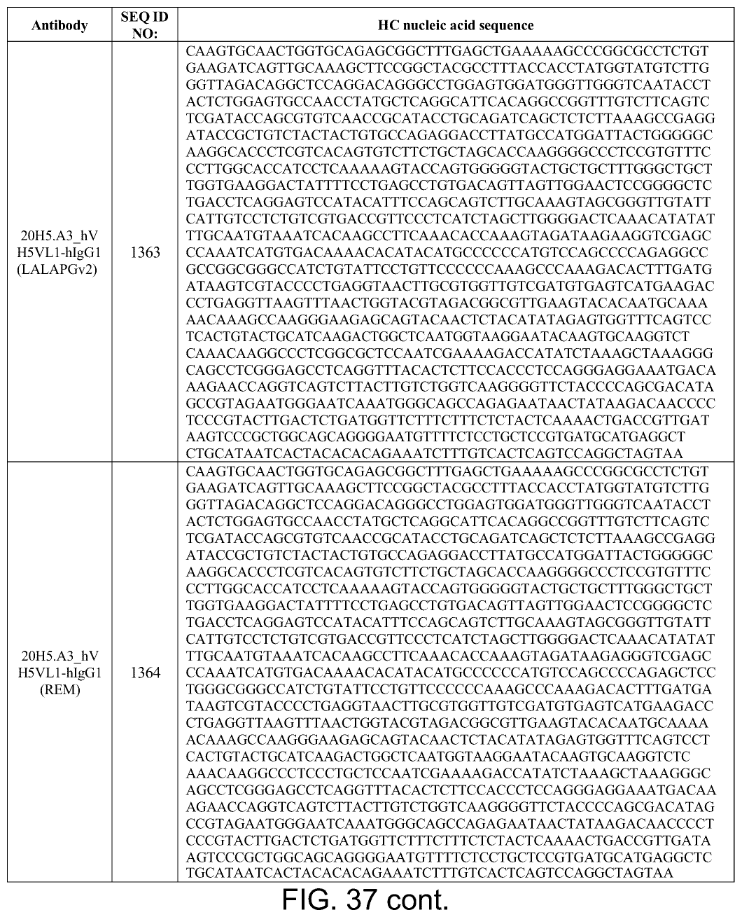

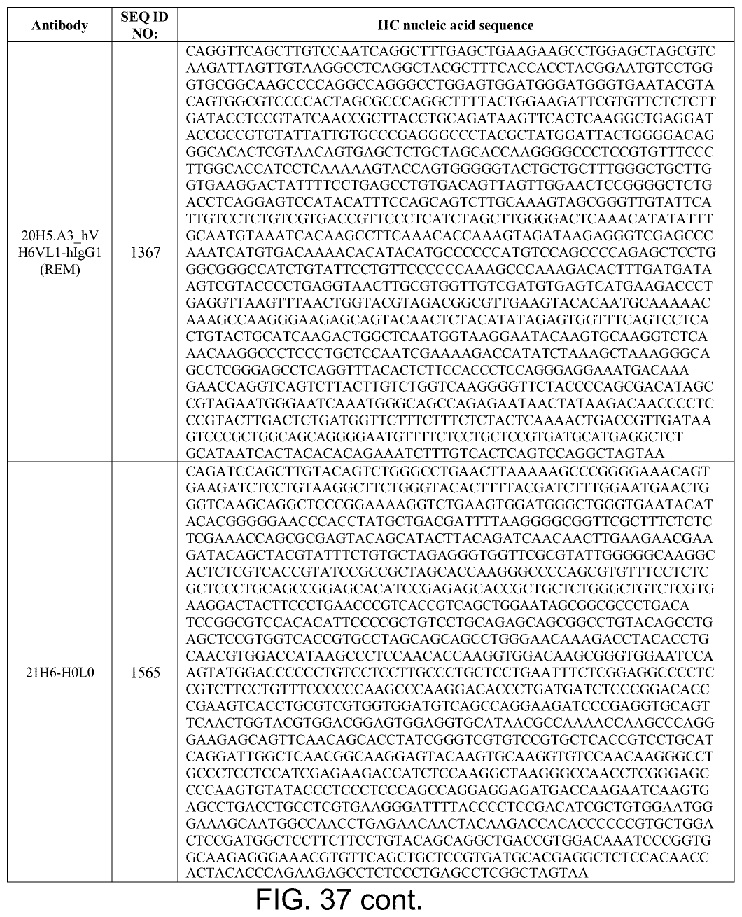

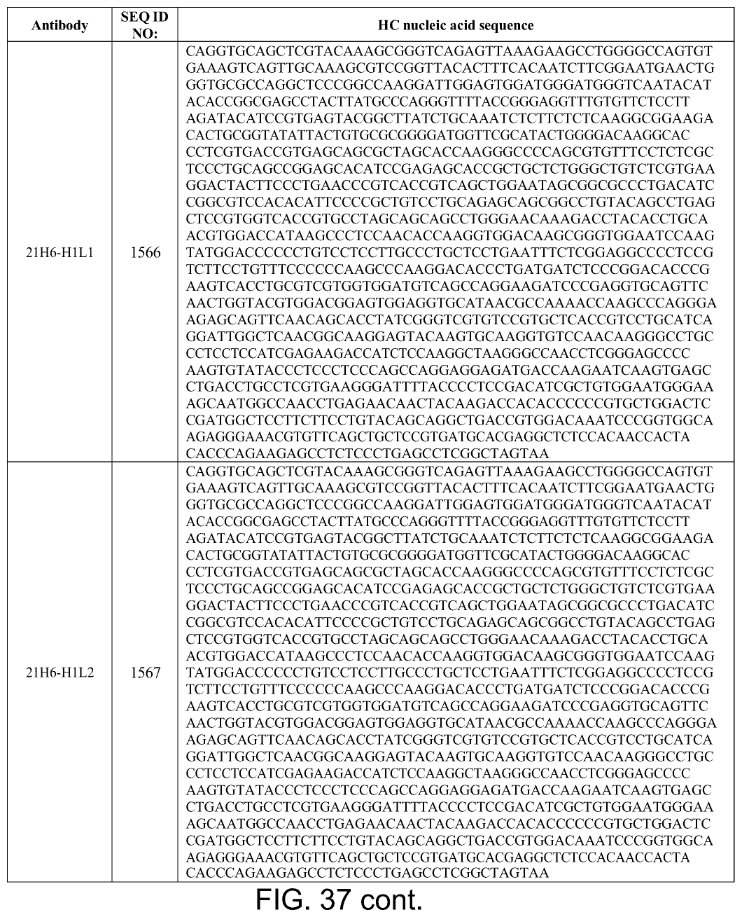

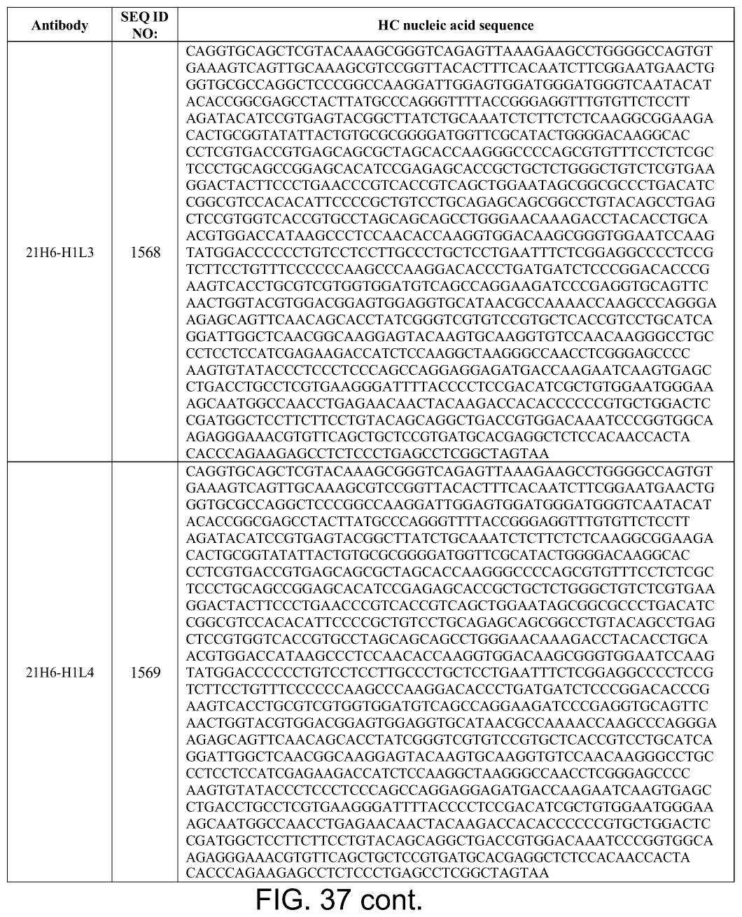

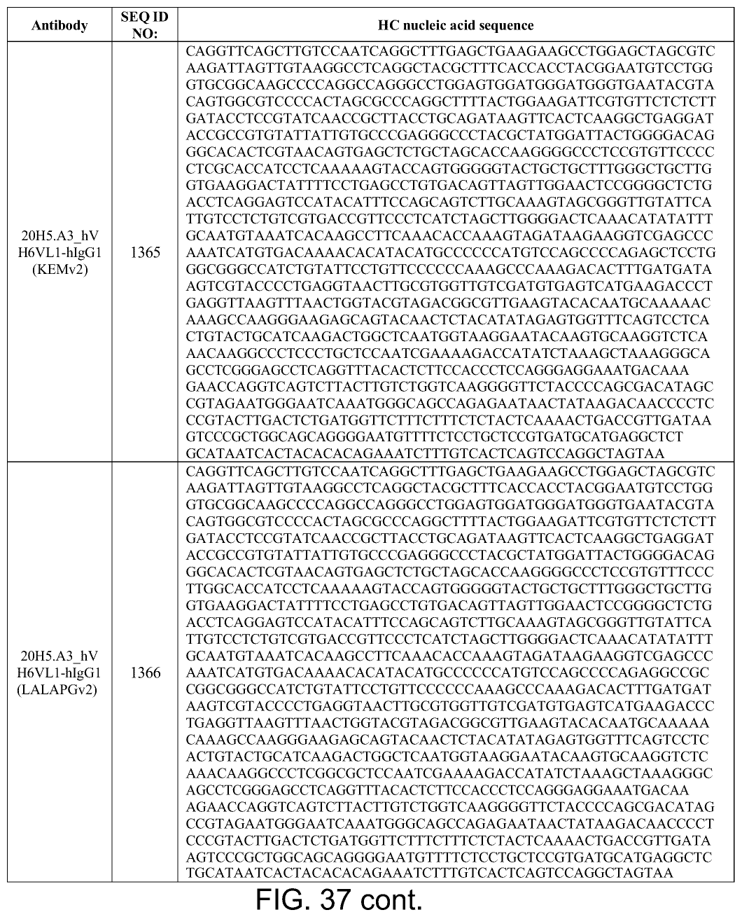

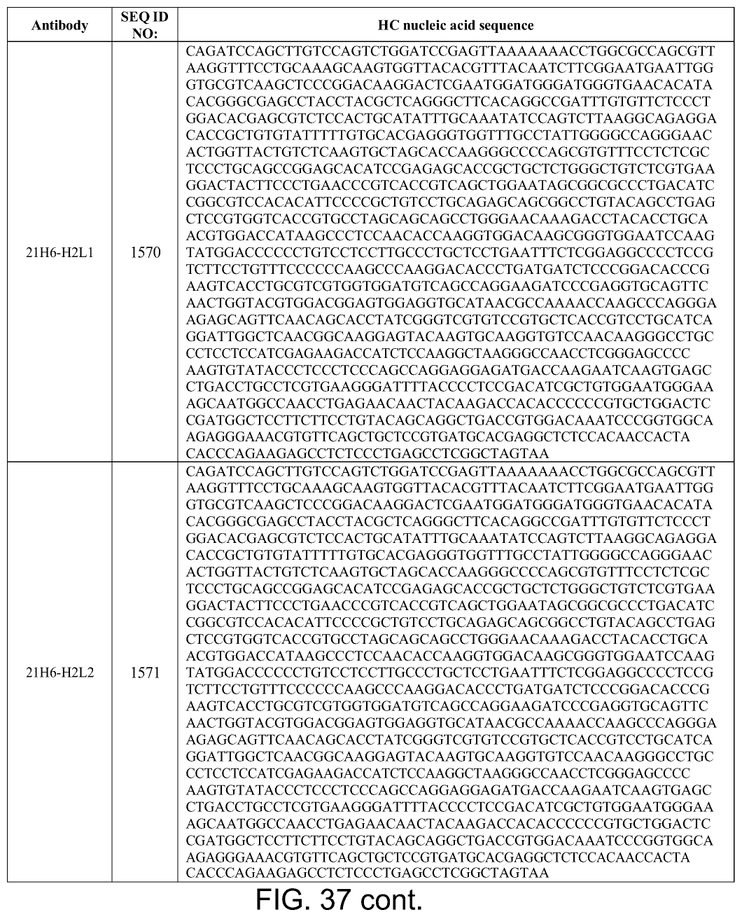

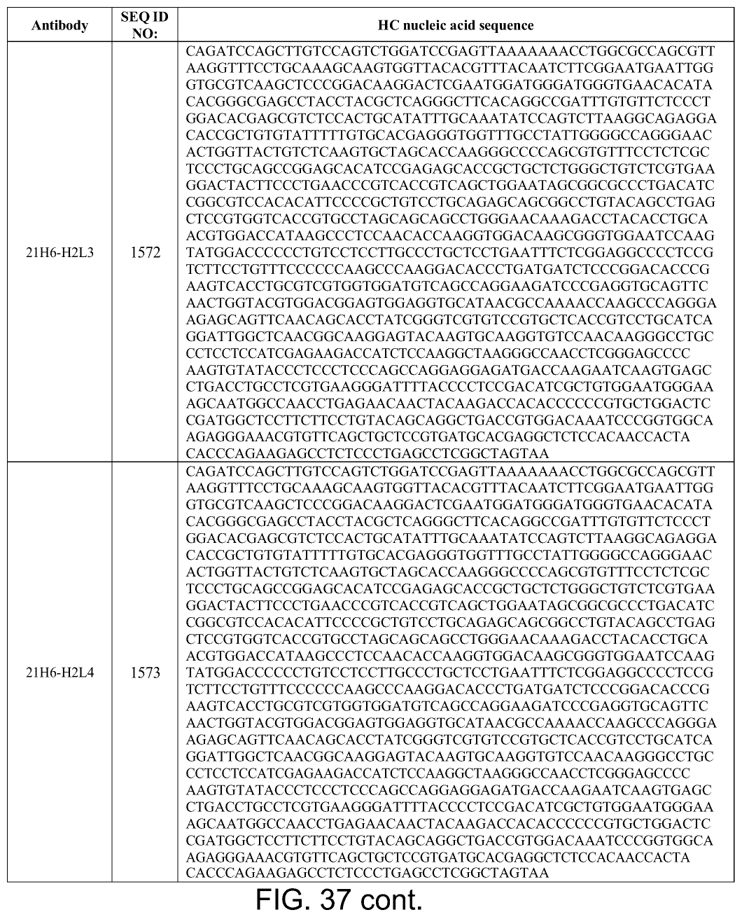

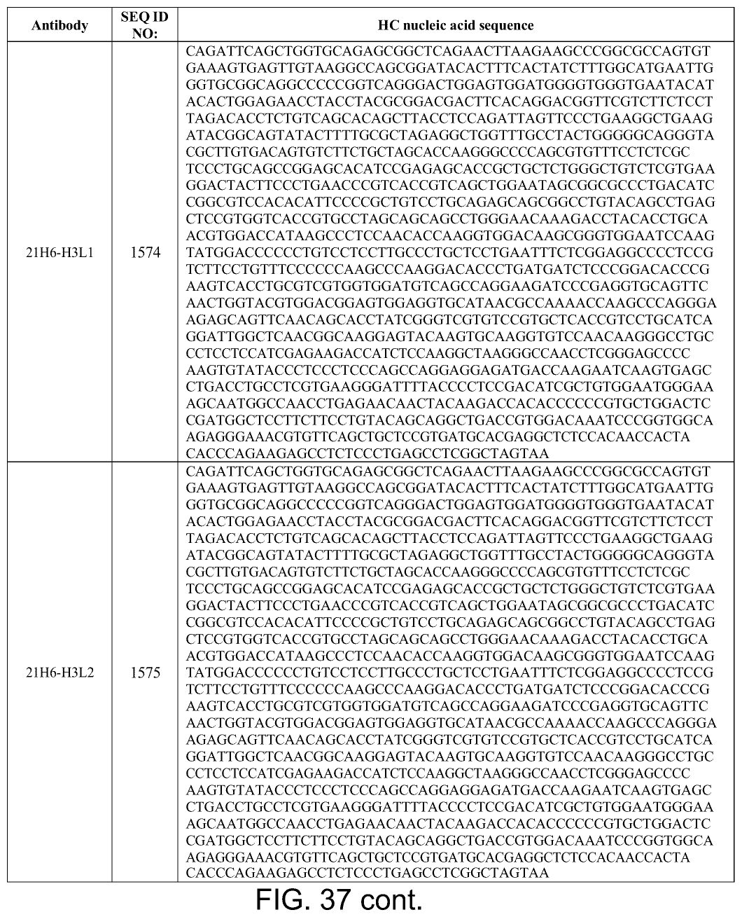

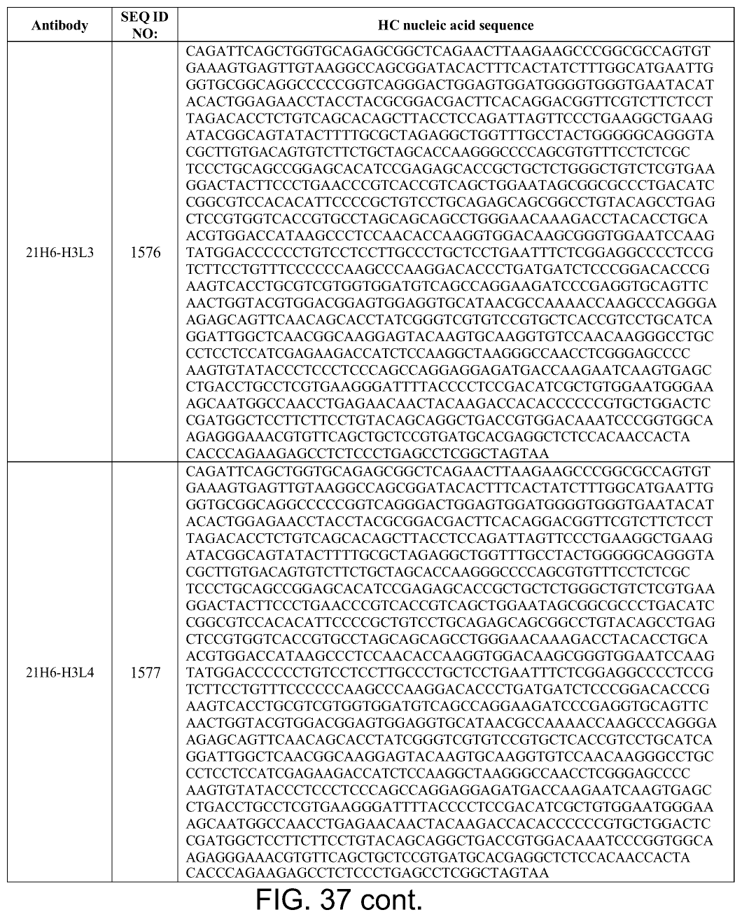

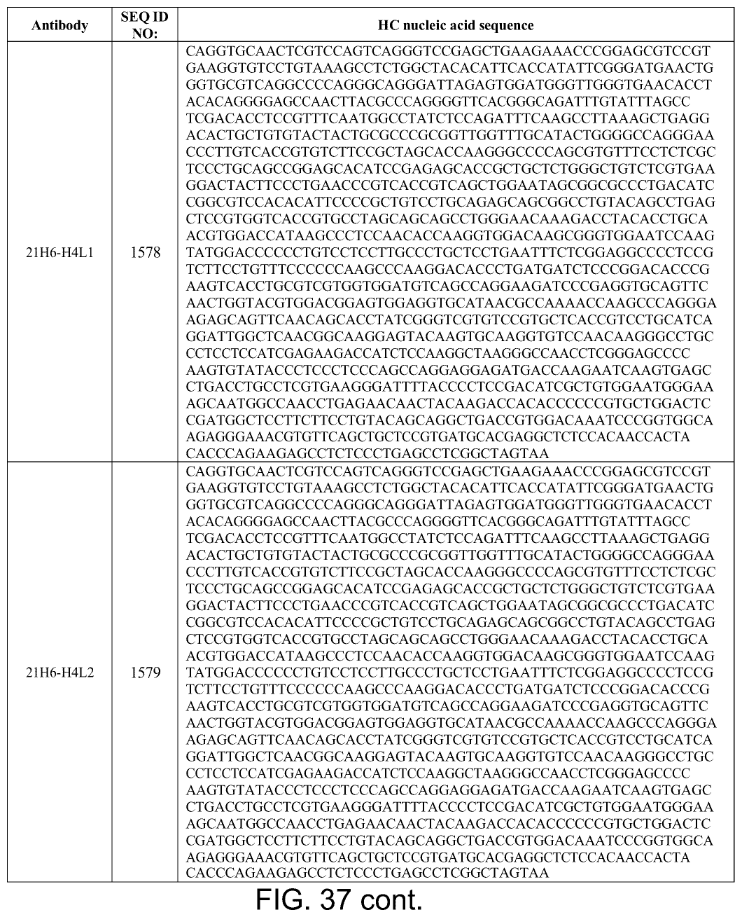

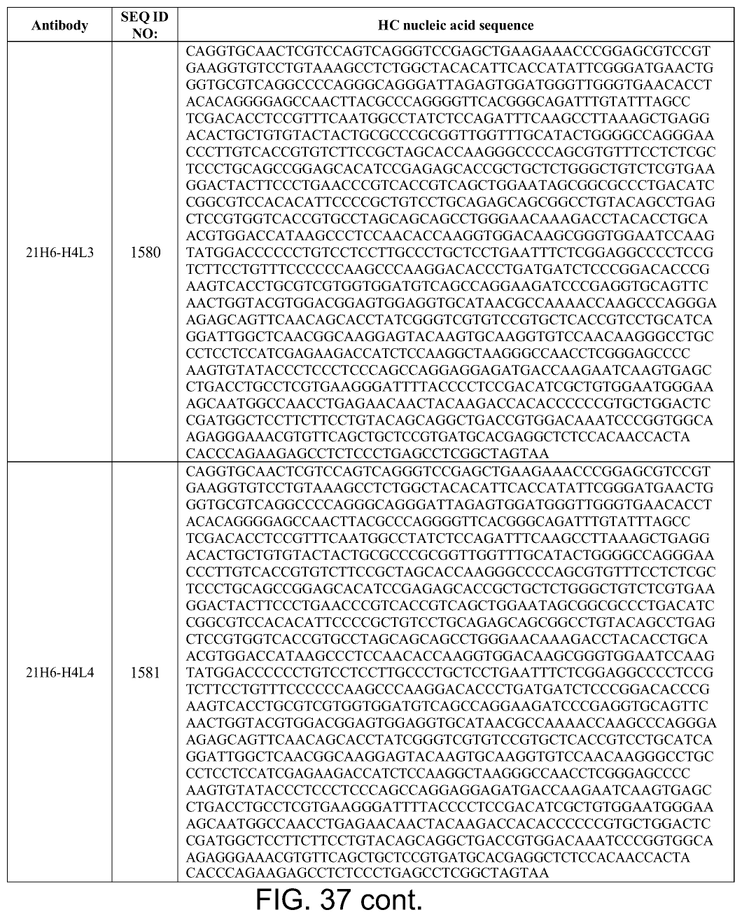

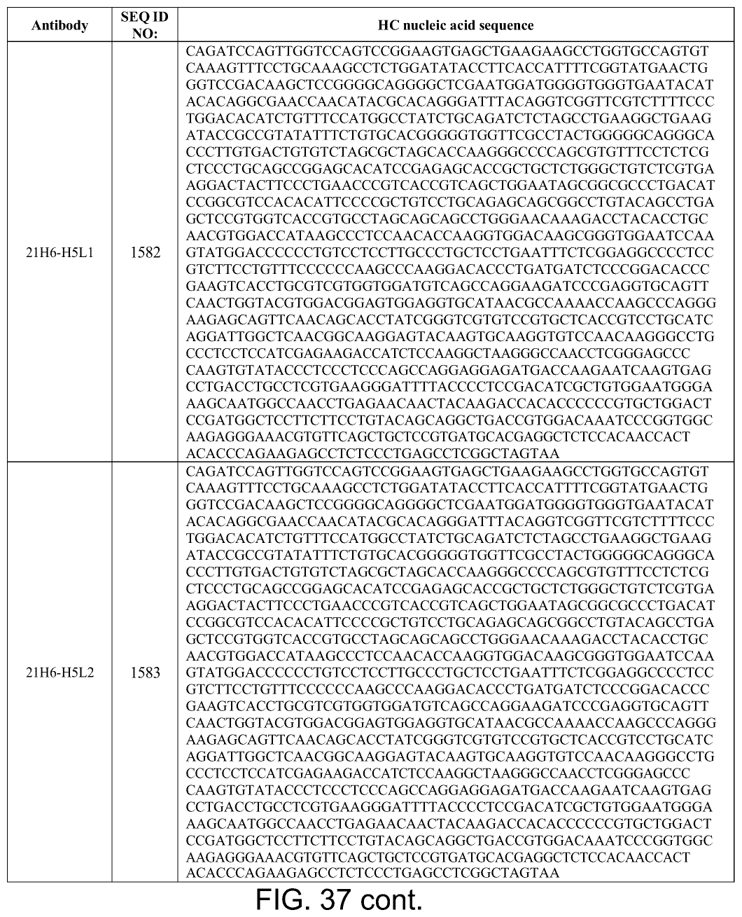

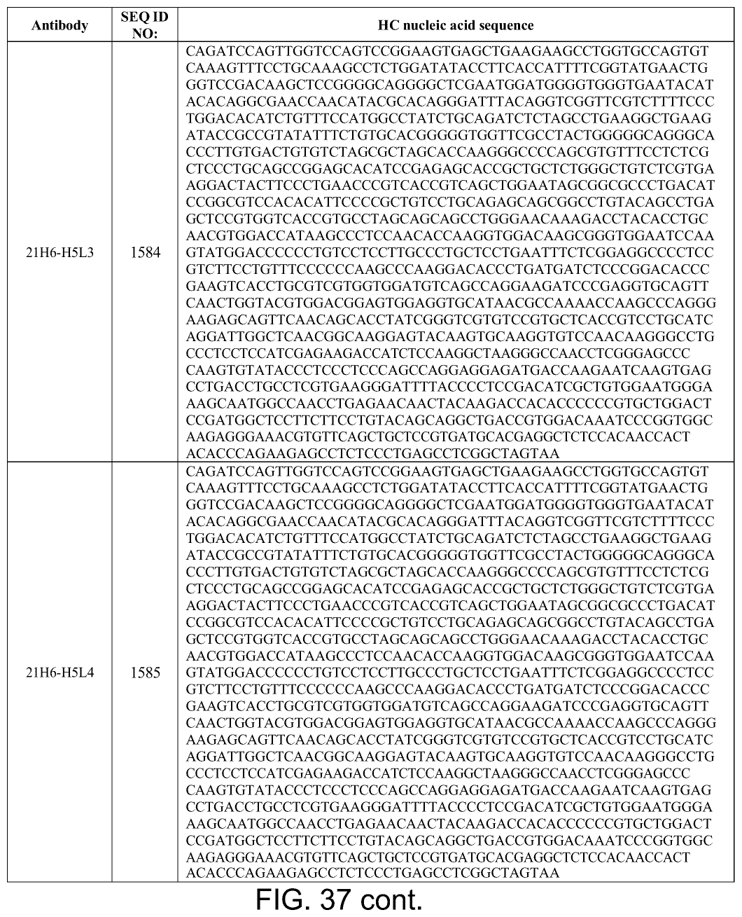

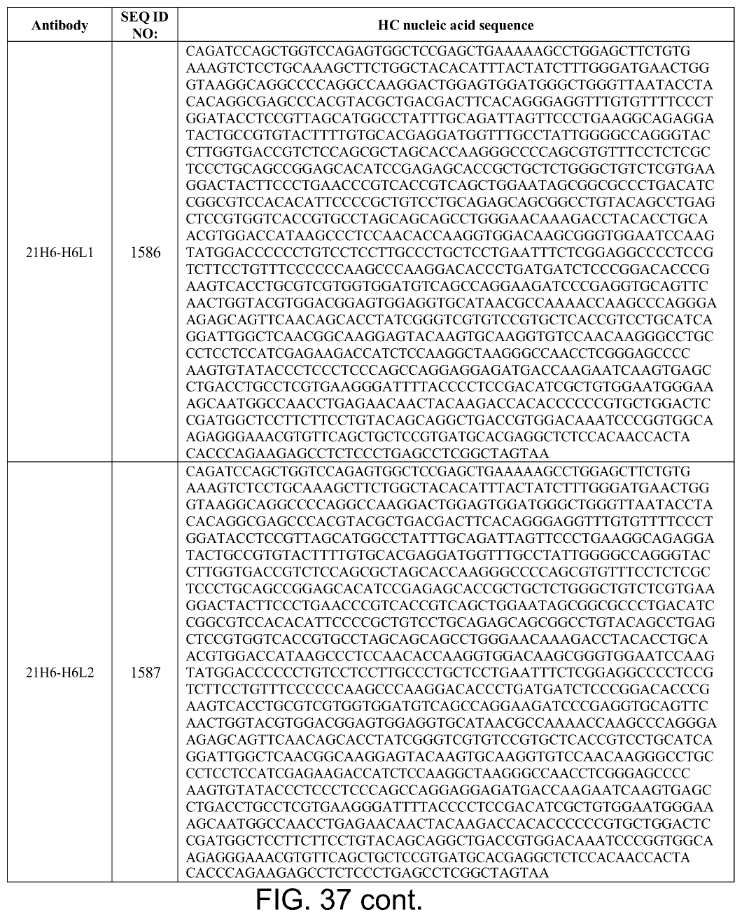

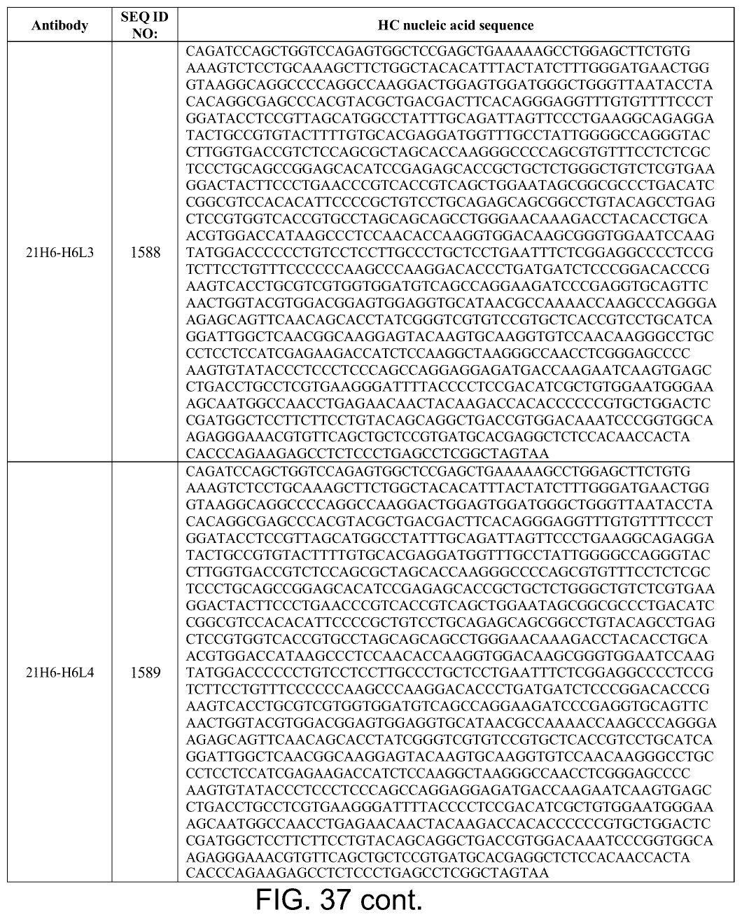

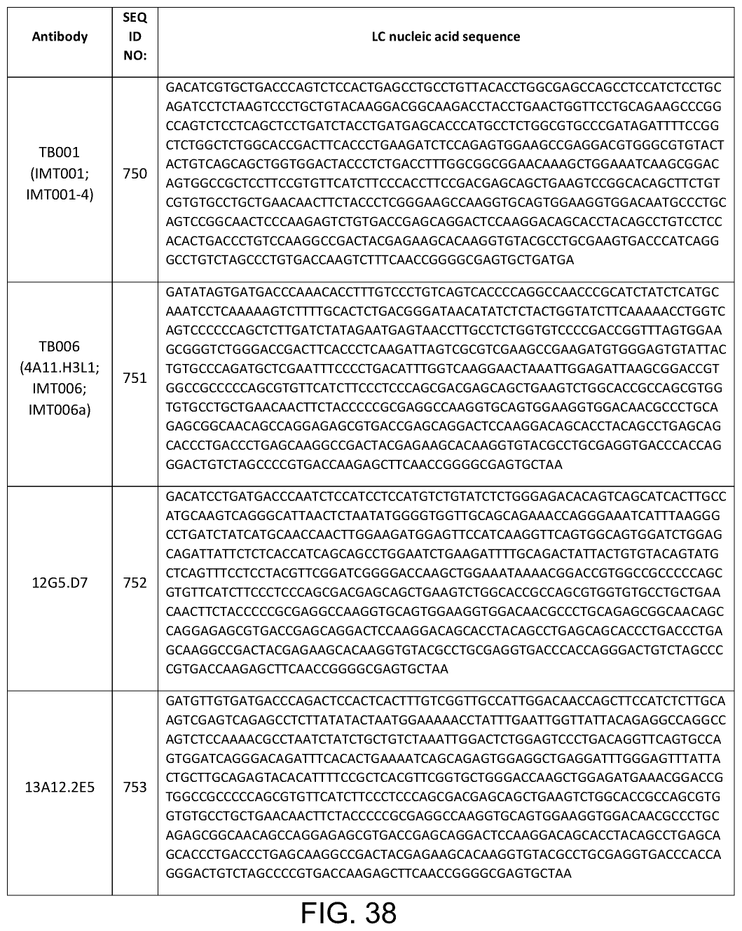

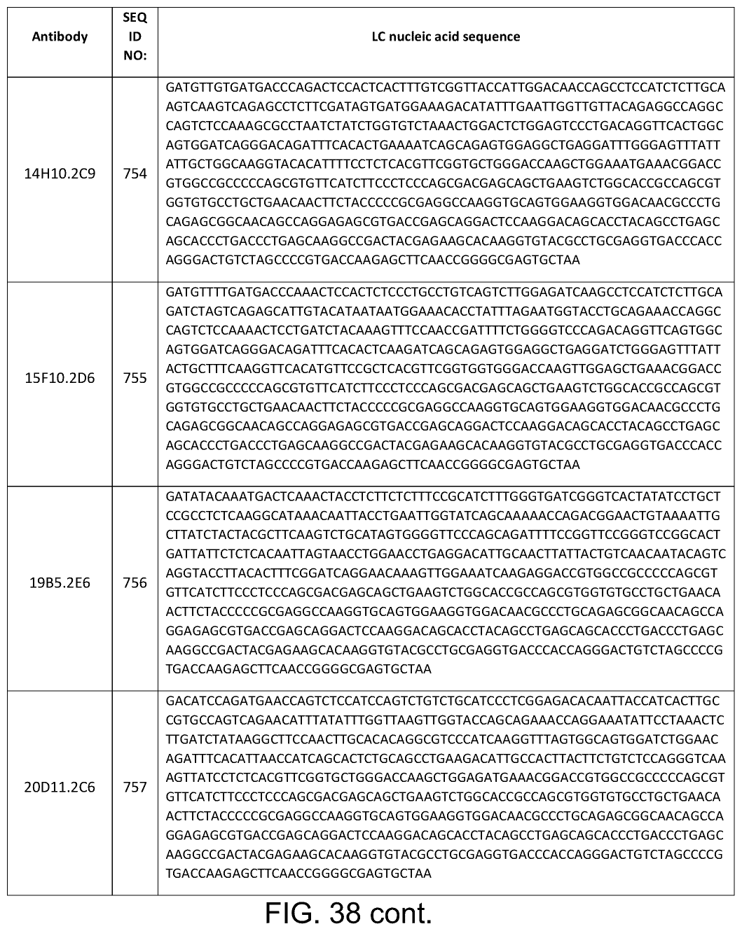

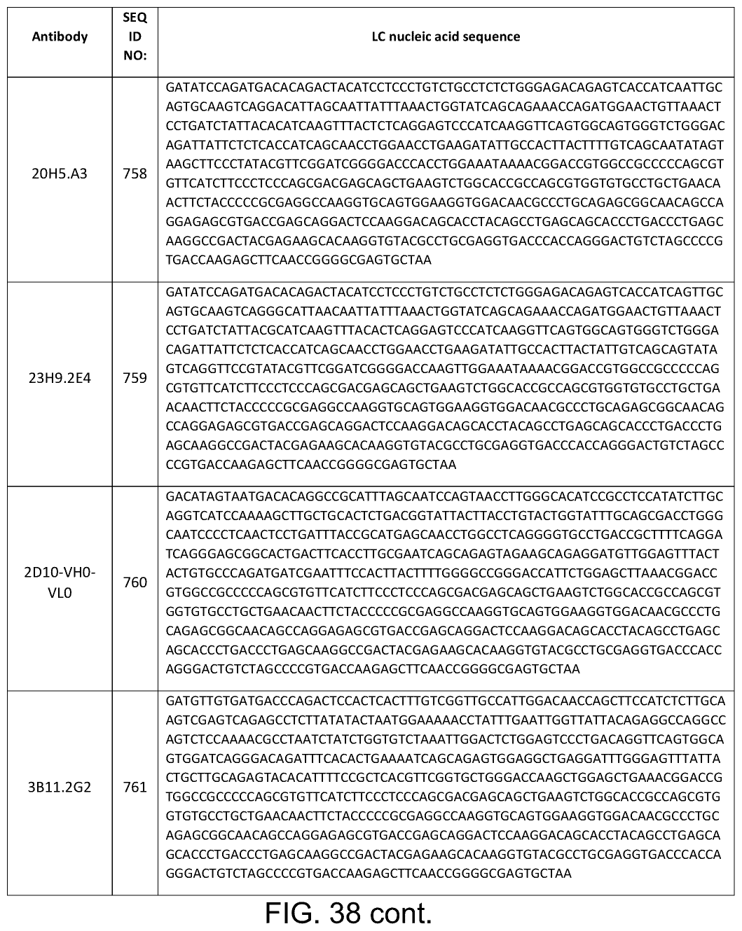

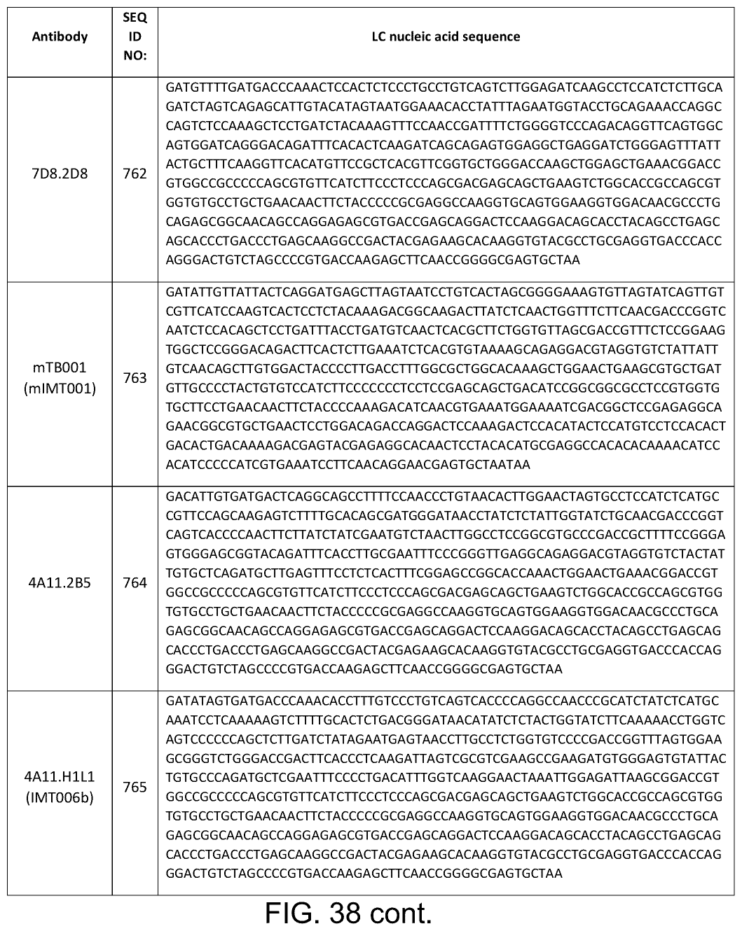

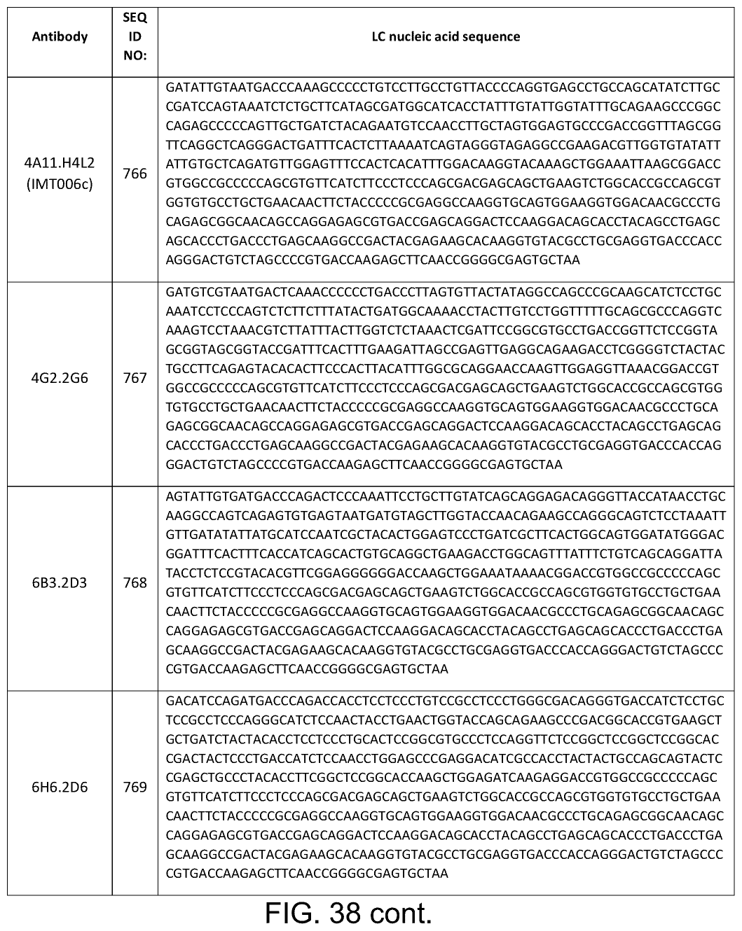

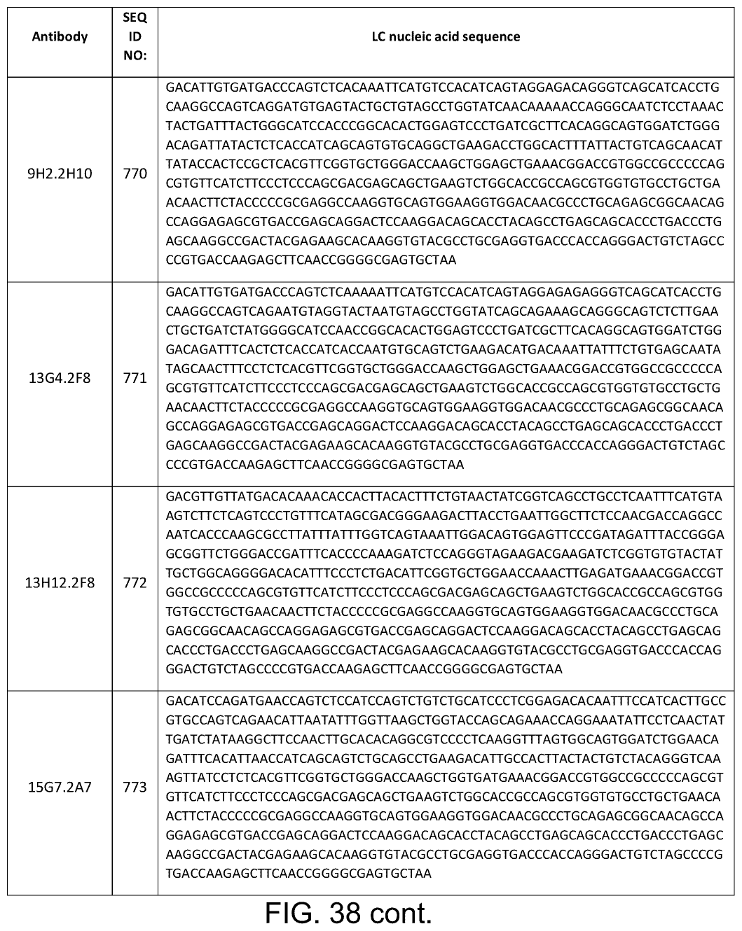

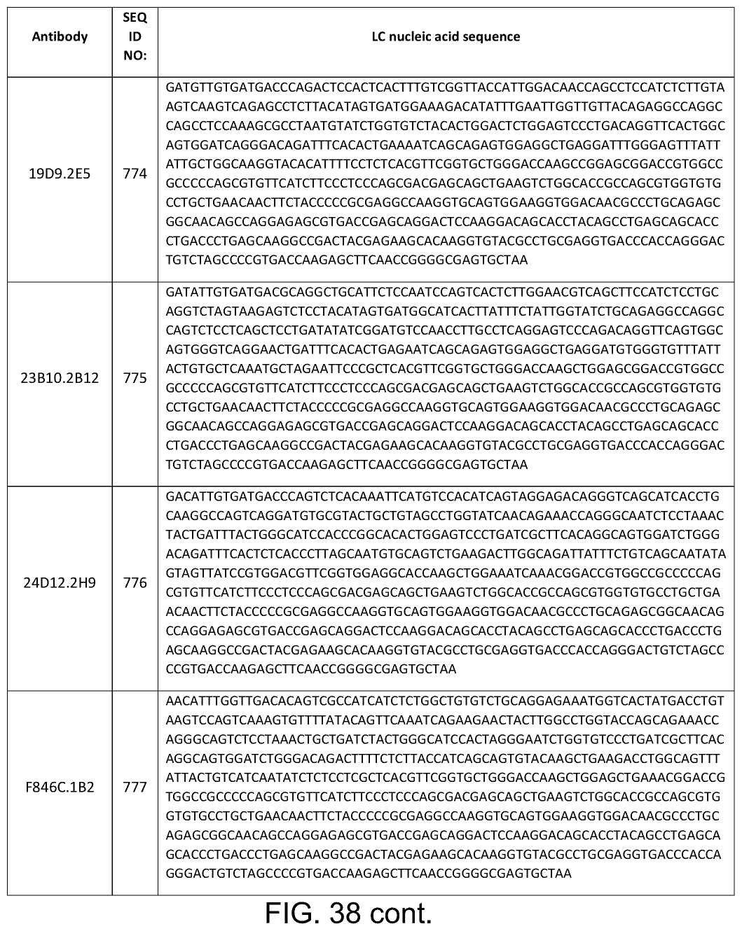

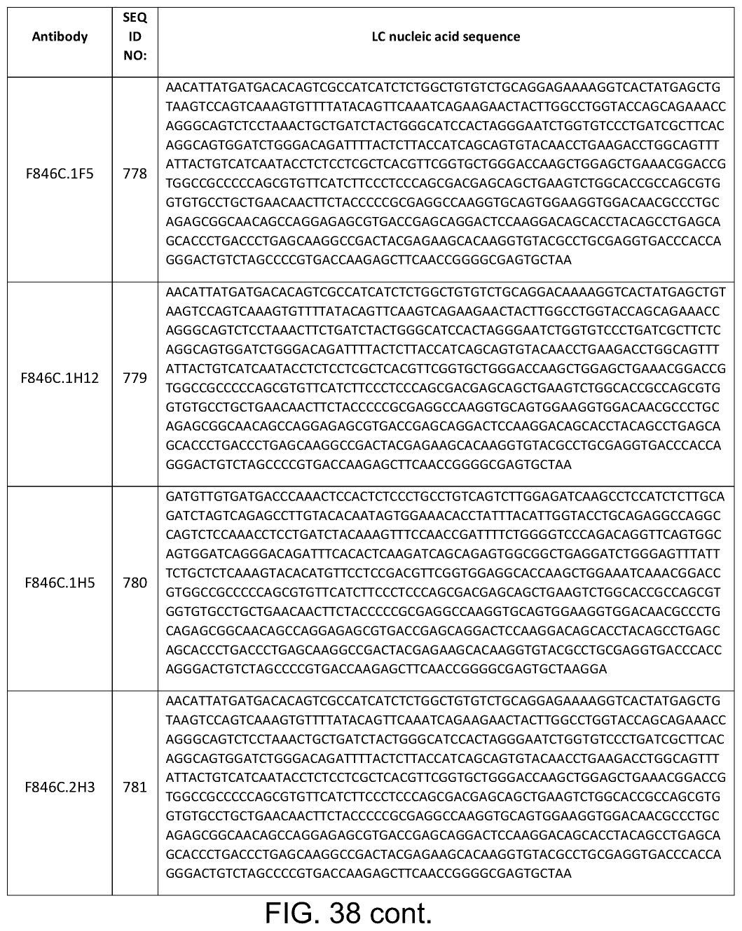

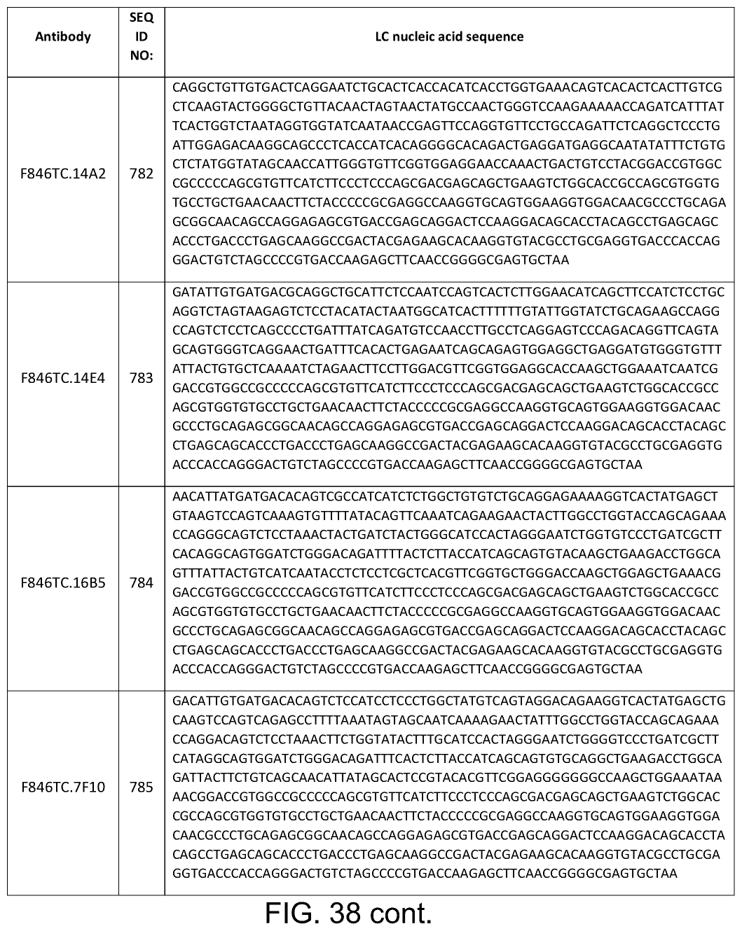

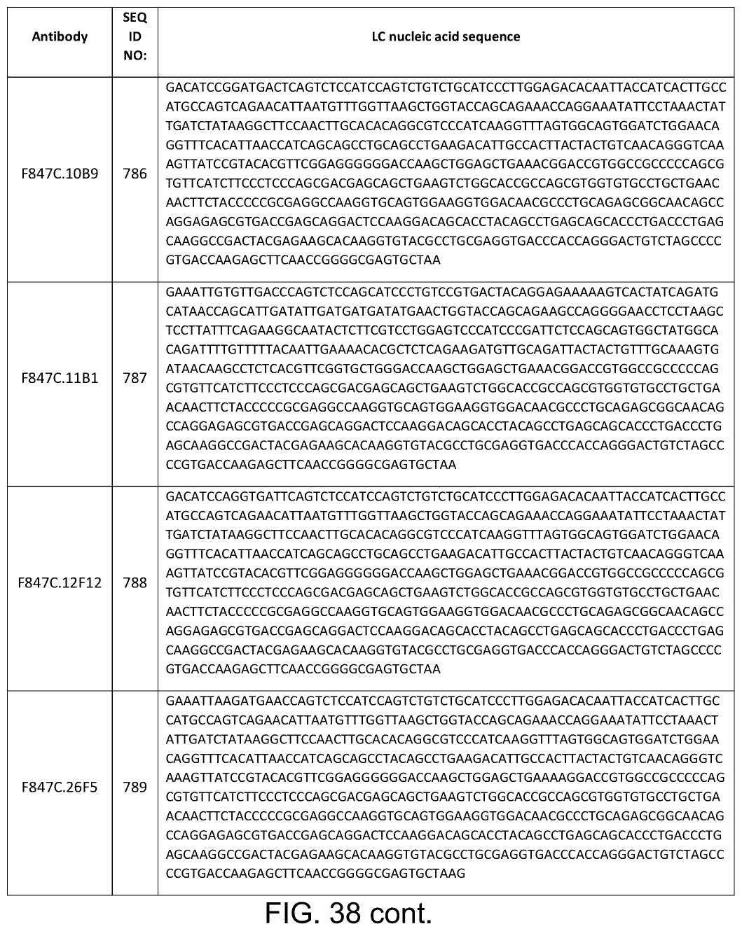

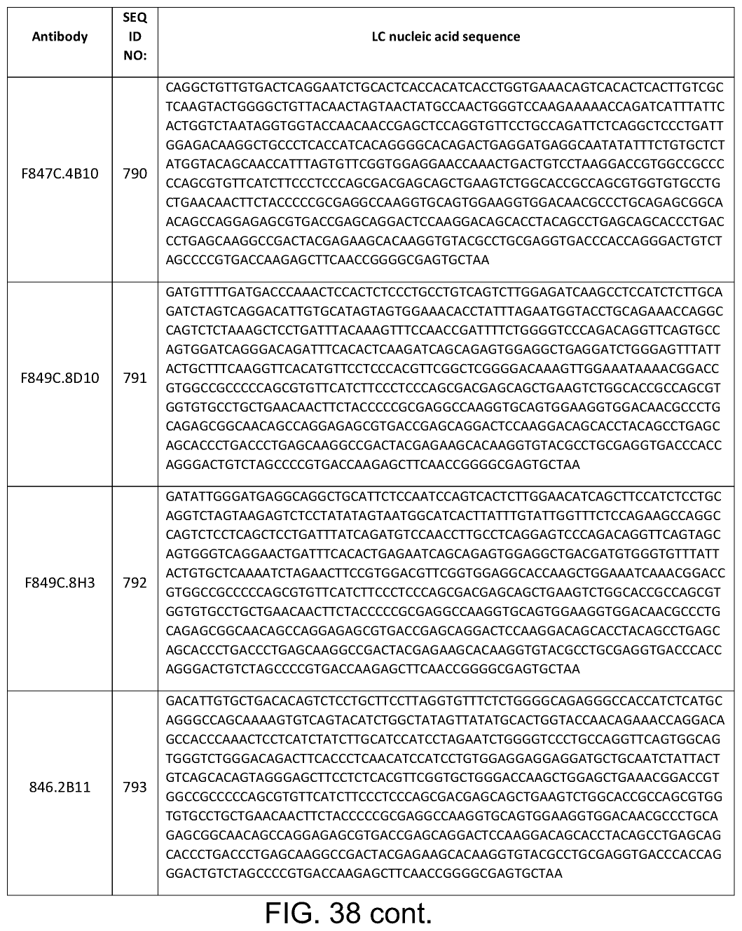

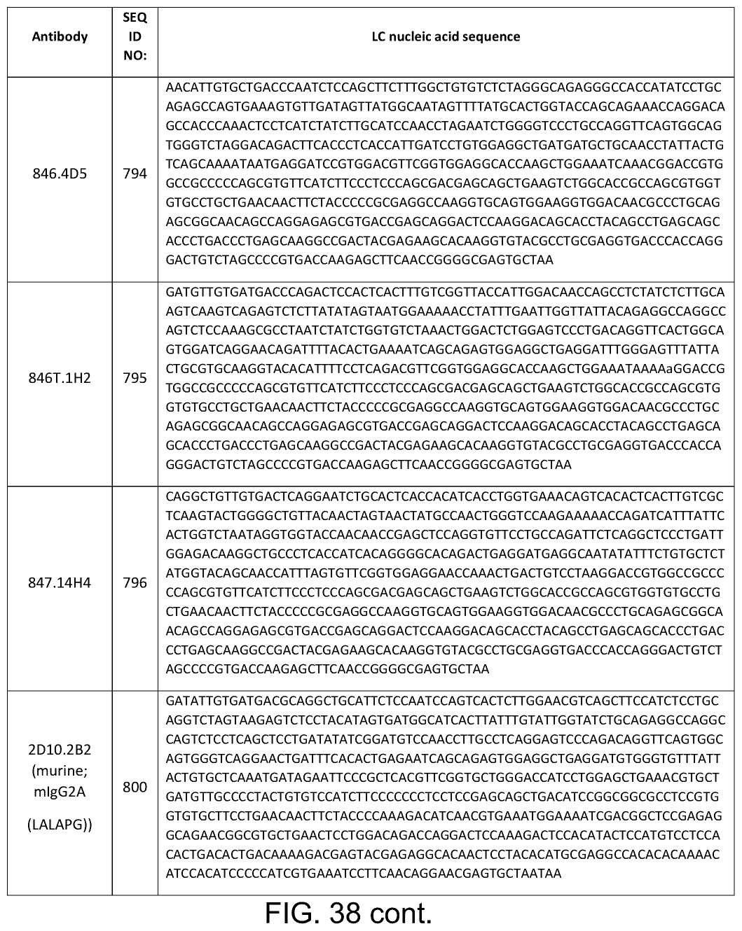

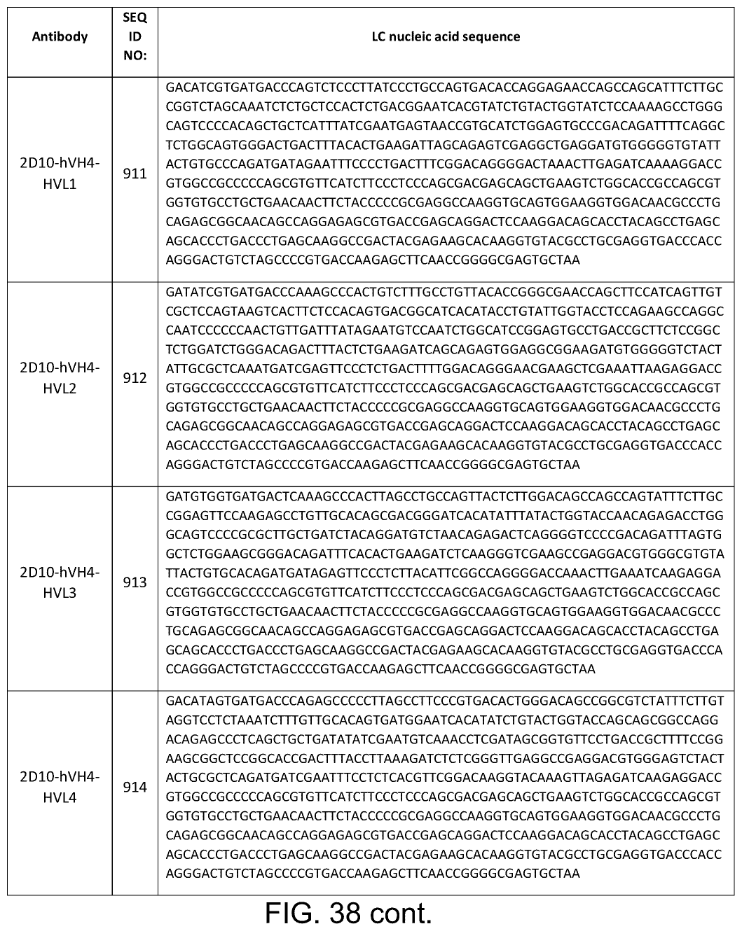

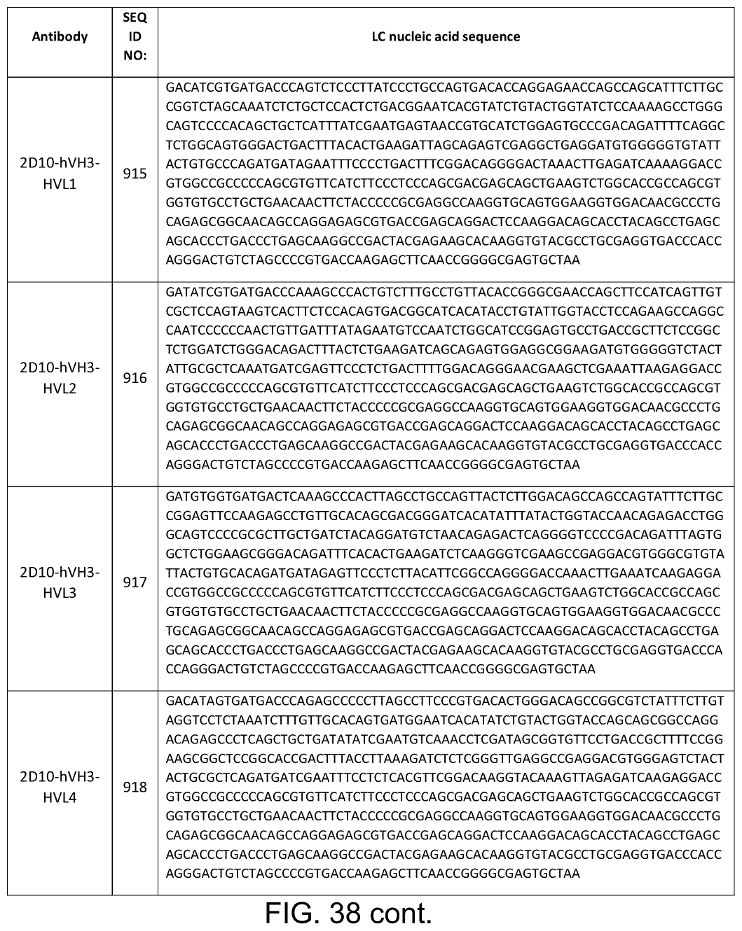

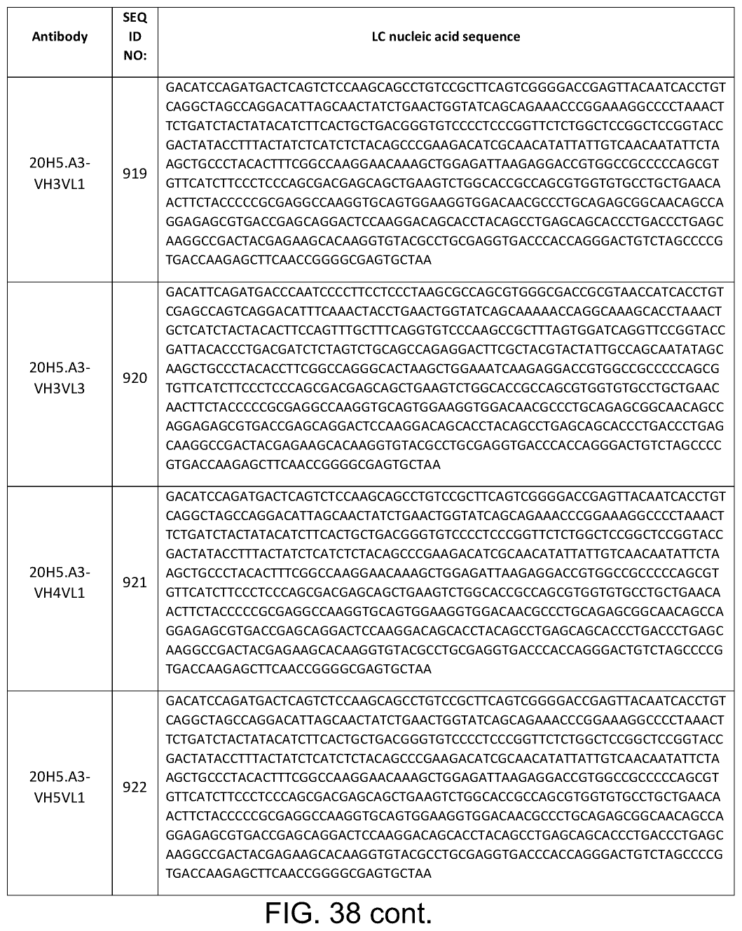

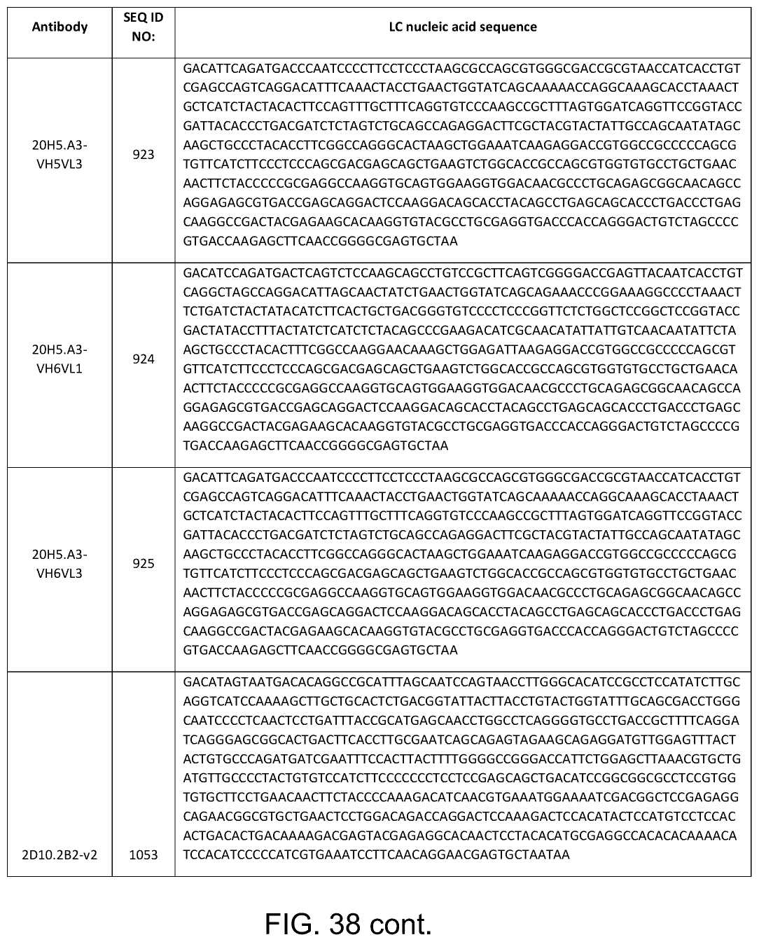

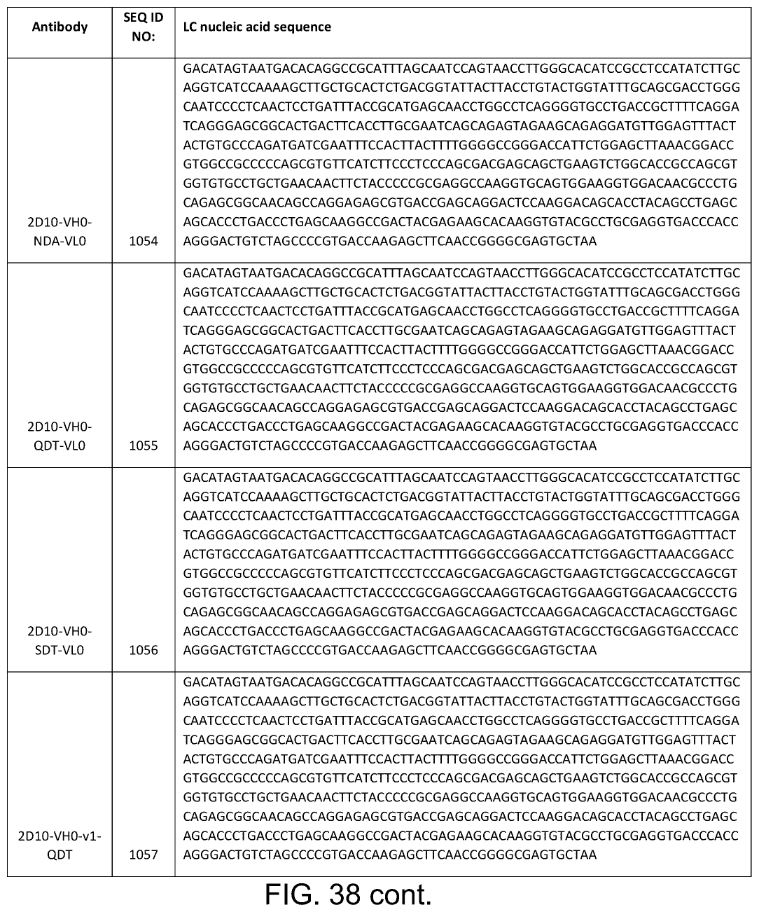

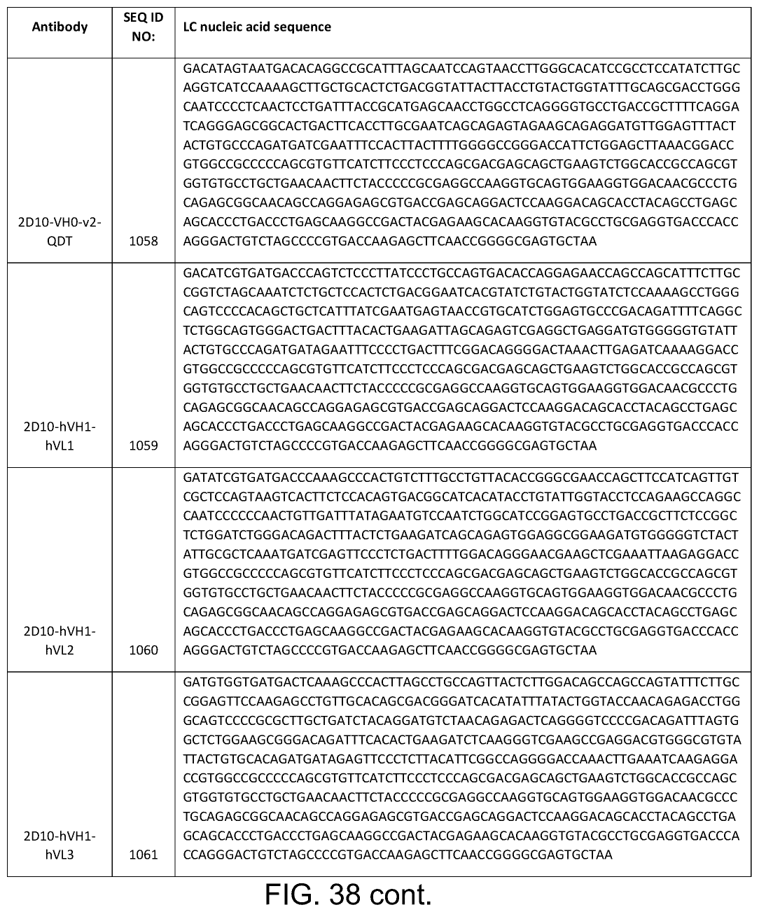

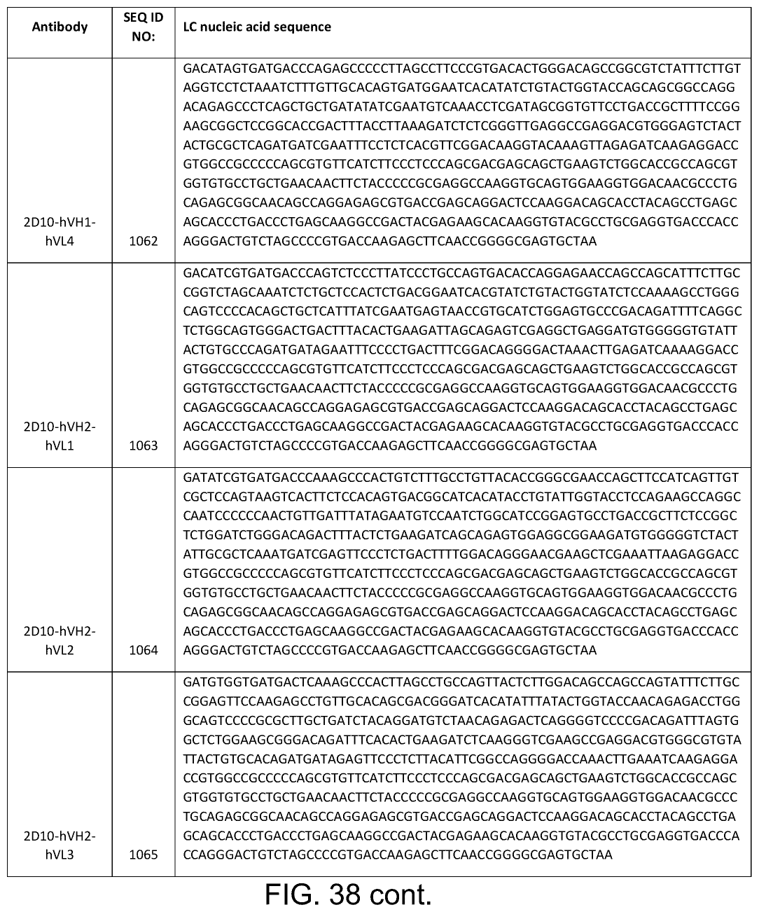

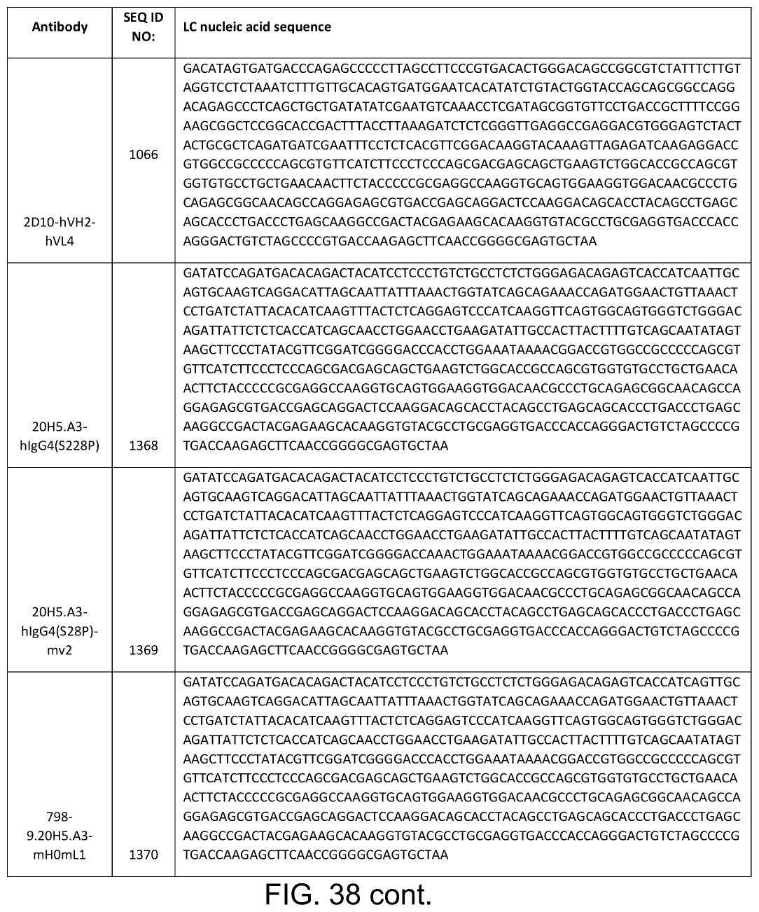

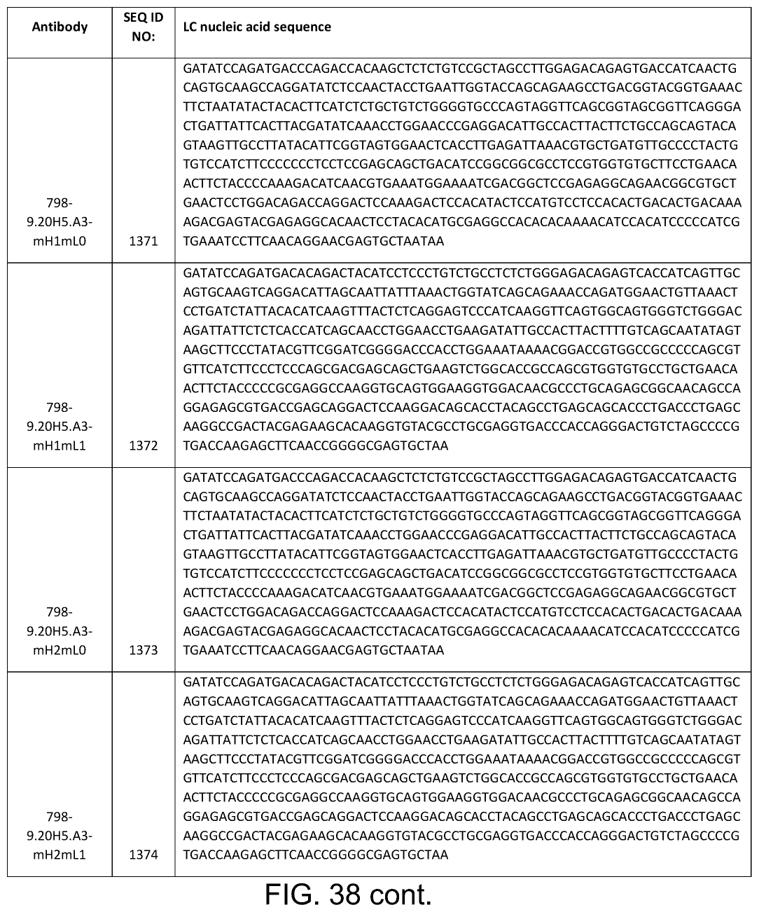

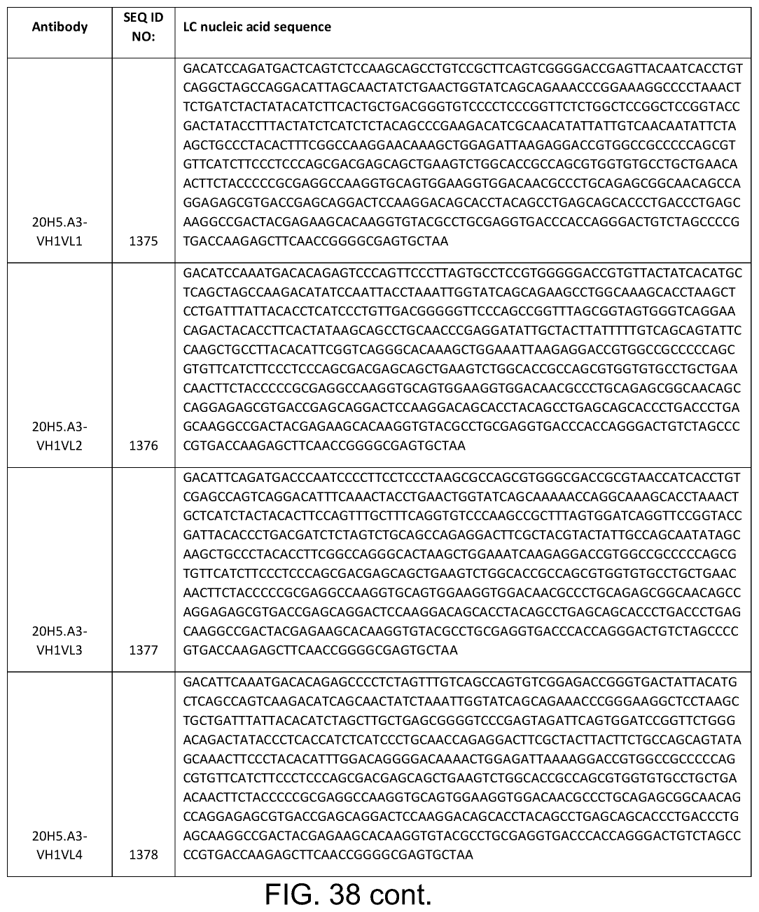

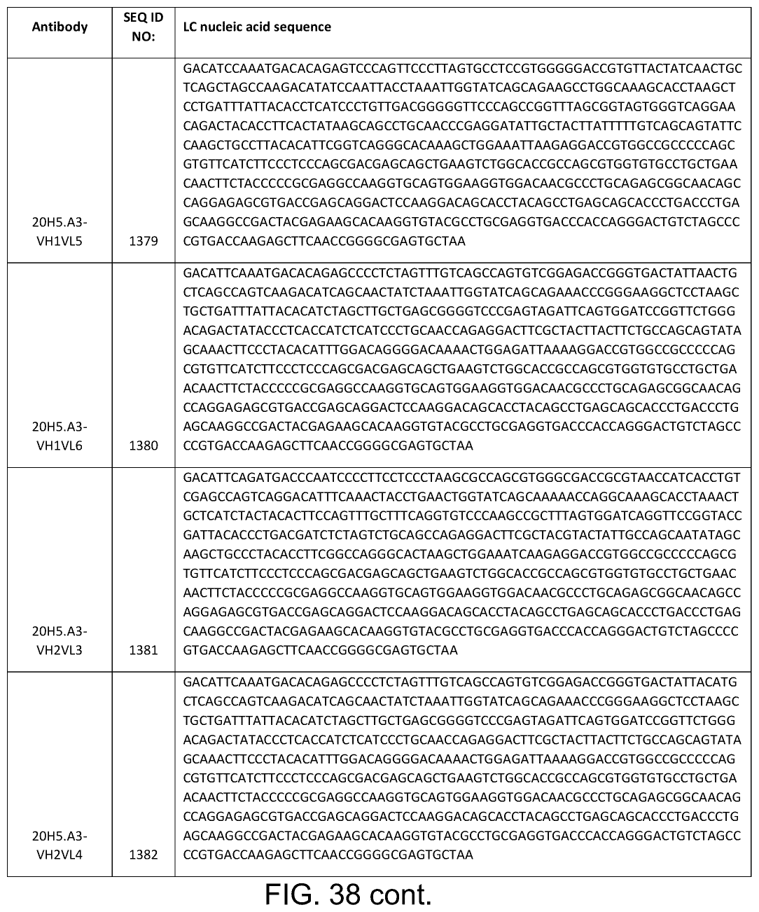

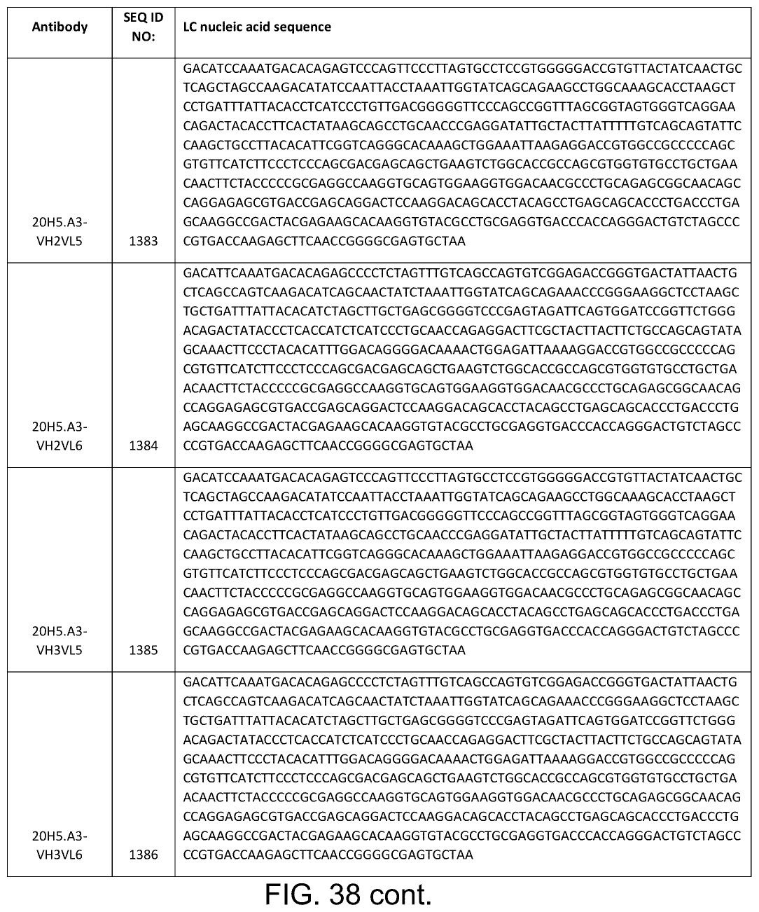

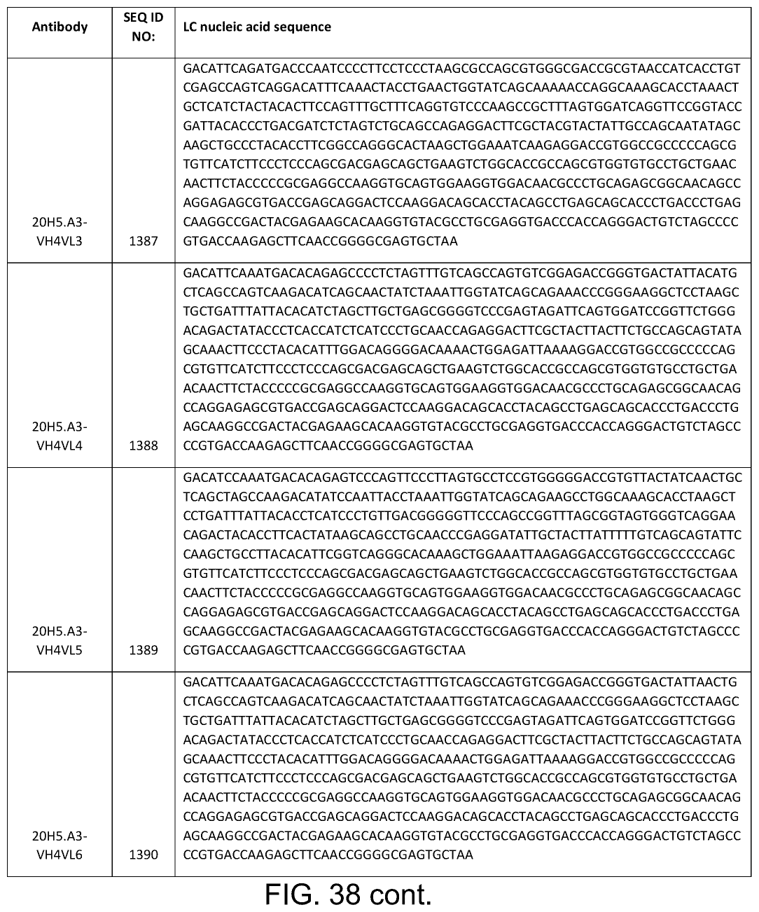

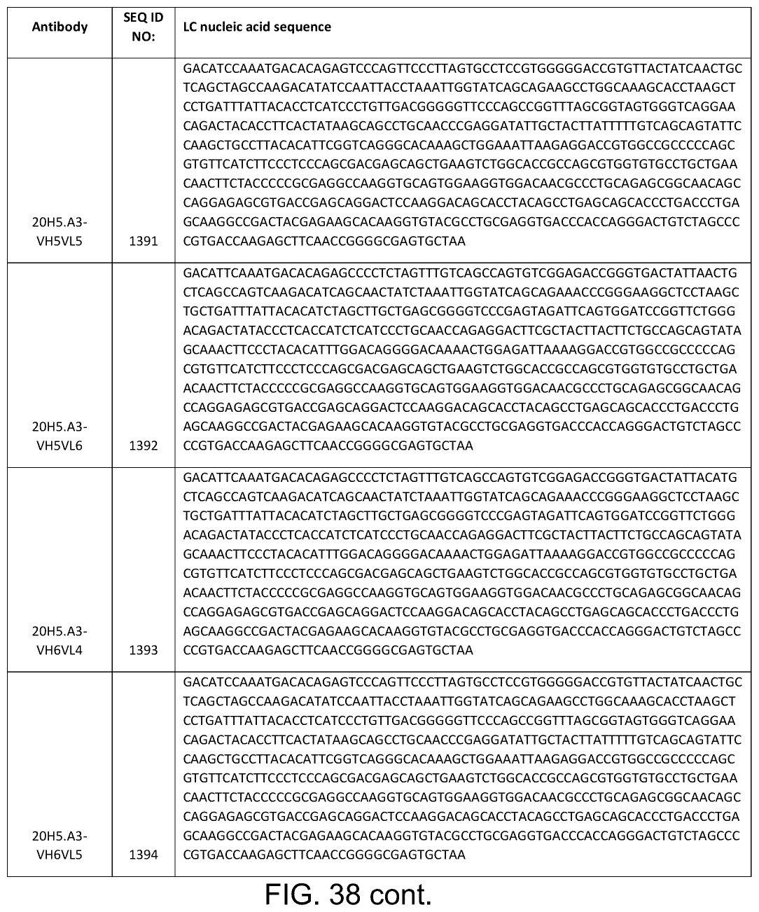

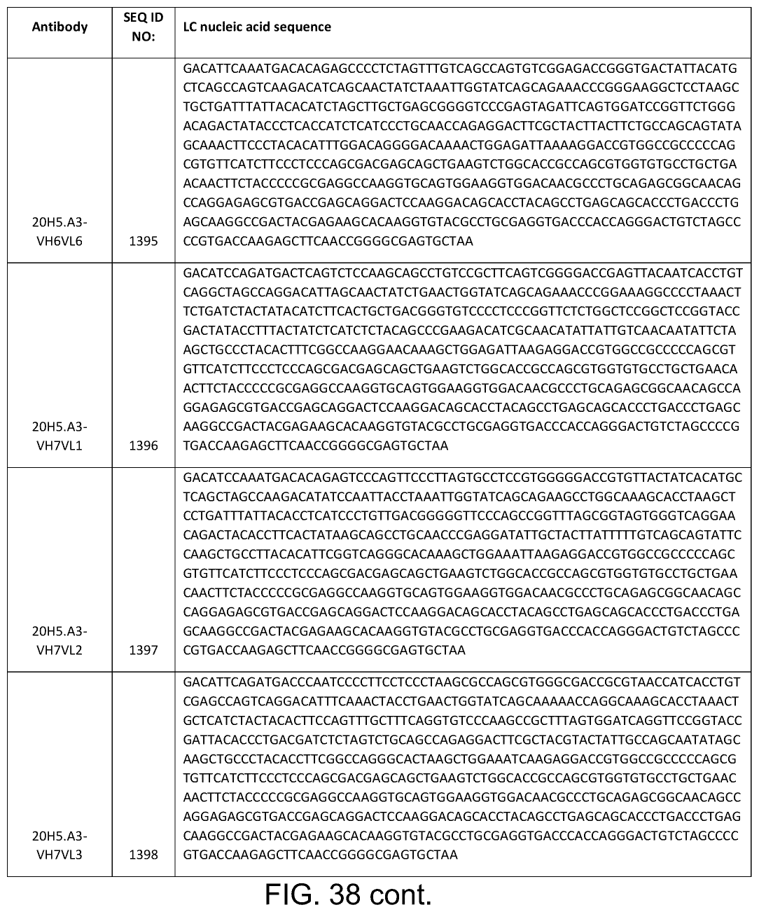

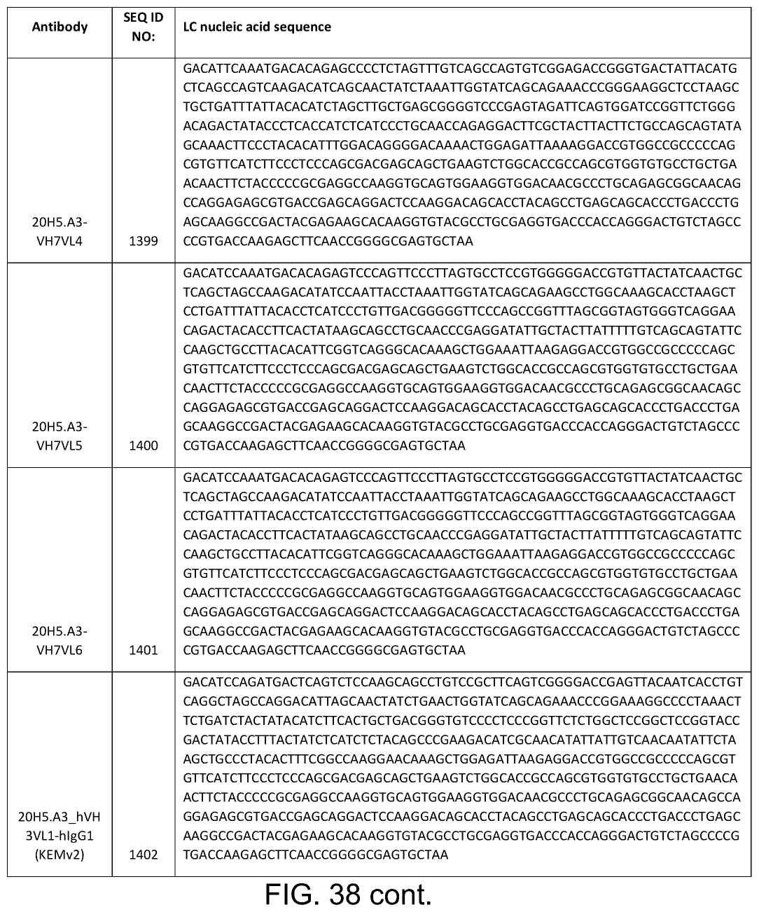

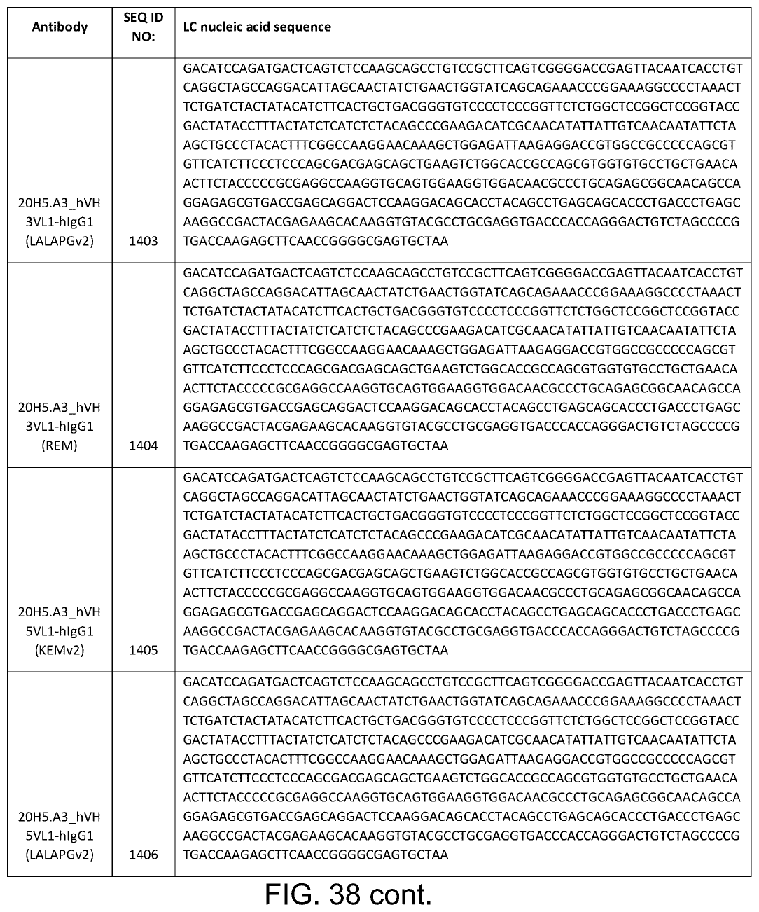

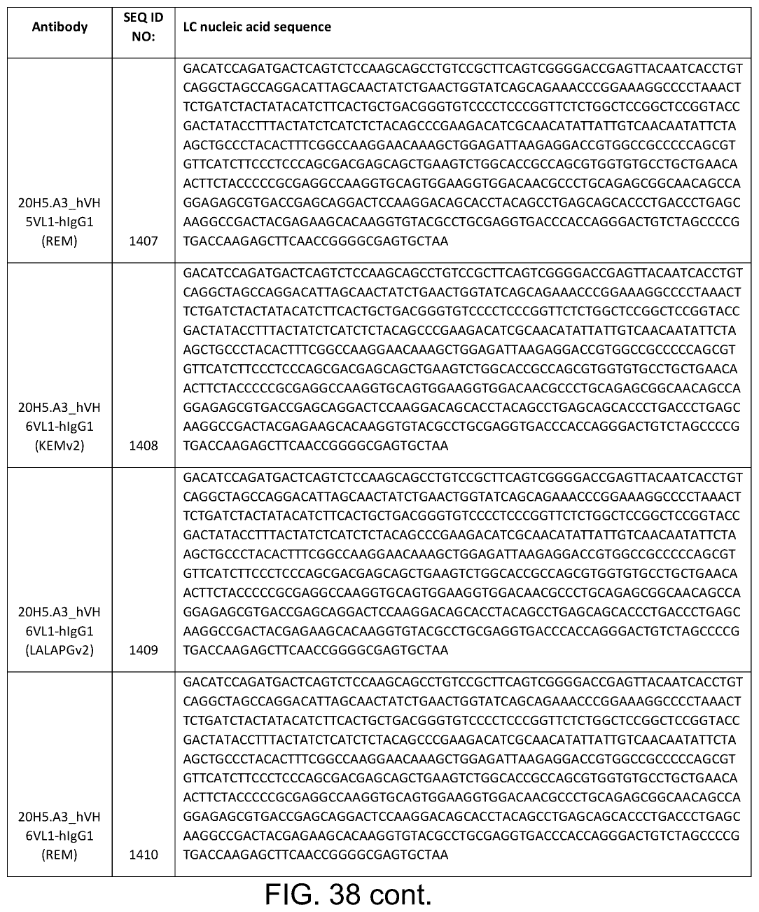

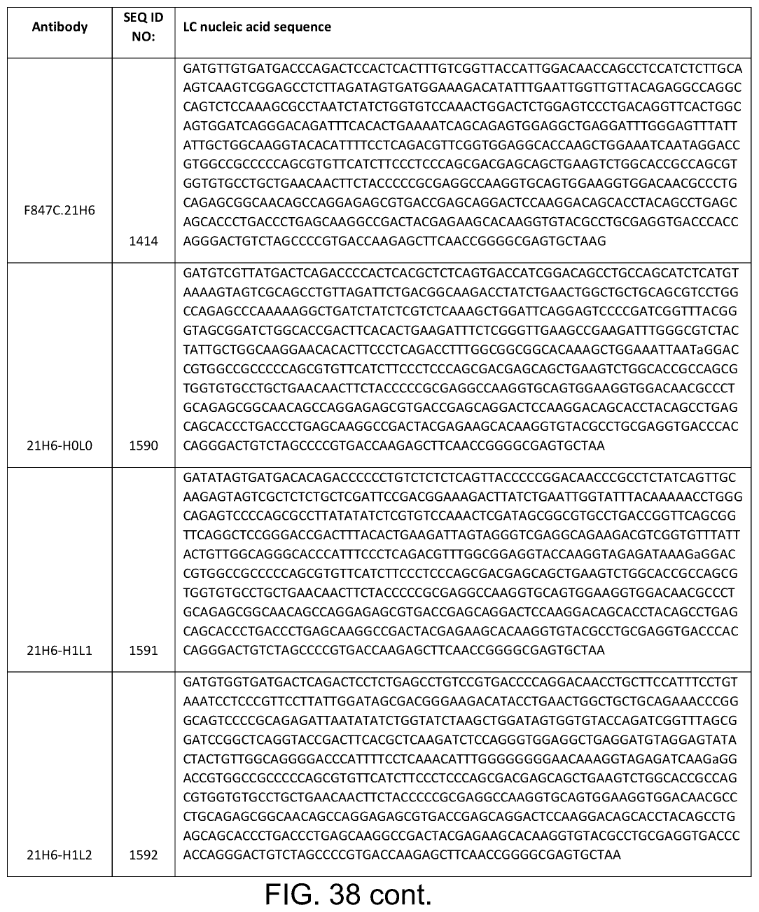

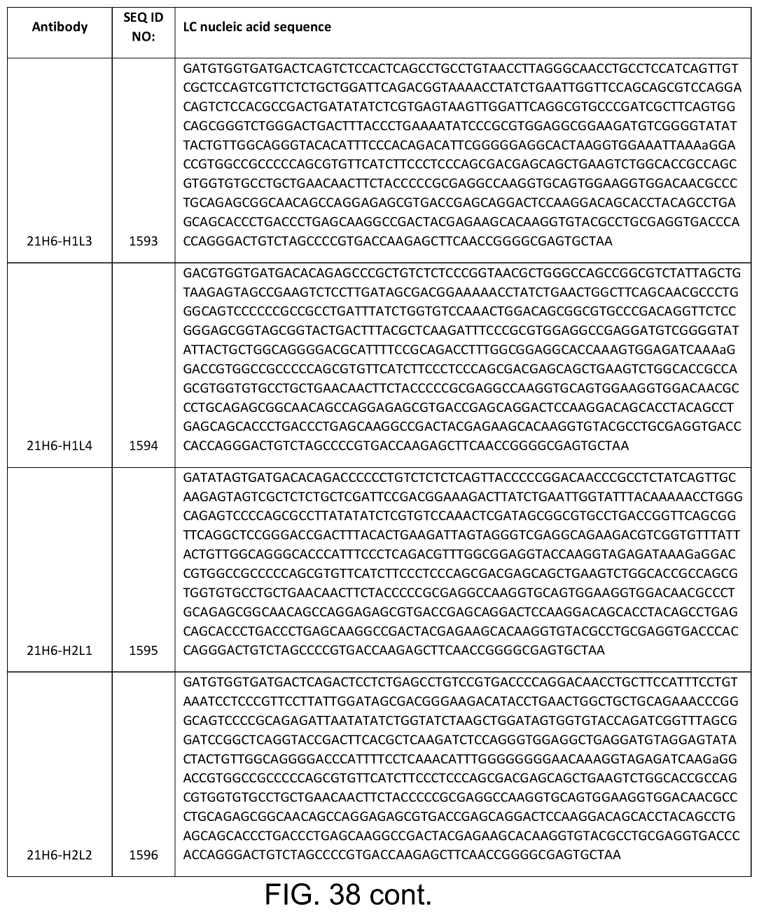

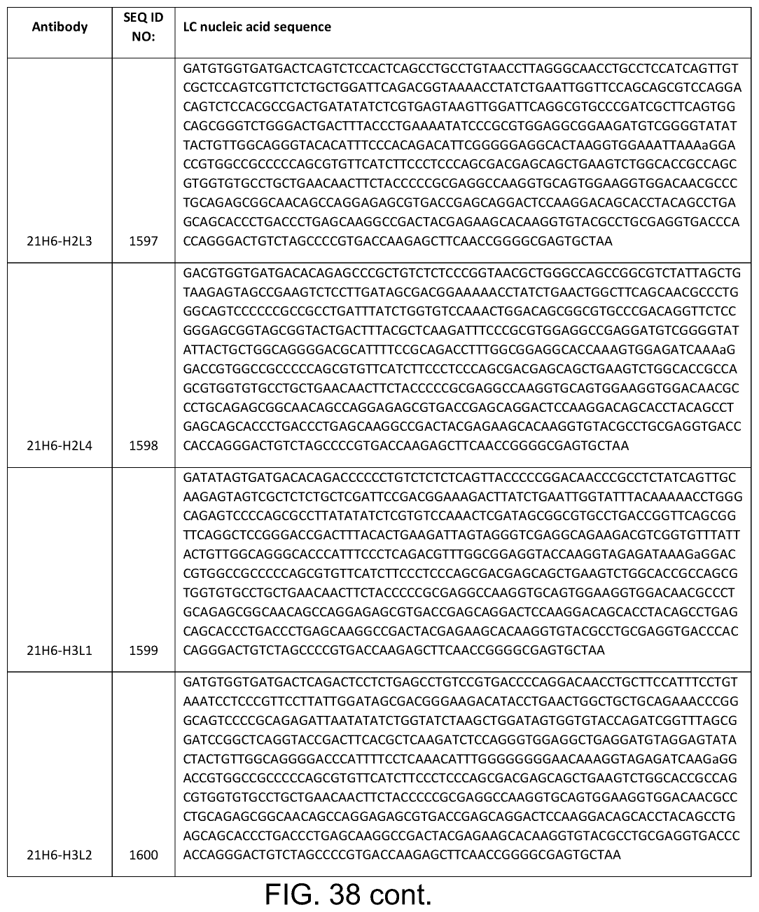

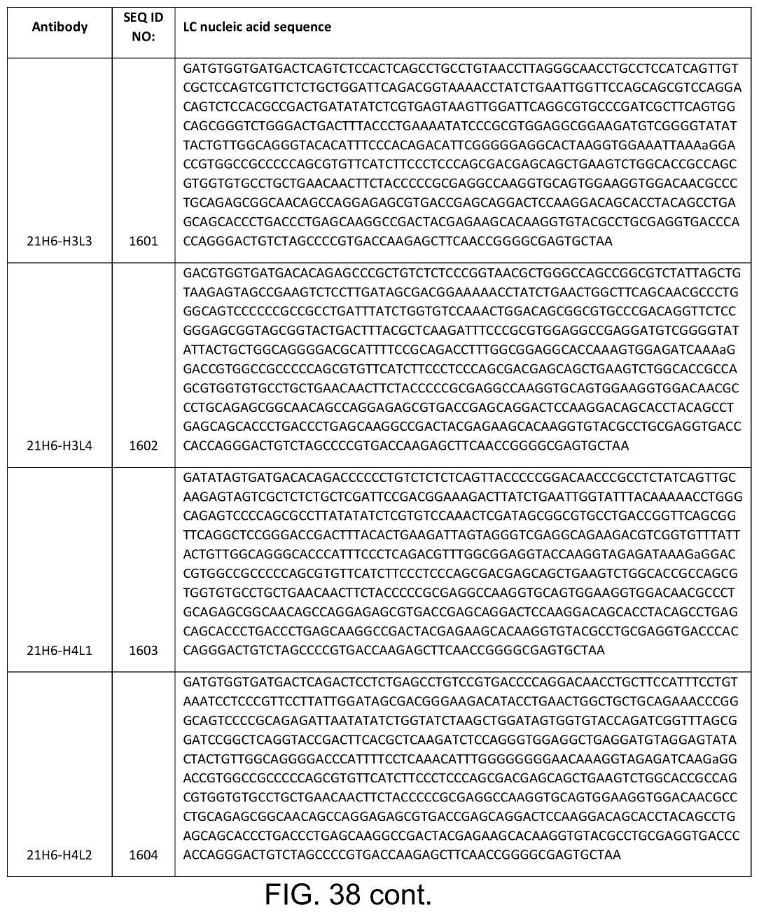

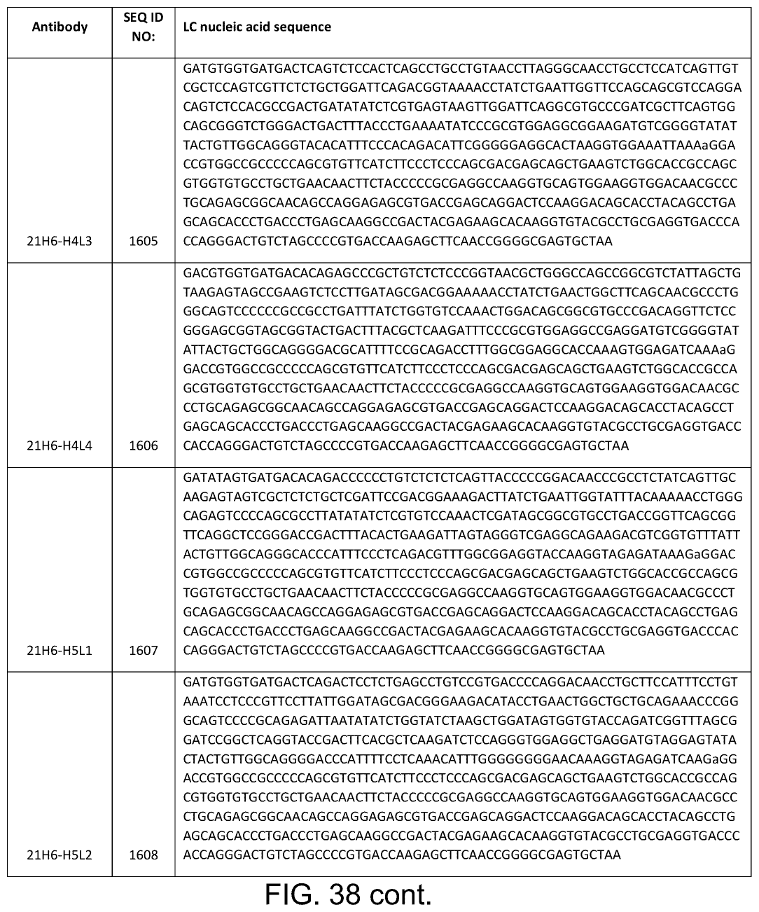

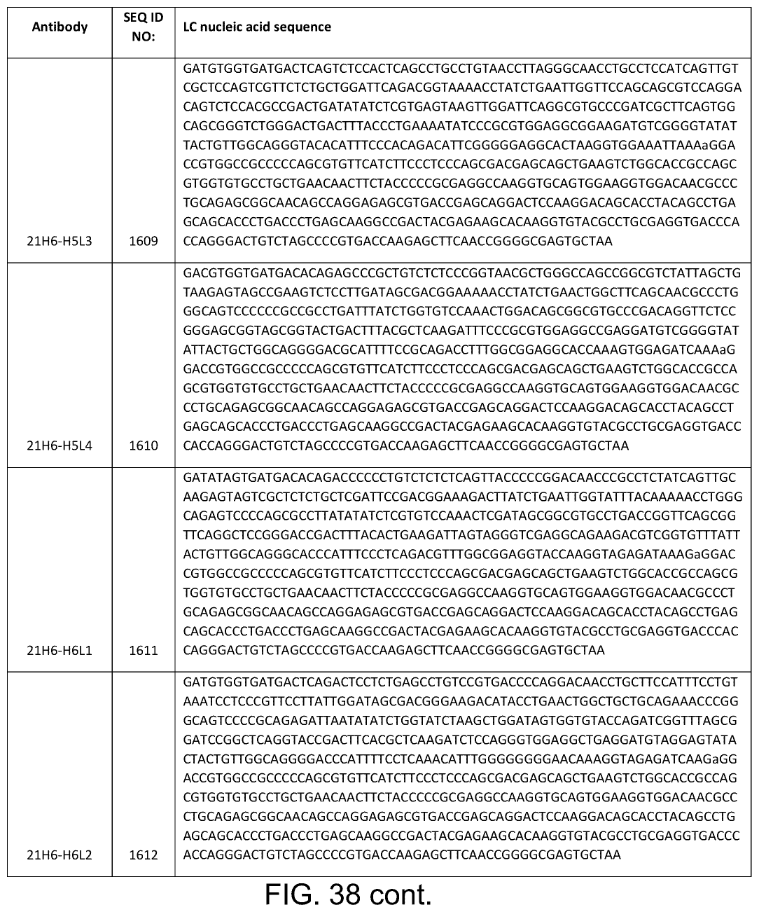

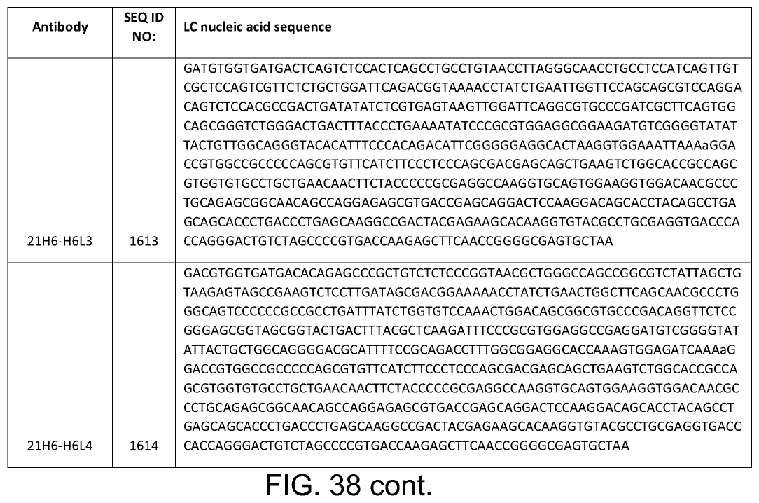

In some embodiments, any one or more of the constructs shown in any one or more of FIG. 24-39 can be used in any one or more of the embodiments provided herein (e.g., methods of treatment or uses in the preparation of a medicament for a therapy, etc.).

### Kit/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-Gal3 antibody as disclosed herein, host cells for producing one or more antibodies described herein, and/or vectors comprising nucleic acid molecules that encode the antibodies described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Numbered Arrangements

Some embodiments provided herein are described by way of the following provided numbered arrangements and also provided as possible combinations or overlapping embodiments:

1. A method of treating microglia mediated inflammation in a subject in need thereof, the method comprising:
identifying a subject as having or at risk of having microglia mediated inflammation, and
administering a therapeutically effective amount of an anti-Gal3 antibody to the subject, wherein
administration of the antibody to the subject alleviates one or more symptoms of microglia mediated inflammation.

2. A method of treating one or more diseases associated with microglia mediated inflammation in a subject in need thereof, the method comprising:
identifying a subject as having or at risk of having one or more diseases associated with microglia mediated inflammation, and
administering a therapeutically effective amount of an anti-Gal3 antibody to the administration of the antibody to the subject's eye alleviates one or more symptoms of a disease microglia mediated inflammation.

3. A method of treating an ocular surface disease in a subject in need thereof, the method comprising:
identifying a subject as having or at risk of having an ocular surface disease, and
administering a therapeutically effective amount of an anti-Gal3 antibody to the subject, wherein
administration of the antibody to the subject alleviates one or more symptoms of ocular surface disease.

4. A method of treating macular degeneration in a subject in need thereof, the method comprising:
identifying a subject as having or at risk of having macular degeneration, and
administering a therapeutically effective amount of an anti-Gal3 antibody to the subject, wherein
administration of the antibody to the subject alleviates one or more symptoms of macular degeneration.

5. A method of treating dry eye disease in a subject in need thereof, the method comprising:
identifying a subject as having or at risk of having dry eye disease, and
administering a therapeutically effective amount of an anti-Gal3 antibody to the subject, wherein
administration of the antibody to the subject alleviates one or more symptoms of dry eye disease.

6. A method of treating age-related vascular dysfunction in a subject in need thereof, the method comprising:
identifying a subject as having or at risk of having age-related vascular dysfunction, and
administering a therapeutically effective amount of an anti-Gal3 antibody to the
administration of the antibody to the subject alleviates one or more symptoms of age-related vascular dysfunction.

7. A method of increasing tear formation in a subject in need thereof, the method comprising:
identifying a subject as likely to benefit from increased tear production,
administering a therapeutically effective amount of an anti-Gal3 antibody to the subject, wherein
administration of the anti-Gal3 antibody to the subject increases tear production in the subject as compared to tear production in the subject prior to administration of the antibody.

8. A method of decreasing corneal sensitivity in a subject, the method comprising:
administering a therapeutically effective amount of an anti-Gal3 antibody to the subject, wherein
administration of the anti-Gal3 antibody to the subject's eye decreases corneal sensitivity in the subject as corneal sensitivity in the subject prior to administration of the antibody.

9. A method of treating cataracts in a subject in need thereof, the method comprising:
identifying a subject as having or at risk of having cataracts, and
administering a therapeutically effective amount of an anti-Gal3 antibody to the subject, wherein
administration of the antibody to the subject reduces the severity of the subject's cataracts as compared to the severity of cataracts in the subjects prior to administration of the anti-Gal3 antibody.

10. A method of preventing or reducing cataract formation in a subject in need thereof, the method comprising:
identifying a subject as having or at risk of having cataracts, and
administering a therapeutically effective amount of an anti-Gal3 antibody to the
administration of the antibody to the subject reduces or prevents cataract formation in the subject acts as compared to formation of cataracts in the subjects prior to administration of the anti-Gal3 antibody.

11. A method of treating diabetic retinopathy in a subject in need thereof, the method comprising:
identifying a subject as having or at risk of having diabetic retinopathy, and
administering a therapeutically effective amount of an anti-Gal3 antibody to the subject, wherein
administration of the antibody to the subject alleviates one or more symptoms of diabetic retinopathy.

12. A method of treating retinal drusen in a subject in need thereof, the method comprising:
identifying a subject as having or at risk of having retinal drusen, and
administering a therapeutically effective amount of an anti-Gal3 antibody to the subject, wherein
administration of the antibody to the subject reduces retinal drusen in the subject as compared to retinal drusen in the subject prior to administration of the anti-Gal3 antibody.

13. The method of any one of the preceding arrangements, wherein the anti-Gal3 antibody is administered intraperitoneally.

14. The method of any one of the preceding arrangements, wherein the anti-Gal3 antibody is administered topically.

15. The method of any one of the preceding arrangements, wherein the one or more symptoms comprise dryness of the eye, foreign body sensation, burning sensation, itching, photophobia, eye redness, blurred vision, fluctuating vision, visual fatigue, or any combination thereof.

16. The method of any one of the preceding arrangements, wherein the ocular surface disease comprises vascular dysfunction, angiogenesis, inflammation, and oxidative stress.

17. The method of any one of the preceding arrangements, wherein the corneal sensitivity of the subject is decreased by up to about 100% as compared to corneal sensitivity in the subject prior to administration of the anti-Gal3 antibody.

18. The method of any one of the preceding arrangements, wherein the corneal sensitivity of the subject is decreased by up to about 0.2 g as compared to corneal sensitivity in the subject prior to administration of the anti-Gal3 antibody.

19. The method of any one of the preceding arrangements, wherein tear production is increased by up to about 10 mm/60 s as compared to tear production in the subject prior to administration of the anti-Gal3 antibody.

20. The method of any one of the preceding arrangements, wherein tear production is increased by up to about 250% as compared to tear production in the subject prior to administration of the anti-Gal3 antibody.

21. The method of any one of the preceding arrangements, wherein the anti-Gal3 antibody is administered, daily, twice daily, weekly, bi-weekly, and/or monthly.

22. The method of any one of the preceding arrangements, wherein the anti-Gal3 antibody is administered for up to about 1, 2, 3, 4, 5, 6, 7, 8, 9, 10, 11, or 12 weeks, or for an amount of time that is in a range defined by any two of the preceding values.

23. The method of any one of the preceding arrangements, wherein the anti-Gal3 antibody is administered on an ongoing basis.

24. The method of any one of the preceding arrangements, wherein the anti-Gal3 antibody is topically administered to the subject's eye twice daily.

25. The method of any one of the preceding arrangements, wherein the anti-Gal3 antibody is topically administered to the subject's eye twice daily for 14 days.

26. The method of any one of the preceding arrangements, wherein 10 mg/kg of anti-Gal3 antibody is topically administered to the subject's eye.

27. The method of any one of the preceding arrangements, wherein 10 mg/kg of anti-Gal3 antibody is topically administered to the subject's eye twice daily.

28. The method of any one of the preceding arrangements, wherein 10 mg/kg of anti-Gal3 antibody is topically administered to the subject's eye twice daily for 14 days.

29. The method of any one of the preceding arrangements, wherein the anti-Gal3 antibody is TB001 or a binding fragment thereof.

30. The method of any one of the preceding arrangements, wherein the anti-Gal3 antibody is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

31. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the antibody comprises at least 1 V_{L}-CDR having at least 90% identity to any one of the amino acid sequences of SEQ ID NOs: 170-296, 1814-1864.

32. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the antibody comprises at least 3 V_{L}-CDR having at least 90% identity to any one of the amino acid sequences of SEQ ID Nos: 170-296, 1814-1864.

33. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
The antibody comprises at least 1 V_{H}-CDR having at least 90% identity to any one of the amino acid sequences of SEQ ID Nos: 27-169, 801, 802, 953, 954, 1763-1813.

34. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the antibody comprises at least 3 V_{H}-CDRs having at least 90% identity to any one of the amino acid sequences of SEQ ID NOs: 27-169, 801, 802, 953, 954, 1763-1813.

35. The method of any one of the preceding arrangements, wherein the antibody comprises at least 1 CDR having at least 90% identity to any one of the amino acid sequences of SEQ ID NOs: 27-296, 801, 802, 953, 954, 1763-1864.

36. The method of any one of the preceding arrangements, wherein the antibody comprises at least 6 CDRs having at least 90% identity to any one of the amino acid sequences of SEQ ID Nos: 27-296, 801, 802, 953, 954, 1763-1864.

37. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 90% identity to any one of the amino acid sequences of SEQ ID NOs: 27-70, 1763-1779;
the V_{H}-CDR2 comprises an amino acid sequence having at least 90% identity to any one of the amino acid sequences of SEQ ID NOs: 71-111, 801, 951, 952, 1780-1796;
the V_{H}-CDR3 comprises an amino acid sequence having at least 90% identity to any one of the amino acid sequences of SEQ ID NO: 112-169, 802, 953, 954, 1797-1813;
the V_{L}-CDR1 comprises an amino acid sequence having at least 90% identity to any one of the amino acid sequences of SEQ ID NOs: 170-220, 1814-1830;
the V_{L}-CDR2 comprises an amino acid sequence having at least 90% identity to any one of the amino acid sequences of SEQ ID NOs: 221-247, 1831-1847; and
the V_{L}-CDR3 comprises an amino acid sequence having at least 90% identity to any one of the amino acid sequences of SEQ ID NOs: 248-296, 1848-1864.

38. The method or composition of any one of the preceding arrangements, wherein the antibody comprises a combination of the V_{H}-CDR1, V_{H}-CDR2, V_{H}-CDR3, V_{L}-CDR1, V_{L}-CDR2, and V_{L}-CDR3 as illustrated in FIG. 32.

39. The method or composition of any one of the preceding arrangements, wherein the heavy chain variable region comprises a sequence having at least 90% identity to the sequence selected from SEQ ID NOs: 297-373, 803, 806-820, 940, 955-968, 1067-1109, 1415-1439, 1882-1898.

40. The method of any one of the preceding arrangements, wherein the light chain variable region comprises a sequence having at least 90% identity to the sequence selected from SEQ ID NOs: 374-447, 821-835, 941-943, 969-982, 1110-1152, 1440-1464, 1899-1914, 1921.

41. The method of any one of the preceding arrangements, wherein the antibody comprises a heavy chain, wherein the heavy chain comprises a sequence having at least 90% identity to the sequence selected from SEQ ID NOs: 448-494, 804, 836-850, 983-996, 1153-1195, 1411, 1465-1489, 1695-1711.

42. The method or composition of any one of the preceding arrangements, wherein the antibody comprises a light chain, wherein the light chain comprises a sequence having at least 90% identity to the sequence selected from SEQ ID NOs: 495-538, 805, 851-865, 997-1010, 1196-1238, 1412, 1490-1514, 1746-1762.

43. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 90% identity to SEQ ID NO: 27;
the V_{H}-CDR2 comprises an amino acid sequence having at least 90% identity to SEQ ID NO: 71;
the V_{H}-CDR3 comprises an amino acid sequence having at least 90% identity to SEQ ID NO: 112;
the V_{H}-CDR1 comprises an amino acid sequence having at least 90% identity to SEQ ID NO: 170;
the V_{H}-CDR1 comprises an amino acid sequence having at least 90% identity to SEQ ID NO: 221; and
the V_{H}-CDR1 comprises an amino acid sequence having at least 90%, to SEQ ID NO: 248.

44. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein

45. the antibody comprises at least 3 V_{H}-CDRs having at least 90% identity to any one of the amino acid sequences of SEQ ID NOs: 27, 71, 112.

46. The method of any one of the preceding arrangements, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the antibody comprises at least 3 V_{H}-CDRs having at least 90% identity to any one of the amino acid sequences of SEQ ID NOs: 170, 221, 248.

47. The method or composition of any one of the preceding arrangements, wherein the heavy chain variable region comprises a sequence having at least 90% identity to SEQ ID NO: 297.

48. The method or composition of any one of the preceding arrangements, wherein the heavy chain variable region comprises a sequence having at least 90% identity to SEQ ID NO: 374.

49. The method of any one of the preceding arrangements, wherein the antibody comprises a heavy chain, wherein the heavy chain comprises a sequence having at least 90% identity to the SEQ ID NO: 448.

50. The method or composition of any one of the preceding arrangements, wherein the antibody comprises a light chain, wherein the light chain comprises a sequence having at least 90% identity to SEQ ID NO: 495.

51. The method of any one of the preceding arrangements, where in the antibody is applied to the subject's eye.

52. The method of arrangement 51, wherein the antibody is one of the antibodies provided in any one of the figures provided herewith.

53. The method of arrangement 52, wherein the antibody is provided in an amount sufficient for the treatment of the eye disorder, when administered to the eye of the subject.

54. The method of arrangement 53, wherein the antibody is applied topically or injected into the subject's eye.

55. The method of any one of the preceding arrangements, wherein the antibody is administered intraperitoneally.

56. The method of any one of the preceding arrangements, wherein the amount of the antibody administered is a therapeutically effective amount, when administered intraperitoneally.

57. The method of any one of the preceding arrangements, wherein:
a. the antibody is applied to the subject's eye,
b. the antibody is one of the antibodies provided in any one of the figures provided herewith,
c. the antibody is provided in an amount sufficient for the treatment of the eye disorder, when administered to the eye of the subject;
d. the antibody is applied topically or injected into the subject's eye,
e. the antibody is administered intraperitoneally, and/or the amount of the antibody administered is a therapeutically effective amount, when administered intraperitoneally.

58. An anti-Gal3 antibody for use in treating microglia mediated inflammation in a subject in need thereof, the use comprising:
identifying a subject as having or at risk of having microglia mediated inflammation, and
administering a therapeutically effective amount of an anti-Gal3 antibody to the subject, wherein
administration of the antibody to the subject alleviates one or more symptoms of microglia mediated inflammation.

59. An anti-Gal3 antibody for use in treating one or more diseases associated with microglia mediated inflammation in a subject in need thereof, the use comprising:
identifying a subject as having or at risk of having one or more diseases associated with microglia mediated inflammation, and
administering a therapeutically effective amount of an anti-Gal3 antibody to the subject, wherein
administration of the antibody to the subject's eye alleviates one or more symptoms of a disease microglia mediated inflammation.

60. An anti-Gal3 antibody for use in treating an ocular surface disease in a subject in need thereof, the use comprising:
identifying a subject as having or at risk of having an ocular surface disease, and
administering a therapeutically effective amount of an anti-Gal3 antibody to the subject, wherein
administration of the antibody to the subject alleviates one or more symptoms of ocular surface disease.

61. An anti-Gal3 antibody for use in treating macular degeneration in a subject in need thereof, the use comprising:
identifying a subject as having or at risk of having macular degeneration, and
administering a therapeutically effective amount of an anti-Gal3 antibody to the
administration of the antibody to the subject alleviates one or more symptoms of macular degeneration.

62. An anti-Gal3 antibody for use in treating dry eye disease in a subject in need thereof, the use comprising:
identifying a subject as having or at risk of having dry eye disease, and
administering a therapeutically effective amount of an anti-Gal3 antibody to the subject, wherein
administration of the antibody to the subject alleviates one or more symptoms of dry eye disease.

63. An anti-Gal3 antibody for use in treating age-related vascular dysfunction in a subject in need thereof, the use comprising:
identifying a subject as having or at risk of having age-related vascular dysfunction, and
administering a therapeutically effective amount of an anti-Gal3 antibody to the subject, wherein
administration of the antibody to the subject alleviates one or more symptoms of age-related vascular dysfunction.

64. An anti-Gal3 antibody for use in increasing tear formation in a subject in need thereof, the use comprising:
identifying a subject as likely to benefit from increased tear production,
administering a therapeutically effective amount of an anti-Gal3 antibody to the subject, wherein
administration of the anti-Gal3 antibody to the subject increases tear production in the subject as compared to tear production in the subject prior to administration of the antibody.

65. An anti-Gal3 antibody for use in decreasing corneal sensitivity in a subject, the use comprising:
administering a therapeutically effective amount of an anti-Gal3 antibody to the
administration of the anti-Gal3 antibody to the subject's eye decreases corneal sensitivity in the subject as corneal sensitivity in the subject prior to administration of the antibody.

66. An anti-Gal3 antibody for use in treating cataracts in a subject in need thereof, the use comprising:
identifying a subject as having or at risk of having cataracts, and
administering a therapeutically effective amount of an anti-Gal3 antibody to the subject, wherein
administration of the antibody to the subject reduces the severity of the subject's cataracts as compared to the severity of cataracts in the subjects prior to administration of the anti-Gal3 antibody.

67. An anti-Gal3 antibody for use in preventing or reducing cataract formation in a subject in need thereof, the use comprising:
identifying a subject as having or at risk of having cataracts, and
administering a therapeutically effective amount of an anti-Gal3 antibody to the subject, wherein
administration of the antibody to the subject reduces or prevents cataract formation in the subject acts as compared to formation of cataracts in the subjects prior to administration of the anti-Gal3 antibody.

68. An anti-Gal3 antibody for use in treating diabetic retinopathy in a subject in need thereof, the use comprising:
identifying a subject as having or at risk of having diabetic retinopathy, and
administering a therapeutically effective amount of an anti-Gal3 antibody to the subject, wherein
administration of the antibody to the subject alleviates one or more symptoms of diabetic retinopathy.

69. An anti-Gal3 antibody for use in treating retinal drusen in a subject in need thereof, the use comprising:
identifying a subject as having or at risk of having retinal drusen, and
administering a therapeutically effective amount of an anti-Gal3 antibody to the
administration of the antibody to the subject reduces retinal drusen in the subject as compared to retinal drusen in the subject prior to administration of the anti-Gal3 antibody.

70. The anti-Gal3 antibody for use of any one of the preceding arrangements, wherein:
a. the anti-Gal3 antibody is administered intraperitoneally;
b. the anti-Gal3 antibody is administered topically;
c. the one or more symptoms comprise dryness of the eye, foreign body sensation, burning sensation, itching, photophobia, eye redness, blurred vision, fluctuating vision, visual fatigue, or any combination thereof;
d. the ocular surface disease comprises vascular dysfunction, angiogenesis, inflammation, and oxidative stress;
e. the corneal sensitivity of the subject is decreased by up to about 100% as compared to corneal sensitivity in the subject prior to administration of the anti-Gal3 antibody;
f. the corneal sensitivity of the subject is decreased by up to about 0.2 g as compared to corneal sensitivity in the subject prior to administration of the anti-Gal3 antibody;
g. tear production is increased by up to about 10 mm/60 s as compared to tear production in the subject prior to administration of the anti-Gal3 antibody;
h. tear production is increased by up to about 250% as compared to tear production in the subject prior to administration of the anti-Gal3 antibody.
i. the anti-Gal3 antibody is administered, daily, twice daily, weekly, bi-weekly, and/or monthly;
j. the anti-Gal3 antibody is administered for up to about 1, 2, 3, 4, 5, 6, 7, 8, 9, 10, 11, or 12 weeks, or for an amount of time that is in a range defined by any two of the preceding values;
k. the anti-Gal3 antibody is administered on an ongoing basis;
l. the anti-Gal3 antibody is topically administered to the subject's eye twice daily;
m. the anti-Gal3 antibody is topically administered to the subject's eye twice daily for 14 days;
n. 10 mg/kg of anti-Gal3 antibody is topically administered to the subject's eye;
o. 10 mg/kg of anti-Gal3 antibody is topically administered to the subject's eye twice daily;
p. 10 mg/kg of anti-Gal3 antibody is topically administered to the subject's eye twice daily for 14 days; and/or
q. the anti-Gal3 antibody is TB001 or a binding fragment thereof.

71. The anti-Gal3 antibody for use of any one of the preceding arrangements, wherein:
a. the anti-Gal3 antibody is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof;
b. the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the antibody comprises at least 1 V_{L}-CDR having at least 90% identity to any one of the amino acid sequences of SEQ ID NOs: 170-296, 1814-1864;
c. the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the antibody comprises at least 3 V_{L}-CDR having at least 90% identity to any one of the amino acid sequences of SEQ ID Nos: 170-296, 1814-1864;
d. the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the antibody comprises at least 1 V_{H}-CDR having at least 90% identity to any one of the amino acid sequences of SEQ ID Nos: 27-169, 801, 802, 953, 954, 1763-1813;
e. the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the antibody comprises at least 3 V_{H}-CDRs having at least 90% identity to any one of the amino acid sequences of SEQ ID NOs: 27-169, 801, 802, 953, 954, 1763-1813;
f. the antibody comprises at least 1 CDR having at least 90% identity to any one of the amino acid sequences of SEQ ID NOs: 27-296, 801, 802, 953, 954, 1763-1864;
g. the antibody comprises at least 6 CDRs having at least 90% identity to any one of the amino acid sequences of SEQ ID Nos: 27-296, 801, 802, 953, 954, 1763-1864;
h. the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein the V_{H}-CDR1 comprises an amino acid sequence having at least 90% identity to any one of the amino acid sequences of SEQ ID NOs: 27-70, 1763-1779;
   the V_{H}-CDR2 comprises an amino acid sequence having at least 90% identity to any one of the amino acid sequences of SEQ ID NOs: 71-111, 801, 951, 952, 1780-1796;
   the V_{H}-CDR3 comprises an amino acid sequence having at least 90% identity to any one of the amino acid sequences of SEQ ID NO: 112-169, 802, 953, 954, 1797-1813;
   the V_{L}-CDR1 comprises an amino acid sequence having at least 90% identity to any one of the amino acid sequences of SEQ ID NOs: 170-220, 1814-1830;
   the V_{L}-CDR2 comprises an amino acid sequence having at least 90% identity to any one of the amino acid sequences of SEQ ID NOs: 221-247, 1831-1847; and the V_{L}-CDR3 comprises an amino acid sequence having at least 90% identity to any one of the amino acid sequences of SEQ ID NOs: 248-296, 1848-1864;
i. the antibody comprises a combination of the V_{H}-CDR1, V_{H}-CDR2, V_{H}-CDR3, V_{L}-CDR1, V_{L}-CDR2, and V_{L}-CDR3 as illustrated in FIG. 32;
j. the heavy chain variable region comprises a sequence having at least 90% identity to the sequence selected from SEQ ID NOs: 297-373, 803, 806-820, 940, 955-968, 1067-1109, 1415-1439, 1882-1898;
k. the light chain variable region comprises a sequence having at least 90% identity to the sequence selected from SEQ ID NOs: 374-447, 821-835, 941-943, 969-982, 1110-1152, 1440-1464, 1899-1914, 1921;
l. the antibody comprises a heavy chain, wherein the heavy chain comprises a sequence having at least 90% identity to the sequence selected from SEQ ID NOs: 448-494, 804, 836-850, 983-996, 1153-1195, 1411, 1465-1489, 1695-1711;
m. the antibody comprises a light chain, wherein the light chain comprises a sequence having at least 90% identity to the sequence selected from SEQ ID NOS: 495-538, 805, 851-865, 997-1010, 1196-1238, 1412, 1490-1514, 1746-1762;
n. the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the V_{H}-CDR1 comprises an amino acid sequence having at least 90% identity to SEQ ID NO: 27;
   the V_{H}-CDR2 comprises an amino acid sequence having at least 90% identity to SEQ ID NO: 71;
   the V_{H}-CDR3 comprises an amino acid sequence having at least 90% identity to SEQ ID NO: 112;
   the V_{H}-CDR1 comprises an amino acid sequence having at least 90% identity to SEQ ID NO: 170;
   the V_{H}-CDR1 comprises an amino acid sequence having at least 90% identity to SEQ ID NO: 221; and
   the V_{H}-CDR1 comprises an amino acid sequence having at least 90%, to SEQ ID NO: 248;
o. the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the antibody comprises at least 3 V_{H}-CDRs having at least 90% identity to any one of the amino acid sequences of SEQ ID NOs: 27, 71, 112;
p. the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
   the antibody comprises at least 3 V_{H}-CDRs having at least 90% identity to any one of the amino acid sequences of SEQ ID NOs: 170, 221, 248;
q. the heavy chain variable region comprises a sequence having at least 90% identity to SEQ ID NO: 297;
r. the heavy chain variable region comprises a sequence having at least 90% identity to SEQ ID NO: 374;
s. the antibody comprises a heavy chain, wherein the heavy chain comprises a sequence having at least 90% identity to the SEQ ID NO: 448; and/or
t. the antibody comprises a light chain, wherein the light chain comprises a sequence having at least 90% identity to SEQ ID NO: 495.

72. The anti-Gal3 antibody for use of any one of the preceding arrangements, wherein:
f. the antibody is applied to the subject's eye,
g. the antibody is one of the antibodies provided in any one of the figures provided herewith,
h. the antibody is provided in an amount sufficient for the treatment of the eye disorder, when administered to the eye of the subject;
i. the antibody is applied topically or injected into the subject's eye,
j. the antibody is administered intraperitoneally, and/or
k. the amount of the antibody administered is a therapeutically effective amount, when administered intraperitoneally.

### EXAMPLES

### Example 1: Mice with Dry Eye Disease have a Decreased Palpebral Opening

The effect of dry eye disease on the palpebral opening was examined.

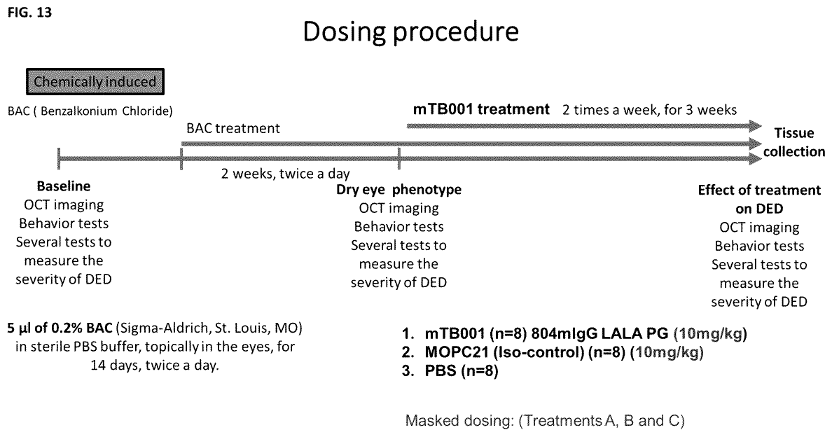

FIG. 13 is a representation of some embodiments of an anti-Gal3 dosing procedure for treatment of dry eye disease (DED).

Dry eye disease was induced in Zucker diabetic fatty (ZDF) mice by twice daily topical application of 5 μl of 0.2% Benzalkonium Chloride (BAC) (Sigma-Aldrich, St. Louis, MO) in sterile PBS buffer, to the eyes, for 14 days. A baseline assessment of the severity of the DED was made prior to induction of DED by using optical coherence tomography (OCT), behavioral tests, and other measures of DED severity. The assessments were repeated at the conclusion of BAC administration. DED mice were then either treated with twice weekly administration of 10 mg/kg TB001 anti-Gal3 antibody (treatment A), 10 mg/kg MOPC21 (treatment B), or PBS (treatment C). Assessments of DED severity was again measured and compared to the DED and baseline phenotypes.

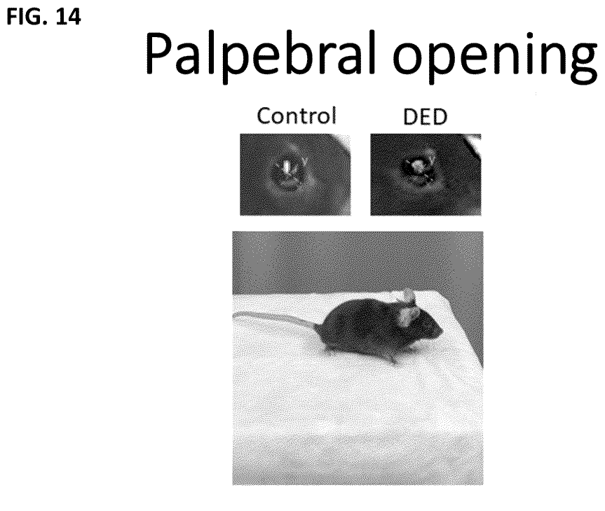

FIG. 14 shows images of some embodiments of palpebral opening in mice with normal eyes and mice with dry eye disease (DED).

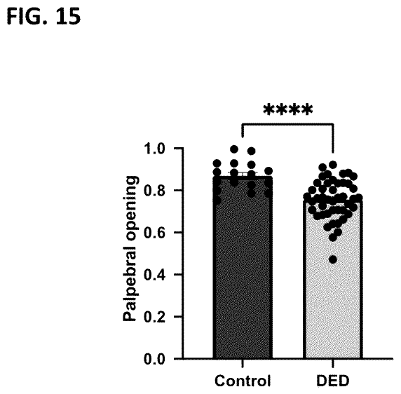

FIG. 15 is a graph quantifying palpebral opening in some embodiments of mice with normal and mice with dry eye disease (DED).

As is clear from FIG. 15, mice with DED have decreased palpebral openings as compared to mice with normal eyes.

### Example 2: Anti-Gal3 Antibodies Decrease Corneal Sensitivity

The effect of anti-Gal3 antibodies on corneal sensitivity was assessed in mice with DED.

Dry eye disease was induced in Zucker diabetic fatty (ZDF) mice by twice daily topical application of 5 μl of 0.2% Benzalkonium Chloride (BAC) (Sigma-Aldrich, St. Louis, MO) in sterile PBS buffer, to the eyes, for 14 days. A baseline assessment of corneal sensitivity was made prior to induction of DED using a von Frey test. The assessments were repeated at the conclusion of BAC administration. DED mice were then either treated with twice weekly administration of 10 mg/kg TB001 anti-Gal3 antibody (treatment A), 10 mg/kg MOPC21 (treatment B), or PBS (treatment C). Assessments of corneal sensitivity was again measured and compared to the DED and baseline phenotypes.

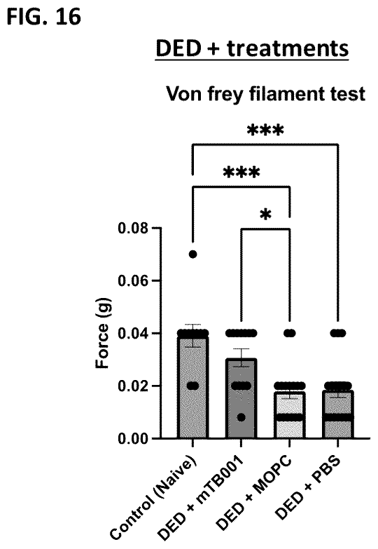

FIG. 16 is a graph depicting the results of a von Frey filament test on some embodiments of mice with normal eyes and mice with dry eye disease treated with or without anti-Gal3 antibody.

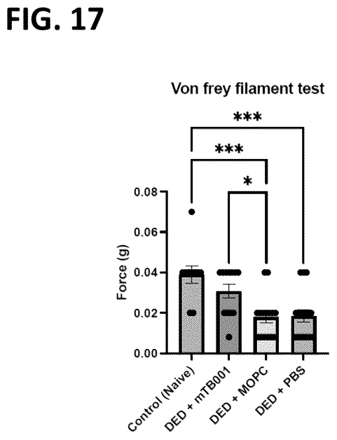

FIG. 17 is a graph depicting the results of a von Frey filament test on some embodiments of mice with normal eyes and mice with dry eye disease treated with or without anti-Gal3 antibody.

As is clear from FIG. 16 and FIG. 17, mice with DED have increased corneal sensitivity, this sensitivity is decreased in mice treated with anti-Gal3 antibody.

### Example 3: Anti-Gal3 Antibodies Increase Tear Formation in Dry Eye Disease Subject's

The effect of anti-Gal3 antibodies on tear formation was assessed in mice with DED.

Dry eye disease was induced in Zucker diabetic fatty (ZDF) mice by twice daily topical application of 5 μl of 0.2% Benzalkonium Chloride (BAC) (Sigma-Aldrich, St. Louis, MO) in sterile PBS buffer, to the eyes, for 14 days. A baseline assessment of tear formation was made prior to induction of DED using a Schirmer's test. The assessments were repeated at the conclusion of BAC administration. DED mice were then either treated with twice weekly administration of 10 mg/kg TB001 anti-Gal3 antibody (treatment A), 10 mg/kg MOPC21 (treatment B), or PBS (treatment C). Assessments of tear formation was again measured and compared to the DED and baseline phenotypes.

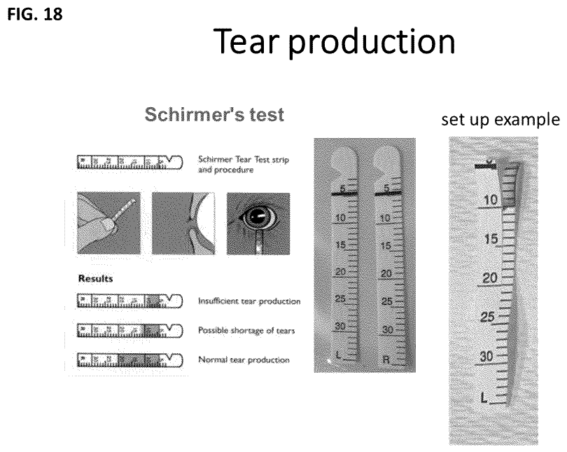

FIG. 18 is an illustration depicting some embodiments of a Schirmer's test for measuring tear production.

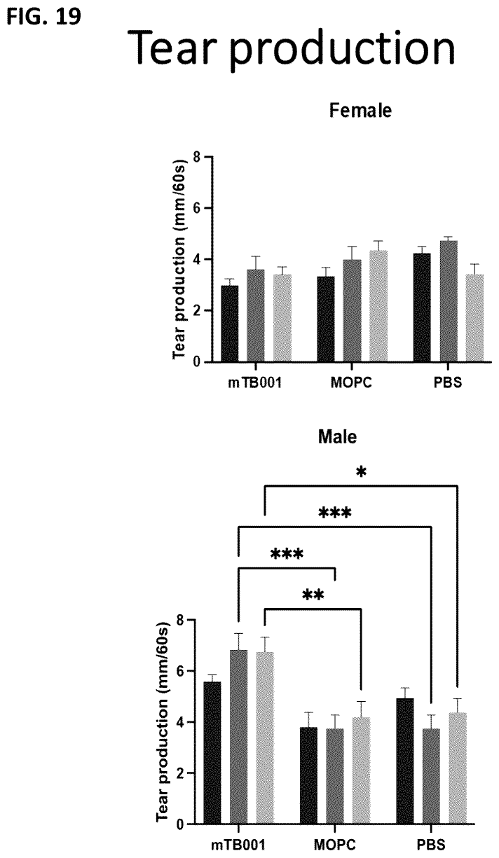

FIG. 19 is a pair of graphs quantifying tear production in female (top) and male (bottom) mice with dry eye disease following treatment with or without anti-Gal3 antibody.

As is clear from FIG. 19, tear production in mice with DED is increased following anti-Gal3 antibody administration.

### Example 4: Anti-Gal3 Antibodies Decrease Gal3 Concentration in Tears

The effect of anti-Gal3 antibodies on Gal3 concentration in tears was assessed in mice with DED.

Dry eye disease was induced in Zucker diabetic fatty (ZDF) mice by twice daily topical application of 5 μl of 0.2% Benzalkonium Chloride (BAC) (Sigma-Aldrich, St. Louis, MO) in sterile PBS buffer, to the eyes, for 14 days. A baseline assessment of Gal3 concentration in tears was made prior to induction of DED. The assessments were repeated at the conclusion of BAC administration. DED mice were then either treated with twice weekly administration of 10 mg/kg TB001 anti-Gal3 antibody (treatment A), 10 mg/kg MOPC21 (treatment B), or PBS (treatment C). Gal3 concentration in tears was again measured and compared to the DED and baseline phenotypes.

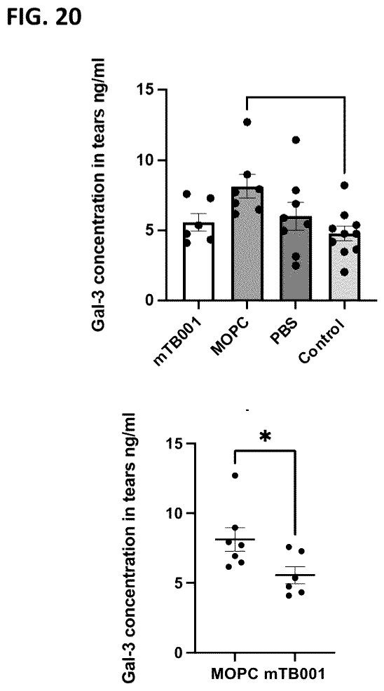

FIG. 20 is a pair of graphs depicting quantification of Gal-3 concentration in the tears of mice with normal eyes and mice with dry eye disease with or without anti-Gal3 antibody administration.

As is clear from FIG. 20, Gal3 concentration is increased in the tears of mice with DED and is decreased following anti-Gal3 antibody administration.

### Example 5: Anti-Gal3 Antibodies have No Effect on Subject Body Weight

The effect of anti-Gal3 antibodies on a subject's body weight was assessed in mice with DED.

Dry eye disease was induced in Zucker diabetic fatty (ZDF) mice by twice daily topical application of 5 μl of 0.2% Benzalkonium Chloride (BAC) (Sigma-Aldrich, St. Louis, MO) in sterile PBS buffer, to the eyes, for 14 days. A baseline assessment of body weight was made prior to induction of DED. The assessments were repeated at the conclusion of BAC administration. DED mice were then either treated with twice weekly administration of 10 mg/kg TB001 anti-Gal3 antibody (treatment A), 10 mg/kg MOPC21 (treatment B), or PBS (treatment C). Body weight was again measured and compared to the DED and baseline phenotypes.

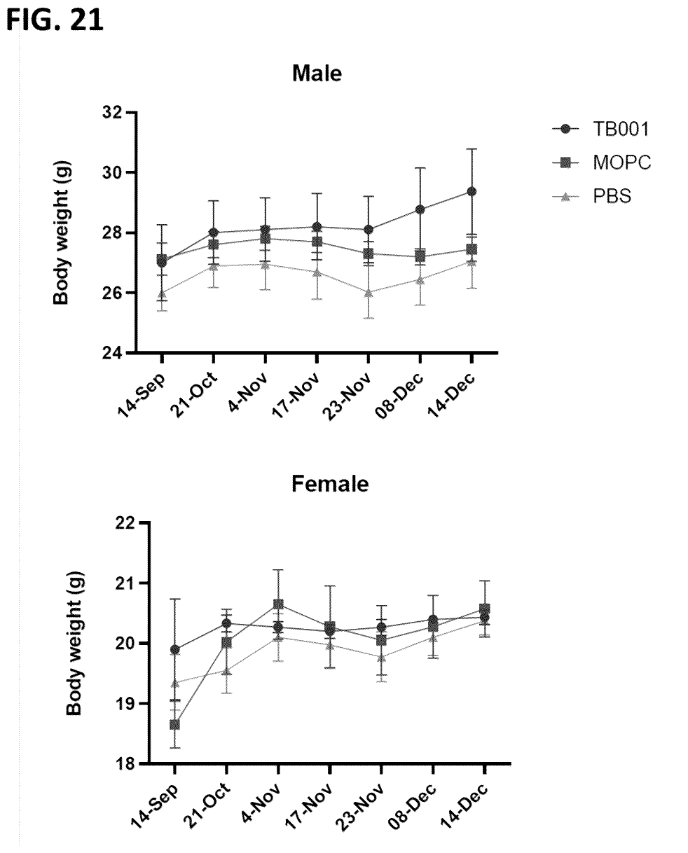

FIG. 21 is a pair of graphs depicting the body weight of female (top) and male (bottom) mice treated with or without anti-Gal3 antibodies.

As is clear from FIG. 21, administration of anti-Gal3 antibodies has no apparent effect on mouse body weight.

### Example 6: Anti-Gal3 Antibodies Decrease Cataract Severity in Dry Eye Disease Subject's

The effect of anti-Gal3 antibodies on cataract severity was assessed in mice with DED.

Dry eye disease was induced in Zucker diabetic fatty (ZDF) mice by twice daily topical application of 5 μl of 0.2% Benzalkonium Chloride (BAC) (Sigma-Aldrich, St. Louis, MO) in sterile PBS buffer, to the eyes, for 14 days. A baseline assessment or staging of cataract severity was made prior to induction of DED. The staging was repeated at the conclusion of BAC administration. DED mice were then either treated with twice weekly administration of 10 mg/kg TB001 anti-Gal3 antibody (treatment A), 10 mg/kg MOPC21 (treatment B), or PBS (treatment C). Cataract severity was again measured and compared to the DED and baseline phenotypes.

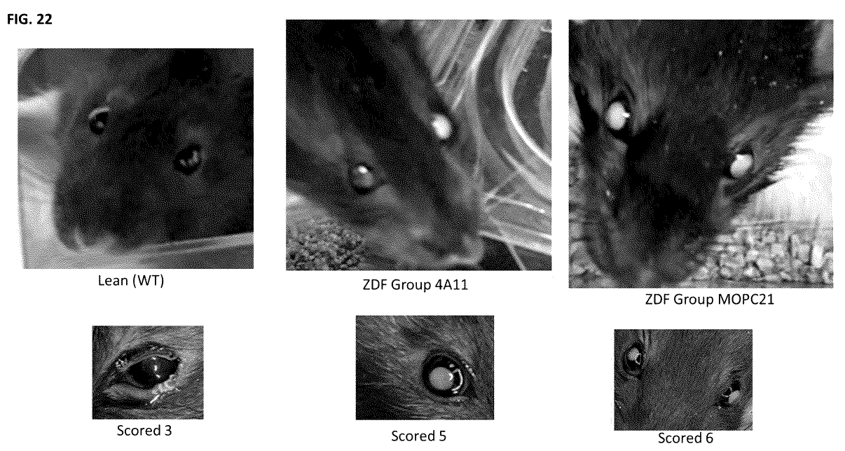

FIG. 22 shows images of some embodiments of cataract staging in mice with normal eyes and mice with dry eye disease (DED).

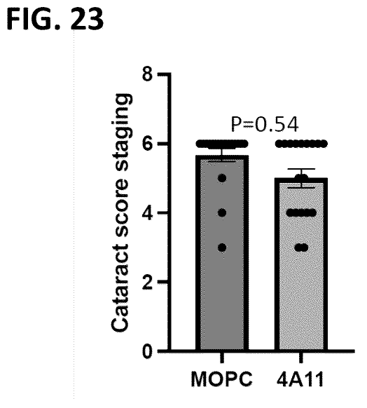

FIG. 23 is a pair of graphs depicting cataract staging in some embodiments of mice treated with or without anti-Gal3 antibody.

1. A method of treating microglia mediated inflammation and/or one or more diseases associated with microglia mediated inflammation in a subject in need thereof, the method comprising:
identifying a subject as having or at risk of having microglia mediated inflammation and/or one or more symptoms of a disease associated with microglia mediated inflammation, and
administering a therapeutically effective amount of an anti-Gal3 antibody to the subject, wherein
administration of the antibody to the subject alleviates one or more symptoms of microglia mediated inflammation and/or one or more one or more symptoms of a disease associated with microglia mediated inflammation.

2. (canceled)

3. A method of treating an ocular surface disease in a subject in need thereof, the method comprising:
identifying a subject as having or at risk of having an ocular surface disease, and
administering a therapeutically effective amount of an anti-Gal3 antibody to the subject, wherein
administration of the antibody to the subject alleviates one or more symptoms of ocular surface disease.

4. A method of treating macular degeneration, age-related macular degeneration, age-related vascular dysfunction, diabetic retinopathy, and/or retinal drusen, in a subject in need thereof, the method comprising:
identifying a subject as having or at risk of having macular degeneration, age-related macular degeneration, age-related vascular dysfunction, diabetic retinopathy, and/or retinal drusen, and
administering a therapeutically effective amount of an anti-Gal3 antibody to the subject, wherein
administration of the antibody to the subject alleviates one or more symptoms of macular degeneration, age-related macular degeneration, age-related vascular dysfunction, diabetic retinopathy, and/or retinal drusen.

5-8. (canceled)

9. A method of treating cataracts in a subject in need thereof, the method comprising:
identifying a subject as having or at risk of having cataracts, and
administering a therapeutically effective amount of an anti-Gal3 antibody to the subject, wherein
administration of the antibody to the subject reduces the severity of the subject's cataracts as compared to the severity of cataracts in the subjects prior to administration of the anti-Gal3 antibody.

10-12. (canceled)

13. The method of claim 4, wherein the anti-Gal3 antibody is administered intravenously or topically.

14. The method of claim 3, wherein the one or more symptoms comprise dryness of the eye, foreign body sensation, burning sensation, itching, photophobia, eye redness, blurred vision, fluctuating vision, visual fatigue, corneal sensitivity, decreased tear formation, or any combination thereof.

15. The method of claim 3, wherein the ocular surface disease comprises age-related vascular dysfunction, angiogenesis, inflammation, and oxidative stress.

16-19. (canceled)

20. The method of claim 4, wherein the anti-Gal3 antibody is administered, daily, twice daily, weekly, bi-weekly, and/or monthly.

21. The method of claim 20, wherein the anti-Gal3 antibody is administered for up to about 1, 2, 3, 4, 5, 6, 7, 8, 9, 10, 11, or 12 weeks, or for an amount of time that is in a range defined by any two of the preceding values.

22. The method of claim 21, wherein the anti-Gal3 antibody is topically administered to the subject's eye twice daily for 14 days.

23. The method of claim 22, wherein 10 mg/kg of anti-Gal3 antibody is topically administered to the subject's eye.

24. (canceled)

25. The method of claim 4, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the antibody comprises at least 1 V_{L}-CDR having at least 90% identity to any one of the amino acid sequences of SEQ ID NOs: 170-296, 1814-1864.

26. The method of claim 4, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the antibody comprises at least 1 V_{H}-CDR having at least 90% identity to any one of the amino acid sequences of SEQ ID NOs: 27-169, 801, 802, 953, 954, 1763-1813.

27. The method of claim 4, wherein the antibody comprises at least 1 CDR having at least 90% identity to any one of the amino acid sequences of SEQ ID Nos: 27-296, 801, 802, 953, 954, 1763-1813.

28. The method of claim 4, wherein the antibody comprises at least 6 CDRs having at least 90% identity to any one of the amino acid sequences of SEQ ID NOs: 27-296, 801, 802, 953, 954, 1763-1864.

29. The method of claim 4, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 90% identity to any one of the amino acid sequences of SEQ ID NOs: 27-70, 1763-1779;
the V_{H}-CDR2 comprises an amino acid sequence having at least 90% identity to any one of the amino acid sequences of SEQ ID NOs: 71-111, 801, 951, 952, 1780-1796;
the V_{H}-CDR3 comprises an amino acid sequence having at least 90% identity to any one of the amino acid sequences of SEQ ID NO: 112-169, 802, 953, 954, 1797-1813;
the V_{L}-CDR1 comprises an amino acid sequence having at least 90% identity to any one of the amino acid sequences of SEQ ID NOs: 170-220, 1814-1830;
the V_{L}-CDR2 comprises an amino acid sequence having at least 90% identity to any one of the amino acid sequences of SEQ ID NOs: 221-247, 1831-1847; and
the V_{L}-CDR3 comprises an amino acid sequence having at least 90% identity to any one of the amino acid sequences of SEQ ID NOs: 248-296, 1848-1864.

30. The method of claim 4, wherein the heavy chain variable region comprises a sequence having at least 90% identity to the sequence selected from SEQ ID NOs: 297-373, 803, 806-820, 940, 955-968, 1067-1109, 1415-1439, 1882-1898.

31. The method of claim 4, wherein the light chain variable region comprises a sequence having at least 90% identity to the sequence selected from SEQ ID NOs: 374-447, 821-835, 941-943, 969-982, 1110-1152, 1440-1464, 1898-1914, 1921.

32. The method of claim 4, wherein the antibody comprises a heavy chain, wherein the heavy chain comprises a sequence having at least 90% identity to the sequence selected from SEQ ID Nos: 448-494, 804, 836-850, 983-996, 1153-1195, 1411, 1465-1489, 1695-1711.

33. The method of claim 4, wherein the antibody comprises a light chain, wherein the light chain comprises a sequence having at least 90% identity to the sequence selected from SEQ ID Nos: 495-538, 805, 851-865, 997-1010, 1196-1238, 1412, 1490-1514, 1746-1762.

34. The method of claim 4, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 90% identity to SEQ ID NO: 27;
the V_{H}-CDR2 comprises an amino acid sequence having at least 90% identity to SEQ ID NO: 71;
the V_{H}-CDR3 comprises an amino acid sequence having at least 90% identity to SEQ ID NO: 112;
the V_{H}-CDR1 comprises an amino acid sequence having at least 90% identity to SEQ ID NO: 170;
the V_{H}-CDR1 comprises an amino acid sequence having at least 90% identity to SEQ ID NO: 221; and
the V_{H}-CDR1 comprises an amino acid sequence having at least 90%, to SEQ ID NO: 248.

35. The method of claim 4, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the antibody comprises at least 3 V_{H}-CDRs having at least 90% identity to any one of the amino acid sequences of SEQ ID Nos: 27, 71, 112.

36. The method of claim 4, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the antibody comprises at least 3 V_{H}-CDRs having at least 90% identity to any one of the amino acid sequences of SEQ ID Nos: 170, 221, 248.

37. The method of claim 4, wherein the heavy chain variable region comprises a sequence having at least 90% identity to SEQ ID NO: 297.

38. The method of claim 4, wherein the heavy chain variable region comprises a sequence having at least 90% identity to SEQ ID NO: 374.

39. The method of claim 4, wherein the antibody comprises a heavy chain, wherein the heavy chain comprises a sequence having at least 90% identity to the SEQ ID NO: 448.

40. The method of claim 4, wherein the antibody comprises a light chain, wherein the light chain comprises a sequence having at least 90% identity to SEQ ID NO: 495.

41. (canceled)

42. (canceled)

43. The method of claim 3, wherein the ocular surface disease is selected from dry eye disease (DED), blepharitis, neurotrophic keratitis, ocular rosacea, meibomian gland dysfunction, decreased tear quality and/or production with age, chemical or thermal burns, side effects of medical treatments or prescription medications, conjunctivitis, Sjogren's disease, allergies and other immunological conditions, and any combinations thereof.
